(19) United States
(12) Patent Application Publication
(10) Pub. No.: US 2026/0285858 A1
(43) Pub. Date: Sep. 24, 2026

(54) GLP-1 RECEPTOR TARGETING COMPOUNDS AND USES THEREOF

(71) Applicant: FORTVITA BIOLOGICS INC., Grand Cayman (KY)

(72) Inventors: Lianhong XU, Suzhou (CN); Nan Yu, Suzhou (CN); Decheng REN, Suzhou (CN); Yao XIONG, Suzhou (CN)

(73) Assignee: FORTVITA BIOLOGICS INC., Grand Cayman (KY)

(21) Appl. No.: 19/667,100

(22) Filed: May 4, 2026

Related U.S. Application Data
(63) Continuation of application No. PCT/CN2024/136693, filed on Dec. 4, 2024.

(30) Foreign Application Priority Data
Dec. 5, 2023 (WO) PCT/CN2023/136412
Jul. 15, 2024 (WO) PCT/CN2024/105568

(51) Int. Cl. C07D 471/04; A61K 31/437; A61K 31/444; A61K 31/497; A61K 31/4985; A61K 31/501; A61K 31/5025; A61K 31/506; A61K 31/519; A61K 31/5377; C07B 59/00; C07D 487/04; C07D 519/00

(52) U.S. Cl. CPC C07D 471/04; A61K 31/437; A61K 31/444; A61K 31/497; A61K 31/4985; A61K 31/501; A61K 31/5025; A61K 31/506; A61K 31/519; A61K 31/5377; C07B 59/002; C07D 487/04; C07D 519/00; C07B 2200/05

(57) ABSTRACT

The present disclosure relates to compounds of Formula (I), or a pharmaceutically acceptable salt thereof, stereoisomer thereof, analog thereof, solvate thereof, prodrug thereof, and/or tautomer thereof, as well as pharmaceutical compositions including such a compound, wherein the variables are as defined in the specification. Also described are method of using such compounds and pharmaceutical compositions to treat GLP-1 associated diseases, disorders, or conditions.

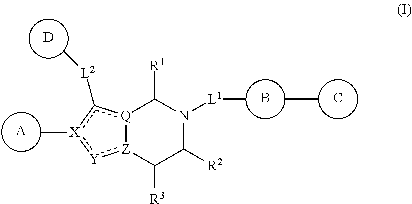

### FIELD

The present disclosure relates to compounds and pharmaceutical compositions for targeting the glucagon-like peptide-1 (GLP-1) receptor, as well as uses thereof.

### BACKGROUND

The glucagon-like peptide-1 (GLP-1) is a peptide hormone deriving from the proglucagon peptide. It is produced and secreted by intestinal enteroendocrine L-cells and certain neurons within the nucleus of the solitary tract in the brainstem upon food consumption.

The most noteworthy effect of GLP-1 is its ability to promote insulin secretion in a glucose-dependent manner. As GLP-1 binds to GLP-1 receptors expressed on the pancreatic R cells, the receptors couples to G-protein subunits and activates adenylate cyclase that increases the production of cAMP from ATP. Subsequently, activation of secondary pathways, including PKA and Epac2, alters the ion channel activity causing elevated levels of cytosolic Ca²⁺ that enhances exocytosis of insulin-containing granules.

The GLP-1R is a seven-transmembrane-spanning, class B, G protein-coupled receptor (GPCR). Class B GPCRs, including the GLP-1R, are activated by endogenous peptide hormones, and the development of small-molecule agonists of these receptors has proven to be particularly challenging. Significant prior efforts across the pharmaceutical industry have generally failed to identify potent and efficacious small-molecule agonists of the GLP-1R. Thus, there is a need for better oral small-molecule agonists of the GLP-1R.

### SUMMARY

The present disclosure relates to compounds and pharmaceutical compositions for targeting the glucagon-like peptide-1 (GLP-1) receptor, as well as uses thereof. In some embodiments, the compound includes a structure of any one of Formulas (I), (IIa)-(IIc), (IIIa)-(IIIc), (IV), (IVa), and (IVb). In other embodiments, the compound includes a structure provided in Table 1 or Table 3c. Additional details follow.

### Definitions

The articles “a” and “an” are used in this disclosure to refer to one or more than one (i.e., at least one) of the grammatical object of the article. By way of example, “an element” means one element or more than one element.

As used herein, the term “about” means+/−10% of any recited value. As used herein, this term modifies any recited value, range of values, or endpoints of one or more ranges.

By “aliphatic group” is meant a hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl, e.g., as described herein), alkenes (or alkenyl), alkynes (or alkynyl), including cyclic versions thereof, and further including straight- and branched-chain arrangements, and all stereo and position isomers as well. Such a hydrocarbon can be unsubstituted or substituted with one or more groups, such as groups described herein for an alkyl group.

By “alkenyl” is meant an optionally substituted C₂₋₂₄ hydrocarbyl group having one or more double bonds. The alkenyl group can be cyclic (e.g., C₃₋₂₄ cycloalkenyl) or acyclic. The alkenyl group can also be substituted or unsubstituted. For example, the alkenyl group can be substituted with one or more substitution groups, as described herein for alkyl. Non-limiting unsubstituted alkenyl groups include allyl and vinyl. In some embodiments, the unsubstituted alkenyl group is a C₂₋₆, C₂₋₈, C₂₋₁₀, C₂₋₁₂, C₂₋₁₆, C₂₋₁₈, C₂₋₂₀, C₂₋₂₄, C₃₋₈, C₃₋₁₀, C₃₋₁₂, C₃₋₁₆, C₃₋₁₈, C₃₋₂₀, or C₃₋₂₄ alkenyl group. Non-limiting examples of alkenyl groups include vinyl or ethenyl (—CH═CH₂), 1-propenyl (—CH═CHCH₃), allyl or 2-propenyl (—CH₂—CH═CH₂), 1-butenyl (—CH═CHCH₂CH₃), 2-butenyl (—CH₂CH═CHCH₃), 3-butenyl (—CH₂CH₂CH═CH₂), 2-butenylidene (═CH—CH═CHCH₃), and the like.

By “alkoxy” is meant —OR, where R is an optionally substituted aliphatic or alkyl group, as described herein. Non-limiting examples of alkoxy groups include methoxy, ethoxy, n- propoxy, isopropoxy, n-butoxy, t-butoxy, sec-butoxy, n-pentoxy, trihaloalkoxy, such as trifluoromethoxy, etc. The alkoxy group can be substituted or unsubstituted. For example, the alkoxy group can be substituted with one or more substitution groups, as described herein for alkyl. Non-limiting examples of unsubstituted alkoxy include C₁₋₃, C₁₋₆, C₁₋₁₂, C₁₋₁₆, C₁₋₁₈, C₁₋₂₀, or C₁₋₂₄ alkoxy groups.

By “alkyl” and the prefix “alk” is meant a branched or unbranched saturated hydrocarbon group of 1 to 24 carbon atoms, such as methyl (Me), ethyl (Et), n-propyl (n-Pr), isopropyl (i-Pr), cyclopropyl, n-butyl (n-Bu), isobutyl (i-Bu), s-butyl (s-Bu), t-butyl (t-Bu), n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be substituted or unsubstituted. For example, the alkyl group can be substituted with one or more alkenyl, alkoxy, alkynyl, amino, aryl, carboxyaldehyde (e.g., —C(O)H), carboxyl (e.g., —CO₂H), cyano (e.g., —CN), halo, nitro (e.g., —NO₂), oxo (e.g., ═O), and the like. In another example, the alkyl group can be substituted with one, two, three or, in the case of alkyl groups of two carbons or more, four substituents independently selected from the group consisting of: (1) C₁₋₆ alkoxy (e.g., —O—R, in which R is C₁₋₆ alkyl); (2) C₁₋₆ alkylsulfinyl (e.g., —S(O)—R, in which R is C₁₋₆ alkyl); (3) C₁₋₆ alkylsulfonyl (e.g., —SO₂—R, in which R is C₁₋₆ alkyl); (4) amide (e.g., —NHCOR¹, where R¹ is selected from hydrogen, aliphatic, heteroaliphatic, haloaliphatic, haloheteroaliphatic, aromatic groups, as defined herein, or any combination thereof); (5) aryl (e.g., C₆₋₁₈ aryl); (6) arylalkoxy (e.g., —O-L-R, in which L is C₁₋₆ alkylene and R is C₆₋₁₈ aryl); (7) aryloyl (e.g., —C(O)—R, in which R is C₆₋₁₈ aryl); (8) azido (e.g., —N₃); (9) cyano (e.g., -CN); (10) aldehyde (e.g., —C(O)H); (11) C₃₋₈ cycloalkyl; (12) halo; (13) heterocyclyl (e.g., as defined herein, such as a 5-, 6- or 7-membered ring containing one, two, three, or four non-carbon heteroatoms); (14) heterocyclyloxy (e.g., —O—R, in which R is heterocyclyl, as defined herein); (15) heterocyclyloyl (e.g., —C(O)—R, in which R is heterocyclyl, as defined herein); (16) hydroxy (e.g., —OH); (17)N-protected amino; (18) nitro (e.g., —NO₂); (19) oxo (e.g., ═O); (20) C₁₋₆ thioalkoxy (e.g., —S—R, in which R is alkyl); (21) thiol (e.g., —SH); (22) —CO₂R¹, where R¹ is selected from the group consisting of (a) hydrogen, (b) C₁₋₆ alkyl, (c) C₆₋₁₈ aryl, and (d) C₁₋₆ alkyl-C₆₋₁₈ aryl (e.g., -L-R, in which L is C₁₋₆ alkylene and R is C₆₋₁₈ aryl); (23) -C(O)NR¹R², where each of R¹ and R² is, independently, selected from the group consisting of (a) hydrogen, (b) C₁₋₆ alkyl, (c) C₆₋₁₈ aryl, and (d) C₁₋₆ alkyl-C₆₋₁₈ aryl (e.g., -L-R, in which L is C₁₋₆ alkylene and R is C₆₋₁₈ aryl); (24) —SO₂R¹, where R¹ is selected from the group consisting of (a) C₁₋₆ alkyl, (b) C₆₋₁₈ aryl, and (c) C₁₋₆ alkyl-C₆₋₁₈ aryl (e.g., -L-R, in which L is C₁₋₆ alkylene and R is C₆₋₁₈ aryl); (25) —SO₂NR¹R², where each of R¹ and R² is, independently, selected from the group consisting of (a) hydrogen, (b) C₁₋₆ alkyl, (c) C₆₋₁₈ aryl, and (d) C₁₋₆ alkyl-C₆₋₁₈ aryl (e.g., -L-R, in which L is C₁₋₆ alkylene and R is C₆₋₁₈ aryl); (26) —NR¹R², where each of R¹ and R² is, independently, selected from the group consisting of (a) hydrogen, (b) an N-protecting group, (c) C₁₋₆ alkyl, (d) C₂₋₆ alkenyl, (e) C₂₋₆ alkynyl, (f) C₆₋₁₈ aryl, (g) C₁₋₆ alkyl-C₆₋₁₈ aryl (e.g., -L-R, in which L is C₁₋₆ alkylene and R is C₆₋₁₈ aryl), (h) C₃₋₈ cycloalkyl, and (i) C₁₋₆ alkyl-C₃₋₈ cycloalkyl (e.g., -L-R, in which L is C₁₋₆ alkylene and R is C₃₋₈ cycloalkyl), wherein in one embodiment no two groups are bound to the nitrogen atom through a carbonyl group or a sulfonyl group; and (27) one or more isotopes, such as one or more isotopes of hydrogen, carbon, nitrogen, oxygen, and/or fluorine (e.g., ³H, ²H, ¹¹C, ¹³C, ¹⁴C, ¹⁵N, ¹⁸O, and/or ¹⁸F). In some embodiments, substituents can include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these. The alkyl group can be a primary, secondary, or tertiary alkyl group substituted with one or more substituents (e.g., one or more halo or alkoxy). In some embodiments, the unsubstituted alkyl group is a C₁₋₃, C₁₋₄, C₁₋₆, C₁₋₈, C₁₋₁₀, C₁₋₁₂, C₁₋₁₆, C₁₋₁₈, C₁₋₂₀, C₁₋₂₄, C₂₋₆, C₂₋₈, C₂₋₁₀, C₂₋₁₂, C₂₋₁₆, C₂₋₁₈, C₂₋₂₀, C₂₋₂₄, C₃₋₈, C₃₋₁₀, C₃₋₁₂, C₃₋₁₆, C₃₋₁₈, C₃₋₂₀, or C₃₋₂₄ alkyl group.

By “alkylene” is meant a multivalent (e.g., bivalent) form of an aliphatic group or alkyl group, as described herein. Non-limiting examples of alkylene groups include methylene, ethylene, propylene, butylene, etc. In some embodiments, the alkylene group is a C₁₋₃, C₁₋₄, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substitution groups, as described herein for alkyl.

By “alkynyl” is meant an optionally substituted C₂₋₂₄hydrocarbyl group having one or more triple bonds. The alkynyl group can be cyclic or acyclic and is exemplified by ethynyl, 1-propynyl, and the like. The alkynyl group can also be substituted or unsubstituted. For example, the alkynyl group can be substituted with one or more substitution groups, as described herein for alkyl. Non-limiting unsubstituted alkynyl groups include C₂₋₈ alkynyl, C₂-6 alkynyl, C₂₋₅ alkynyl, C₂₄ alkynyl, or C₂₋₃ alkynyl. Non-limiting examples of alkynyl groups include ethynyl (—C≡CH), 1-propynyl (—C≡CCH₃), 2-propynyl or propargyl (—CH₂C≡CH), 1-butynyl (—C≡CCH₂CH₃), 2-butynyl (—CH₂C≡CCH₃), 3-butynyl (—CH₂CH₂C≡CH), and the like. In some embodiments, the unsubstituted alkynyl group is a C₂₋₆, C₂₋₈, C₂₋₁₀, C₂₋₁₂, C₂₋₁₆, C₂₋₁₈, C₂₋₂₀, C₂₋₂₄, C₃₋₈, C₃₋₁₀, C₃₋₁₂, C₃₋₁₆, C₃₋₁₈, C₃₋₂₀, or C₃₋₂₄ alkynyl group.

By “amino” is meant a —NR^{N1}R^{N2} group, a —NR^{N1}— group, or a compound having such a group, where each of R^{N1} and R^{N2} is, independently, H, optionally substituted aliphatic, alkyl, heteroaliphatic, heteroalkyl, aromatic, or aryl; or where R^{N1} and R^{N2}, taken together with the nitrogen atom to which each are attached, form a heterocyclyl group, as defined herein.

By “aromatic” is meant a cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Huckel rule (4n+2). The point of attachment to the parent structure typically is through an aromatic portion of the condensed ring system.

By “aryl” is meant an aromatic carbocyclic group comprising at least five carbon atoms to 18 carbon atoms (C₅₋₁₈), such as five to ten carbon atoms (C₅₋₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as alkyl, as well as any substitution groups described herein for alkyl. Non-limiting examples of aryl groups include, but are not limited to, naphthalene, phenyl, biphenyl, phenoxybenzene, and the like. The aryl group can be substituted with one, two, three, four, or five substituents provided herein for alkyl. In particular embodiments, an unsubstituted aryl group is a C₆₋₁₈, C₆₋₁₄, C₆₋₁₂, or C₆₋₁₀ aryl group.

By “carbonyl” is meant a —C(O)— group, which can also be represented as >C═O, or a —CO— group.

By “cycloaliphatic” is meant an aliphatic group, as defined herein, that is cyclic.

By “cycloalkyl” is meant a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, and the like. Cycloalkyl can include fused/bridged forms, such as, e.g., bicyclo[1.1.0]butane, bicyclo[2.1.0]pentane,bicyclo[1.1.1]pentane,bicyclo[3.1.0]hexane,bicyclo[2.1.1]hexane,bicyclo[3.2.0]heptane,bicyclo[4.1.0]heptane,bicyclo[2.2.1]heptane,bicyclo[3.1.1]heptane,bicyclo[4 0.2.0]octane,bicyclo[3.2.1]octane, bicyclo[2.2.2]octane, and the like. Cycloalkyl can also include spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[4.4]nonane, spiro[2.6]nonane, spiro[4.5]decane, spiro[3.6]decane, spiro[5.5]undecane, and the like. The cycloalkyl group can also be substituted or unsubstituted. For example, the cycloalkyl group can be substituted with one or more groups including those described herein for alkyl.

By “cycloheteroaliphatic” is meant a heteroaliphatic group, as defined herein, that is cyclic.

By “halo” is meant F, Cl, Br, or I.

By “haloaliphatic” is meant an aliphatic group, as defined herein, substituted with one or more halo.

By “haloalkoxy” is meant an alkoxy group, as defined herein, substituted with one or more halo.

By “haloalkyl” is meant an alkyl group, as defined herein, substituted with one or more halo. Non-limiting examples include monofluoromethyl, difluoromethyl, trifluoromethyl, fluorochloromethyl, difluorochloromethyl, 2-fluoroethyl, 2,2-difluoroethyl, 2,2,2-trifluoroethyl, monochloromethyl, dichloromethyl, chloroethyl, trichloroethyl, monobromomethyl, monoiodomethyl, and the like.

By “halocycloalkyl” is meant a cycloalkyl group, as defined herein, substituted with one or more halo.

By “haloheteroaliphatic” is meant a heteroaliphatic, as defined herein, in which one or more hydrogen atoms, such as one to 10 hydrogen atoms, independently is replaced with a halogen atom, such as fluoro, bromo, chloro, or iodo.

By “heteroaliphatic” is meant an aliphatic group, as defined herein, including at least one heteroatom to 20 heteroatoms, such as one to 15 heteroatoms, or one to 5 heteroatoms, which can be selected from, but not limited to boron, halo, nitrogen, oxygen, phosphorus, selenium, silicon, sulfur, and, if applicable, oxidized forms thereof within the group.

By “heteroalkyl,” “heteroalkenyl,” and “heteroalkynyl” is meant an alkyl, alkenyl, or alkynyl group (which can be branched, straight-chain, or cyclic), respectively, as defined herein, including at least one heteroatom to 20 heteroatoms, such as one to 15 heteroatoms, or one to 5 heteroatoms, which can be selected from, but not limited to, boron, halo, nitrogen (e.g., as present in imino), oxygen, phosphorus, selenium, silicon, sulfur, and, if applicable, oxidized forms thereof within the group.

By “heteroaromatic” is meant an aromatic group, as defined herein, including at least one heteroatom to 20 heteroatoms, such as one to 15 heteroatoms, or one to 5 heteroatoms, which can be selected from, but not limited to boron, nitrogen, oxygen, phosphorus, selenium, silicon, sulfur, and oxidized forms thereof within the group.

By “heteroaryl” is meant an aromatic group including at least one heteroatom to six heteroatoms, such as one to four heteroatoms, which can be selected from, but not limited to, boron, nitrogen, oxygen, phosphorus, selenium, silicon, sulfur, and oxidized forms thereof within the ring. Such heteroaryl groups can have a single ring or multiple condensed rings, where the condensed rings may or may not be aromatic or may contain a heteroatom, provided that the point of attachment is through an atom of the aromatic heteroaryl group. Heteroaryl groups may be substituted with one or more groups other than hydrogen, such as alkyl, as well as any substitution groups described herein for alkyl. A non-limiting example of heteroaryl includes a subset of heterocyclyl groups, as defined herein, which are aromatic, i.e., they contain 4n+2 pi electrons within the mono- or multicyclic ring system.

By “heterocyclyl” is meant a 3-, 4-, 5-, 6- or 7-membered ring (e.g., a 5-, 6- or 7-membered ring), unless otherwise specified, containing one, two, three, or four non-carbon heteroatoms (e.g., independently selected from the group consisting of nitrogen, oxygen, phosphorus, selenium, silicon, or sulfur). The 3-membered ring has zero to one double bonds, the 4- and 5-membered ring has zero to two double bonds, and the 6- and 7-membered rings have zero to three double bonds. The term “heterocyclyl” also includes bicyclic, tricyclic, tetracyclic, or other multicyclic groups. Heterocyclyl includes acridinyl, adenyl, alloxazinyl, azaadamantanyl, azabenzimidazolyl, azabicyclononyl, azacycloheptyl, azacyclooctyl, azacyclononyl, azahypoxanthinyl, azaindazolyl, azaindolyl, azecinyl, azepanyl, azepinyl, azetidinyl, azetyl, aziridinyl, azirinyl, azocanyl, azocinyl, azonanyl, benzimidazolyl, benzisothiazolyl, benzisoxazolyl, benzodiazepinyl, benzodiazocinyl, benzodihydrofuryl, benzodioxepinyl, benzodioxinyl, benzodioxanyl, benzodioxocinyl, benzodioxolyl, benzodithiepinyl, benzodithiinyl, benzodioxocinyl, benzofuranyl, benzophenazinyl, benzopyranonyl, benzopyranyl, benzopyrenyl, benzopyronyl, benzoquinolinyl, benzoquinolizinyl, benzothiadiazepinyl, benzothiadiazolyl, benzothiazepinyl, benzothiazocinyl, benzothiazolyl, benzothienyl, benzothiophenyl, benzothiazinonyl, benzothiazinyl, benzothiopyranyl, benzothiopyronyl, benzotriazepinyl, benzotriazinonyl, benzotriazinyl, benzotriazolyl, benzoxathiinyl, benzotrioxepinyl, benzoxadiazepinyl, benzoxathiazepinyl, benzoxathiepinyl, benzoxathiocinyl, benzoxazepinyl, benzoxazinyl, benzoxazocinyl, benzoxazolinonyl, benzoxazolinyl, benzoxazolyl, benzylsultamyl, benzylsultimyl, bipyrazinyl, bipyridinyl, carbazolyl (e.g., 4H-carbazolyl), carbolinyl (e.g., O-carbolinyl), chromanonyl, chromanyl, chromenyl, cinnolinyl, coumarinyl, cytidinyl, cytosinyl, decahydroisoquinolinyl, decahydroquinolinyl, diazabicyclooctyl, diazetyl, diaziridinethionyl, diaziridinonyl, diaziridinyl, diazirinyl, dibenzisoquinolinyl, dibenzoacridinyl, dibenzocarbazolyl, dibenzofuranyl, dibenzophenazinyl, dibenzopyranonyl, dibenzopyronyl (xanthonyl), dibenzoquinoxalinyl, dibenzothiazepinyl, dibenzothiepinyl, dibenzothiophenyl, dibenzoxepinyl, dihydroazepinyl, dihydroazetyl, dihydrofuranyl, dihydrofuryl, dihydroisoquinolinyl, dihydropyranyl, dihydropyridinyl, dihydroypyridyl, dihydroquinolinyl, dihydrothienyl, dihydroindolyl, dioxanyl, dioxazinyl, dioxindolyl, dioxiranyl, dioxenyl, dioxinyl, dioxobenzofuranyl, dioxolyl, dioxotetrahydrofuranyl, dioxothiomorpholinyl, dithianyl, dithiazolyl, dithienyl, dithiinyl, furanyl, furazanyl, furoyl, furyl, guaninyl, homopiperazinyl, homopiperidinyl, hypoxanthinyl, hydantoinyl, imidazolidinyl, imidazolinyl, imidazolyl, indazolyl (e.g., 1H-indazolyl), indolenyl, indolinyl, indolizinyl, indolyl (e.g., 1H-indolyl or 3H-indolyl), isatinyl, isatyl, isobenzofuranyl, isochromanyl, isochromenyl, isoindazoyl, isoindolinyl, isoindolyl, isopyrazolonyl, isopyrazolyl, isoxazolidiniyl, isoxazolyl, isoquinolinyl, isoquinolinyl, isothiazolidinyl, isothiazolyl, morpholinyl, naphthindazolyl, naphthindolyl, naphthiridinyl, naphthopyranyl, naphthothiazolyl, naphthothioxolyl, naphthotriazolyl, naphthoxindolyl, naphthyridinyl, octahydroisoquinolinyl, oxabicycloheptyl, oxauracil, oxadiazolyl, oxazinyl, oxaziridinyl, oxazolidinyl, oxazolidonyl, oxazolinyl, oxazolonyl, oxazolyl, oxepanyl, oxetanonyl, oxetanyl, oxetyl, oxtenayl, oxindolyl, oxiranyl, oxobenzoisothiazolyl, oxochromenyl, oxoisoquinolinyl, oxoquinolinyl, oxothiolanyl, phenanthridinyl, phenanthrolinyl, phenazinyl, phenothiazinyl, phenothienyl (benzothiofuranyl), phenoxathiinyl, phenoxazinyl, phthalazinyl, phthalazonyl, phthalidyl, phthalimidinyl, piperazinyl, piperidinyl, piperidonyl (e.g., 4-piperidonyl), pteridinyl, purinyl, pyranyl, pyrazinyl, pyrazolidinyl, pyrazolinyl, pyrazolopyrimidinyl, pyrazolyl, pyridazinyl, pyridinyl, pyridopyrazinyl, pyridopyrimidinyl, pyridyl, pyrimidinyl, pyrimidyl, pyronyl, pyrrolidinyl, pyrrolidonyl (e.g., 2-pyrrolidonyl), pyrrolinyl, pyrrolizidinyl, pyrrolyl (e.g., 2H-pyrrolyl), pyrylium, quinazolinyl, quinolinyl, quinolizinyl (e.g., 4H-quinolizinyl), quinoxalinyl, quinuclidinyl, selenazinyl, selenazolyl, selenophenyl, succinimidyl, sulfolanyl, tetrahydrofuranyl, tetrahydrofuryl, tetrahydroisoquinolinyl, tetrahydroisoquinolyl, tetrahydropyridinyl, tetrahydropyridyl (piperidyl), tetrahydropyranyl, tetrahydropyronyl, tetrahydroquinolinyl, tetrahydroquinolyl, tetrahydrothienyl, tetrahydrothiophenyl, tetrazinyl, tetrazolyl, thiadiazinyl (e.g., 6H-1,2,5- thiadiazinyl or 2H,6H-1,5,2-dithiazinyl), thiadiazolyl, thianthrenyl, thianyl, thianaphthenyl, thiazepinyl, thiazinyl, thiazolidinedionyl, thiazolidinyl, thiazolyl, thienyl, thiepanyl, thiepinyl, thietanyl, thietyl, thiiranyl, thiocanyl, thiochromanonyl, thiochromanyl, thiochromenyl, thiodiazinyl, thiodiazolyl, thioindoxyl, thiomorpholinyl, thiophenyl, thiopyranyl, thiopyronyl, thiotriazolyl, thiourazolyl, thioxanyl, thioxolyl, thymidinyl, thyminyl, triazinyl, triazolyl, trithianyl, urazinyl, urazolyl, uretidinyl, uretinyl, uricyl, uridinyl, xanthenyl, xanthinyl, xanthionyl, and the like, as well as modified forms thereof (e.g., including one or more oxo and/or amino) and salts thereof.

The heterocyclyl group may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3 0.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane,2-oxabicyclo[2.1.0]pentane,2-oxabicyclo[1.1.1]pentane,3-oxabicyclo[3.1.0]hexane,5-oxabicyclo[2.1.1]hexane,3-oxabicyclo[3.2.0]heptane,3-oxabicyclo[4.1.0]heptane,7-oxabicyclo[2.2.1]heptane,6-oxabicyclo[3.1.1]heptane,7-oxabicyclo[4.2.0]octane, 2-oxabicyclo[2.2.2]octane, 3-oxabicyclo[3.2.1]octane, and the like. The heterocyclyl group can include spirocyclic rings (e.g., a spirocyclic bicyclic structure, in which two rings are connected through just one atom). Non-limiting examples of spirocyclic heterocyclyls include, e.g., 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane, 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane,2,5-dioxaspiro[3.6]decane, 1-oxaspiro[5.5]undecane,3-oxaspiro[5.5]undecane, 3-oxa-9-azaspiro[5.5]undecane, and the like. The heterocyclyl group can be substituted or unsubstituted. For example, the heterocyclyl group can be substituted with one or more substitution groups, as described herein for alkyl.

By “hydroxyl” is meant —OH.

By “oxy” is meant —O—.

By “sulfinyl” is meant an —S(O)— group.

By “sulfonyl” is meant an —S(O)₂— group.

By “thio” is meant —S—.

As used herein and unless otherwise stated, the term “analog” refers to a compound that is structurally similar to another compound but differs slightly in composition (e.g., as in the replacement of one atom by an atom of a different element or in the presence of a particular functional group, or the replacement of one functional group by another functional group). Thus, an analog is a compound that is similar or comparable in function and appearance, but not in structure or origin to the reference compound. Non-limiting examples of analogs include, e.g., deuterated analogs, in which one or more hydrogen (H) atoms are replaced with deuterium (D) atoms. Other isotopes may be employed. Analogs may include isotopically-labeled compounds having one or more isotopes of hydrogen, carbon, nitrogen, oxygen, and/or fluorine (e.g., ³H, ²H, ¹¹C, ¹³C, ¹⁴C, ¹⁵N, ¹⁸O, and/or ¹⁸F). In particular, radioisotopes which emit radiation as they decay, such as ³H or ¹⁴C, are useful in pharmaceutical preparations or in vivo topographic tests of compounds. The stable isotope neither decays nor changes in their amount, nor have radioactivity, so they can be used safely. When the atom constituting the compound molecule of the present disclosure is an isotope, it may be transformed according to the common method by replacing the reagent used in synthesis with a reagent containing the corresponding isotope.

As used herein and unless otherwise stated, the term “prodrug” means a compound that is convertible in vivo by metabolic means (e.g., by hydrolysis, oxidation, acidification, and the like) to a disclosed compound. In some embodiments, the compounds may be supplied as “prodrugs” or protected forms, which release the compound after administration to a subject. For example, the compound may carry a protective group which is split off by hydrolysis in body fluids, e.g., in the bloodstream, thus releasing the active compound or is oxidized or reduced in body fluids to release the compound. Accordingly, a “prodrug” is meant to indicate a compound that may be converted under physiological conditions or by solvolysis to a biologically active compound of the invention. Thus, the term “prodrug” refers to a metabolic precursor of a compound of the invention that is pharmaceutically acceptable. Prodrugs of any compound herein may be prepared by modifying functional groups present in the compound in such a way that the modifications are cleaved, either in routine manipulation or in vivo, to release the parent compound described herein. Prodrugs include compounds (e.g., any described herein) wherein a hydroxyl, amino, or other group is bonded to any group that, when the prodrug of the compound is administered to a subject, cleaves to form a free hydroxy, free amino, or free other group, respectively. Examples of prodrugs include, but are not limited to, acetate, formate and benzoate derivatives of alcohol and acetamide, formamide, and benzamide derivatives of amine functional groups in any compound herein (e.g., such as a compound of Formulas (I), (IIa)-(IIc), (IIIa)-(IIIc), (IV), (IVa), and (IVb), or in Table 1 or Table 3c).

As used herein and unless otherwise stated, the term “solvate” includes any combination which may be formed by a derivative of this invention with a suitable inorganic solvent (e.g., hydrates) or organic solvent, such as but not limited to alcohols, ketones, esters and the like.

As used herein and unless otherwise stated, the term “stereoisomer” refers to all possible different isomeric as well as conformational forms which the compounds of this invention may possess, in particular all possible stereochemically and conformationally isomeric forms, all diastereomers, enantiomers and/or conformers of the basic molecular structure.

As used herein and unless otherwise stated, the term “tautomer” refers to compounds whose structures differ in arrangement of atoms but which exist in equilibrium, and it is to be understood that compounds provided herein may be depicted as different tautomers. When compounds have tautomeric forms, all tautomeric forms are intended to be within the scope of the disclosure, and the naming of the compounds does not exclude any tautomer.

By “salt” is meant an ionic form of a compound or structure (e.g., any formulas, compounds, or compositions described herein), which includes a cation or anion compound to form an electrically neutral compound or structure. Salts (e.g., simple salts having binary compounds, double salts, triple salts, etc.) are well known in the art. For example, salts are described in Berge S M et al., “Pharmaceutical salts,” J. Pharm. Sci. 1977 January; 66(1):1-19; International Union of Pure and Applied Chemistry, “Nomenclature of Inorganic Chemistry,” Butterworth & Co. (Publishers) Ltd., London, England, 1971 (2nd ed.); and in “Handbook of Pharmaceutical Salts: Properties, Selection, and Use,” Wiley-VCH, April 2011 (2nd rev. ed., eds. P. H. Stahl and C. G. Wermuth). The salts can be prepared in situ during the final isolation and purification of the compounds of the disclosure or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecyl sulfate, edetate, ethanesulfonate, fumarate, glucoheptonate, glucomate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth metal salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine.

By “pharmaceutically acceptable salt” is meant a salt that is, within the scope of sound medical judgment, suitable for use in contact with the tissues of humans and animals without undue toxicity, irritation, allergic response and the like and are commensurate with a reasonable benefit/risk ratio.

By “pharmaceutically acceptable excipient” is meant any ingredient other than a compound or structure (e.g., any formulas, compounds, or compositions described herein) and having the properties of being non-toxic and non-inflammatory in a subject. Exemplary, non-limiting excipients include adjuvants, antiadherents, antioxidants, binders, carriers, coatings, compression aids, diluents, disintegrants, dispersing agents, dyes (colors), emollients, emulsifiers, fillers (diluents), film formers or coatings, flavors, fragrances, glidants (flow enhancers), isotonic carriers, lubricants, preservatives, printing inks, solvents, sorbents, stabilizers, suspensing or dispersing agents, surfactants, sweeteners, waters of hydration, or wetting agents. Any of the excipients can be selected from those approved, for example, by the United States Food and Drug Administration or other governmental agency as being acceptable for use in humans or domestic animals. Exemplary excipients include, but are not limited to alcohol, butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, glycerol, hydroxypropyl cellulose, hydroxypropyl methylcellulose, lactated Ringer's solution, lactose, magnesium stearate, maltitol, maltose, mannitol, methionine, methylcellulose, methyl paraben, microcrystalline cellulose, polyethylene glycol, polyol, polyvinyl pyrrolidone, povidone, pregelatinized starch, propyl paraben, retinyl palmitate, Ringer's solution, shellac, silicon dioxide, sodium carboxymethyl cellulose, sodium chloride injection, sodium citrate, sodium starch glycolate, sorbitol, starch (corn), stearic acid, stearic acid, sucrose, talc, titanium dioxide, vegetable oil, vitamin A, vitamin E, vitamin C, water, and xylitol.

As used herein, “administration,” “administering,” and variants thereof refers to introducing a compound or a composition into a subject and includes concurrent and sequential introduction of a compound or a composition. “Administration” can refer, e.g., to therapeutic, pharmacokinetic, diagnostic, research, placebo, and experimental methods. “Administration” also encompasses in vitro and ex vivo treatments. The introduction of a compound or a composition into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. Administration can be carried out by any suitable route. A suitable route of administration allows the compound or the composition to perform its intended function.

As used herein, the terms “subject,” “individual,” and “patient” are used interchangeably herein and refer to any animal subject for whom diagnosis, treatment, or therapy is desired, particularly humans. In some embodiments, the subject is a mammal (e.g., a human subject). In some embodiments, the subject is a non-human mammal (e.g., mouse, rat, guinea pig, dog, cat, horse, cow, pig, rabbit, sheep, or non-human primate, such as a monkey, chimpanzee, or baboon).

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

As used herein, the terms “therapeutically effective amount” and “effective amount” are used interchangeably to refer to an amount of a compound or a composition that is sufficient to provide the intended benefit (e.g., prevention, prophylaxis, delay of onset of symptoms, or amelioration of symptoms of a disease). In prophylactic or preventative applications, an effective amount can be administered to a subject susceptible to, or otherwise at risk of developing a disease, disorder or condition to eliminate or reduce the risk, lessen the severity, or delay the onset of the disease, disorder or condition, including a biochemical, histologic and/or behavioral symptoms of the disease, disorder or condition, its complications, and intermediate pathological phenotypes.

As used herein, the terms “treat,” “treating,” and/or “treatment” include abrogating, substantially inhibiting, slowing or reversing the progression of a disorder, disease or condition, substantially ameliorating clinical symptoms of a disorder, disease or condition, or substantially preventing the appearance of clinical symptoms of a disorder, disease or condition, obtaining beneficial or desired clinical results. Treating further refers to accomplishing one or more of the following: (a) reducing the severity of the disorder, disease or condition; (b) limiting development of symptoms characteristic of the disorder, disease or condition(s) being treated; (c) limiting worsening of symptoms characteristic of the disorder, disease or condition(s) being treated; (d) limiting recurrence of the disorder, disease or condition(s) in subjects that have previously had the disorder, disease or condition(s); and (e) limiting recurrence of symptoms in subjects that were previously asymptomatic for the disorder, disease or condition(s). Beneficial or desired clinical results, such as pharmacologic and/or physiologic effects include, but are not limited to, preventing the disease, disorder or condition from occurring in a subject predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (e.g., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

Other features and advantages of the present disclosure will be apparent from the following detailed description and the claims.

### DETAILED DESCRIPTION

The present disclosure relates to compounds of any described herein (e.g., such as a compound of Formulas (I), (IIa)-(IIc), (IIIa)-(IIIc), (IV), (IVa), and (IVb), or in Table 1 or Table 3c), as well as pharmaceutically acceptable salts thereof, stereoisomers thereof, analogs thereof, solvates thereof, prodrugs thereof, or tautomers thereof. In some non-limiting examples, the compound (e.g., any described herein) is characterized as being a GLP-1 receptor agonist.

The present disclosure relates, in part, to a compound of Formula (I):

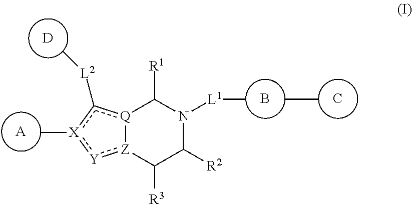

or a pharmaceutically acceptable salt thereof, stereoisomer thereof, tautomer thereof, analog thereof (e.g., deuterated analog thereof), or solvate thereof, wherein:
   each of R¹, R², and R³ is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy);
   each of L¹ and L² is, independently, a linker;
   each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z);
   A is an optionally substituted aryl (e.g., an optionally substituted C₆₋₁₀ aryl, such as optionally substituted phenyl) (e.g., wherein optional substituents include halo, cyano, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these);
B is an optionally substituted heterocyclyl (e.g., an optionally substituted 5-10 membered heterocyclyl, such as optionally substituted bicyclic, 5-10 membered heterocyclyl; an optionally substituted 5-10 membered heteroaryl, such as an optionally substituted indole) (e.g., wherein B may be optionally substituted with V¹, wherein V¹ is an optionally substituted heterocyclyl (e.g., an optionally substituted 5-10 membered heterocyclyl));
C is

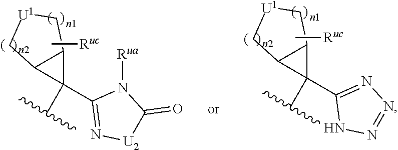

wherein
U¹ is oxy (e.g., —O—), thio (e.g., —S—), sulfinyl (e.g., —S(O)—), sulfonyl (e.g., —S(O)₂—), optionally substituted amino (e.g., —N(R^{ua})—), or optionally substituted alkylene (e.g., optionally substituted C₁₋₆ alkylene);
U² is O, —N(R^{ua})—, or S;
each of n1 and n2 is, independently, 0, 1, 2, or 3 (e.g., wherein at least one of n1 and n2 is not 0);
each R^{ua} is, independently, hydrogen (H), optionally substituted alkyl (e.g., optionally substituted C₁₋₆ alkyl), optionally substituted heterocyclyl (e.g., an optionally substituted 5-10 membered heterocyclyl) (e.g., wherein the optional substituents include C₁₋₆ alkyl, halo, C₁₋₆ haloalkyl), optionally substituted alkoxy (e.g., optionally substituted C₁₋₆ alkoxy), optionally substituted sulfonyl (e.g., —S(O)₂—), or optionally substituted cycloalkyl (e.g., optionally substituted C₃₋₈ cycloalkyl); and
each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached, and
D is optionally substituted heterocyclyl (e.g., an optionally substituted 5-10 membered heterocyclyl, such as optionally substituted bicyclic, 5-10 membered heterocyclyl) (e.g., wherein optionally substituents include halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, C is

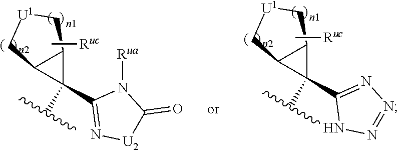

In some embodiments, C is

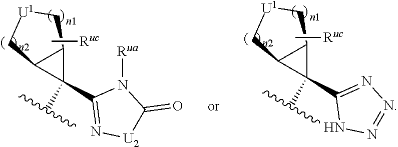

The present disclosure relates, in part, to a compound of Formula (I), as well as pharmaceutically acceptable salt thereof, stereoisomers thereof, analogs thereof, solvates thereof, prodrugs thereof, or tautomers thereof.

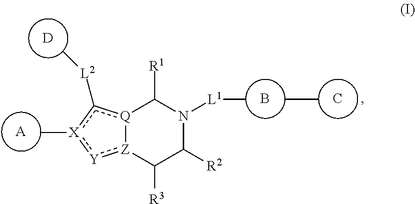

wherein:
each of R¹, R², and R³ is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy);
each of L¹ and L² is, independently, a linker;
each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z);
A comprises an optionally substituted aryl (e.g., an optionally substituted C₆₋₁₀ aryl, such as optionally substituted phenyl) (e.g., wherein optionally substituents include halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these);
B comprises an optionally substituted heterocyclyl (e.g., an optionally substituted 5-10 membered heterocyclyl, such as optionally substituted bicyclic, 5-10 membered heterocyclyl) (e.g., wherein B may be optionally substituted with V¹, as described herein);
C comprises

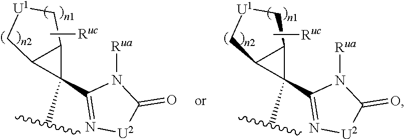

wherein U¹ is oxy (e.g., —O—), thio (e.g., —S—), sulfinyl (e.g., —S(O)—), sulfonyl (e.g., —S(O)₂—), optionally substituted amino (e.g., —N(R^{ua})—), or optionally substituted alkylene (e.g., optionally substituted C₁₋₆ alkylene); U² is O or S; each of n1 and n2 is, independently, 0, 1, 2, or 3 (e.g., wherein at least one of n1 and n2 is not 0); each R^{ua} is, independently, hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl); and each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached; and
D comprises optionally substituted heterocyclyl (e.g., an optionally substituted 5-10 membered heterocyclyl, such as optionally substituted bicyclic, 5-10 membered heterocyclyl) (e.g., wherein optionally substituents include halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

A compound of Formula (I) can be prepared in any useful manner. Scheme I provides a non-limiting, generalized method for preparing a compound. Each of A, B, C, D, R¹, R², R³, X, Y, Z, Q, L², and L¹ can be any described herein. LG indicates a leaving group (e.g., alkoxy, hydroxyl, halo, and the like).

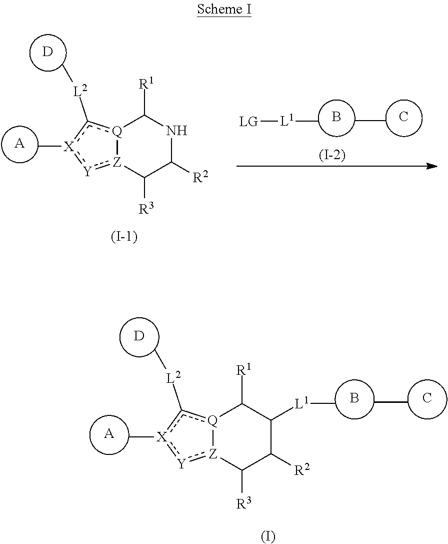

Referring to Scheme I, coupling of reagent I-1 with I-2 under standard coupling conditions, such as amide bond forming conditions when L¹ is —C(═O)—, e.g., in the presence of a carboxyl activation agent such as EDCI, DCC, HATU, HBTU, HCTU, PyBOP, DPPA, especially HATU (2-(7-Azabenzotriazol-1-yl)-N,N,N′,N′-tetramethyluronium hexafluorophosphate) (e.g., in a polar aprotic solvent such as DMF), can provide compounds of Formula I.

Scheme II provides a non-limiting, generalized method for preparing a compound (I-2′). Each of B, L¹, U¹, U², n1, n2, and R^{ua} can be any described herein. LG indicates a leaving group (e.g., alkoxy, hydroxyl, halo, and the like). In turn, I-2′ can be reacted with I-1 to provide a non-limiting example of a compound of formula (I) (e.g., compound (I′)), which includes C as

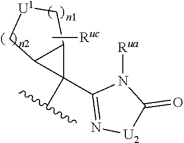

or a stereoisomer thereof. Reacting of reagent I-3 with I-2a under basic conditions provides reagent I-2b. Treatment of I-2b with an amine source, followed by reaction with a carbonyl equivalent such as carbonyl diimidazole (CDI) provided reagent I-2′, which is a non-limiting example of a reagent I-2. Then, coupling of reagent I-1 with I-2′ under standard coupling conditions, such as amide bond forming conditions when L¹ is —C(═O)—, e.g., in the presence of a carboxyl activation agent such as HATU (e.g., in a polar aprotic solvent such as DMF) can provide non-limiting examples of compounds (e.g., compound I′) encompassed by Formula I. Protecting and deprotecting chemistry can be employed as needed. Reagents and compounds may be provided in any useful form, such as stereoisomers thereof, tautomers thereof, prodrug forms thereof, analogs thereof, salts thereof, and/or solvates thereof. Accordingly, one or more chiral centers may or may not be present in such reagents and/or compounds. Other synthetic strategies may be employed.

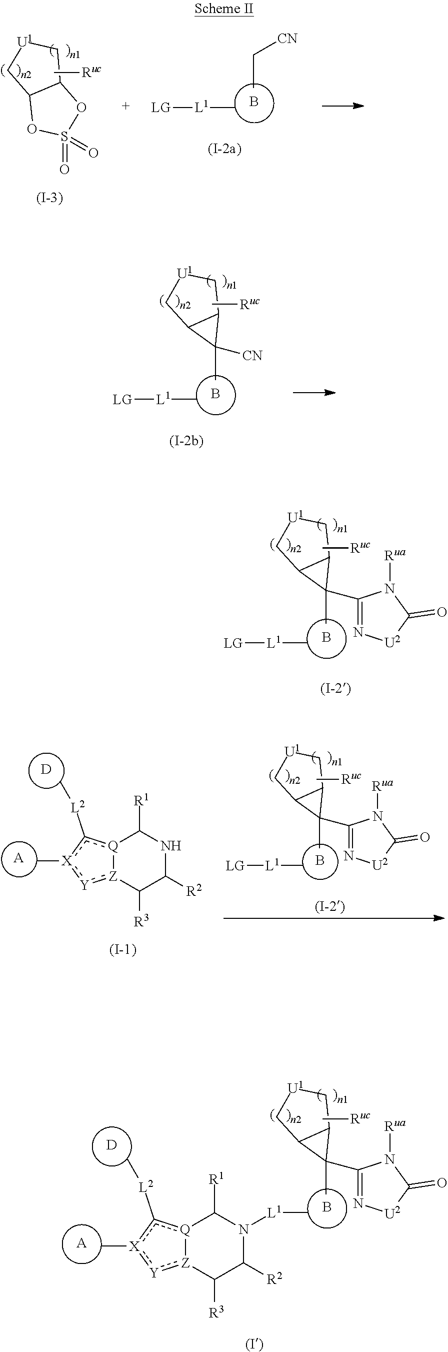

The present disclosure relates, in part, to a compound having a structure of Formulas (I), (IIa)-(IIc), (IIIa)-(IIIc), (IV), (IVa), and (IVb) as well as pharmaceutically acceptable salt thereof, stereoisomers thereof, analogs thereof, solvates thereof, prodrugs thereof, or tautomers thereof.

In some embodiments, the compound comprises a structure of Formula (IIa):

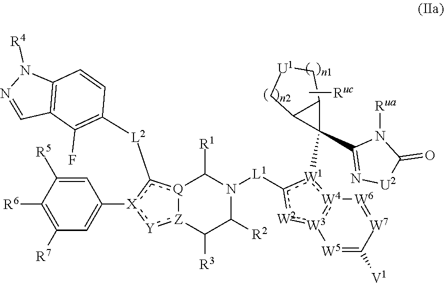

or a pharmaceutically acceptable salt thereof, stereoisomer thereof, analog thereof, solvate thereof, prodrug thereof, or tautomer thereof. Each of R¹, R², R³, R⁴, R⁵, R⁶, R⁷, L¹, L², X, Y, Z, Q, W¹, W², W³, W⁴, W⁵, W⁶, W⁷, R^{wa}, U¹, U², R^{ua}, R^{uc}, n1, n2, and V¹ can be any described herein.

In some embodiments, each of R¹, R², and R³ is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy). In some embodiments, R¹ and R² are independently H, optionally substituted alkyl, or optionally substituted alkoxy. In some embodiments, R¹ and R² are independently hydrogen (H) or optionally substituted alkyl.

In some embodiments, R⁴ is hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl).

In some embodiments, each of R⁵, R⁶, and R⁷ is, independently, hydrogen (H), halo, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), or optionally substituted halocycloalkyl (e.g., optionally substituted C₃₋₈ halocycloalkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, each of L¹ and L² is, independently, a linker.

In some embodiments, each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z).

In some embodiments, each of W¹, W³, and W⁴ is, independently, C or N.

In some embodiments, W² is CR^{wa}, NR^{wa}, N, O, or S, and the dashed line represents an optional double bond (e.g., wherein two double bonds are not attached to the same atom).

In some embodiments, each of W⁵, W⁶, and W⁷ is, independently, CR^{wa} or N.

In some embodiments, each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, U¹ is —N(R^{ua})—.

In some embodiments, U¹ is oxy (e.g., —O—), thio (e.g., —S—), sulfinyl (e.g., —S(O)—), sulfonyl (e.g., —S(O)₂—), optionally substituted amino (e.g., —N(R^{ua})—), or optionally substituted alkylene (e.g., optionally substituted C₁₋₆ alkylene).

In some embodiments, U² is O. In some embodiments, U² is S. In some embodiments, U² is —N(R^{ua})—.

In some embodiments, R^{ua} is, independently, hydrogen (H), optionally substituted alkyl (e.g., optionally substituted C₁₋₆ alkyl), optionally substituted heterocyclyl (e.g., an optionally substituted 5-10 membered heterocyclyl) (e.g., wherein the optional substituents include C₁₋₆ alkyl, halo, C₁₋₆ haloalkyl), optionally substituted alkoxy (e.g., optionally substituted C₁₋₆ alkoxy), optionally substituted sulfonyl (e.g., —S(O)₂—), or optionally substituted cycloalkyl (e.g., optionally substituted C₃₋₈ cycloalkyl). In some embodiments, R^{ua} is, independently, hydrogen (H), C₁₋₆ alkyl, C₁₋₆ alkoxy, 5-10 membered heterocyclyl, —S(O)₂—, or C₃₋₈ cycloalkyl, wherein each C₁₋₆ alkyl, C₁₋₆ alkoxy, 5-10 membered heterocyclyl, —S(O)₂—, or C₃₋₈ cycloalkyl of R^{ua} is independently optionally substituted with halo, oxo, hydroxyl, amino, C₁₋₆ alkyl, C₁₋₆ haloalkyl, or C₃₋₈ cycloalkyl. In some embodiments, R^{ua} is hydrogen. In some embodiments, R^{ua} is unsubstituted 5-10 membered heteroaryl. In some embodiments, R^{ua} is unsubstituted 5-10 membered heterocyclyl. In some embodiments, R^{ua} is 6 membered heterocyclyl. R^{ua} is 6 membered heteroaryl, e.g., pyridinyl or pyrimidinyl.

In some embodiments, each R^{ua} is, independently, hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl).

In some embodiments, each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached.

In some embodiments, each of n1 and n2 is, independently, 0, 1, 2, or 3 (e.g., wherein at least one of n1 and n2 is not 0).

In some embodiments, V¹ comprises an optionally substituted heterocyclyl (e.g., an optionally substituted heteroaryl).

In some embodiments, the compound comprises a structure of Formula (IIb):

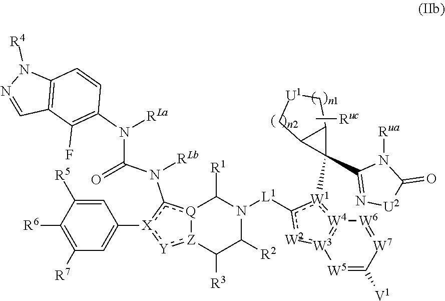

or a pharmaceutically acceptable salt thereof, stereoisomer thereof, analog thereof, solvate thereof, prodrug thereof, or tautomer thereof. Each of R¹, R², R³, R⁴, R⁵, R⁶, R⁷, R^{La}, R^{Lb}, X, Y, Z, Q, W¹, W², W³, W⁴, W⁵, W⁶, W⁷, R^{wa}, U¹, U², R^{ua}, R^{uc}, n1, n2, and V¹ can be any described herein.

In some embodiments, each of R¹, R², and R³ is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy). In some embodiments, R¹ and R² are independently H, optionally substituted alkyl, or optionally substituted alkoxy. In some embodiments, R¹ and R² are independently hydrogen (H) or optionally substituted alkyl.

In some embodiments, R⁴ is hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl).

In some embodiments, each of R⁵, R⁶, and R⁷ is, independently, hydrogen (H), halo, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), or optionally substituted halocycloalkyl (e.g., optionally substituted C₃₋₈ halocycloalkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, each of R^{La} and R^{Lb}, if present, is, independently, hydrogen (H), optionally substituted alkyl (e.g., optionally substituted C₁₋₆ alkyl), or optionally substituted alkoxy (e.g., optionally substituted C₁₋₆ alkoxy), and optionally each of R^{La} and R^{Lb} taken together with the nitrogen atoms to which each is attached, forms an optionally substituted heterocyclyl.

In some embodiments, each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z).

In some embodiments, each of W¹, W³, and W⁴ is, independently, C or N.

In some embodiments, W² is CR^{wa}, NR^{wa}, N, O, or S, and the dashed line represents an optional double bond (e.g., wherein two double bonds are not attached to the same atom).

In some embodiments, each of W⁵, W⁶, and W⁷ is, independently, CR^{wa} or N.

In some embodiments, each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, U¹ is oxy (e.g., —O—), thio (e.g., —S—), sulfinyl (e.g., —S(O)—), sulfonyl (e.g., —S(O)₂—), optionally substituted amino (e.g., —N(R^{ua})—), or optionally substituted alkylene (e.g., optionally substituted C₁₋₆ alkylene).

In some embodiments, U² is O. In some embodiments, U² is S. In some embodiments, U² is —N(R^{ua})—.

In some embodiments, each R^{ua} is, independently, hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl).

In some embodiments, each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached.

In some embodiments, each of n1 and n2 is, independently, 0, 1, 2, or 3 (e.g., wherein at least one of n1 and n2 is not 0).

In some embodiments, V¹ comprises an optionally substituted heterocyclyl (e.g., an optionally substituted heteroaryl).

In some embodiments, V¹ is an optionally substituted heterocyclyl. In some embodiments, V¹ is 5-10 membered heterocyclyl. In some embodiments V¹ is

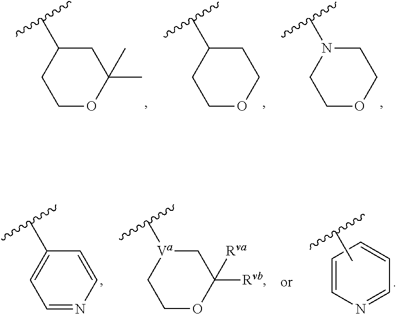

In some embodiments, V¹ is

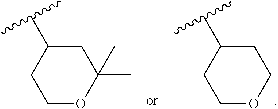

In some embodiments, V¹ is

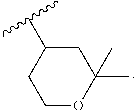 [SMILES: *C1CCOC(C)(C)C1]

In some embodiments, the compound comprises a structure of Formula (IIc):

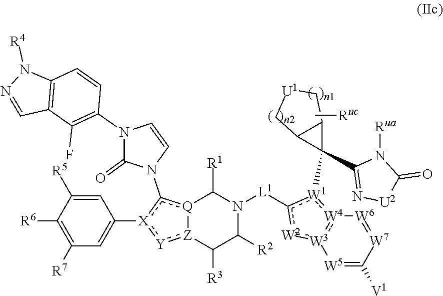

or a pharmaceutically acceptable salt thereof, stereoisomer thereof, analog thereof, solvate thereof, prodrug thereof, or tautomer thereof. Each of R¹, R², R³, R⁴, R⁵, R⁶, R⁷, X, Y, Z, Q, W¹, W², W³, W⁴, W⁵, W⁶, W⁷, R^{wa}, U¹, U², R^{ua}, R^{uc}, n1, n2, and V¹ can be any described herein.

In some embodiments, each of R¹, R², and R³ is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy). In some embodiments, R¹ and R² are independently H, optionally substituted alkyl, or optionally substituted alkoxy. In some embodiments, R¹ and R² are independently hydrogen (H) or optionally substituted alkyl.

In some embodiments, R⁴ is hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl).

In some embodiments, each of R⁵, R⁶, and R⁷ is, independently, hydrogen (H), halo, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), or optionally substituted halocycloalkyl (e.g., optionally substituted C₃₋₈ halocycloalkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z).

In some embodiments, each of W¹, W³, and W⁴ is, independently, C or N.

In some embodiments, W² is CR^{wa}, NR^{wa}, N, O, or S, and the dashed line represents an optional double bond (e.g., wherein two double bonds are not attached to the same atom).

In some embodiments, each of W⁵, W⁶, and W⁷ is, independently, CR^{wa} or N.

In some embodiments, each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, U¹ is oxy (e.g., —O—), thio (e.g., —S—), sulfinyl (e.g., —S(O)—), sulfonyl (e.g., —S(O)₂—), optionally substituted amino (e.g., —N(R^{ua})—), or optionally substituted alkylene (e.g., optionally substituted C₁₋₆ alkylene).

In some embodiments, U² is O. In some embodiments, U² is S. In some embodiments, U² is —N(R^{ua})—.

In some embodiments, each R^{ua} is, independently, hydrogen (H), optionally substituted alkyl (e.g., optionally substituted C₁₋₆ alkyl), or optionally substituted alkoxy (e.g., optionally substituted C₁₋₆ alkoxy).

In some embodiments, each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached.

In some embodiments, each of n1 and n2 is, independently, 0, 1, 2, or 3 (e.g., wherein at least one of n1 and n2 is not 0).

In some embodiments, V¹ comprises an optionally substituted heterocyclyl (e.g., an optionally substituted heteroaryl).

In some embodiments, the compound comprises a structure of Formula (IIIa):

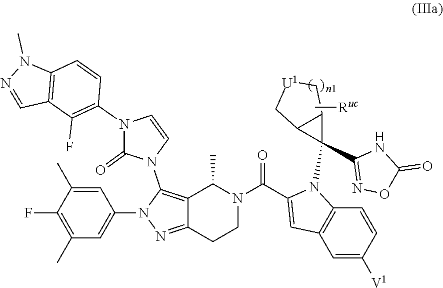

or a pharmaceutically acceptable salt thereof, stereoisomer thereof, analog thereof, solvate thereof, prodrug thereof, or tautomer thereof. Each of U¹, R^{uc}, n1, and V¹ can be any described herein.

In some embodiments, U¹ is oxy (e.g., —O—), thio (e.g., —S—), sulfinyl (e.g., —S(O)—), sulfonyl (e.g., —S(O)₂—), optionally substituted amino (e.g., —N(R^{ua})—), or optionally substituted alkylene (e.g., optionally substituted C₁₋₆ alkylene).

In some embodiments, each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached.

In some embodiments, n1 is 0, 1, 2, or 3.

In some embodiments, V¹ is an optionally substituted heterocyclyl.

In some embodiments, the compound comprises a structure of Formula (IIIb):

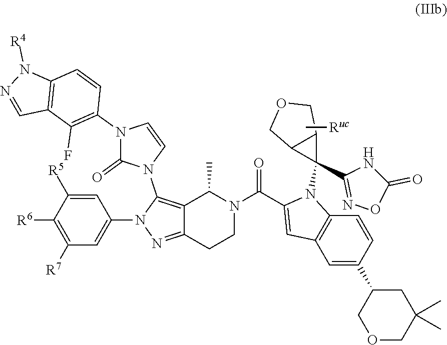

or a pharmaceutically acceptable salt thereof, stereoisomer thereof, analog thereof, solvate thereof, prodrug thereof, or tautomer thereof. Each of R⁴, R⁵, R⁶, R⁷, and R^{uc} can be any described herein.

In some embodiments, R⁴ is hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl).

In some embodiments, each of R⁵, R⁶, and R⁷ is, independently, hydrogen (H), halo, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), or optionally substituted halocycloalkyl (e.g., optionally substituted C₃₋₈ halocycloalkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached.

In some embodiments, the compound comprises a structure of Formula (IIIc):

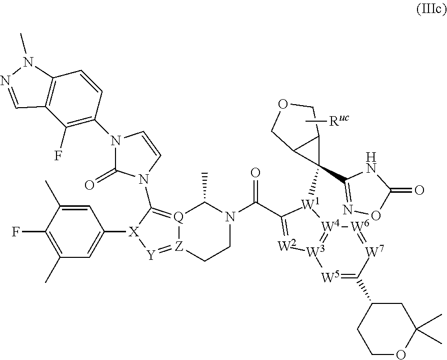

or a pharmaceutically acceptable salt thereof, stereoisomer thereof, analog thereof, solvate thereof, prodrug thereof, or tautomer thereof. Each of X, Y, Z, Q, W¹, W², W³, W⁴, W⁵, W⁶, W⁷, R^{wa}, and R^{uc} can be any described herein.

In some embodiments, each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z).

In some embodiments, each of W¹, W³, and W⁴ is, independently, C or N.

In some embodiments, W² is CR^{wa}, NR^{wa}, N, O, or S, and the dashed line represents an optional double bond (e.g., wherein two double bonds are not attached to the same atom).

In some embodiments, each of W⁵, W⁶, and W⁷ is, independently, CR^{wa} or N.

In some embodiments, each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached.

In any embodiment herein, each of R¹, R², and R³ is, independently, hydrogen (H) or optionally substituted alkyl.

In any embodiment herein, each of X and Y is N, each of Y and Z is N, or each of Y and Q is N.

In any embodiment herein, X is N, and a first double bond is present between Q and the adjacent carbon atom and a second double bond is present between Y and Z.

In any embodiment herein, Z is N, and a first double bond is present between Q and the adjacent carbon atom and a second double bond is present between X and Y.

In any embodiment herein, Q is N, and a first double bond is present between X and the adjacent carbon atom and a second double bond is present between Y and Z.

In any embodiment herein, L¹ is carbonyl (e.g., —C(O)—), alkylene (e.g., optionally substituted C₁₋₆ alkylene), sulfinyl (e.g., —S(O)—), or sulfonyl (e.g., —S(O)₂—).

In any embodiment herein, L² is

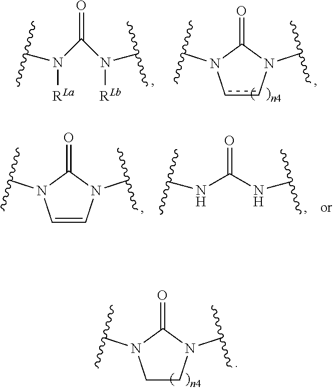

Each of R^{La}, R^{Lb}, and n4 can be any described herein.

In some embodiments, n4 is 1, 2, or 3, and the dashed line represents an optional double bond (e.g., wherein two double bonds are not attached to the same atom); and each of R^{La} and R^{Lb}, if present, is, independently, hydrogen (H), halo, optionally substituted alkyl (e.g., optionally substituted C₁₋₆ alkyl), or optionally substituted alkoxy (e.g., optionally substituted C₁₋₆ alkoxy), and optionally each of R^{La} and R^{Lb}, taken together with the nitrogen atoms to which each is attached, forms an optionally substituted heterocyclyl.

In any embodiment herein, A comprises an optionally substituted C₆₋₁₀ aryl, wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, or C₃₋₈ halocycloalkyl.

In any embodiment herein, A comprises

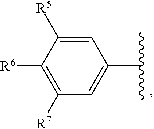

wherein each of R⁵, R⁶, and R⁷ is, independently, hydrogen (H), halo, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), or optionally substituted halocycloalkyl (e.g., optionally substituted C₃₋₈ halocycloalkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, A is

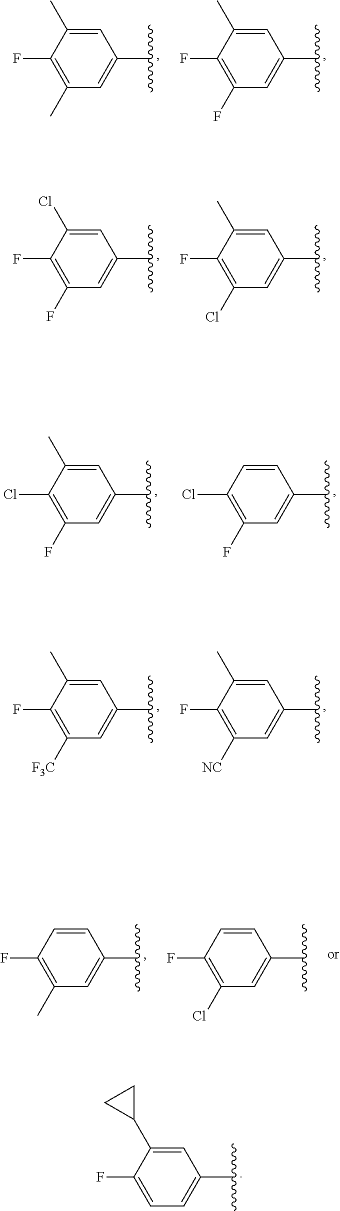

In some embodiments, A is

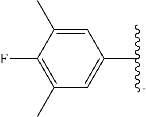 [SMILES: *c1cc(C)c(F)c(C)c1]

In any embodiment herein, A comprises one or more of the following:

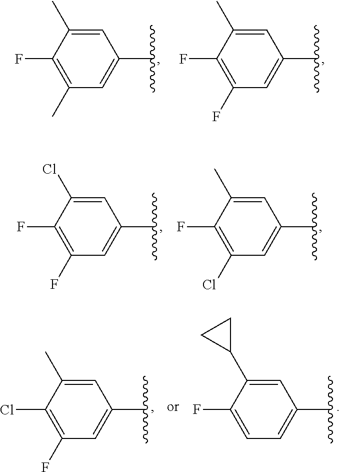

In some embodiments, B is an optionally substituted heterocyclyl (e.g., an optionally substituted 5-10 membered heterocyclyl, such as optionally substituted bicyclic, 5-membered heterocyclyl; an optionally substituted 5-10 membered heteroaryl, such as an optionally substituted indole) (e.g., wherein B may be optionally substituted with V¹, wherein V¹ is an optionally substituted heterocyclyl (e.g., an optionally substituted 5-10 membered heterocyclyl)). In some embodiments, B is an optionally substituted 5-10 membered heteroaryl, such as an optionally substituted indole). In some embodiments, B is an optionally substituted indole. In some embodiments, B is

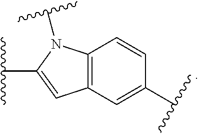 [SMILES: *c1ccc2c(c1)cc(*)n2*]

In any embodiment herein, B comprises the following:

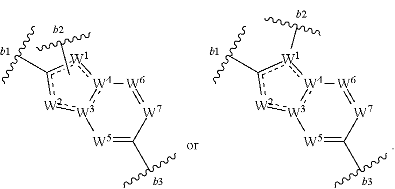

Each of W¹-W⁷ can be any described herein.

In some embodiments, each of W¹, W³, and W⁴ is, independently, C or N.

In some embodiments, W² is CR^{wa}, NR^{wa}, N, O, or S, and the dashed line represents an optional double bond (e.g., wherein two double bonds are not attached to the same atom).

In some embodiments, each of W⁵, W⁶, and W⁷ is, independently, CR^{wa} or N (e.g., wherein b1, b2, and b3 is connected to L¹, C, and V¹ in any order or position).

In some embodiments, each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In any embodiment herein, B comprises one or more of the following:

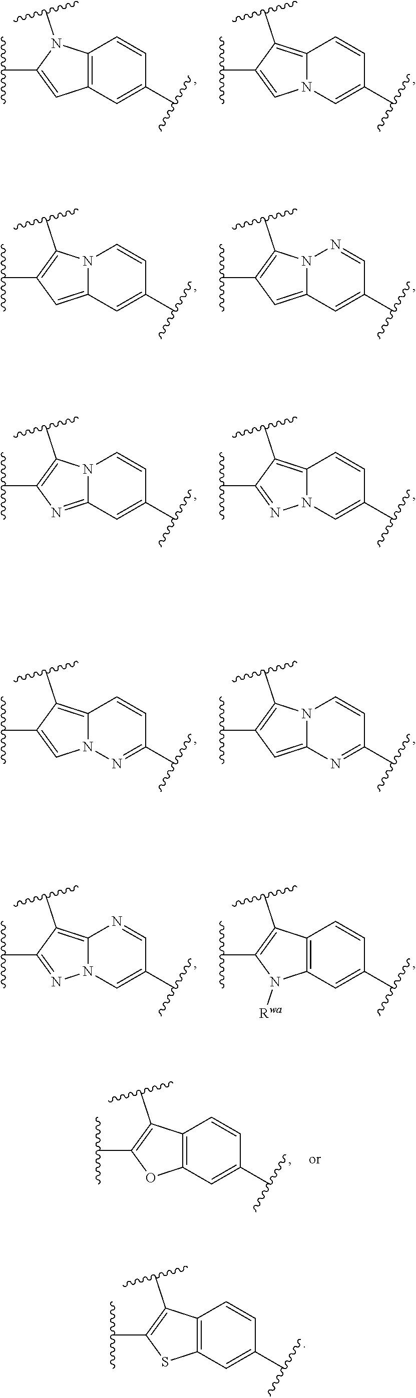

In any embodiment herein, B comprises the following:

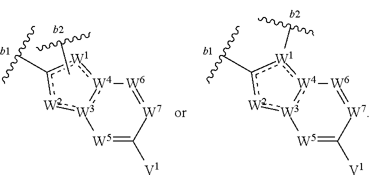

Each of W¹-W⁷ and V¹ can be any described herein.

In some embodiments, each of W¹, W³, and W⁴ is, independently, C or N.

In some embodiments, W² is CR^{wa}, NR^{wa}, N, O, or S, and the dashed line represents an optional double bond (e.g., wherein two double bonds are not attached to the same atom).

In some embodiments, each of W⁵, W⁶, and W⁷ is, independently, CR^{wa} or N (e.g., wherein b1 and b2 is connected to L¹ and C in any order or position).

In some embodiments, each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, V¹ comprises an optionally substituted heterocyclyl (e.g., an optionally substituted heteroaryl).

In any embodiment herein, B comprises one or more of the following:

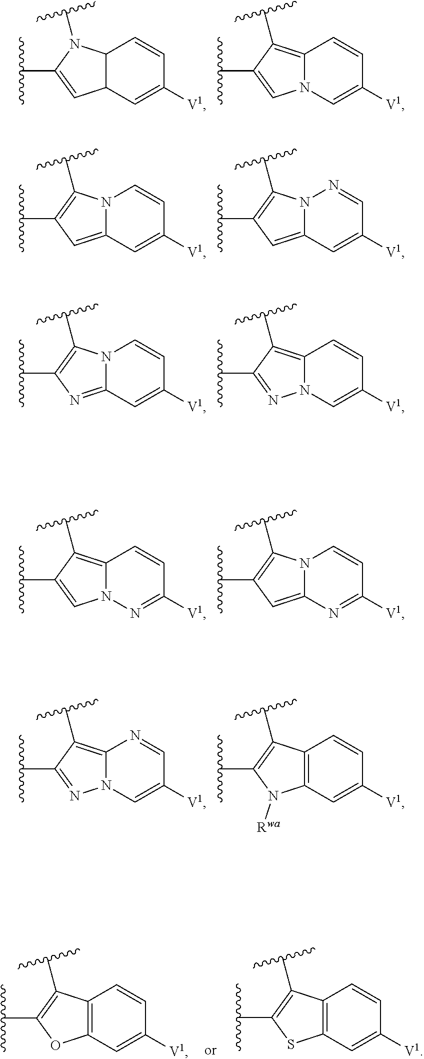

V¹ can be any described herein.

In some embodiments, V¹ comprises the following:

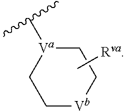

In some embodiments, V^{a} is N or CR^{vc}.

In some embodiments, V^{b} is NR^{Vc}, O, or S.

In some embodiments, each of R^{va} and R^{vc}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these), and one or more R^{va} may be present on V¹.

In some embodiments, V¹ comprises one or more of the following:

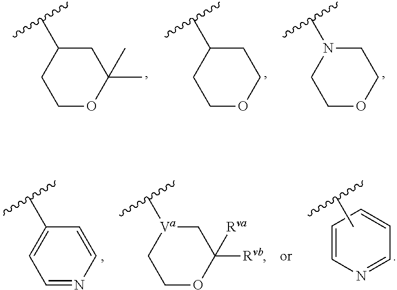

Each of V^{a} and R^{va}-R^{vc} can be any described herein.

In some embodiments, V^{a} is N or CR^{vc}.

In some embodiments, each of R^{va}, R^{vb}, and R^{vc}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In some embodiments, C is

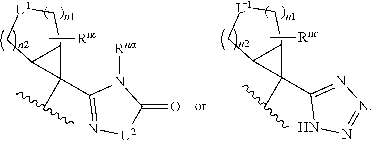

In some embodiments, C is

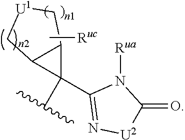

In some embodiments, C is

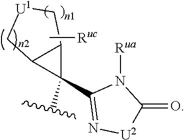

In some embodiment, C is

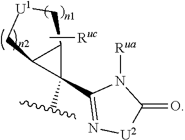

In some embodiments, C is

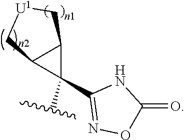

In some embodiments, C is

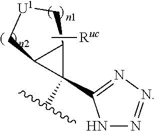

In some embodiments, C is

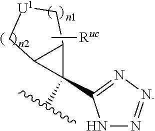

In some embodiments, C is

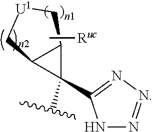

In some embodiments, C is

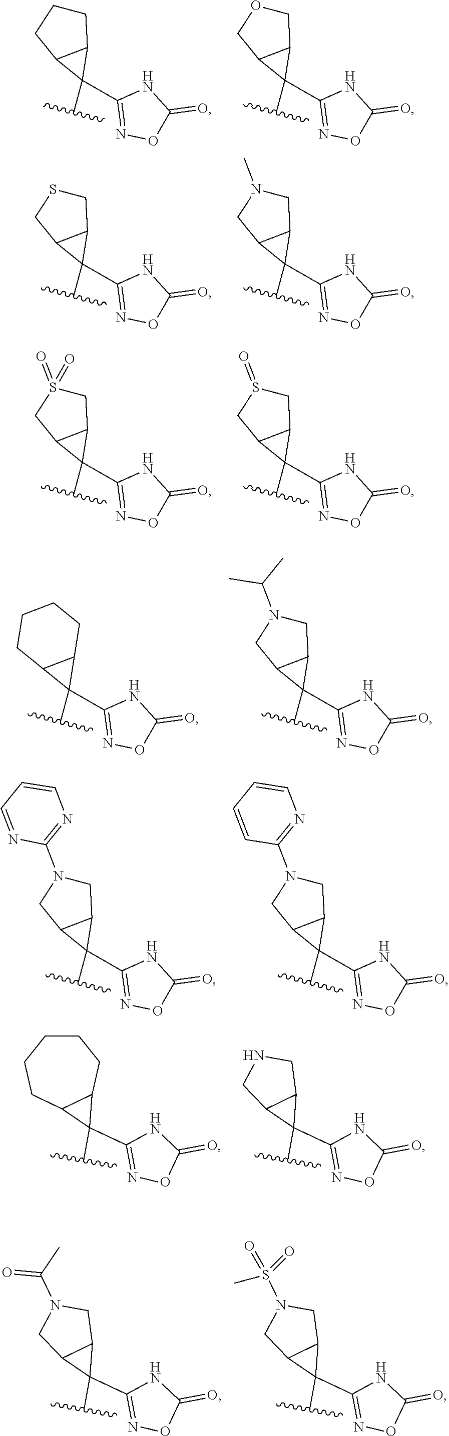

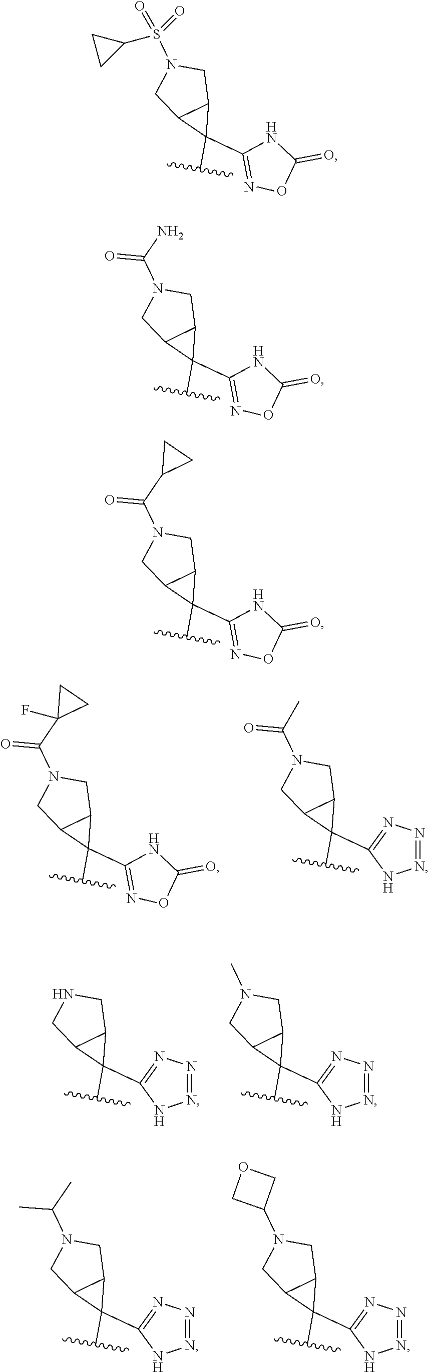

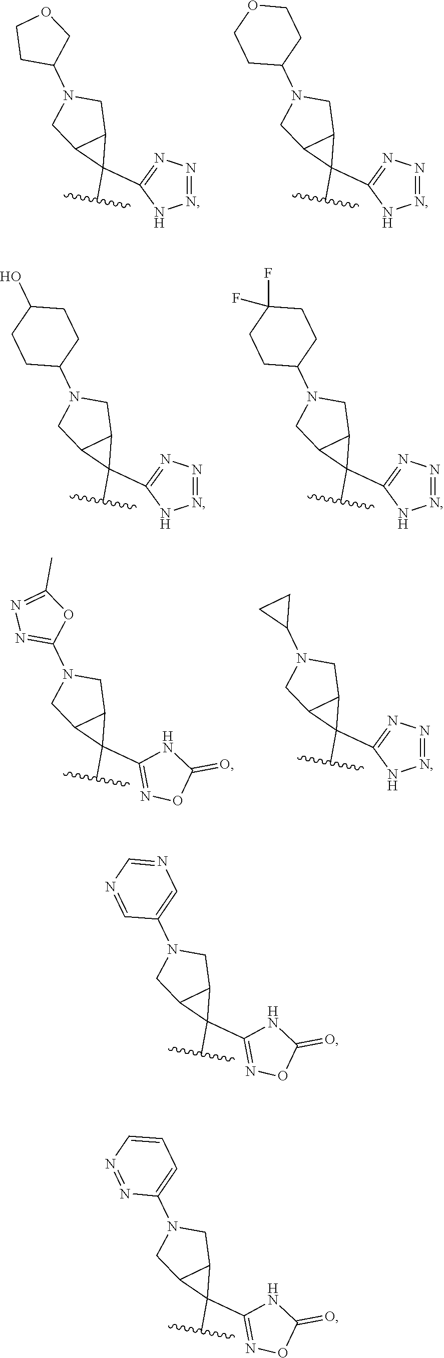

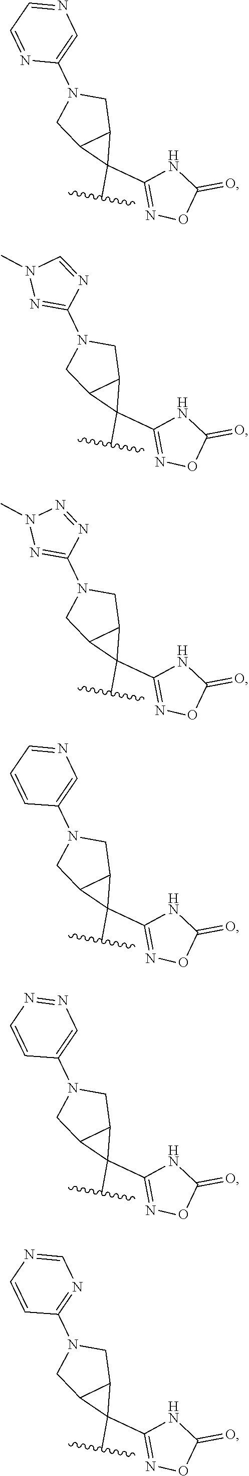 [SMILES: *C1(c2noc(=O)[nH]2)C2CN(c3cnccn3)CC21 ; *C1(c2noc(=O)[nH]2)C2CN(c3ncn(C)n3)CC21 ; *C1(c2noc(=O)[nH]2)C2CN(c3nnn(C)n3)CC21 ; *C1(c2noc(=O)[nH]2)C2CN(c3cccnc3)CC21 ; *C1(c2noc(=O)[nH]2)C2CN(c3ccnnc3)CC21 ; *C1(c2noc(=O)[nH]2)C2CN(c3ccncn3)CC21]

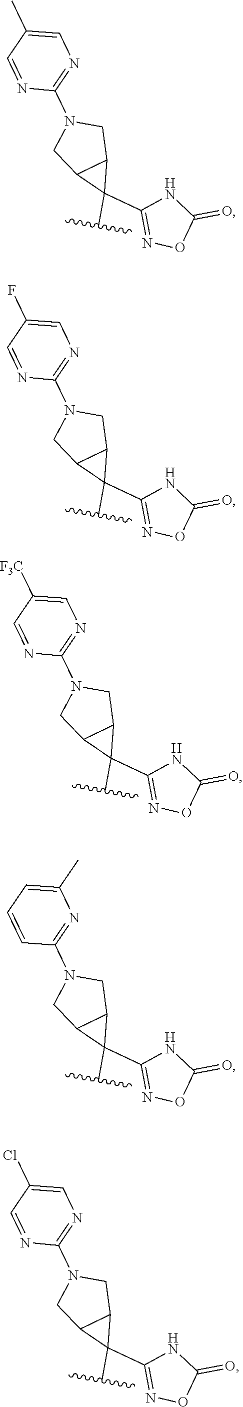 [SMILES: *C1(c2noc(=O)[nH]2)C2CN(c3ncc(C)cn3)CC21 ; *C1(c2noc(=O)[nH]2)C2CN(c3ncc(F)cn3)CC21 ; *C1(c2noc(=O)[nH]2)C2CN(c3ncc(C(F)(F)F)cn3)CC21 ; *C1(c2noc(=O)[nH]2)C2CN(c3cccc(C)n3)CC21 ; *C1(c2noc(=O)[nH]2)C2CN(c3ncc(Cl)cn3)CC21]

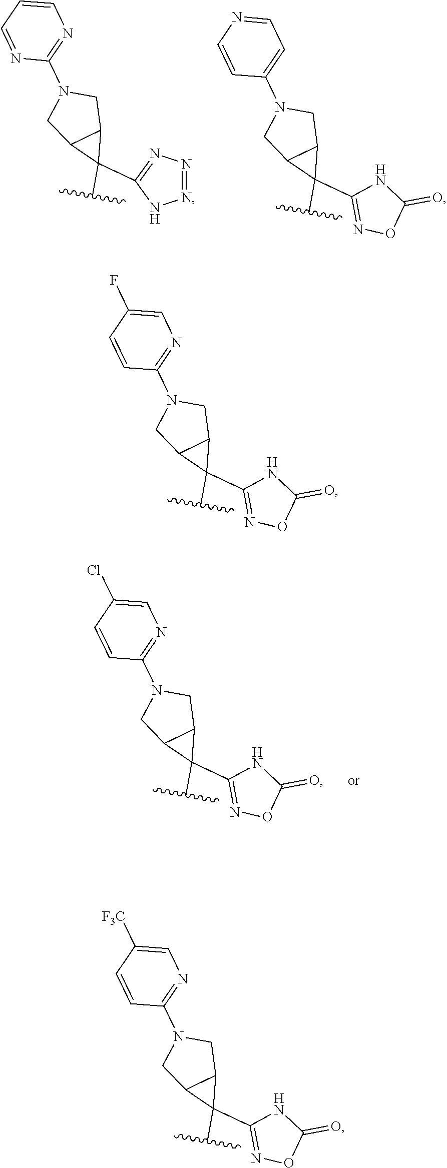 [SMILES: *C1(c2nnn[nH]2)C2CN(c3ncccn3)CC21 ; *C1(c2noc(=O)[nH]2)C2CN(c3ccncc3)CC21 ; *C1(c2noc(=O)[nH]2)C2CN(c3ccc(F)cn3)CC21 ; *C1(c2noc(=O)[nH]2)C2CN(c3ccc(Cl)cn3)CC21 ; "or" ; *C1(c2noc(=O)[nH]2)C2CN(c3ccc(C(F)(F)F)cn3)CC21]

or, or a stereoisomer thereof.

In any embodiment herein, C comprises the following:

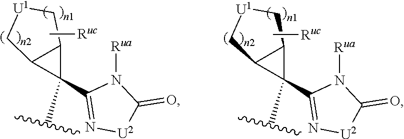

or a stereoisomer thereof. Each of U¹, U², R^{uc}, R^{ua}, n1, and n2 can be any described herein. Any number of R^{uc} may be present (e.g., 1, 2, 3, 4, 5, 6, or more) to substitute a hydrogen within the ring. In some embodiments, one or more R^{uc} can be selected from hydrogen (H), halo, hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), an isotope, and the like. In some embodiments, one or more R^{uc} can be any substituent described herein (e.g., a substituent provided herein for alkyl, such as any one of substituent (1) to (27), as well as optionally substituted aliphatic, optionally substituted heteroaliphatic, and the like).

In any embodiment herein, C comprises the following:

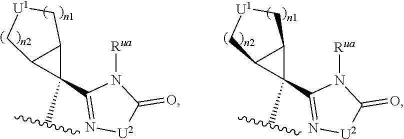

or a stereoisomer thereof. Each of U¹, U², R^{ua}, n1, and n2 can be any described herein.

In any embodiment herein, C comprises the following:

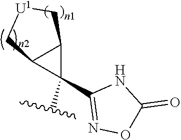

or a stereoisomer thereof. Each of U¹, n1, and n2 can be any described herein.

In some embodiments, U¹ is an optionally substituted C₁₋₆ alkylene. In some embodiments, optionally substituted C₁₋₆ alkylene is —CR^{Ua}R^{Ub}— or —[CR^{Ua}R^{Ub}]n3-, wherein n3 is 1, 2, or 3, and wherein each of R^{ua} and R^{ub}, if present, is, independently, hydrogen (H), optionally substituted alkyl (e.g., optionally substituted C₁₋₆ alkyl), or optionally substituted alkoxy (e.g., optionally substituted C₁₋₆ alkoxy).

In any embodiment herein, C comprises one or more of the following:

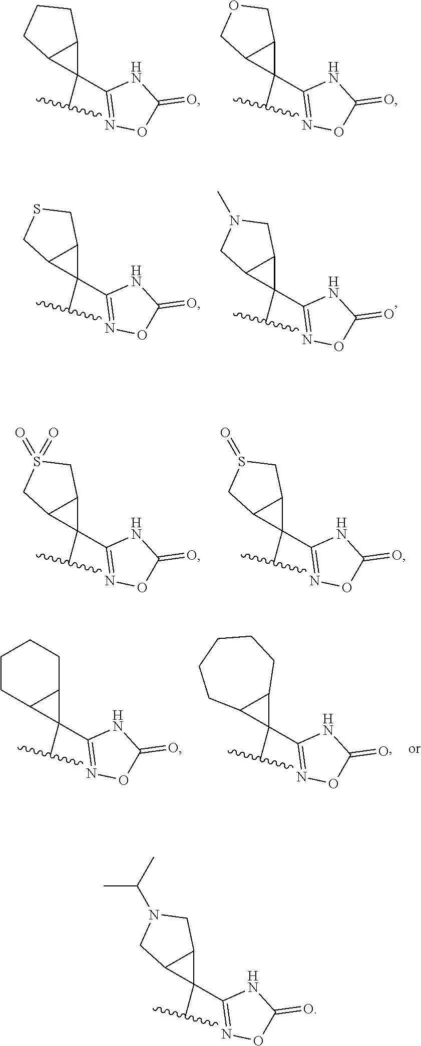

In some embodiments, D is

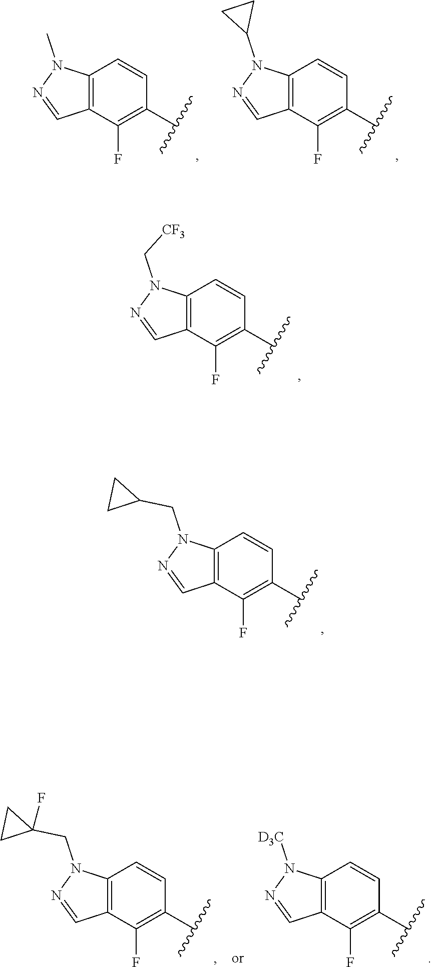 [SMILES: *c1ccc2c(cnn2C)c1F ; *c1ccc2c(cnn2C2CC2)c1F ; *c1ccc2c(cnn2CC(F)(F)F)c1F ; *c1ccc2c(cnn2CC2CC2)c1F ; *c1ccc2c(cnn2CC2(F)CC2)c1F ; *c1ccc2c(cnn2C([2H])([2H])[2H])c1F]

In some embodiments, D is

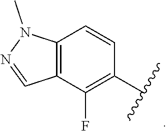 [SMILES: *c1ccc2c(cnn2C)c1F]

In any embodiment herein, D comprises the following:

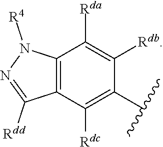

Each of R⁴, R^{da}, R^{bd}, R^{dc}, and R^{dd} can be any described herein.

In some embodiments, each of R^{da}, R^{bd}, R^{de}, and R^{dd} is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy).

In some embodiments, R⁴ is hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl) (e.g., wherein optionally substituents include halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In any embodiment herein, D comprises the following:

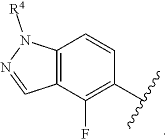

R⁴ can be any described herein.

In any embodiment herein, D comprises one or more of the following:

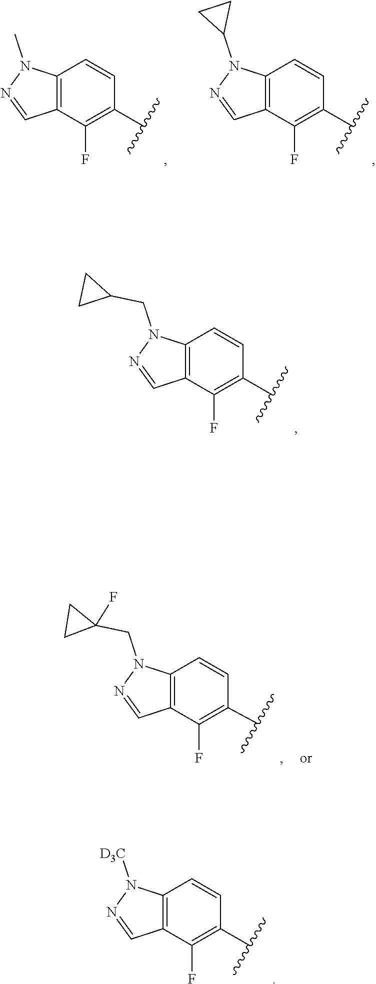 [SMILES: *c1ccc2c(cnn2C)c1F ; *c1ccc2c(cnn2C2CC2)c1F ; *c1ccc2c(cnn2CC2CC2)c1F ; *c1ccc2c(cnn2CC2(F)CC2)c1F ; *c1ccc2c(cnn2C([2H])([2H])[2H])c1F]

In any embodiment herein, X is N; X and Y is N; Y and Z is N; or Y and W is N.

In any embodiment herein, at least one of W¹, W², W³, W⁴, W⁵, W⁶, or W⁷ is, independently, N or NR^{Wa}. In some embodiments, R^{wa} is hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In any embodiment herein, R¹ is optionally substituted alkyl (e.g., optionally substituted C₁₋₆ alkyl).

In any embodiment herein, at least one of R¹, R², and R³ is not H.

In any embodiment herein, R⁴ is C₁₋₆ alkyl, deuterated C₁₋₆ alkyl, C₃₋₈ cycloalkyl, C₃₋₈ halocycloalkyl, C₁₋₆ alkyl substituted with one or more C₃₋₈ cycloalkyl, or C₁₋₆ alkyl substituted with one or more C₃₋₈ halocycloalkyl.

In any embodiment herein, each of R⁵, R⁶, and R⁷ is, independently, halo, C₁₋₆ alkyl, C₃₋₈ cycloalkyl, C₃₋₈ halocycloalkyl, C₁₋₆ alkyl substituted with one or more C₃₋₈ cycloalkyl, or C₁₋₆ alkyl substituted with one or more C₃₋₈ halocycloalkyl.

In any embodiment herein, R⁶ is halo (e.g., fluoro or chloro).

In any embodiment herein, each of R⁵ and R⁷ is, independently, halo, optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl), (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In any embodiment herein, at least one of R⁵, R⁶, and R⁷is not H.

In any embodiment herein, U¹ is O or methylene (CH₂).

In some embodiments, U¹ is O.

In any embodiment herein, n1 is 1, and/or n2 is 1.

In any embodiment herein, U² is 0, and/or R^{ua} is H or C₁₋₆ alkyl.

In some embodiments, U² is O.

In any embodiment herein, V¹ comprises one or more of the following:

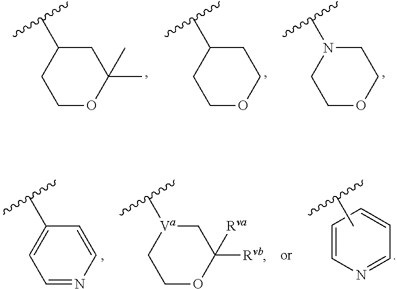

In some embodiments, V^{a} is N or CR^{Vc}.

In some embodiments, each of R^{va}, R^{vb}, and R^{vc}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In any embodiment herein, V¹ comprises the following:

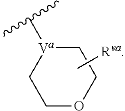

In some embodiments, V^{a} is N or CR^{vc}.

In some embodiments, each of R^{va} and R^{vc}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₃₋₈ cycloalkyl, or a combination of any of these), and one or more R^{va} may be present on V¹.

In any embodiment herein, V¹ comprises one or more of the following:

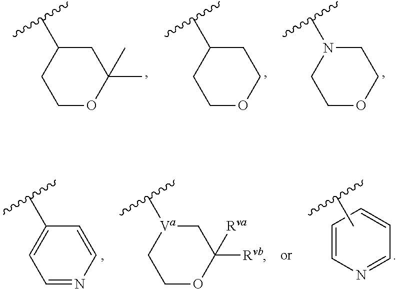

In some embodiments, V^{a} is N or CR^{vc}.

In some embodiments, each of R^{va}, R^{vb}, and R^{vc}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

In any embodiment herein, R⁴ is C₁₋₆ alkyl (e.g., methyl), deuterated C₁₋₆ alkyl (e.g., deuterated methyl, such as CD₃), C₃₋₈ cycloalkyl (e.g., cyclopropyl), C₁₋₆ alkyl substituted with one or more C₃₋₈ cycloalkyl (e.g., cyclopropylalkyl, including cyclopropylmethyl), C₃₋₈ halocycloalkyl (e.g., 1-halocyclopropyl, including 1-fluorocyclopropyl), or C₁₋₆ alkyl substituted with one or more C₃₋₈ halocycloalkyl (e.g., 1-halocyclopropylalkyl, including 1-fluorocyclopropylmethyl).

In any embodiment herein, each of R⁵, R⁶, and R⁷ is, independently, halo (e.g., fluoro or chloro), C₁₋₆ alkyl (e.g., methyl), C₃₋₈ cycloalkyl (e.g., cyclopropyl, such as C₃₋₈ cycloalkyl (e.g., cyclopropyl), C₁₋₆ alkyl substituted with one or more C₃₋₈ cycloalkyl (e.g., cyclopropylalkyl, including cyclopropylmethyl), C₃₋₈ halocycloalkyl (e.g., 1-halocyclopropyl, including 1-fluorocyclopropyl), or C₁₋₆ alkyl substituted with one or more C₃₋₈ halocycloalkyl (e.g., 1-halocyclopropylalkyl, including 1-fluorocyclopropylmethyl).

In any embodiment herein,

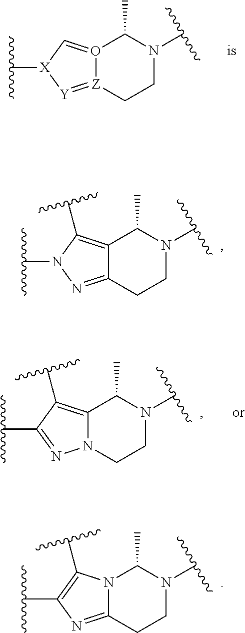

In some embodiments, X, Y, Z, and Q can be any described herein.

In any embodiment herein,

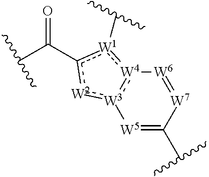

is

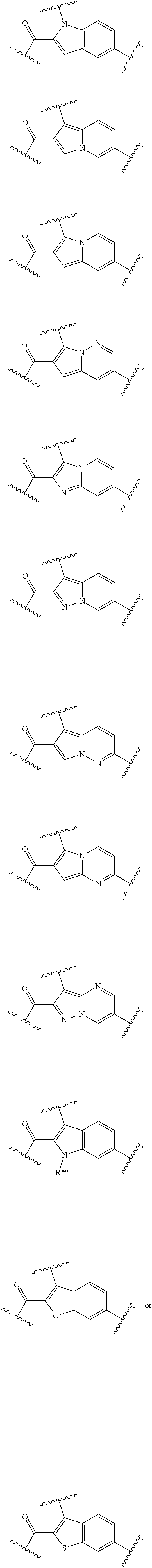

The present disclosure relates, in part, to a compound of Formula (IV):

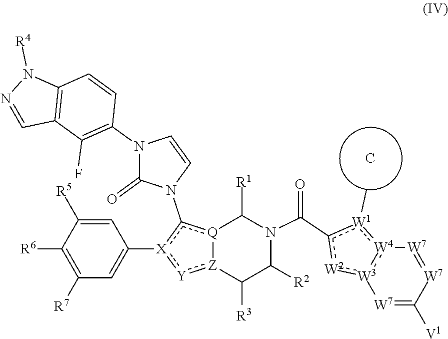

or a pharmaceutically acceptable salt thereof, stereoisomer thereof, tautomer thereof, analog thereof (e.g., deuterated analog thereof), or solvate thereof, wherein:
   each of R¹, R², and R³ is, independently, hydrogen (H), halo, hydroxyl, C₁₋₆ alkyl, or C₁₋₆ alkoxy;
   R⁴ is hydrogen (H), halo, C₁₋₆ alkyl, C₁₋₆ haloalkyl, C₁₋₆ alkoxy, C₁₋₆ haloalkoxy, C₃₋₈ cycloalkyl, or C₃₋₈ halocycloalkyl;
   each of R⁵, R⁶, and R⁷ is, independently, hydrogen (H), halo, CN, C₁₋₆ alkyl, C₁₋₆ haloalkyl, C₁₋₆ alkoxy, C₁₋₆ haloalkoxy, C₃₋₈ cycloalkyl, or C₃₋₈ halocycloalkyl; each of X, Y, Z, and Q is, independently, C or N, and the dashed line represents an optional double bond;
   each of W¹, W³, and W⁴ is, independently, C or N;
   W² is CR^{wa}, NR^{wa}, N, O, or S, and the dashed line represents an optional double bond;
   each of W⁵, W⁶, and W⁷ is, independently, CR^{wa} or N;
   each R^{wa}, if present, is, independently, hydrogen (H), halo, or C₁₋₆ alkyl;
   C is N

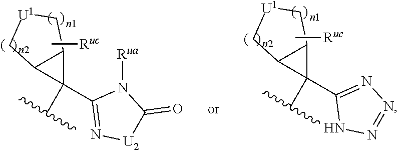

optionally wherein C is

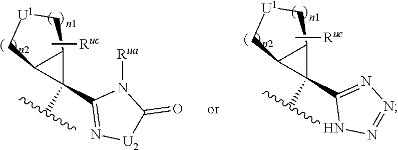

U¹ is —O—, —S—, —S(O)—, —S(O)₂—, —N(R^{ua})—, or —CH₂—;
U² is O, —N(R^{ua})—, or S; optionally, U² is O or S;
each R^{ua} is, independently, hydrogen (H), C₁₋₆ alkyl, C₁₋₆ alkoxy, 5-10 membered heterocyclyl, —S(O)₂—, or C₃₋₈ cycloalkyl, wherein each C₁₋₆ alkyl, C₁₋₆ alkoxy, 5-10 membered heterocyclyl, —S(O)₂—, or C₃₋₈ cycloalkyl of R^{ua} is independently optionally substituted with halo, oxo, hydroxyl, amino, C₁₋₆ alkyl, C₁₋₆ haloalkyl, or C₃₋₈ cycloalkyl; each R^{uc} is, independently, hydrogen (H), hydroxyl, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, —NR^{N1}R^{N2}, or an isotope, wherein one or more R^{uc} can be present on the ring to which it is attached; wherein each of R^{N1} and R^{N2} is, independently, H, C₁₋₆ aliphatic, C₁₋₆ alkyl, C₁₋₆ heteroaliphatic, C₁₋₆ heteroalkyl, aromatic, or aryl; or where R^{N1} and R^{N2}, taken together with the nitrogen atom to which each are attached, form a heterocyclyl group;
each of n1 and n2 is, independently, 0, 1, 2, or 3; and
V¹ is

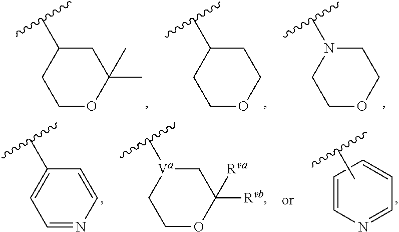

wherein:
V^{a} is N or CR^{vc}, and
each of R^{va}, R^{vb}, and R^{vc}, if present, is, independently, hydrogen (H), halo, or C₁₋₆ alkyl.

In some embodiments, the compound of Formula (IV) is a compound of Formula (IVa):

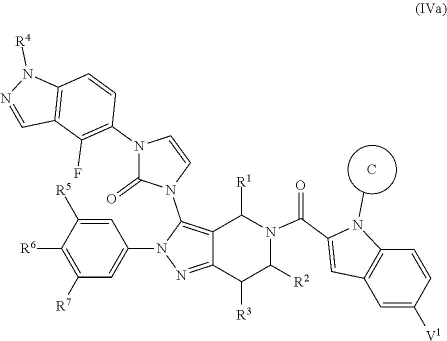

or a pharmaceutically acceptable salt thereof, stereoisomer thereof, tautomer thereof, analog thereof (e.g., deuterated analog thereof), or solvate thereof.

In some embodiments, the compound is a compound of Formula (IVa), wherein C is

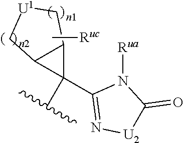

### and V¹ is

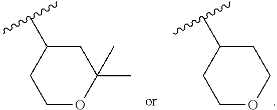

In some embodiments, C is

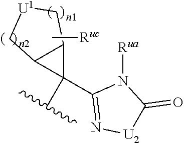

### and V¹ is

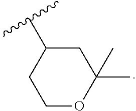 [SMILES: *C1CCOC(C)(C)C1]

In some embodiments, C is

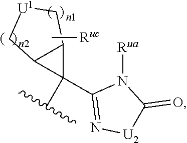

### V¹ is

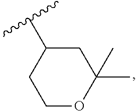 [SMILES: *C1CCOC(C)(C)C1]

and U¹ is —N(R^{ua})—. In some embodiments and in conjunction with the embodiments above or below, C is

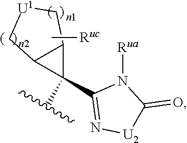

U¹ is —N(R^{ua})—, and U² is O. In some embodiments and in conjunction with the embodiments above or below, U¹ is N(R^{ua})— and R^{ua} is a substituted or unsubstituted 5 or 6 membered heteroaryl. In some embodiments and in conjunction with the embodiments above or below, U¹ is N(R^{ua})— and R^{ua} is an unsubstituted 5 or 6 membered heteroaryl. In some embodiments and in conjunction with the embodiments above or below, U¹ is N(R^{ua})— and R^{ua} is an unsubstituted 6 membered heteroaryl (e.g., pyridine or pyrimidine). In some embodiments and in conjunction with the embodiments above or below, V¹ is

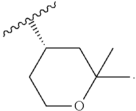 [SMILES: *[C@H]1CCOC(C)(C)C1]

In some embodiments and in conjunction with the embodiments above or below, each of R⁵, R⁶, and R⁷ is, independently, halo, CN, C₁₋₆ alkyl, C₁₋₆ haloalkyl, C₁₋₆ alkoxy, C₁₋₆ haloalkoxy, C₃₋₈ cycloalkyl, or C₃₋₈ halocycloalkyl. In some embodiments and in conjunction with the embodiments above or below, R⁶ is halo (e.g., fluoro).

In some embodiments, the compound of Formula (IV) is a compound of Formula (IVb)

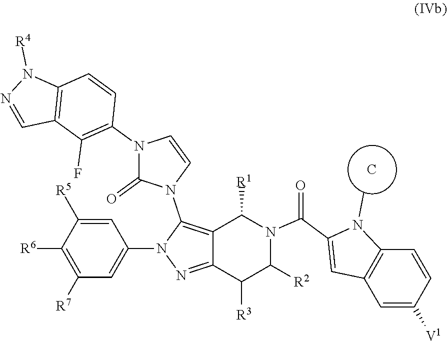

or a pharmaceutically acceptable salt thereof, stereoisomer thereof, tautomer thereof, analog thereof (e.g., deuterated analog thereof), or solvate thereof.

In some embodiments, R⁴ is —CH₃.

In some embodiments, R⁵ and R⁷ are each —CH₃.

In some embodiments, R⁶ is halo.

The present disclosure relates, in part, to a compound having a structure in Table 1, as well as pharmaceutically acceptable salt thereof, stereoisomers thereof, analogs thereof, solvates thereof, prodrugs thereof, or tautomers thereof. In some embodiments, the compound is a compound having a structure in Table 1 or a pharmaceutically acceptable salt thereof.

TABLE 1
Non-limiting compounds

| No. | Structure |
|---|---|
| 1 | 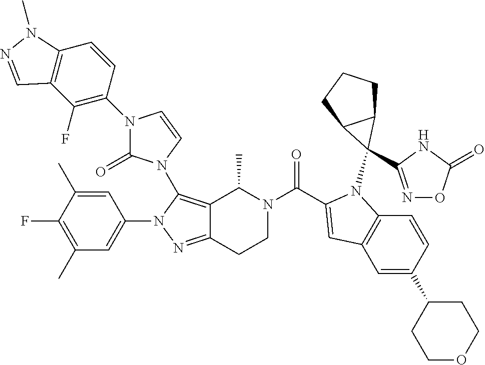 |
| 2 | 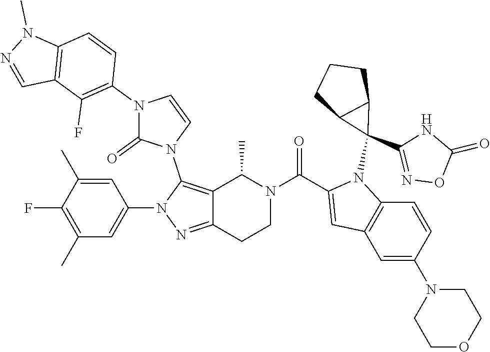 [SMILES: Cc1cc(-n2nc3c(c2-n2ccn(-c4ccc5c(cnn5C)c4F)c2=O)[C@H](C)N(C(=O)c2cc4cc(N5CCOCC5)ccc4n2[C@@]2(c4noc(=O)[nH]4)[C@@H]4CCC[C@@H]42)CC3)cc(C)c1F] |
| 3 | 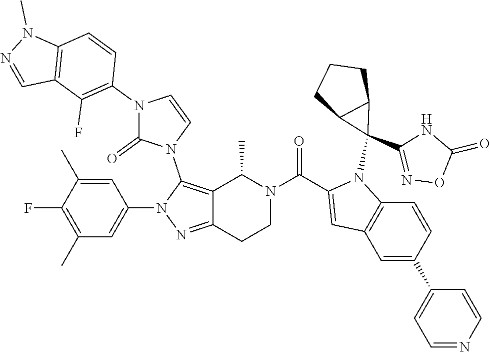 |
| 4 | 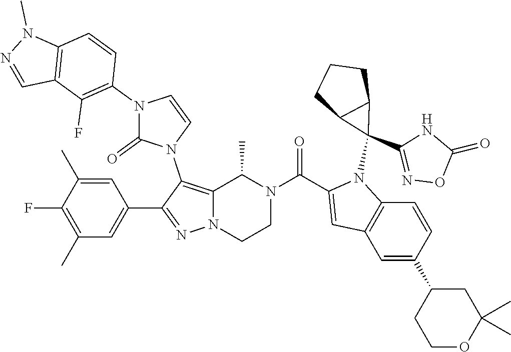 [SMILES: Cc1cc(-c2nn3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CCC[C@@H]42)CC3)cc(C)c1F] |
| 5 | 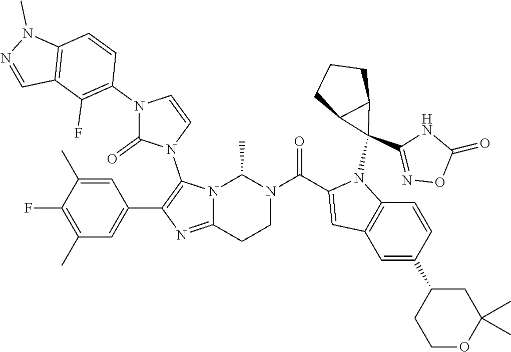 [SMILES: Cc1cc(-c2nc3n(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CCC[C@@H]42)CC3)cc(C)c1F] |
| 6 | 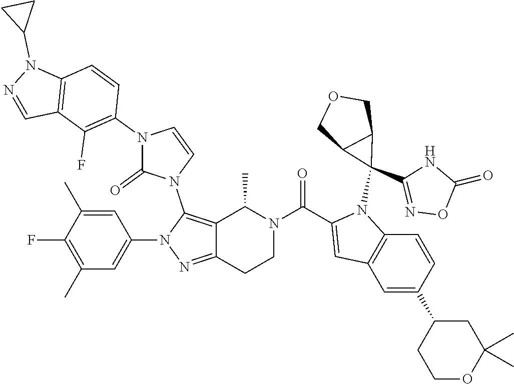 [SMILES: Cc1cc(-n2nc3c(c2-n2ccn(-c4ccc5c(cnn5C5CC5)c4F)c2=O)[C@H](C)N(C(=O)c2cc4cc([C@H]5CCOC(C)(C)C5)ccc4n2[C@@]2(c4noc(=O)[nH]4)[C@@H]4COC[C@@H]42)CC3)cc(C)c1F] |
| 7 | 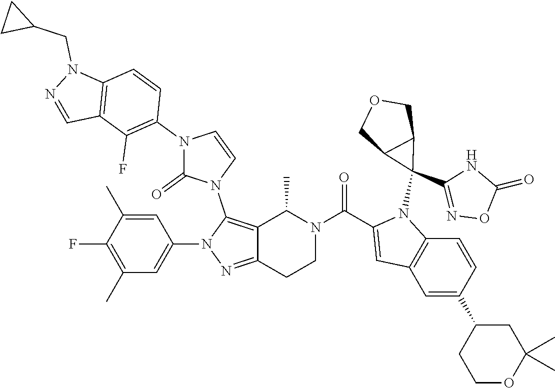 [SMILES: Cc1cc(-n2nc3c(c2-n2ccn(-c4ccc5c(cnn5CC5CC5)c4F)c2=O)[C@H](C)N(C(=O)c2cc4cc([C@H]5CCOC(C)(C)C5)ccc4n2[C@@]2(c4noc(=O)[nH]4)[C@@H]4COC[C@@H]42)CC3)cc(C)c1F] |
| 8 | 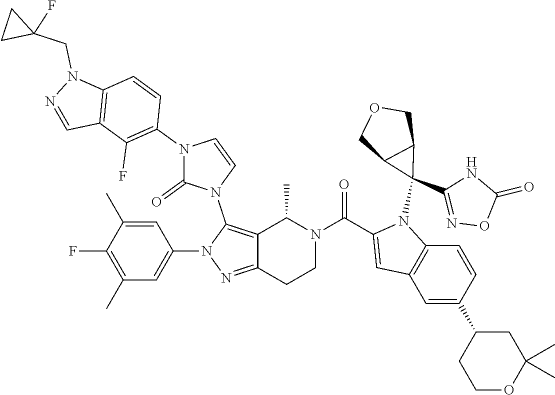 [SMILES: Cc1cc(-n2nc3c(c2-n2ccn(-c4ccc5c(cnn5CC5(F)CC5)c4F)c2=O)[C@H](C)N(C(=O)c2cc4cc([C@H]5CCOC(C)(C)C5)ccc4n2[C@@]2(c4noc(=O)[nH]4)[C@@H]4COC[C@@H]42)CC3)cc(C)c1F] |
| 9 | 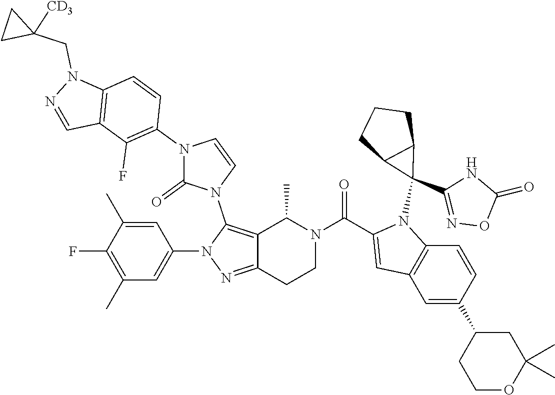 [SMILES: [2H]C([2H])([2H])C1(Cn2ncc3c(F)c(-n4ccn(-c5c6c(nn5-c5cc(C)c(F)c(C)c5)CCN(C(=O)c5cc7cc([C@H]8CCOC(C)(C)C8)ccc7n5[C@@]5(c7noc(=O)[nH]7)[C@@H]7CCC[C@@H]75)[C@H]6C)c4=O)ccc32)CC1] |
| 10 | 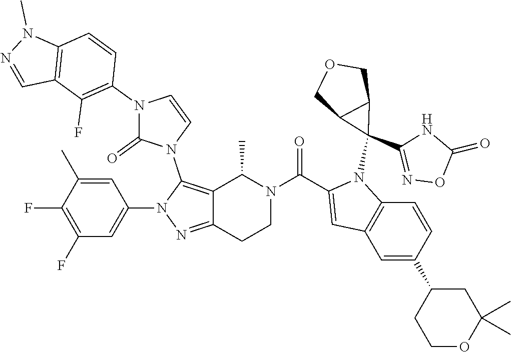 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4COC[C@@H]42)CC3)cc(F)c1F] |
| 11 | 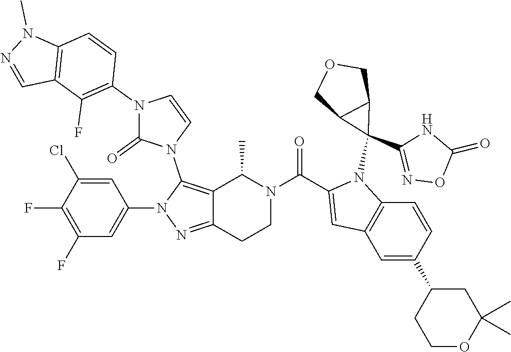 [SMILES: C[C@H]1c2c(nn(-c3cc(F)c(F)c(Cl)c3)c2-n2ccn(-c3ccc4c(cnn4C)c3F)c2=O)CCN1C(=O)c1cc2cc([C@H]3CCOC(C)(C)C3)ccc2n1[C@@]1(c2noc(=O)[nH]2)[C@@H]2COC[C@@H]21] |
| 12 | 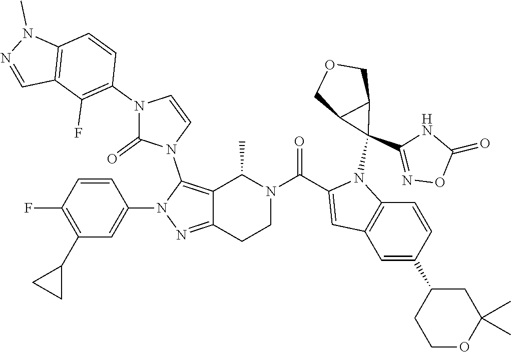 [SMILES: C[C@H]1c2c(nn(-c3ccc(F)c(C4CC4)c3)c2-n2ccn(-c3ccc4c(cnn4C)c3F)c2=O)CCN1C(=O)c1cc2cc([C@H]3CCOC(C)(C)C3)ccc2n1[C@@]1(c2noc(=O)[nH]2)[C@@H]2COC[C@@H]21] |
| 13 | 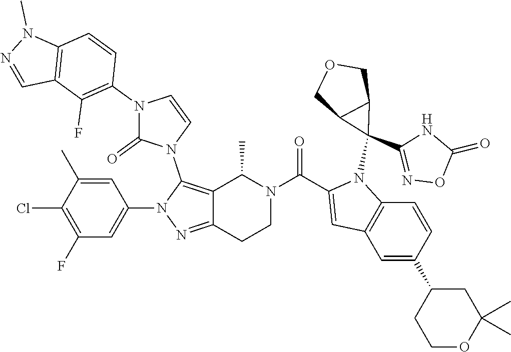 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4COC[C@@H]42)CC3)cc(F)c1Cl] |
| 14 | 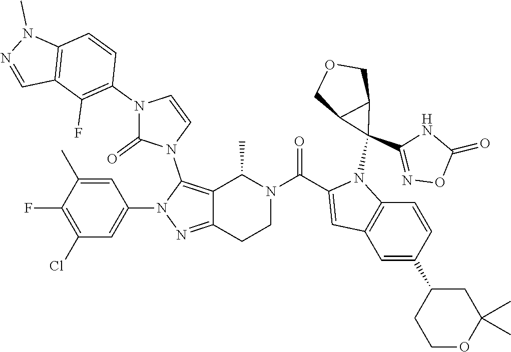 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4COC[C@@H]42)CC3)cc(Cl)c1F] |
| 15 | 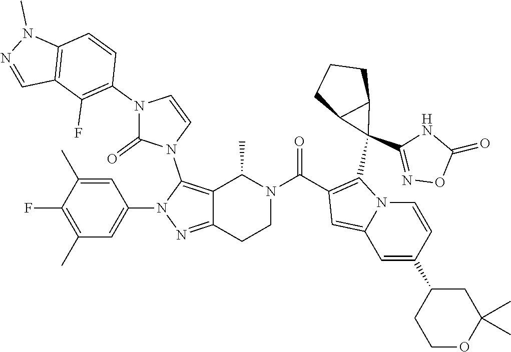 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(C)c1F] |
| 16 | 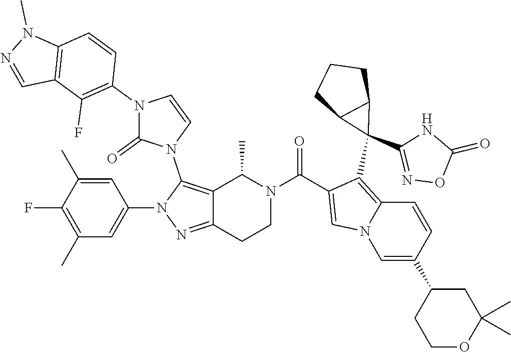 [SMILES: Cc1cc(-n2nc3c(c2-n2ccn(-c4ccc5c(cnn5C)c4F)c2=O)[C@H](C)N(C(=O)c2cn4cc([C@H]5CCOC(C)(C)C5)ccc4c2[C@@]2(c4noc(=O)[nH]4)[C@@H]4CCC[C@@H]42)CC3)cc(C)c1F] |
| 17 | 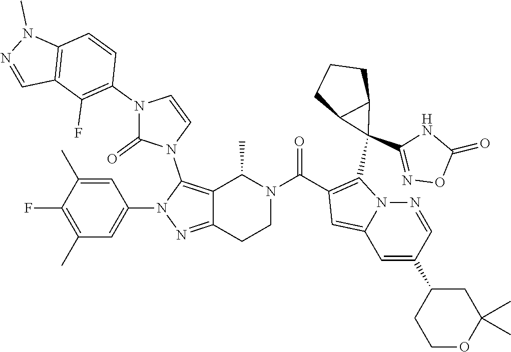 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)cnn4c2[C@@]2(c4noc(=O)[nH]4)[C@@H]4CCC[C@@H]42)CC3)cc(C)c1F] |
| 18 | 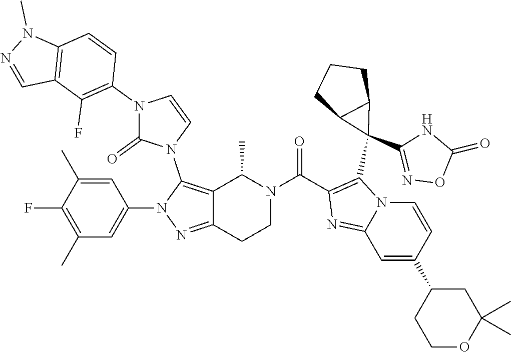 [SMILES: Cc1cc(-n2nc3c(c2-n2ccn(-c4ccc5c(cnn5C)c4F)c2=O)[C@H](C)N(C(=O)c2nc4cc([C@H]5CCOC(C)(C)C5)ccn4c2[C@@]2(c4noc(=O)[nH]4)[C@@H]4CCC[C@@H]42)CC3)cc(C)c1F] |
| 19 | 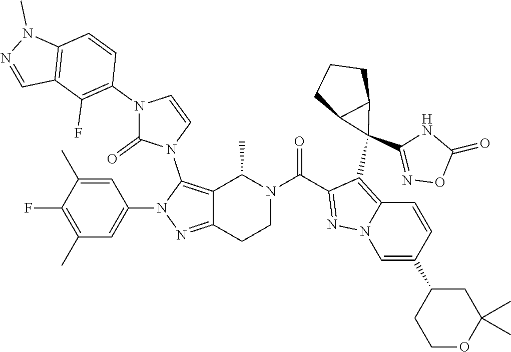 [SMILES: Cc1cc(-n2nc3c(c2-n2ccn(-c4ccc5c(cnn5C)c4F)c2=O)[C@H](C)N(C(=O)c2nn4cc([C@H]5CCOC(C)(C)C5)ccc4c2[C@@]2(c4noc(=O)[nH]4)[C@@H]4CCC[C@@H]42)CC3)cc(C)c1F] |
| 20 | 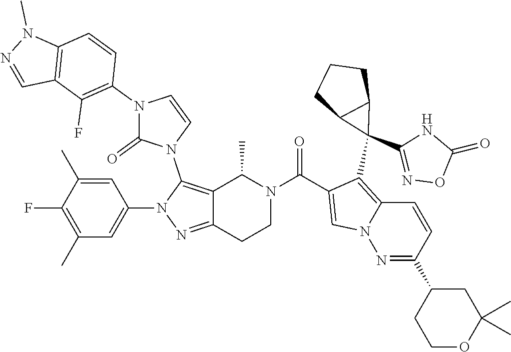 [SMILES: Cc1cc(-n2nc3c(c2-n2ccn(-c4ccc5c(cnn5C)c4F)c2=O)[C@H](C)N(C(=O)c2cn4nc([C@H]5CCOC(C)(C)C5)ccc4c2[C@@]2(c4noc(=O)[nH]4)[C@@H]4CCC[C@@H]42)CC3)cc(C)c1F] |
| 21 | 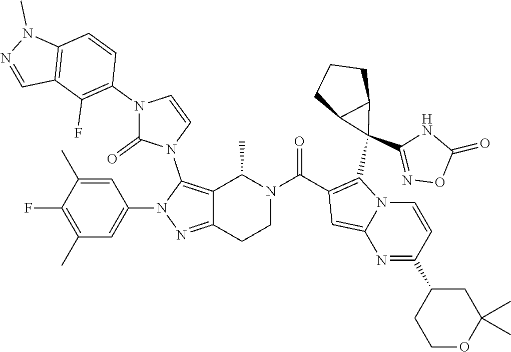 [SMILES: Cc1cc(-n2nc3c(c2-n2ccn(-c4ccc5c(cnn5C)c4F)c2=O)[C@H](C)N(C(=O)c2cc4nc([C@H]5CCOC(C)(C)C5)ccn4c2[C@@]2(c4noc(=O)[nH]4)[C@@H]4CCC[C@@H]42)CC3)cc(C)c1F] |
| 22 | 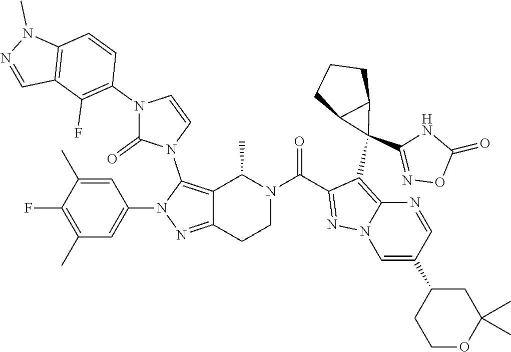 [SMILES: Cc1cc(-n2nc3c(c2-n2ccn(-c4ccc5c(cnn5C)c4F)c2=O)[C@H](C)N(C(=O)c2nn4cc([C@H]5CCOC(C)(C)C5)cnc4c2[C@@]2(c4noc(=O)[nH]4)[C@@H]4CCC[C@@H]42)CC3)cc(C)c1F] |
| 23 | 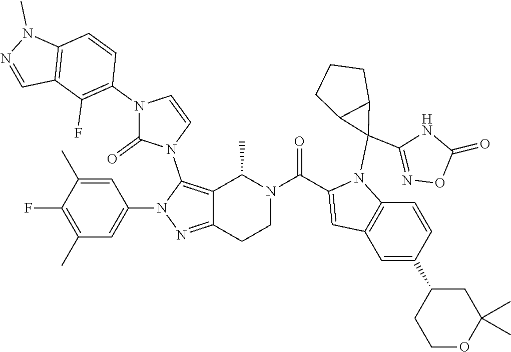 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2C2(c4noc(=O)[nH]4)C4CCCC42)CC3)cc(C)c1F] |
| 24 | 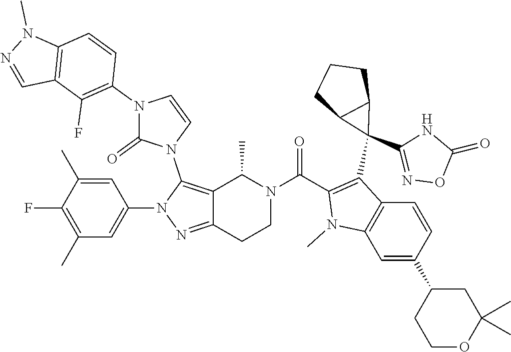 [SMILES: Cc1cc(-n2nc3c(c2-n2ccn(-c4ccc5c(cnn5C)c4F)c2=O)[C@H](C)N(C(=O)c2c([C@@]4(c5noc(=O)[nH]5)[C@@H]5CCC[C@@H]54)c4ccc([C@H]5CCOC(C)(C)C5)cc4n2C)CC3)cc(C)c1F] |
| 25 | 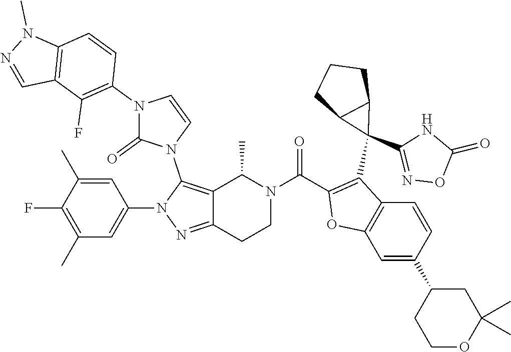 [SMILES: Cc1cc(-n2nc3c(c2-n2ccn(-c4ccc5c(cnn5C)c4F)c2=O)[C@H](C)N(C(=O)c2oc4cc([C@H]5CCOC(C)(C)C5)ccc4c2[C@@]2(c4noc(=O)[nH]4)[C@@H]4CCC[C@@H]42)CC3)cc(C)c1F] |
| 26 | 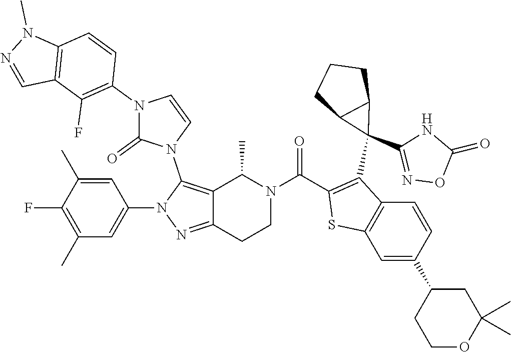 [SMILES: Cc1cc(-n2nc3c(c2-n2ccn(-c4ccc5c(cnn5C)c4F)c2=O)[C@H](C)N(C(=O)c2sc4cc([C@H]5CCOC(C)(C)C5)ccc4c2[C@@]2(c4noc(=O)[nH]4)[C@@H]4CCC[C@@H]42)CC3)cc(C)c1F] |
| 27 | 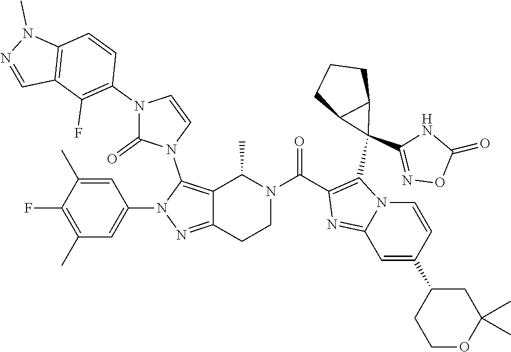 [SMILES: Cc1cc(-n2nc3c(c2-n2ccn(-c4ccc5c(cnn5C)c4F)c2=O)[C@H](C)N(C(=O)c2nc4cc([C@H]5CCOC(C)(C)C5)ccn4c2[C@@]2(c4noc(=O)[nH]4)[C@@H]4CCC[C@@H]42)CC3)cc(C)c1F] |
| 28 | 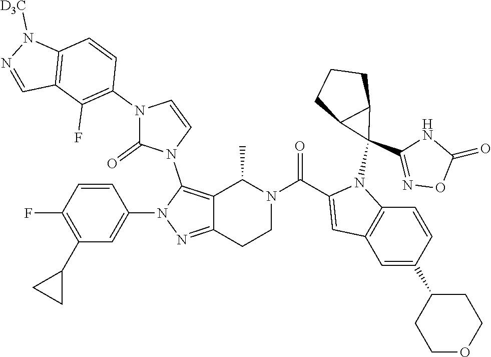 |
| 29 | 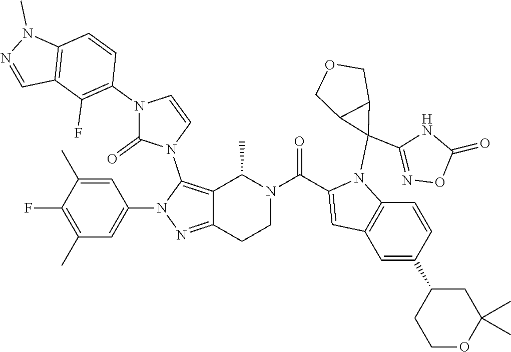 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2C2(c4noc(=O)[nH]4)C4COCC42)CC3)cc(C)c1F] |
| 30 | 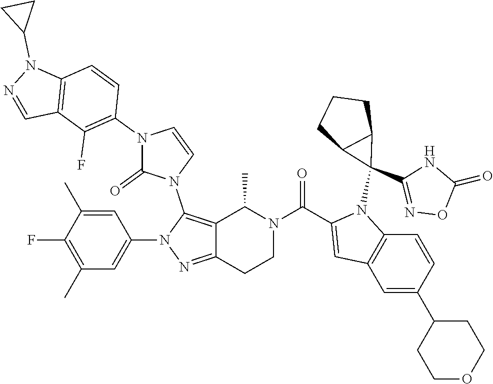 [SMILES: Cc1cc(-n2nc3c(c2-n2ccn(-c4ccc5c(cnn5C5CC5)c4F)c2=O)[C@H](C)N(C(=O)c2cc4cc(C5CCOCC5)ccc4n2[C@@]2(c4noc(=O)[nH]4)[C@@H]4CCC[C@@H]42)CC3)cc(C)c1F] |
| 31 | 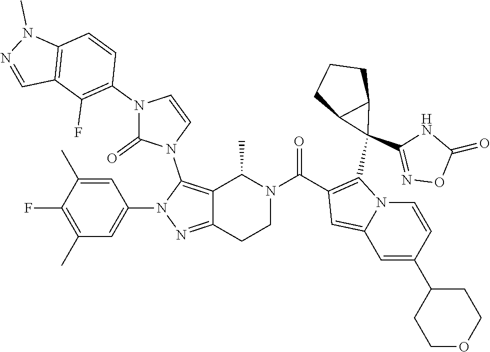 [SMILES: Cc1cc(-n2nc3c(c2-n2ccn(-c4ccc5c(cnn5C)c4F)c2=O)[C@H](C)N(C(=O)c2cc4cc(C5CCOCC5)ccn4c2[C@@]2(c4noc(=O)[nH]4)[C@@H]4CCC[C@@H]42)CC3)cc(C)c1F] |
| 32 | 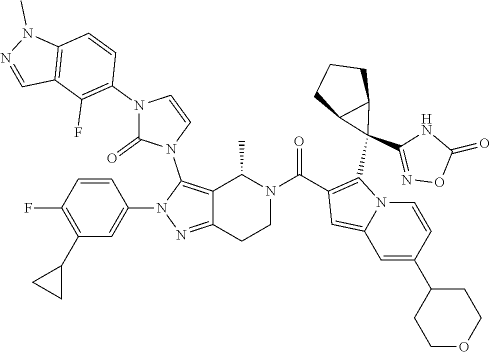 [SMILES: C[C@H]1c2c(nn(-c3ccc(F)c(C4CC4)c3)c2-n2ccn(-c3ccc4c(cnn4C)c3F)c2=O)CCN1C(=O)c1cc2cc(C3CCOCC3)ccn2c1[C@@]1(c2noc(=O)[nH]2)[C@@H]2CCC[C@@H]21] |
| 33 | 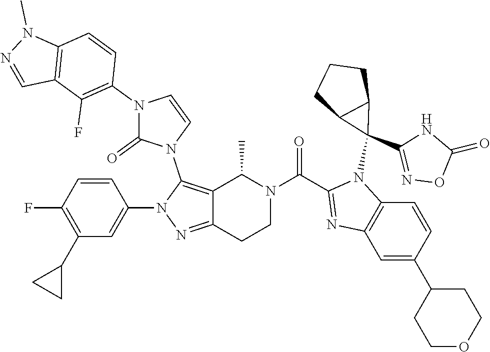 [SMILES: C[C@H]1c2c(nn(-c3ccc(F)c(C4CC4)c3)c2-n2ccn(-c3ccc4c(cnn4C)c3F)c2=O)CCN1C(=O)c1nc2cc(C3CCOCC3)ccc2n1[C@@]1(c2noc(=O)[nH]2)[C@@H]2CCC[C@@H]21] |
| 34 | 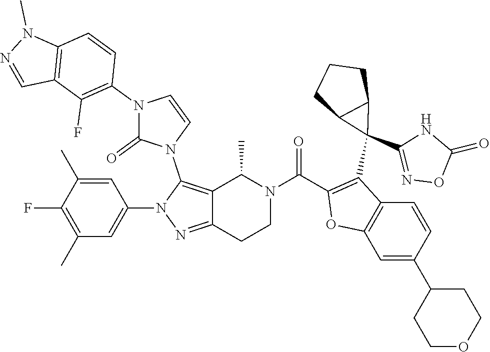 [SMILES: Cc1cc(-n2nc3c(c2-n2ccn(-c4ccc5c(cnn5C)c4F)c2=O)[C@H](C)N(C(=O)c2oc4cc(C5CCOCC5)ccc4c2[C@@]2(c4noc(=O)[nH]4)[C@@H]4CCC[C@@H]42)CC3)cc(C)c1F] |
| 35 | 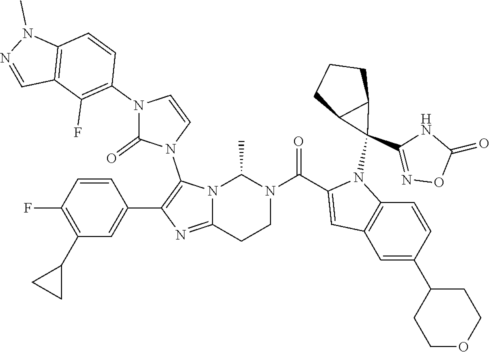 [SMILES: C[C@@H]1N(C(=O)c2cc3cc(C4CCOCC4)ccc3n2[C@@]2(c3noc(=O)[nH]3)[C@@H]3CCC[C@@H]32)CCc2nc(-c3ccc(F)c(C4CC4)c3)c(-n3ccn(-c4ccc5c(cnn5C)c4F)c3=O)n21] |
| 36 | 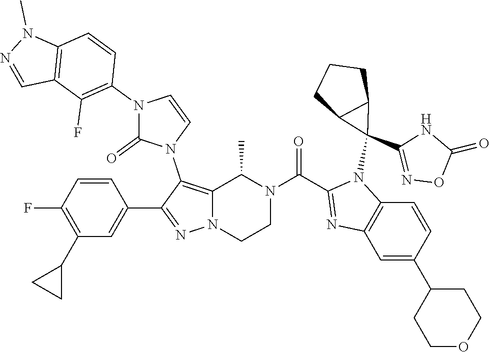 [SMILES: C[C@H]1c2c(-n3ccn(-c4ccc5c(cnn5C)c4F)c3=O)c(-c3ccc(F)c(C4CC4)c3)nn2CCN1C(=O)c1nc2cc(C3CCOCC3)ccc2n1[C@@]1(c2noc(=O)[nH]2)[C@@H]2CCC[C@@H]21] |
| 37 | 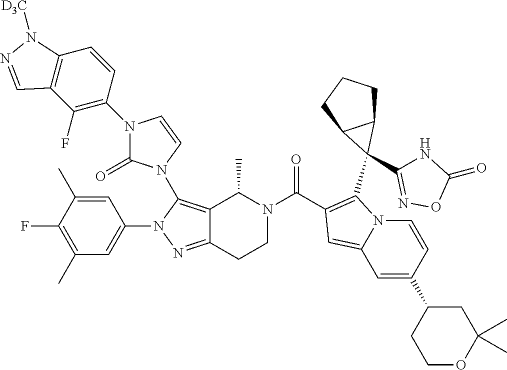 [SMILES: [2H]C([2H])([2H])n1ncc2c(F)c(-n3ccn(-c4c5c(nn4-c4cc(C)c(F)c(C)c4)CCN(C(=O)c4cc6cc([C@H]7CCOC(C)(C)C7)ccn6c4[C@@]4(c6noc(=O)[nH]6)[C@@H]6CCC[C@@H]64)[C@H]5C)c3=O)ccc21] |
| 38 | 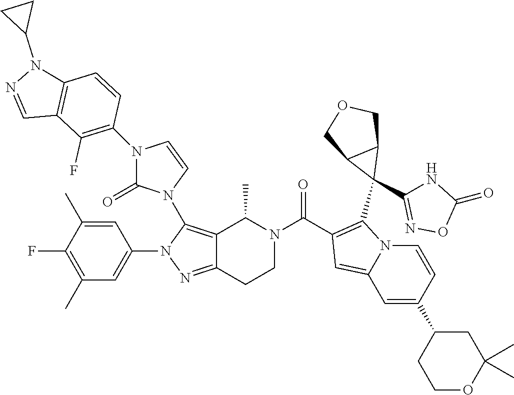 [SMILES: Cc1cc(-n2nc3c(c2-n2ccn(-c4ccc5c(cnn5C5CC5)c4F)c2=O)[C@H](C)N(C(=O)c2cc4cc([C@H]5CCOC(C)(C)C5)ccn4c2[C@@]2(c4noc(=O)[nH]4)[C@@H]4COC[C@@H]42)CC3)cc(C)c1F] |
| 39 | 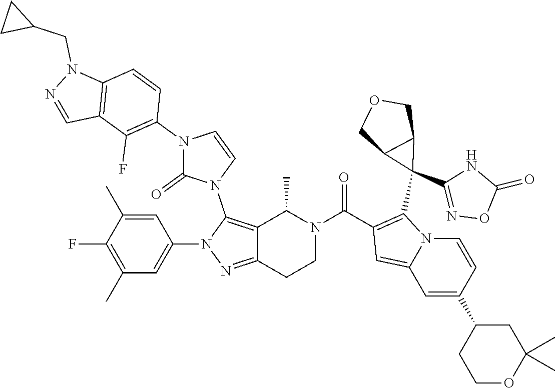 [SMILES: Cc1cc(-n2nc3c(c2-n2ccn(-c4ccc5c(cnn5CC5CC5)c4F)c2=O)[C@H](C)N(C(=O)c2cc4cc([C@H]5CCOC(C)(C)C5)ccn4c2[C@@]2(c4noc(=O)[nH]4)[C@@H]4COC[C@@H]42)CC3)cc(C)c1F] |
| 40 | 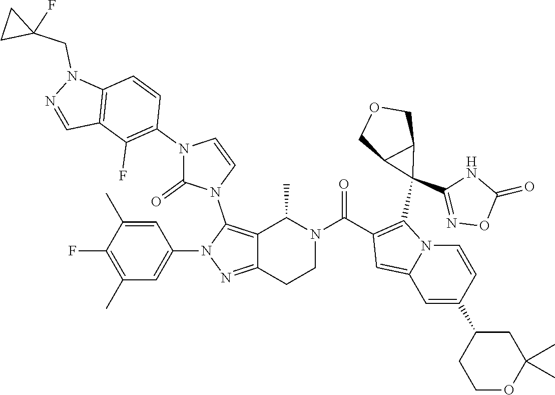 [SMILES: Cc1cc(-n2nc3c(c2-n2ccn(-c4ccc5c(cnn5CC5(F)CC5)c4F)c2=O)[C@H](C)N(C(=O)c2cc4cc([C@H]5CCOC(C)(C)C5)ccn4c2[C@@]2(c4noc(=O)[nH]4)[C@@H]4COC[C@@H]42)CC3)cc(C)c1F] |
| 41 | 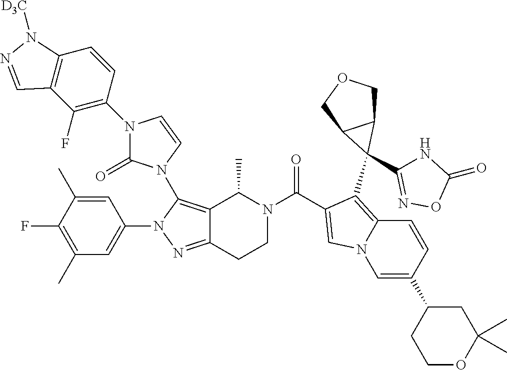 [SMILES: [2H]C([2H])([2H])n1ncc2c(F)c(-n3ccn(-c4c5c(nn4-c4cc(C)c(F)c(C)c4)CCN(C(=O)c4cn6cc([C@H]7CCOC(C)(C)C7)ccc6c4[C@@]4(c6noc(=O)[nH]6)[C@@H]6COC[C@@H]64)[C@H]5C)c3=O)ccc21] |
| 42 | 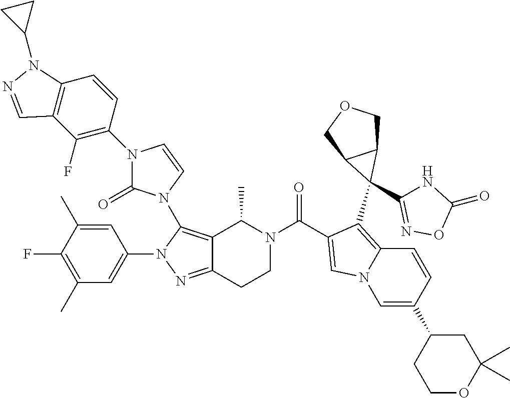 [SMILES: Cc1cc(-n2nc3c(c2-n2ccn(-c4ccc5c(cnn5C5CC5)c4F)c2=O)[C@H](C)N(C(=O)c2cn4cc([C@H]5CCOC(C)(C)C5)ccc4c2[C@@]2(c4noc(=O)[nH]4)[C@@H]4COC[C@@H]42)CC3)cc(C)c1F] |
| 43 | 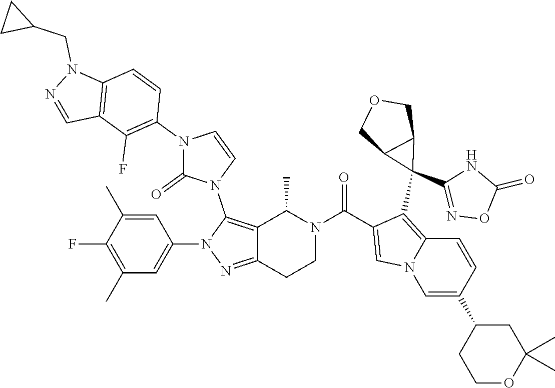 [SMILES: Cc1cc(-n2nc3c(c2-n2ccn(-c4ccc5c(cnn5CC5CC5)c4F)c2=O)[C@H](C)N(C(=O)c2cn4cc([C@H]5CCOC(C)(C)C5)ccc4c2[C@@]2(c4noc(=O)[nH]4)[C@@H]4COC[C@@H]42)CC3)cc(C)c1F] |
| 44 | 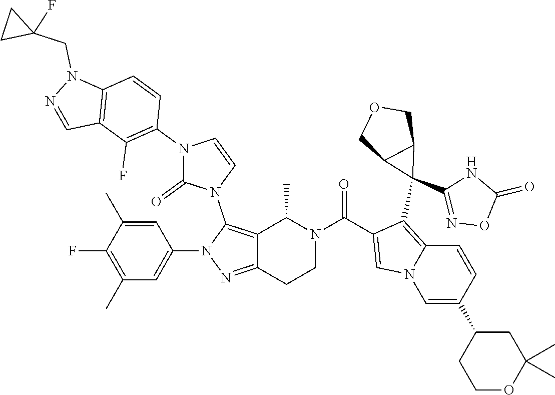 [SMILES: Cc1cc(-n2nc3c(c2-n2ccn(-c4ccc5c(cnn5CC5(F)CC5)c4F)c2=O)[C@H](C)N(C(=O)c2cn4cc([C@H]5CCOC(C)(C)C5)ccc4c2[C@@]2(c4noc(=O)[nH]4)[C@@H]4COC[C@@H]42)CC3)cc(C)c1F] |
| 45 | 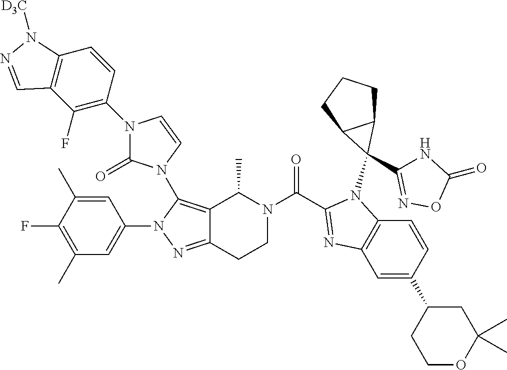 [SMILES: [2H]C([2H])([2H])n1ncc2c(F)c(-n3ccn(-c4c5c(nn4-c4cc(C)c(F)c(C)c4)CCN(C(=O)c4nc6cc([C@H]7CCOC(C)(C)C7)ccc6n4[C@@]4(c6noc(=O)[nH]6)[C@@H]6CCC[C@@H]64)[C@H]5C)c3=O)ccc21] |
| 46 | 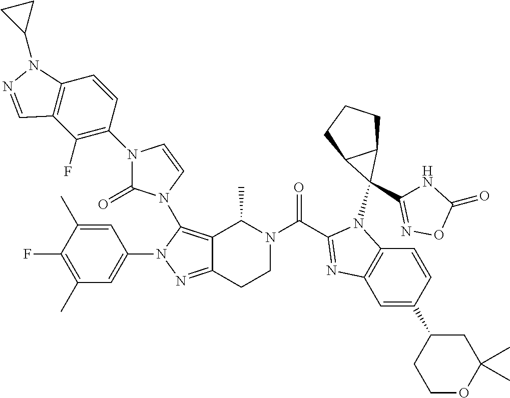 [SMILES: Cc1cc(-n2nc3c(c2-n2ccn(-c4ccc5c(cnn5C5CC5)c4F)c2=O)[C@H](C)N(C(=O)c2nc4cc([C@H]5CCOC(C)(C)C5)ccc4n2[C@@]2(c4noc(=O)[nH]4)[C@@H]4CCC[C@@H]42)CC3)cc(C)c1F] |
| 47 | 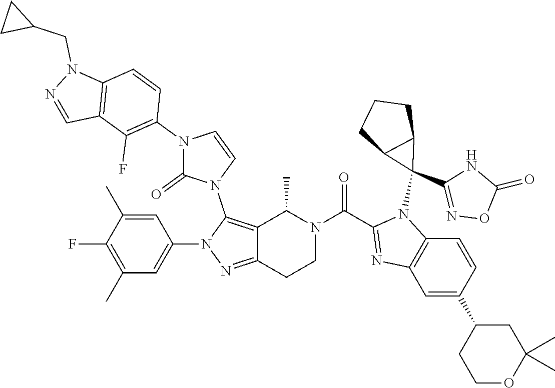 [SMILES: Cc1cc(-n2nc3c(c2-n2ccn(-c4ccc5c(cnn5CC5CC5)c4F)c2=O)[C@H](C)N(C(=O)c2nc4cc([C@H]5CCOC(C)(C)C5)ccc4n2[C@@]2(c4noc(=O)[nH]4)[C@@H]4CCC[C@@H]42)CC3)cc(C)c1F] |
| 48 | 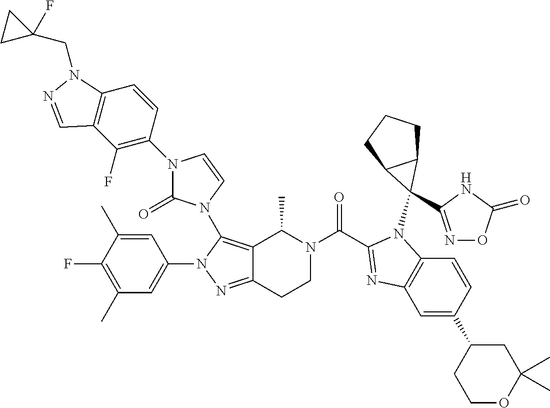 [SMILES: Cc1cc(-n2nc3c(c2-n2ccn(-c4ccc5c(cnn5CC5(F)CC5)c4F)c2=O)[C@H](C)N(C(=O)c2nc4cc([C@H]5CCOC(C)(C)C5)ccc4n2[C@@]2(c4noc(=O)[nH]4)[C@@H]4CCC[C@@H]42)CC3)cc(C)c1F] |
| 49 | 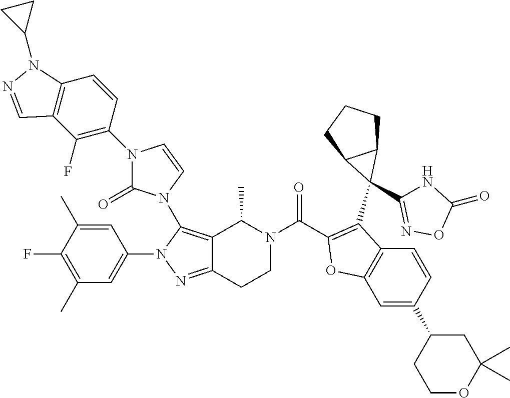 [SMILES: Cc1cc(-n2nc3c(c2-n2ccn(-c4ccc5c(cnn5C5CC5)c4F)c2=O)[C@H](C)N(C(=O)c2oc4cc([C@H]5CCOC(C)(C)C5)ccc4c2[C@@]2(c4noc(=O)[nH]4)[C@@H]4CCC[C@@H]42)CC3)cc(C)c1F] |
| 50 | 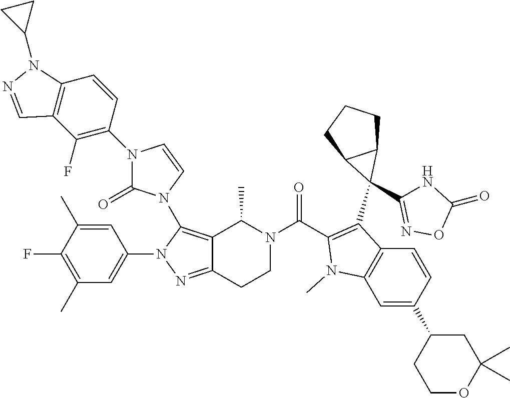 [SMILES: Cc1cc(-n2nc3c(c2-n2ccn(-c4ccc5c(cnn5C5CC5)c4F)c2=O)[C@H](C)N(C(=O)c2c([C@@]4(c5noc(=O)[nH]5)[C@@H]5CCC[C@@H]54)c4ccc([C@H]5CCOC(C)(C)C5)cc4n2C)CC3)cc(C)c1F] |
| 51 | 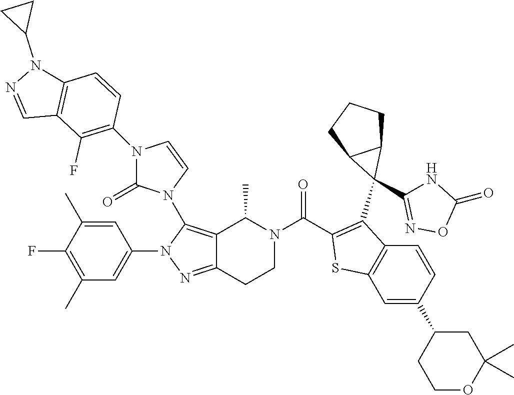 [SMILES: Cc1cc(-n2nc3c(c2-n2ccn(-c4ccc5c(cnn5C5CC5)c4F)c2=O)[C@H](C)N(C(=O)c2sc4cc([C@H]5CCOC(C)(C)C5)ccc4c2[C@@]2(c4noc(=O)[nH]4)[C@@H]4CCC[C@@H]42)CC3)cc(C)c1F] |
| 52 | 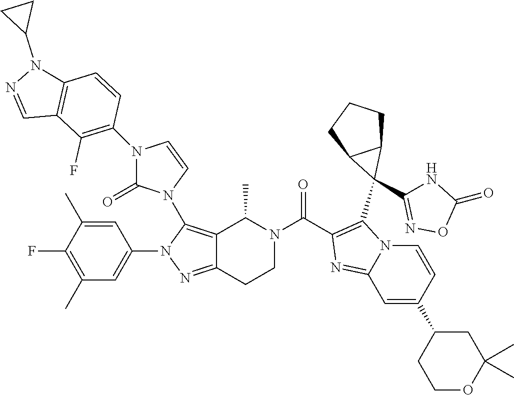 [SMILES: Cc1cc(-n2nc3c(c2-n2ccn(-c4ccc5c(cnn5C5CC5)c4F)c2=O)[C@H](C)N(C(=O)c2nc4cc([C@H]5CCOC(C)(C)C5)ccn4c2[C@@]2(c4noc(=O)[nH]4)[C@@H]4CCC[C@@H]42)CC3)cc(C)c1F] |
| 53 | 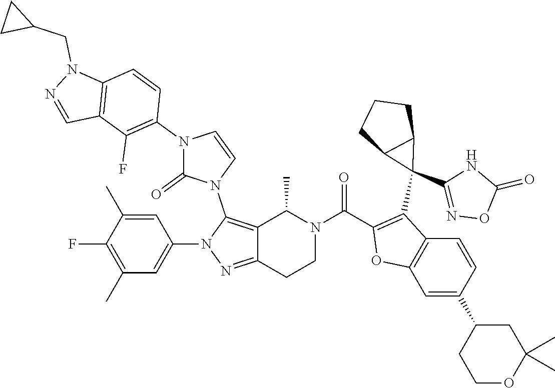 [SMILES: Cc1cc(-n2nc3c(c2-n2ccn(-c4ccc5c(cnn5CC5CC5)c4F)c2=O)[C@H](C)N(C(=O)c2oc4cc([C@H]5CCOC(C)(C)C5)ccc4c2[C@@]2(c4noc(=O)[nH]4)[C@@H]4CCC[C@@H]42)CC3)cc(C)c1F] |
| 54 | 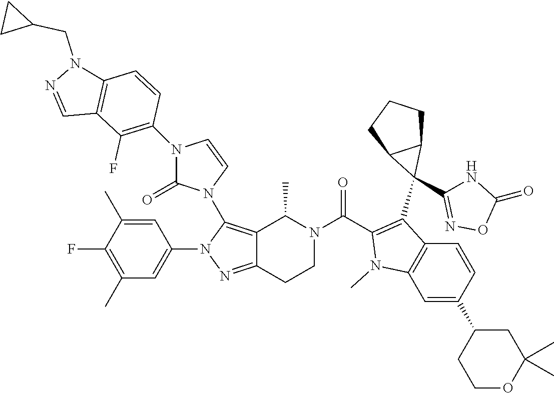 [SMILES: Cc1cc(-n2nc3c(c2-n2ccn(-c4ccc5c(cnn5CC5CC5)c4F)c2=O)[C@H](C)N(C(=O)c2c([C@@]4(c5noc(=O)[nH]5)[C@@H]5CCC[C@@H]54)c4ccc([C@H]5CCOC(C)(C)C5)cc4n2C)CC3)cc(C)c1F] |
| 55 | 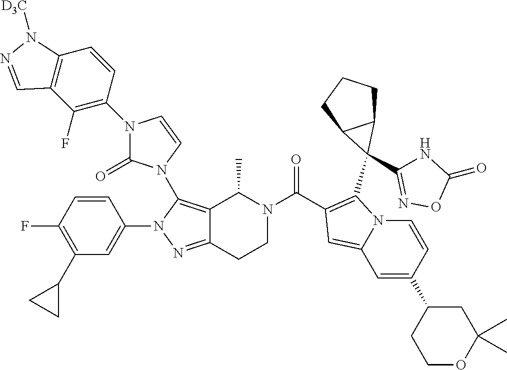 [SMILES: [2H]C([2H])([2H])n1ncc2c(F)c(-n3ccn(-c4c5c(nn4-c4ccc(F)c(C6CC6)c4)CCN(C(=O)c4cc6cc([C@H]7CCOC(C)(C)C7)ccn6c4[C@@]4(c6noc(=O)[nH]6)[C@@H]6CCC[C@@H]64)[C@H]5C)c3=O)ccc21] |
| 56 | 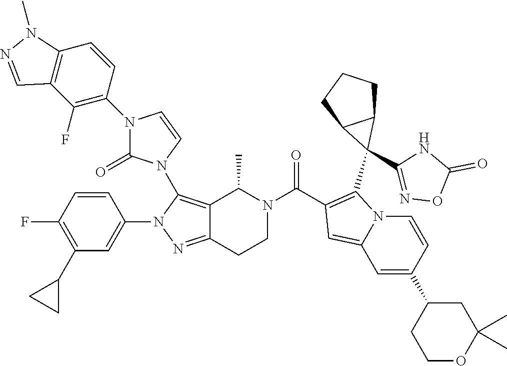 [SMILES: C[C@H]1c2c(nn(-c3ccc(F)c(C4CC4)c3)c2-n2ccn(-c3ccc4c(cnn4C)c3F)c2=O)CCN1C(=O)c1cc2cc([C@H]3CCOC(C)(C)C3)ccn2c1[C@@]1(c2noc(=O)[nH]2)[C@@H]2CCC[C@@H]21] |
| 57 | 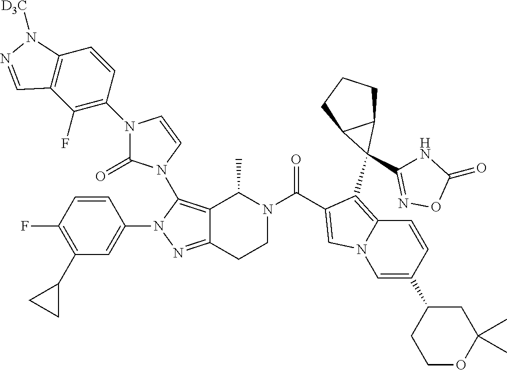 [SMILES: [2H]C([2H])([2H])n1ncc2c(F)c(-n3ccn(-c4c5c(nn4-c4ccc(F)c(C6CC6)c4)CCN(C(=O)c4cn6cc([C@H]7CCOC(C)(C)C7)ccc6c4[C@@]4(c6noc(=O)[nH]6)[C@@H]6CCC[C@@H]64)[C@H]5C)c3=O)ccc21] |
| 58 | 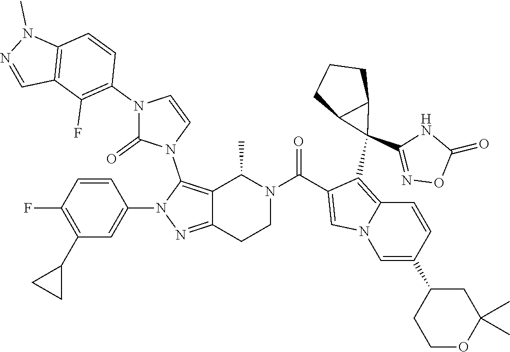 [SMILES: C[C@H]1c2c(nn(-c3ccc(F)c(C4CC4)c3)c2-n2ccn(-c3ccc4c(cnn4C)c3F)c2=O)CCN1C(=O)c1cn2cc([C@H]3CCOC(C)(C)C3)ccc2c1[C@@]1(c2noc(=O)[nH]2)[C@@H]2CCC[C@@H]21] |
| 59 | 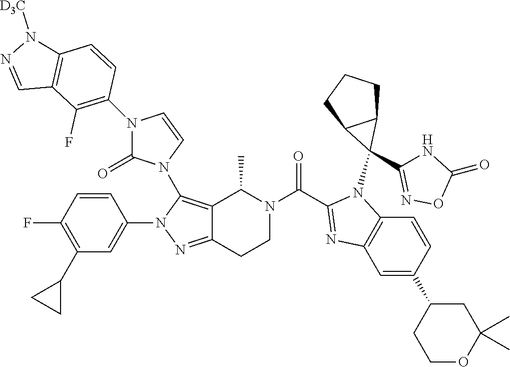 [SMILES: [2H]C([2H])([2H])n1ncc2c(F)c(-n3ccn(-c4c5c(nn4-c4ccc(F)c(C6CC6)c4)CCN(C(=O)c4nc6cc([C@H]7CCOC(C)(C)C7)ccc6n4[C@@]4(c6noc(=O)[nH]6)[C@@H]6CCC[C@@H]64)[C@H]5C)c3=O)ccc21] |
| 60 | 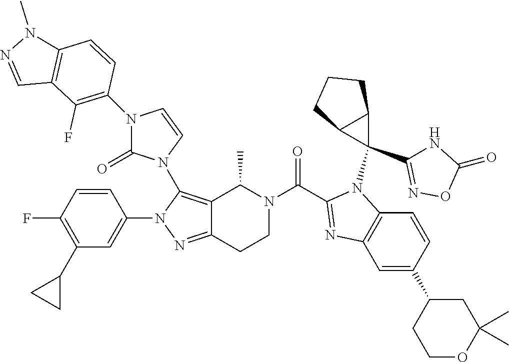 [SMILES: C[C@H]1c2c(nn(-c3ccc(F)c(C4CC4)c3)c2-n2ccn(-c3ccc4c(cnn4C)c3F)c2=O)CCN1C(=O)c1nc2cc([C@H]3CCOC(C)(C)C3)ccc2n1[C@@]1(c2noc(=O)[nH]2)[C@@H]2CCC[C@@H]21] |
| 61 | 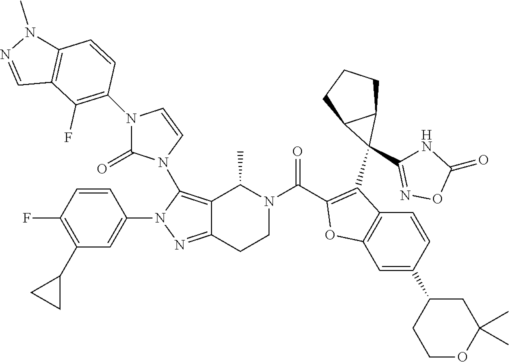 [SMILES: C[C@H]1c2c(nn(-c3ccc(F)c(C4CC4)c3)c2-n2ccn(-c3ccc4c(cnn4C)c3F)c2=O)CCN1C(=O)c1oc2cc([C@H]3CCOC(C)(C)C3)ccc2c1[C@@]1(c2noc(=O)[nH]2)[C@@H]2CCC[C@@H]21] |
| 62 | 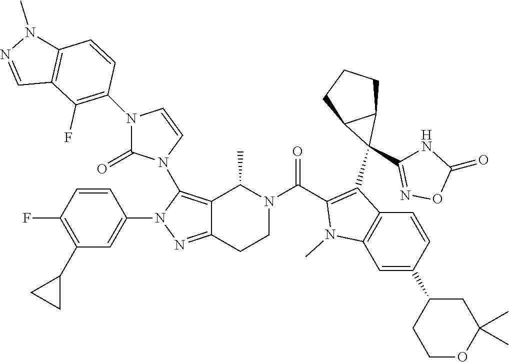 [SMILES: C[C@H]1c2c(nn(-c3ccc(F)c(C4CC4)c3)c2-n2ccn(-c3ccc4c(cnn4C)c3F)c2=O)CCN1C(=O)c1c([C@@]2(c3noc(=O)[nH]3)[C@@H]3CCC[C@@H]32)c2ccc([C@H]3CCOC(C)(C)C3)cc2n1C] |
| 63 | 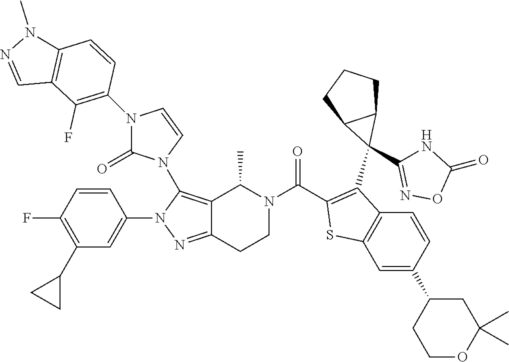 [SMILES: C[C@H]1c2c(nn(-c3ccc(F)c(C4CC4)c3)c2-n2ccn(-c3ccc4c(cnn4C)c3F)c2=O)CCN1C(=O)c1sc2cc([C@H]3CCOC(C)(C)C3)ccc2c1[C@@]1(c2noc(=O)[nH]2)[C@@H]2CCC[C@@H]21] |
| 64 | 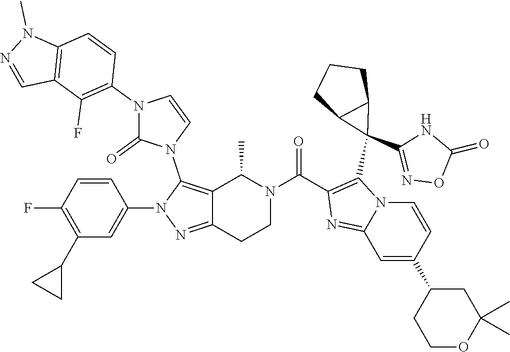 [SMILES: C[C@H]1c2c(nn(-c3ccc(F)c(C4CC4)c3)c2-n2ccn(-c3ccc4c(cnn4C)c3F)c2=O)CCN1C(=O)c1nc2cc([C@H]3CCOC(C)(C)C3)ccn2c1[C@@]1(c2noc(=O)[nH]2)[C@@H]2CCC[C@@H]21] |
| 65 | 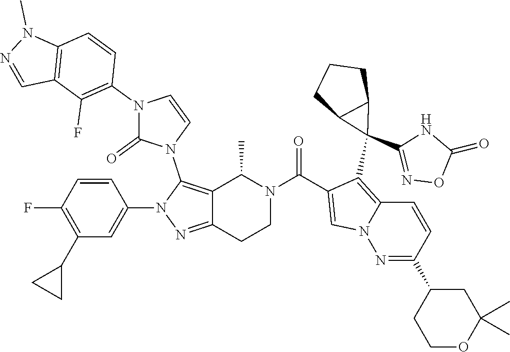 [SMILES: C[C@H]1c2c(nn(-c3ccc(F)c(C4CC4)c3)c2-n2ccn(-c3ccc4c(cnn4C)c3F)c2=O)CCN1C(=O)c1cn2nc([C@H]3CCOC(C)(C)C3)ccc2c1[C@@]1(c2noc(=O)[nH]2)[C@@H]2CCC[C@@H]21] |
| 66 | 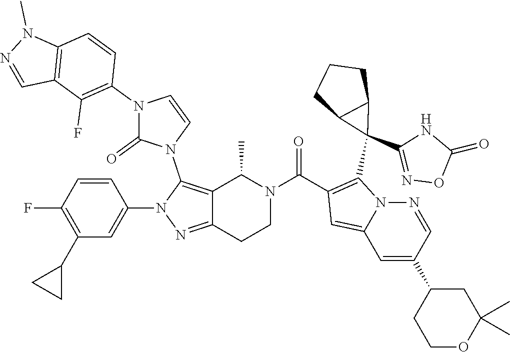 [SMILES: C[C@H]1c2c(nn(-c3ccc(F)c(C4CC4)c3)c2-n2ccn(-c3ccc4c(cnn4C)c3F)c2=O)CCN1C(=O)c1cc2cc([C@H]3CCOC(C)(C)C3)cnn2c1[C@@]1(c2noc(=O)[nH]2)[C@@H]2CCC[C@@H]21] |
| 67 | 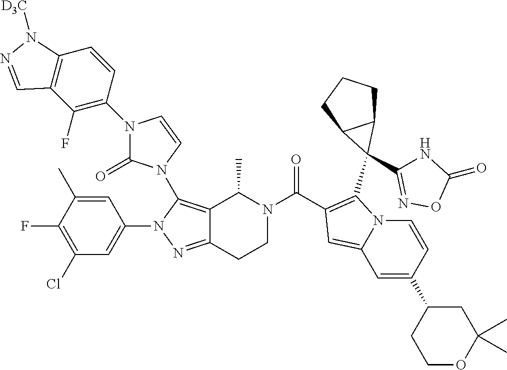 [SMILES: [2H]C([2H])([2H])n1ncc2c(F)c(-n3ccn(-c4c5c(nn4-c4cc(C)c(F)c(Cl)c4)CCN(C(=O)c4cc6cc([C@H]7CCOC(C)(C)C7)ccn6c4[C@@]4(c6noc(=O)[nH]6)[C@@H]6CCC[C@@H]64)[C@H]5C)c3=O)ccc21] |
| 68 | 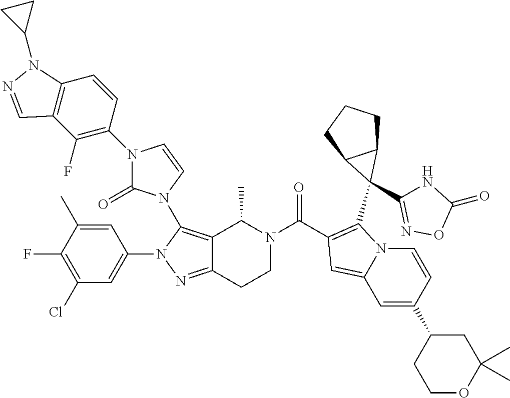 [SMILES: Cc1cc(-n2nc3c(c2-n2ccn(-c4ccc5c(cnn5C5CC5)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(Cl)c1F] |
| 69 | 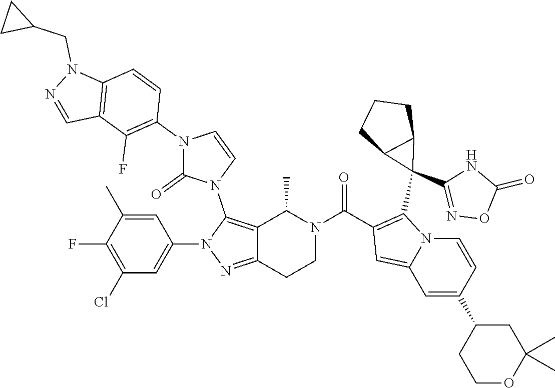 [SMILES: Cc1cc(-n2nc3c(c2-n2ccn(-c4ccc5c(cnn5CC5CC5)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(Cl)c1F] |
| 70 | 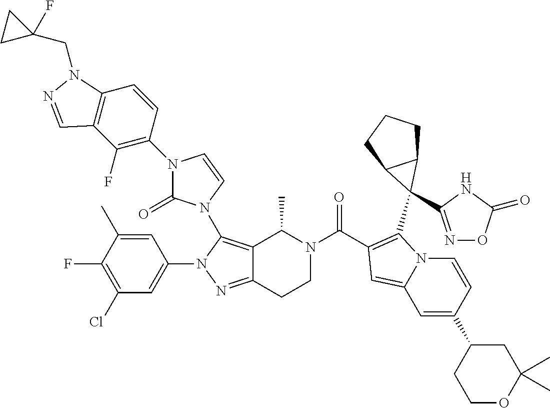 [SMILES: Cc1cc(-n2nc3c(c2-n2ccn(-c4ccc5c(cnn5CC5(F)CC5)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(Cl)c1F] |
| 71 | 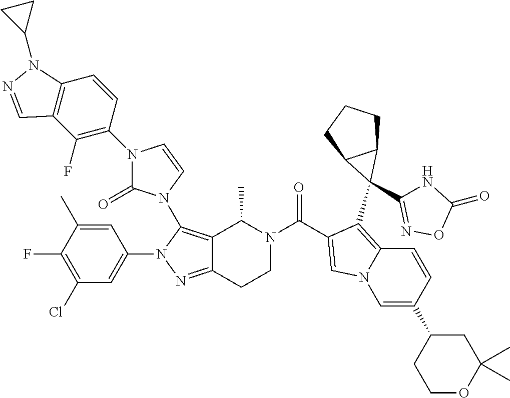 [SMILES: Cc1cc(-n2nc3c(c2-n2ccn(-c4ccc5c(cnn5C5CC5)c4F)c2=O)[C@H](C)N(C(=O)c2cn4cc([C@H]5CCOC(C)(C)C5)ccc4c2[C@@]2(c4noc(=O)[nH]4)[C@@H]4CCC[C@@H]42)CC3)cc(Cl)c1F] |
| 72 | 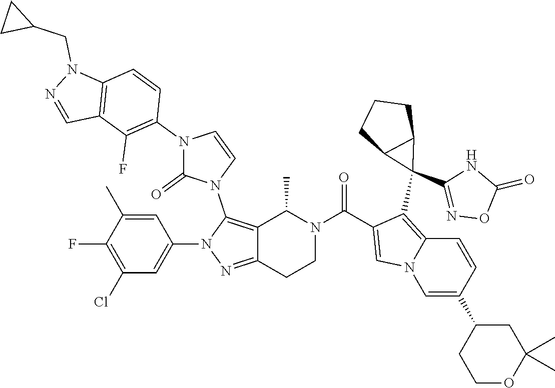 [SMILES: Cc1cc(-n2nc3c(c2-n2ccn(-c4ccc5c(cnn5CC5CC5)c4F)c2=O)[C@H](C)N(C(=O)c2cn4cc([C@H]5CCOC(C)(C)C5)ccc4c2[C@@]2(c4noc(=O)[nH]4)[C@@H]4CCC[C@@H]42)CC3)cc(Cl)c1F] |
| 73 | 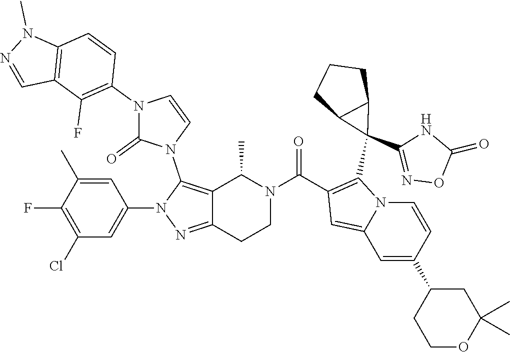 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(Cl)c1F] |
| 74 | 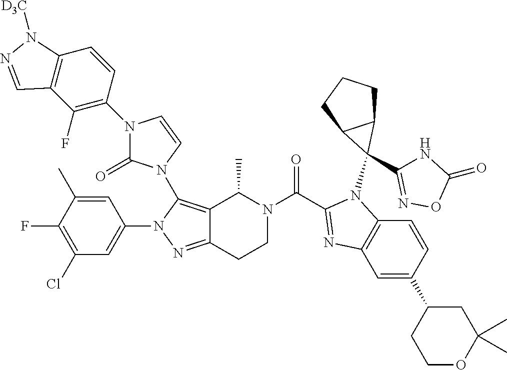 [SMILES: [2H]C([2H])([2H])n1ncc2c(F)c(-n3ccn(-c4c5c(nn4-c4cc(C)c(F)c(Cl)c4)CCN(C(=O)c4nc6cc([C@H]7CCOC(C)(C)C7)ccc6n4[C@@]4(c6noc(=O)[nH]6)[C@@H]6CCC[C@@H]64)[C@H]5C)c3=O)ccc21] |
| 75 | 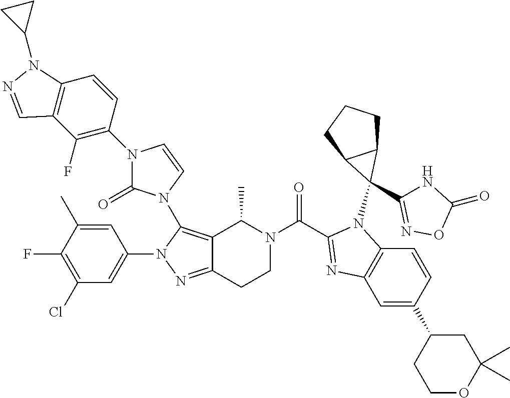 [SMILES: Cc1cc(-n2nc3c(c2-n2ccn(-c4ccc5c(cnn5C5CC5)c4F)c2=O)[C@H](C)N(C(=O)c2nc4cc([C@H]5CCOC(C)(C)C5)ccc4n2[C@@]2(c4noc(=O)[nH]4)[C@@H]4CCC[C@@H]42)CC3)cc(Cl)c1F] |
| 76 | 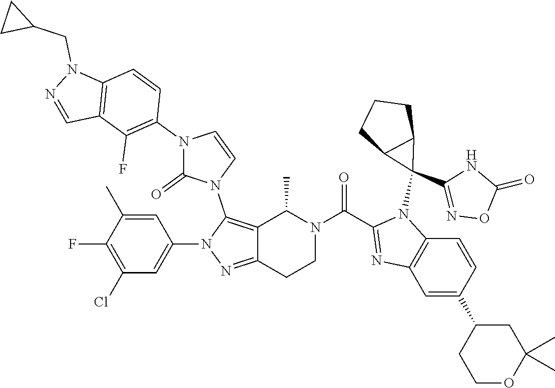 [SMILES: Cc1cc(-n2nc3c(c2-n2ccn(-c4ccc5c(cnn5CC5CC5)c4F)c2=O)[C@H](C)N(C(=O)c2nc4cc([C@H]5CCOC(C)(C)C5)ccc4n2[C@@]2(c4noc(=O)[nH]4)[C@@H]4CCC[C@@H]42)CC3)cc(Cl)c1F] |
| 77 | 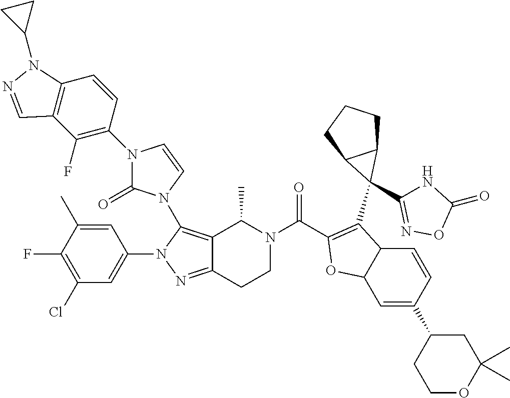 [SMILES: Cc1cc(-n2nc3c(c2-n2ccn(-c4ccc5c(cnn5C5CC5)c4F)c2=O)[C@H](C)N(C(=O)C2=C([C@@]4(c5noc(=O)[nH]5)[C@@H]5CCC[C@@H]54)C4C=CC([C@H]5CCOC(C)(C)C5)=CC4O2)CC3)cc(Cl)c1F] |
| 78 | 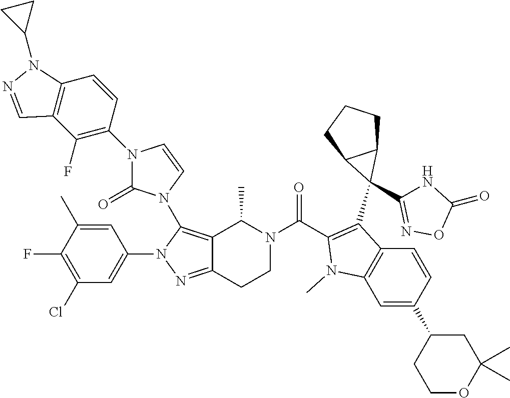 [SMILES: Cc1cc(-n2nc3c(c2-n2ccn(-c4ccc5c(cnn5C5CC5)c4F)c2=O)[C@H](C)N(C(=O)c2c([C@@]4(c5noc(=O)[nH]5)[C@@H]5CCC[C@@H]54)c4ccc([C@H]5CCOC(C)(C)C5)cc4n2C)CC3)cc(Cl)c1F] |
| 79 | 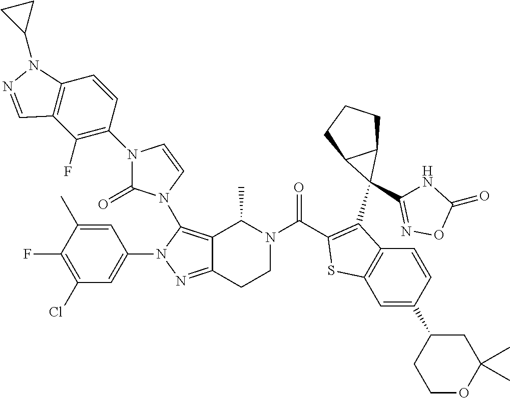 [SMILES: Cc1cc(-n2nc3c(c2-n2ccn(-c4ccc5c(cnn5C5CC5)c4F)c2=O)[C@H](C)N(C(=O)c2sc4cc([C@H]5CCOC(C)(C)C5)ccc4c2[C@@]2(c4noc(=O)[nH]4)[C@@H]4CCC[C@@H]42)CC3)cc(Cl)c1F] |
| 80 | 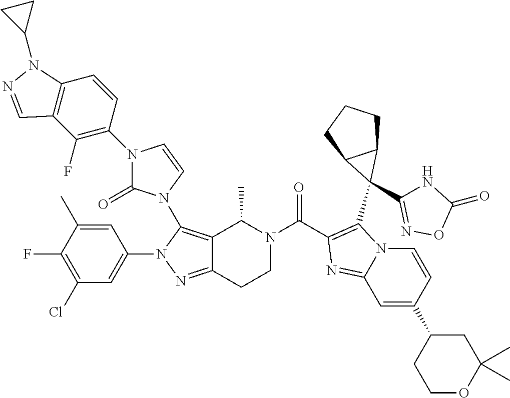 [SMILES: Cc1cc(-n2nc3c(c2-n2ccn(-c4ccc5c(cnn5C5CC5)c4F)c2=O)[C@H](C)N(C(=O)c2nc4cc([C@H]5CCOC(C)(C)C5)ccn4c2[C@@]2(c4noc(=O)[nH]4)[C@@H]4CCC[C@@H]42)CC3)cc(Cl)c1F] |
| 81 | 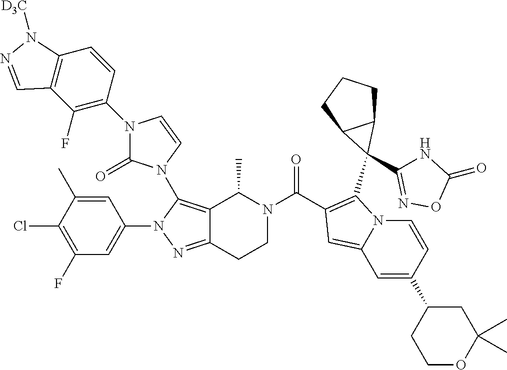 [SMILES: [2H]C([2H])([2H])n1ncc2c(F)c(-n3ccn(-c4c5c(nn4-c4cc(C)c(Cl)c(F)c4)CCN(C(=O)c4cc6cc([C@H]7CCOC(C)(C)C7)ccn6c4[C@@]4(c6noc(=O)[nH]6)[C@@H]6CCC[C@@H]64)[C@H]5C)c3=O)ccc21] |
| 82 | 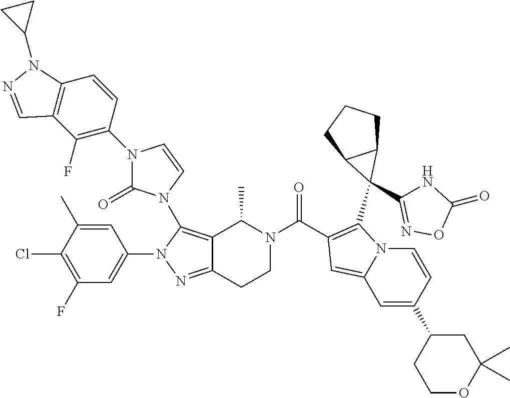 [SMILES: Cc1cc(-n2nc3c(c2-n2ccn(-c4ccc5c(cnn5C5CC5)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(F)c1Cl] |
| 83 | 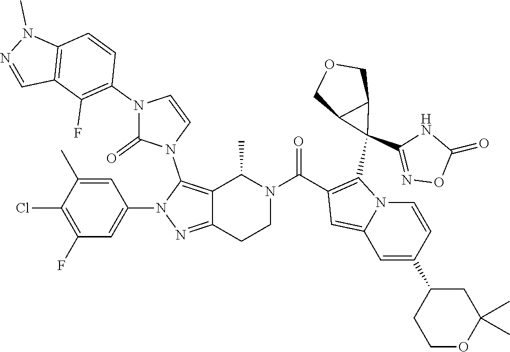 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccn4c2[C@@]2(c4noc(=O)[nH]4)[C@@H]4COC[C@@H]42)CC3)cc(F)c1Cl] |
| 84 | 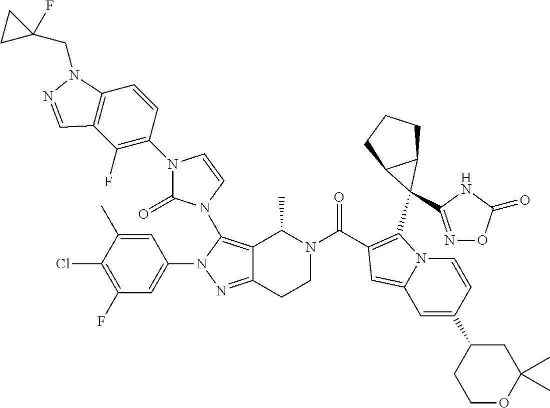 [SMILES: Cc1cc(-n2nc3c(c2-n2ccn(-c4ccc5c(cnn5CC5(F)CC5)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(F)c1Cl] |
| 85 | 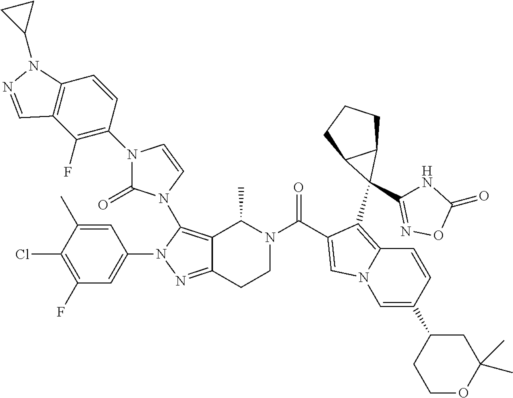 [SMILES: Cc1cc(-n2nc3c(c2-n2ccn(-c4ccc5c(cnn5C5CC5)c4F)c2=O)[C@H](C)N(C(=O)c2cn4cc([C@H]5CCOC(C)(C)C5)ccc4c2[C@@]2(c4noc(=O)[nH]4)[C@@H]4CCC[C@@H]42)CC3)cc(F)c1Cl] |
| 86 | 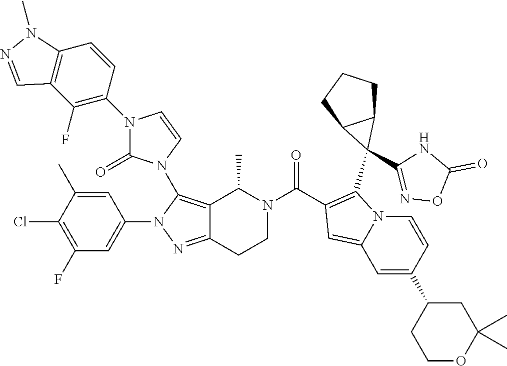 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(F)c1Cl] |
| 87 | 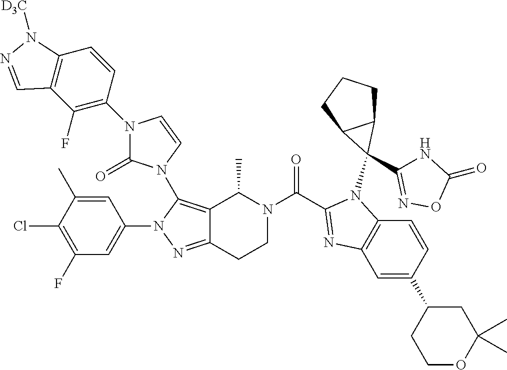 [SMILES: [2H]C([2H])([2H])n1ncc2c(F)c(-n3ccn(-c4c5c(nn4-c4cc(C)c(Cl)c(F)c4)CCN(C(=O)c4nc6cc([C@H]7CCOC(C)(C)C7)ccc6n4[C@@]4(c6noc(=O)[nH]6)[C@@H]6CCC[C@@H]64)[C@H]5C)c3=O)ccc21] |
| 88 | 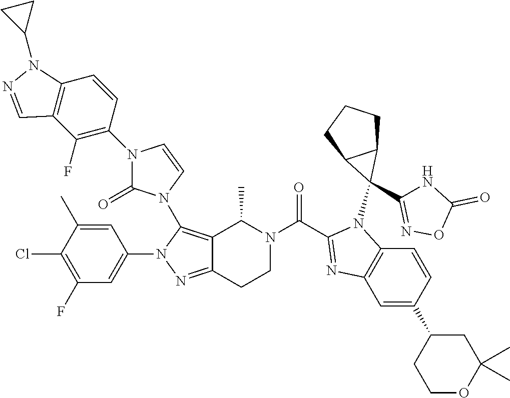 [SMILES: Cc1cc(-n2nc3c(c2-n2ccn(-c4ccc5c(cnn5C5CC5)c4F)c2=O)[C@H](C)N(C(=O)c2nc4cc([C@H]5CCOC(C)(C)C5)ccc4n2[C@@]2(c4noc(=O)[nH]4)[C@@H]4CCC[C@@H]42)CC3)cc(F)c1Cl] |
| 89 | 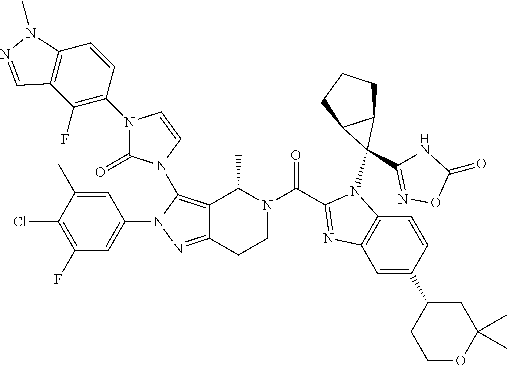 [SMILES: Cc1cc(-n2nc3c(c2-n2ccn(-c4ccc5c(cnn5C)c4F)c2=O)[C@H](C)N(C(=O)c2nc4cc([C@H]5CCOC(C)(C)C5)ccc4n2[C@@]2(c4noc(=O)[nH]4)[C@@H]4CCC[C@@H]42)CC3)cc(F)c1Cl] |
| 90 | 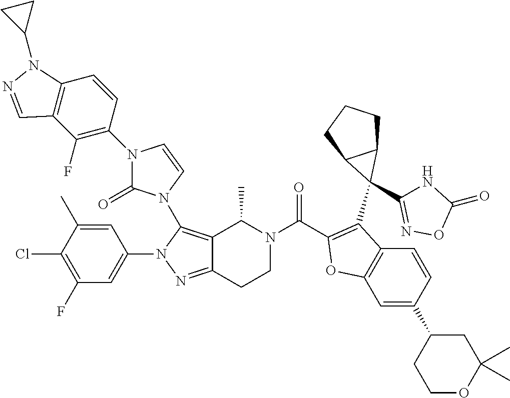 [SMILES: Cc1cc(-n2nc3c(c2-n2ccn(-c4ccc5c(cnn5C5CC5)c4F)c2=O)[C@H](C)N(C(=O)c2oc4cc([C@H]5CCOC(C)(C)C5)ccc4c2[C@@]2(c4noc(=O)[nH]4)[C@@H]4CCC[C@@H]42)CC3)cc(F)c1Cl] |
| 91 | 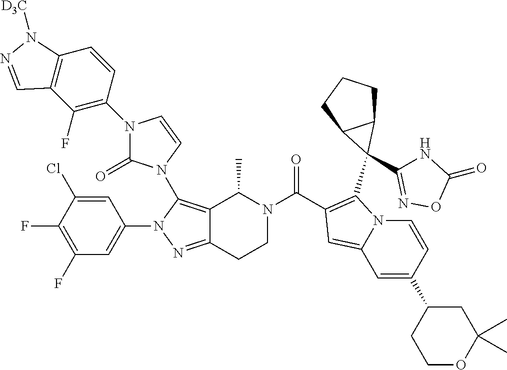 [SMILES: [2H]C([2H])([2H])n1ncc2c(F)c(-n3ccn(-c4c5c(nn4-c4cc(F)c(F)c(Cl)c4)CCN(C(=O)c4cc6cc([C@H]7CCOC(C)(C)C7)ccn6c4[C@@]4(c6noc(=O)[nH]6)[C@@H]6CCC[C@@H]64)[C@H]5C)c3=O)ccc21] |
| 92 | 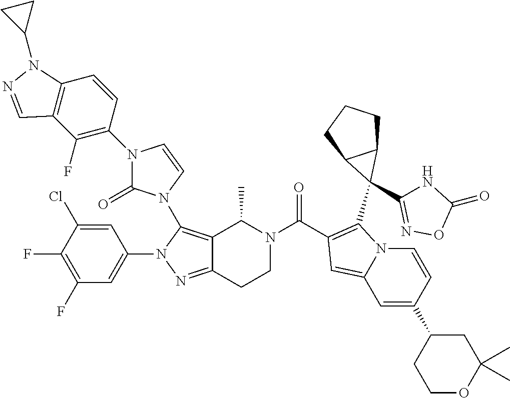 [SMILES: C[C@H]1c2c(nn(-c3cc(F)c(F)c(Cl)c3)c2-n2ccn(-c3ccc4c(cnn4C4CC4)c3F)c2=O)CCN1C(=O)c1cc2cc([C@H]3CCOC(C)(C)C3)ccn2c1[C@@]1(c2noc(=O)[nH]2)[C@@H]2CCC[C@@H]21] |
| 93 | 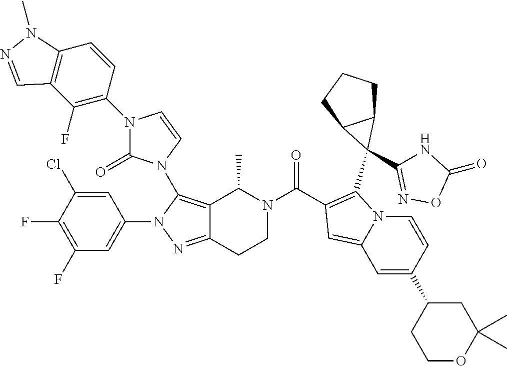 [SMILES: C[C@H]1c2c(nn(-c3cc(F)c(F)c(Cl)c3)c2-n2ccn(-c3ccc4c(cnn4C)c3F)c2=O)CCN1C(=O)c1cc2cc([C@H]3CCOC(C)(C)C3)ccn2c1[C@@]1(c2noc(=O)[nH]2)[C@@H]2CCC[C@@H]21] |
| 94 | 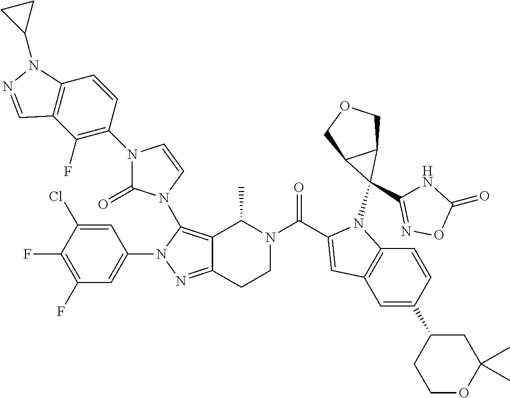 [SMILES: C[C@H]1c2c(nn(-c3cc(F)c(F)c(Cl)c3)c2-n2ccn(-c3ccc4c(cnn4C4CC4)c3F)c2=O)CCN1C(=O)c1cc2cc([C@H]3CCOC(C)(C)C3)ccc2n1[C@@]1(c2noc(=O)[nH]2)[C@@H]2COC[C@@H]21] |
| 95 | 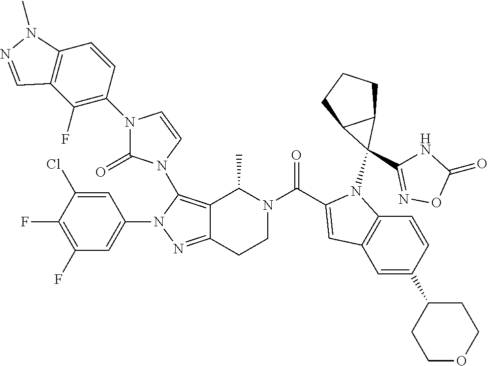 |
| 96 | 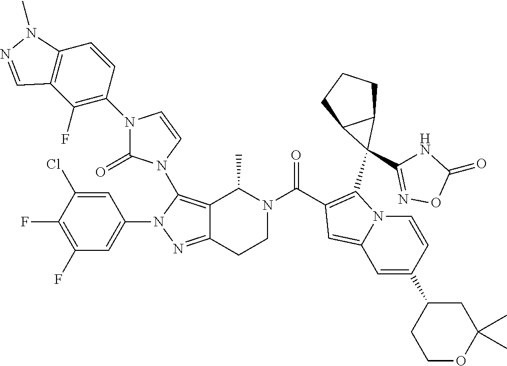 [SMILES: C[C@H]1c2c(nn(-c3cc(F)c(F)c(Cl)c3)c2-n2ccn(-c3ccc4c(cnn4C)c3F)c2=O)CCN1C(=O)c1cc2cc([C@H]3CCOC(C)(C)C3)ccn2c1[C@@]1(c2noc(=O)[nH]2)[C@@H]2CCC[C@@H]21] |
| 97 | 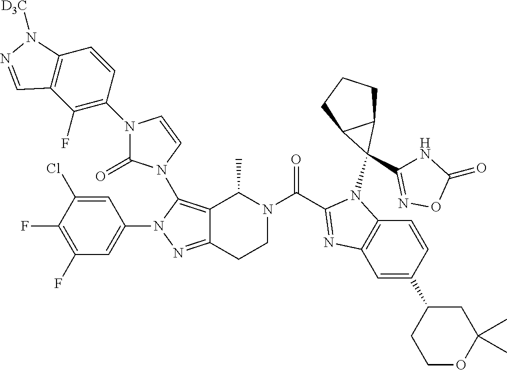 [SMILES: [2H]C([2H])([2H])n1ncc2c(F)c(-n3ccn(-c4c5c(nn4-c4cc(F)c(F)c(Cl)c4)CCN(C(=O)c4nc6cc([C@H]7CCOC(C)(C)C7)ccc6n4[C@@]4(c6noc(=O)[nH]6)[C@@H]6CCC[C@@H]64)[C@H]5C)c3=O)ccc21] |
| 98 | 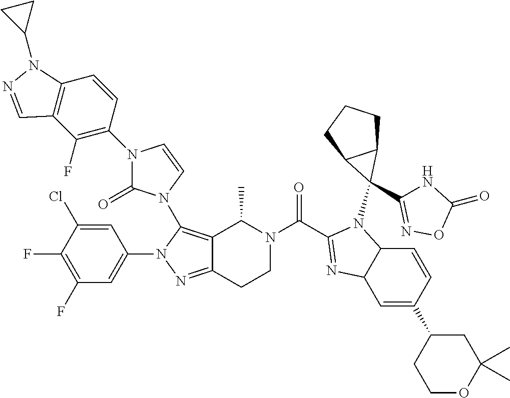 [SMILES: C[C@H]1c2c(nn(-c3cc(F)c(F)c(Cl)c3)c2-n2ccn(-c3ccc4c(cnn4C4CC4)c3F)c2=O)CCN1C(=O)C1=NC2C=C([C@H]3CCOC(C)(C)C3)C=CC2N1[C@@]1(c2noc(=O)[nH]2)[C@@H]2CCC[C@@H]21] |
| 99 | 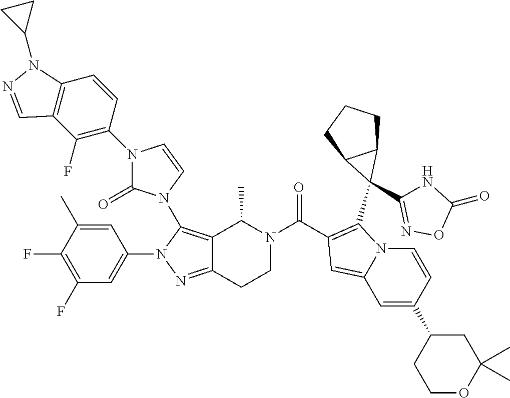 [SMILES: Cc1cc(-n2nc3c(c2-n2ccn(-c4ccc5c(cnn5C5CC5)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(F)c1F] |
| 100 | 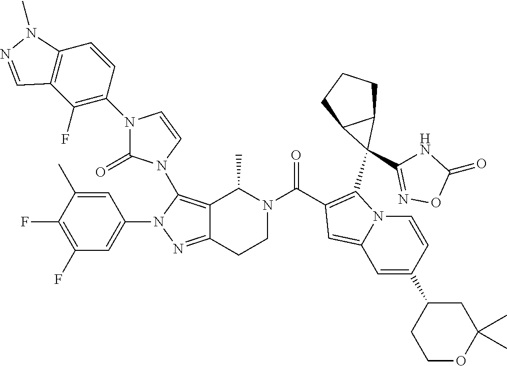 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(F)c1F] |
| 101 | 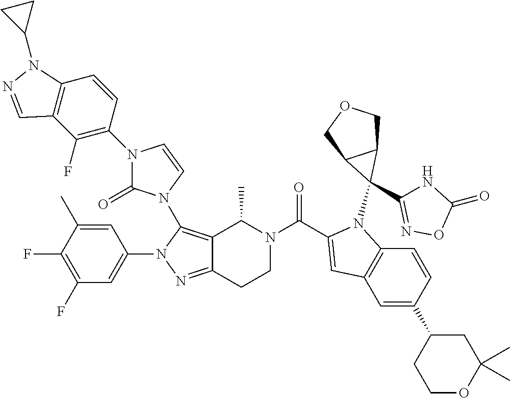 [SMILES: Cc1cc(-n2nc3c(c2-n2ccn(-c4ccc5c(cnn5C5CC5)c4F)c2=O)[C@H](C)N(C(=O)c2cc4cc([C@H]5CCOC(C)(C)C5)ccc4n2[C@@]2(c4noc(=O)[nH]4)[C@@H]4COC[C@@H]42)CC3)cc(F)c1F] |
| 102 | 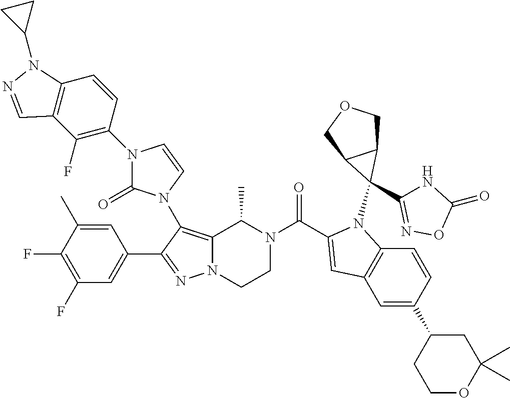 [SMILES: Cc1cc(-c2nn3c(c2-n2ccn(-c4ccc5c(cnn5C5CC5)c4F)c2=O)[C@H](C)N(C(=O)c2cc4cc([C@H]5CCOC(C)(C)C5)ccc4n2[C@@]2(c4noc(=O)[nH]4)[C@@H]4COC[C@@H]42)CC3)cc(F)c1F] |
| 103 | 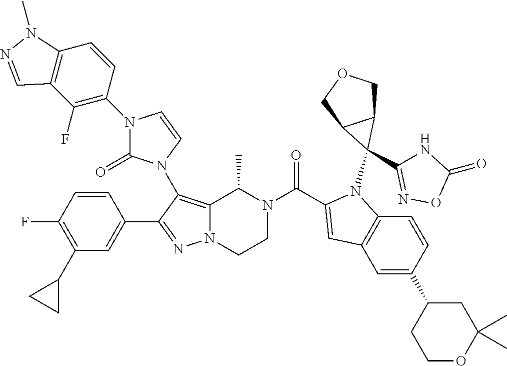 [SMILES: C[C@H]1c2c(-n3ccn(-c4ccc5c(cnn5C)c4F)c3=O)c(-c3ccc(F)c(C4CC4)c3)nn2CCN1C(=O)c1cc2cc([C@H]3CCOC(C)(C)C3)ccc2n1[C@@]1(c2noc(=O)[nH]2)[C@@H]2COC[C@@H]21] |
| 104 | 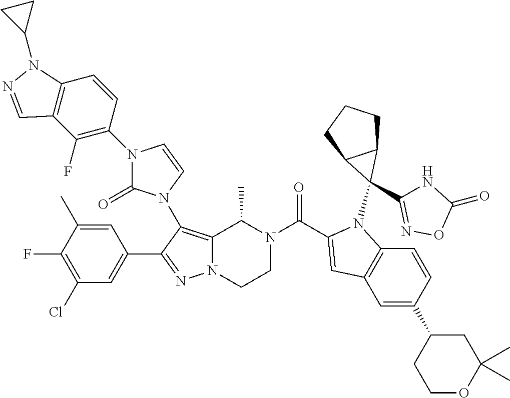 [SMILES: Cc1cc(-c2nn3c(c2-n2ccn(-c4ccc5c(cnn5C5CC5)c4F)c2=O)[C@H](C)N(C(=O)c2cc4cc([C@H]5CCOC(C)(C)C5)ccc4n2[C@@]2(c4noc(=O)[nH]4)[C@@H]4CCC[C@@H]42)CC3)cc(Cl)c1F] |
| 105 | 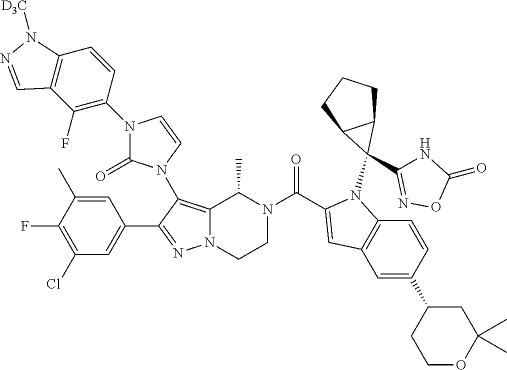 [SMILES: [2H]C([2H])([2H])n1ncc2c(F)c(-n3ccn(-c4c(-c5cc(C)c(F)c(Cl)c5)nn5c4[C@H](C)N(C(=O)c4cc6cc([C@H]7CCOC(C)(C)C7)ccc6n4[C@@]4(c6noc(=O)[nH]6)[C@@H]6CCC[C@@H]64)CC5)c3=O)ccc21] |
| 106 | 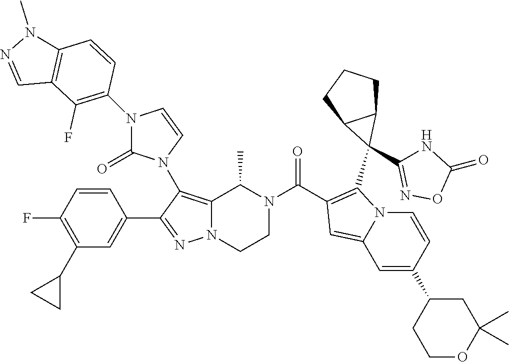 [SMILES: C[C@H]1c2c(-n3ccn(-c4ccc5c(cnn5C)c4F)c3=O)c(-c3ccc(F)c(C4CC4)c3)nn2CCN1C(=O)c1cc2cc([C@H]3CCOC(C)(C)C3)ccn2c1[C@@]1(c2noc(=O)[nH]2)[C@@H]2CCC[C@@H]21] |
| 107 | 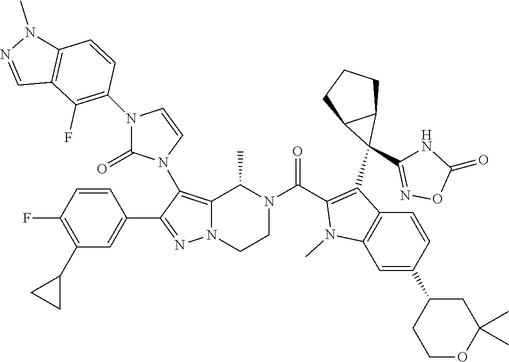 [SMILES: C[C@H]1c2c(-n3ccn(-c4ccc5c(cnn5C)c4F)c3=O)c(-c3ccc(F)c(C4CC4)c3)nn2CCN1C(=O)c1c([C@@]2(c3noc(=O)[nH]3)[C@@H]3CCC[C@@H]32)c2ccc([C@H]3CCOC(C)(C)C3)cc2n1C] |
| 108 | 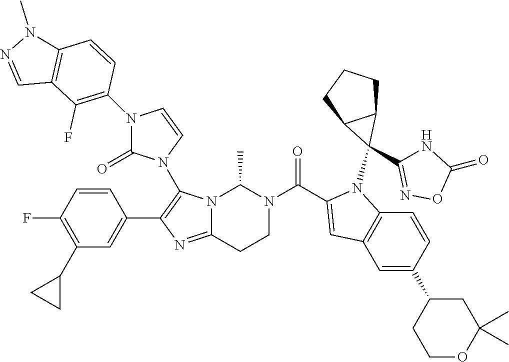 [SMILES: C[C@@H]1N(C(=O)c2cc3cc([C@H]4CCOC(C)(C)C4)ccc3n2[C@@]2(c3noc(=O)[nH]3)[C@@H]3CCC[C@@H]32)CCc2nc(-c3ccc(F)c(C4CC4)c3)c(-n3ccn(-c4ccc5c(cnn5C)c4F)c3=O)n21] |
| 109 | 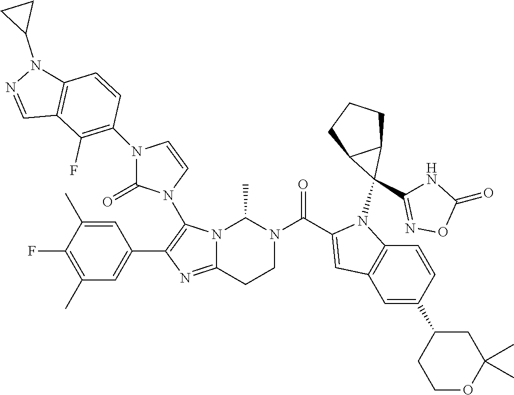 [SMILES: Cc1cc(-c2nc3n(c2-n2ccn(-c4ccc5c(cnn5C5CC5)c4F)c2=O)[C@H](C)N(C(=O)c2cc4cc([C@H]5CCOC(C)(C)C5)ccc4n2[C@@]2(c4noc(=O)[nH]4)[C@@H]4CCC[C@@H]42)CC3)cc(C)c1F] |
| 110 | 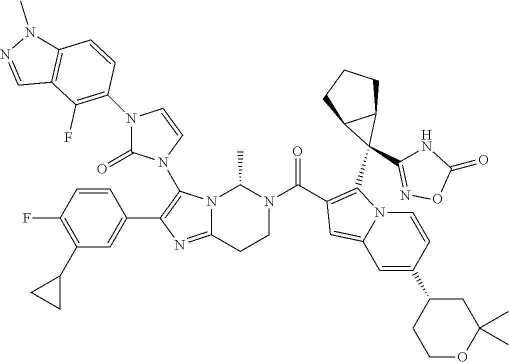 [SMILES: C[C@@H]1N(C(=O)c2cc3cc([C@H]4CCOC(C)(C)C4)ccn3c2[C@@]2(c3noc(=O)[nH]3)[C@@H]3CCC[C@@H]32)CCc2nc(-c3ccc(F)c(C4CC4)c3)c(-n3ccn(-c4ccc5c(cnn5C)c4F)c3=O)n21] |
| 111 | 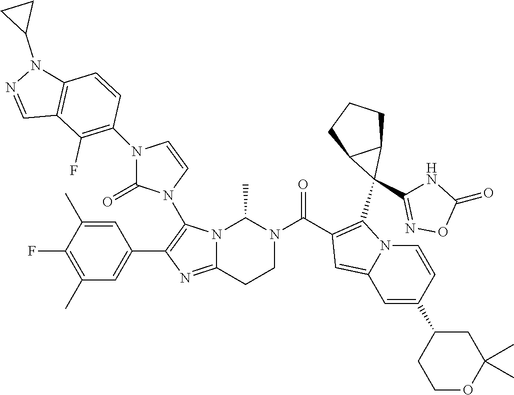 [SMILES: Cc1cc(-c2nc3n(c2-n2ccn(-c4ccc5c(cnn5C5CC5)c4F)c2=O)[C@H](C)N(C(=O)c2cc4cc([C@H]5CCOC(C)(C)C5)ccn4c2[C@@]2(c4noc(=O)[nH]4)[C@@H]4CCC[C@@H]42)CC3)cc(C)c1F] |
| 112 | 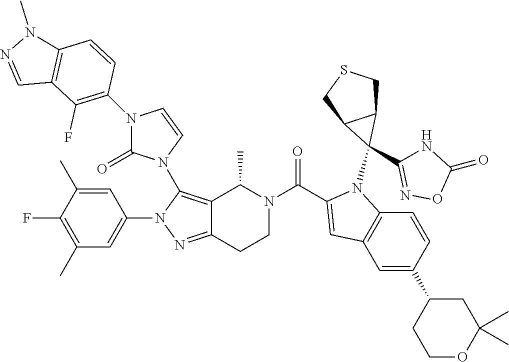 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CSC[C@@H]42)CC3)cc(C)c1F] |
| 113 | 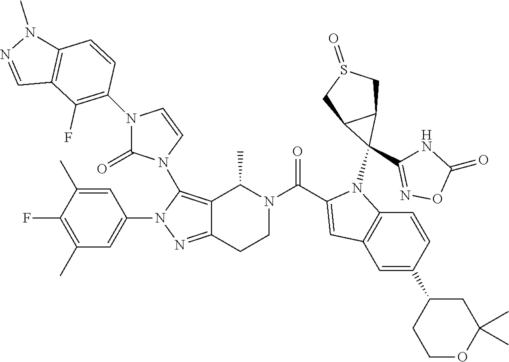 |
| 114 | 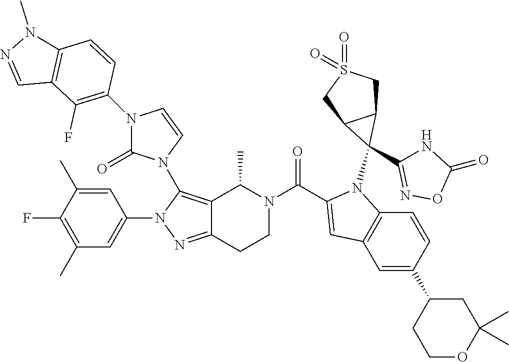 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CS(=O)(=O)C[C@@H]42)CC3)cc(C)c1F] |
| 115 | 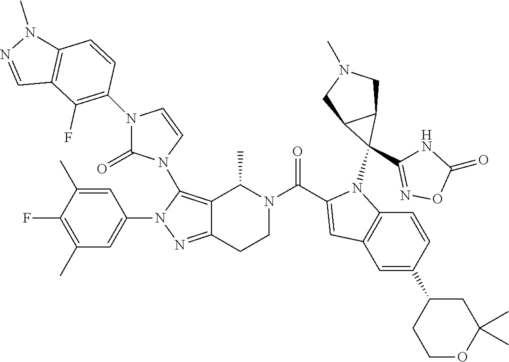 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(C)C[C@@H]42)CC3)cc(C)c1F] |
| 116 | 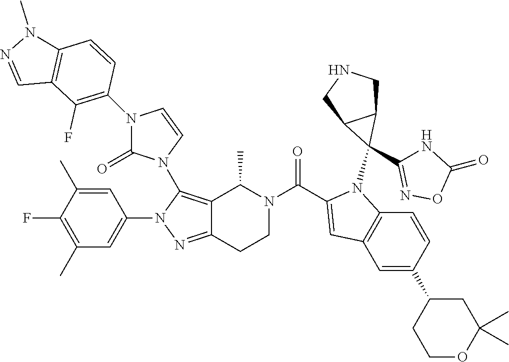 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CNC[C@@H]42)CC3)cc(C)c1F] |
| 117 | 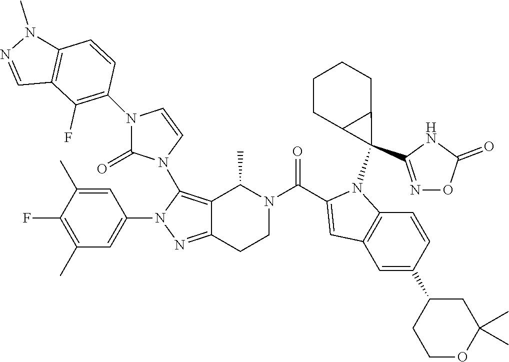 |
| 118 | 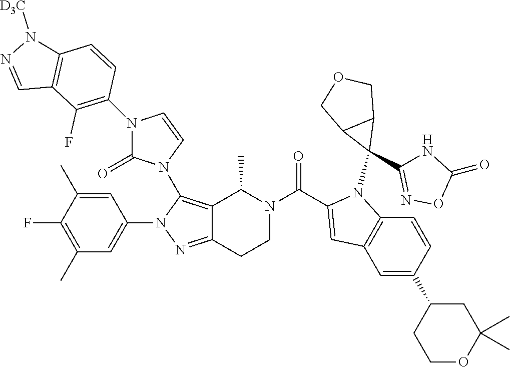 |
| 119 | 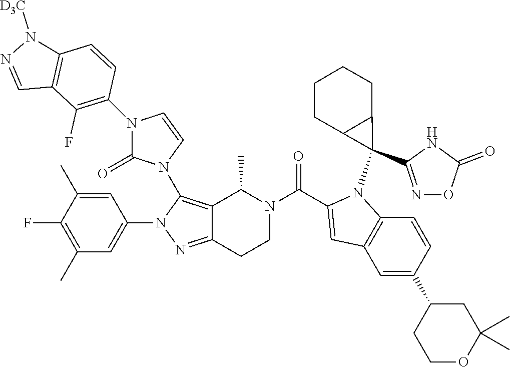 |
| 120 | 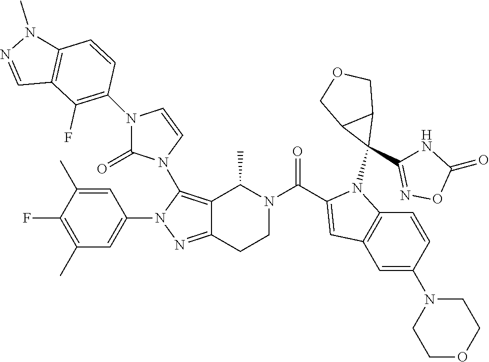 |
| 121 | 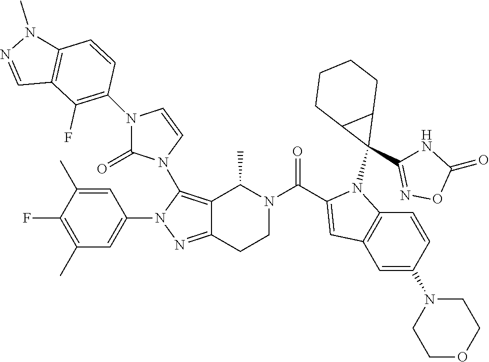 |
| 122 | 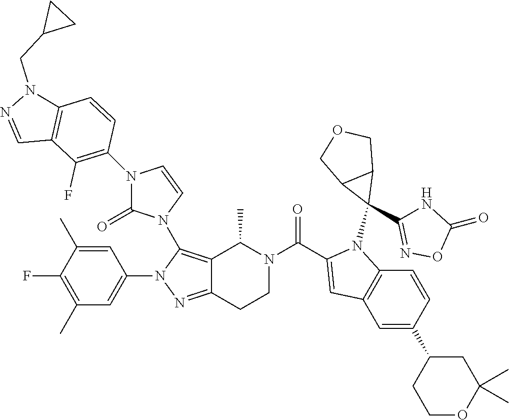 |
| 123 | 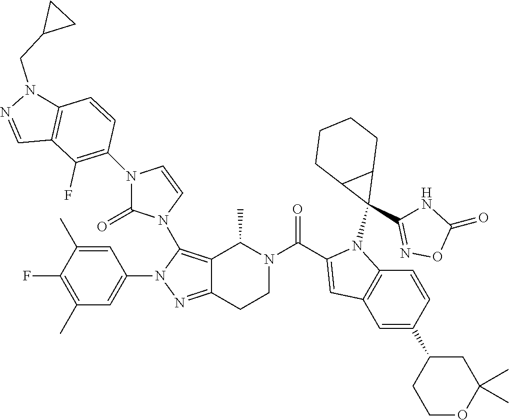 |
| 124 | 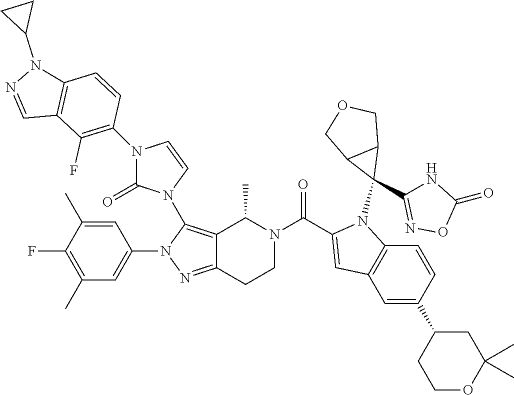 |
| 125 | 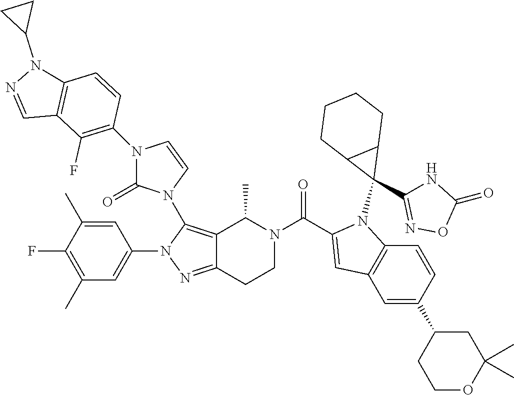 |
| 126 | 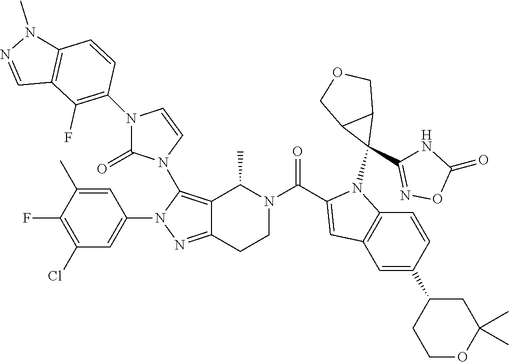 |
| 127 | 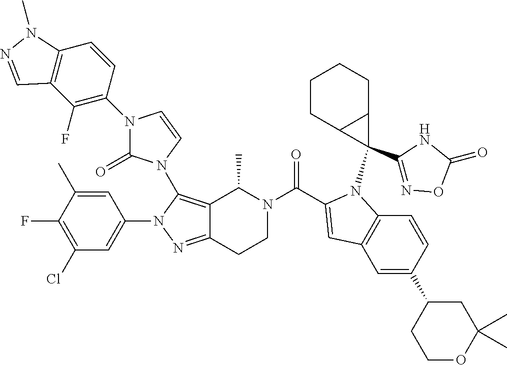 |
| 128 | 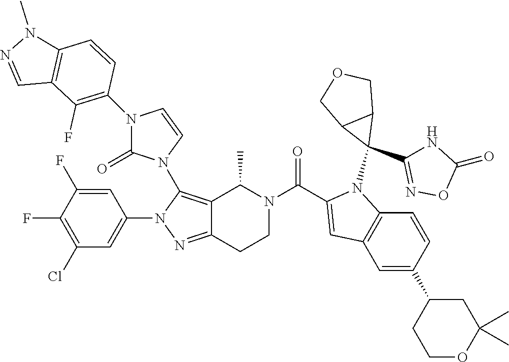 |
| 129 | 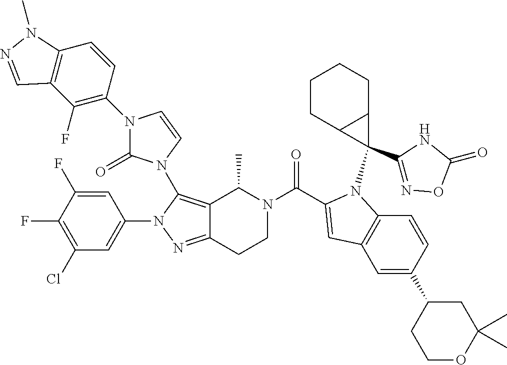 |
| 130 | 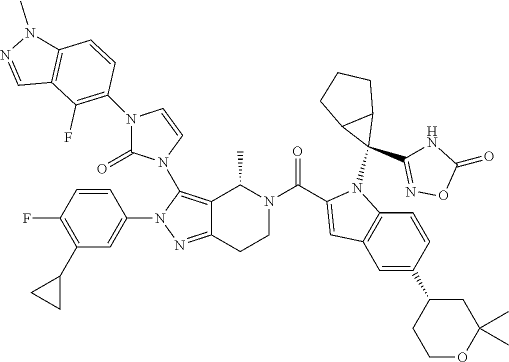 |
| 131 | 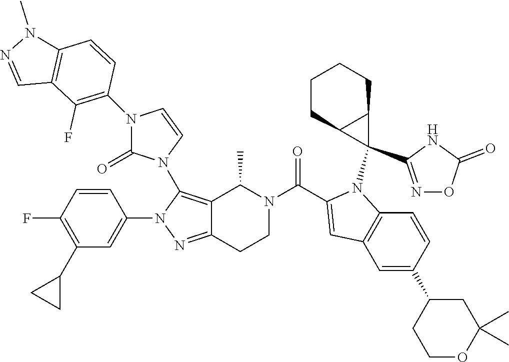 [SMILES: C[C@H]1c2c(nn(-c3ccc(F)c(C4CC4)c3)c2-n2ccn(-c3ccc4c(cnn4C)c3F)c2=O)CCN1C(=O)c1cc2cc([C@H]3CCOC(C)(C)C3)ccc2n1[C@@]1(c2noc(=O)[nH]2)[C@@H]2CCCC[C@@H]21] |
| 132 | 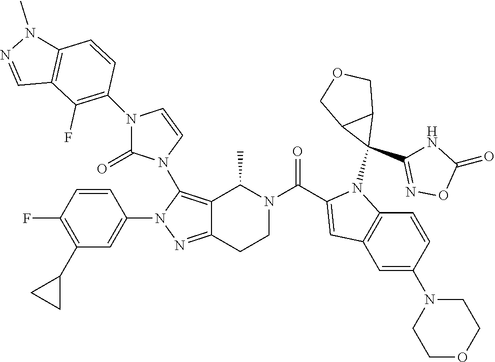 |
| 133 | 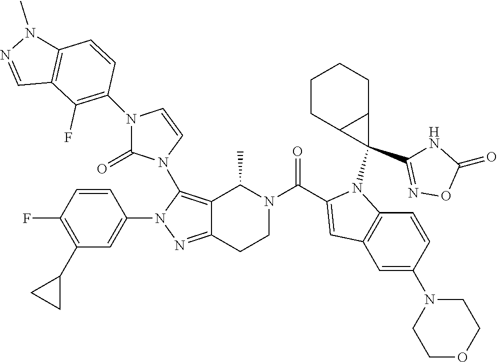 |

The present disclosure relates, in part, to a compound having a structure in Table 3c, as well as pharmaceutically acceptable salt thereof, stereoisomers thereof, analogs thereof, solvates thereof, prodrugs thereof, or tautomers thereof. In some embodiments, the compound is a compound having a structure in Table 3c or a pharmaceutically acceptable salt thereof.

TABLE 3c
Non-Limiting compounds

| No. | Structure |
|---|---|
| C-1 | 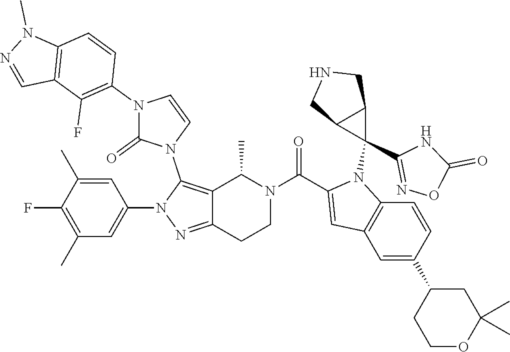 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CNC[C@@H]42)CC3)cc(C)c1F] |
| C-2 | 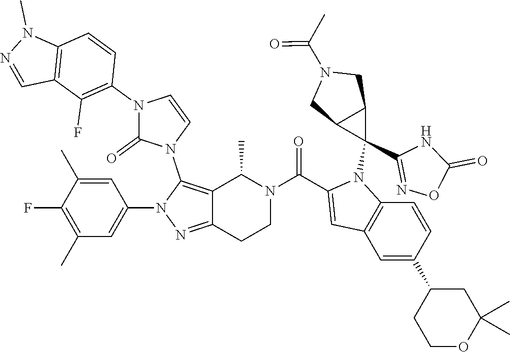 [SMILES: CC(=O)N1C[C@@H]2[C@H](C1)[C@@]2(c1noc(=O)[nH]1)n1c(C(=O)N2CCc3nn(-c4cc(C)c(F)c(C)c4)c(-n4ccn(-c5ccc6c(cnn6C)c5F)c4=O)c3[C@@H]2C)cc2cc([C@H]3CCOC(C)(C)C3)ccc21] |
| C-3 | 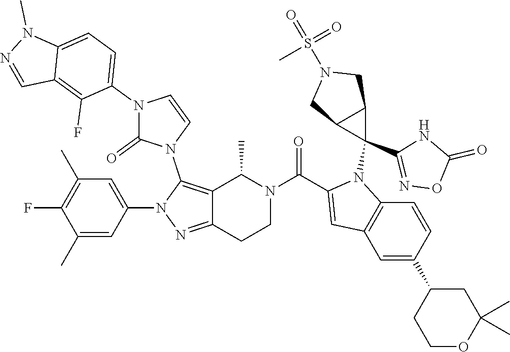 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(S(C)(=O)=O)C[C@@H]42)CC3)cc(C)c1F] |
| C-4 | 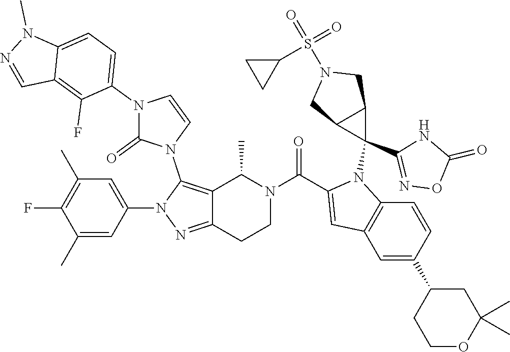 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(S(=O)(=O)C5CC5)C[C@@H]42)CC3)cc(C)c1F] |
| C-5 | 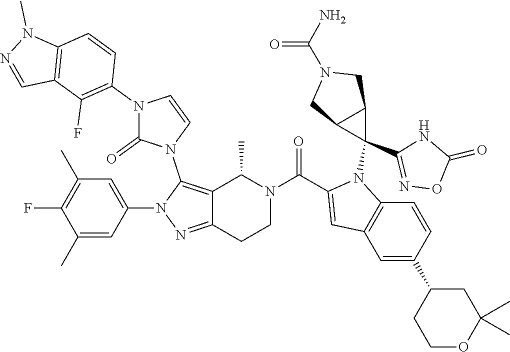 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(C(N)=O)C[C@@H]42)CC3)cc(C)c1F] |
| C-6 | 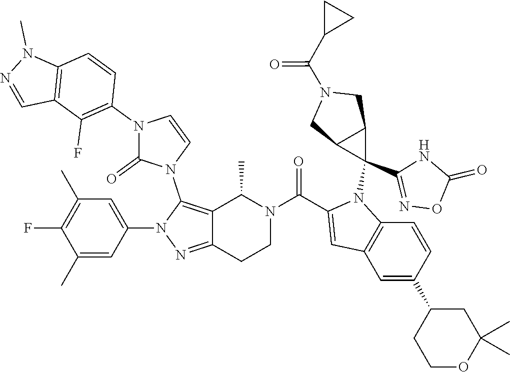 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(C(=O)C5CC5)C[C@@H]42)CC3)cc(C)c1F] |
| C-7 | 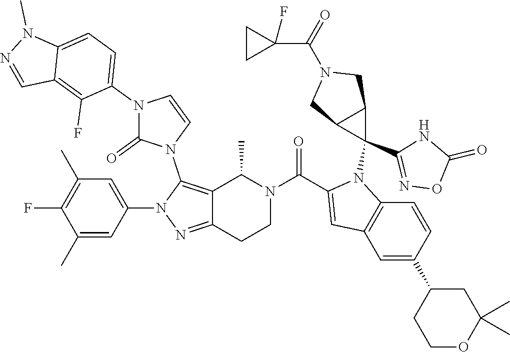 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(C(=O)C5(F)CC5)C[C@@H]42)CC3)cc(C)c1F] |
| C-8 | 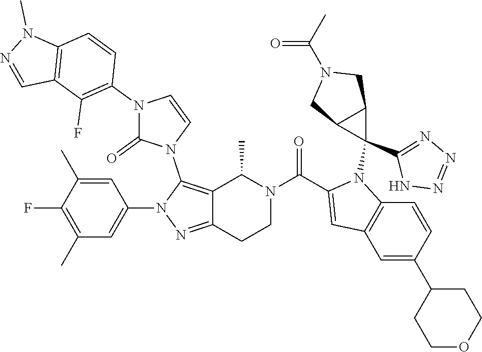 [SMILES: CC(=O)N1C[C@@H]2[C@H](C1)[C@@]2(c1nnn[nH]1)n1c(C(=O)N2CCc3nn(-c4cc(C)c(F)c(C)c4)c(-n4ccn(-c5ccc6c(cnn6C)c5F)c4=O)c3[C@@H]2C)cc2cc(C3CCOCC3)ccc21] |
| C-9 | 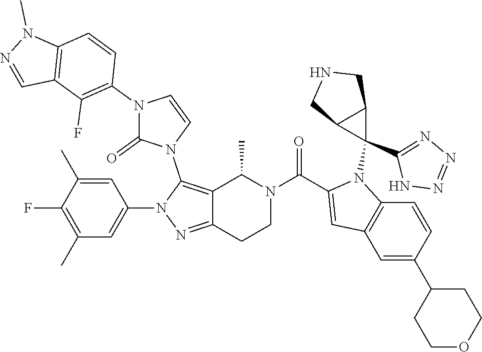 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4nnn[nH]4)[C@@H]4CNC[C@@H]42)CC3)cc(C)c1F] |
| C-10 | 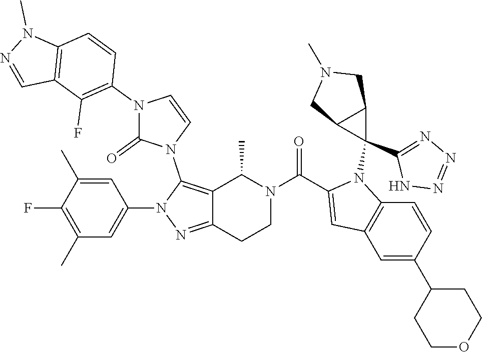 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4nnn[nH]4)[C@@H]4CN(C)C[C@@H]42)CC3)cc(C)c1F] |
| C-11 | 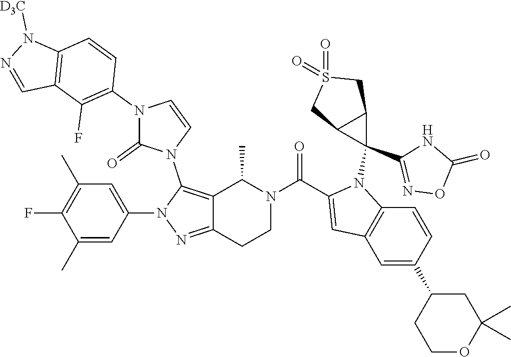 [SMILES: [2H]C([2H])([2H])n1ncc2c(F)c(-n3ccn(-c4c5c(nn4-c4cc(C)c(F)c(C)c4)CCN(C(=O)c4cc6cc([C@H]7CCOC(C)(C)C7)ccc6n4[C@@]4(c6noc(=O)[nH]6)[C@@H]6CS(=O)(=O)C[C@@H]64)[C@H]5C)c3=O)ccc21] |
| C-12 | 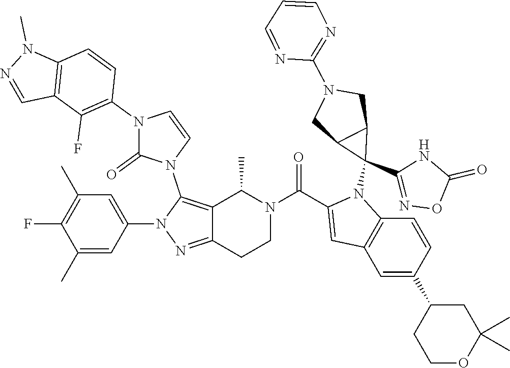 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(c5ncccn5)C[C@@H]42)CC3)cc(C)c1F] |
| C-13 | 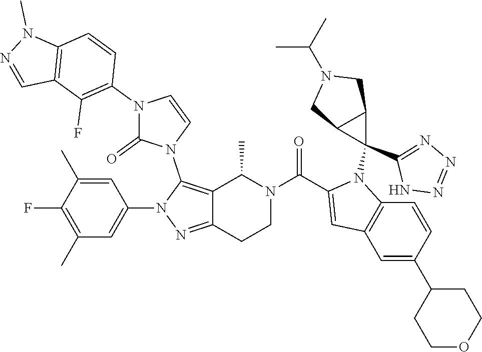 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4nnn[nH]4)[C@@H]4CN(C(C)C)C[C@@H]42)CC3)cc(C)c1F] |
| C-14 | 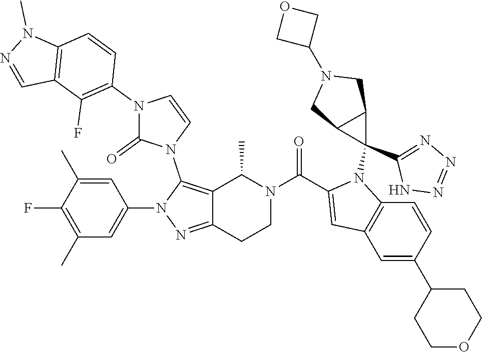 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4nnn[nH]4)[C@@H]4CN(C5COC5)C[C@@H]42)CC3)cc(C)c1F] |
| C-15 | 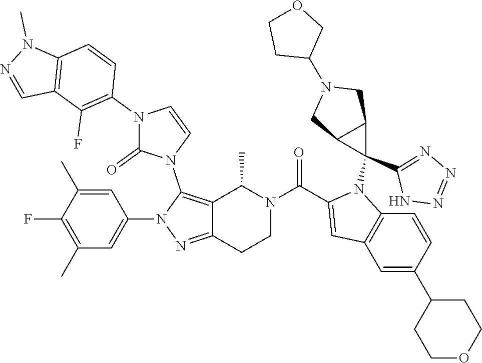 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4nnn[nH]4)[C@@H]4CN(C5CCOC5)C[C@@H]42)CC3)cc(C)c1F] |
| C-16 | 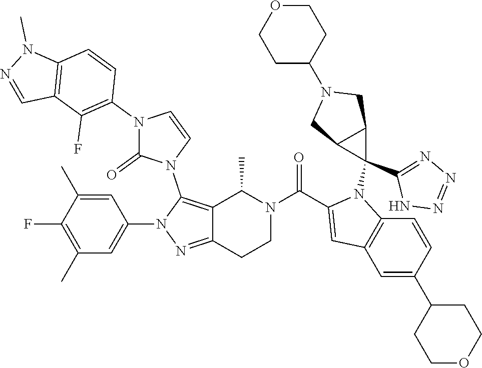 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4nnn[nH]4)[C@@H]4CN(C5CCOCC5)C[C@@H]42)CC3)cc(C)c1F] |
| C-17 OR C-18 | 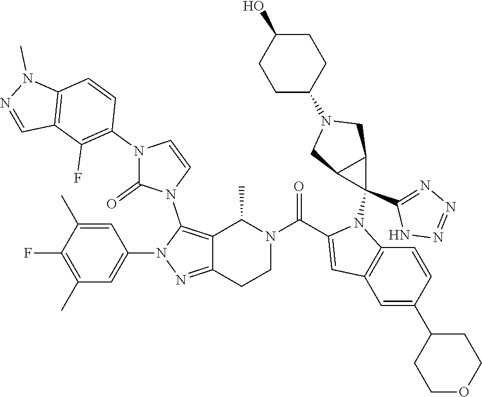 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4nnn[nH]4)[C@@H]4CN([C@H]5CC[C@H](O)CC5)C[C@@H]42)CC3)cc(C)c1F] |
| C-17 OR C-18 | 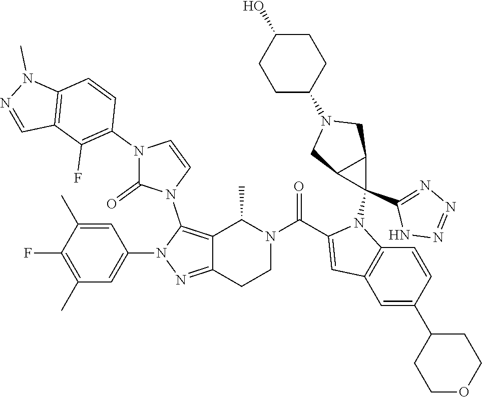 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4nnn[nH]4)[C@@H]4CN([C@H]5CC[C@@H](O)CC5)C[C@@H]42)CC3)cc(C)c1F] |
| C-19 | 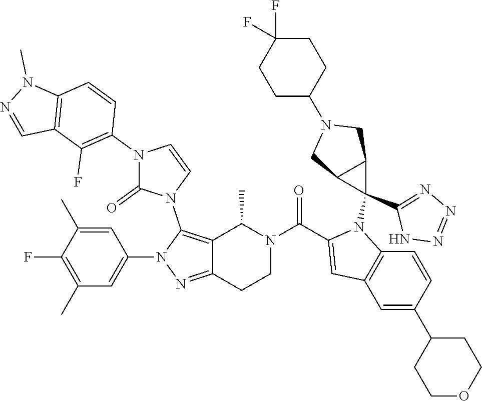 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4nnn[nH]4)[C@@H]4CN(C5CCC(F)(F)CC5)C[C@@H]42)CC3)cc(C)c1F] |
| C-20 | 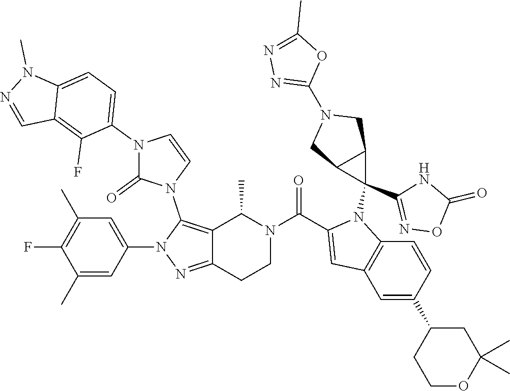 [SMILES: Cc1nnc(N2C[C@@H]3[C@H](C2)[C@@]3(c2noc(=O)[nH]2)n2c(C(=O)N3CCc4nn(-c5cc(C)c(F)c(C)c5)c(-n5ccn(-c6ccc7c(cnn7C)c6F)c5=O)c4[C@@H]3C)cc3cc([C@H]4CCOC(C)(C)C4)ccc32)o1] |
| C-21 | 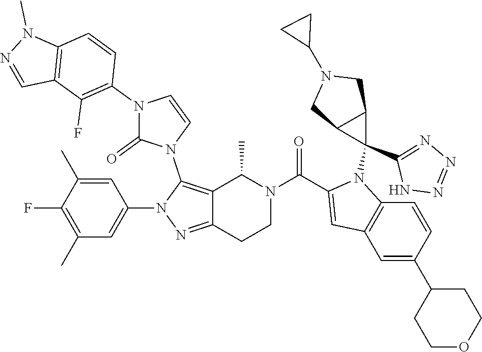 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4nnn[nH]4)[C@@H]4CN(C5CC5)C[C@@H]42)CC3)cc(C)c1F] |
| C-22 | 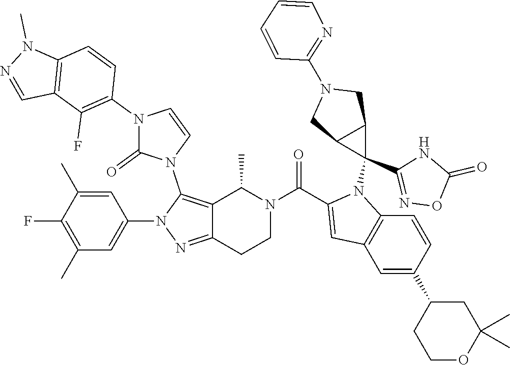 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(c5ccccn5)C[C@@H]42)CC3)cc(C)c1F] |
| C-23 | 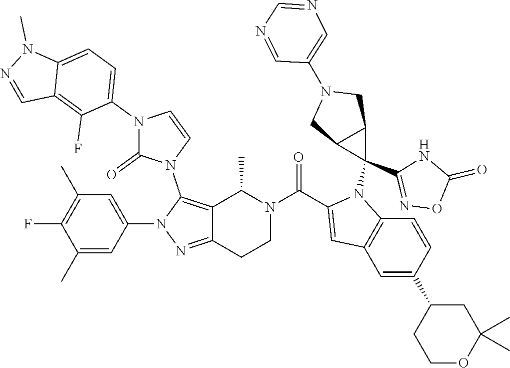 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(c5cncnc5)C[C@@H]42)CC3)cc(C)c1F] |
| C-24 | 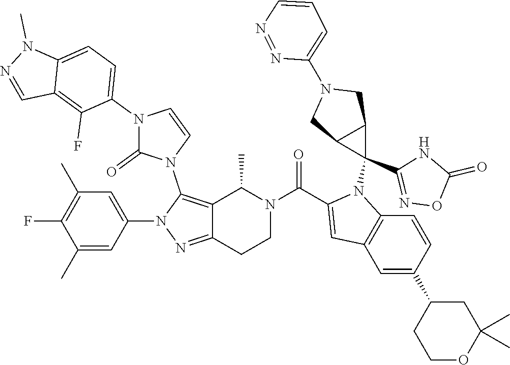 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(c5cccnn5)C[C@@H]42)CC3)cc(C)c1F] |
| C-25 | 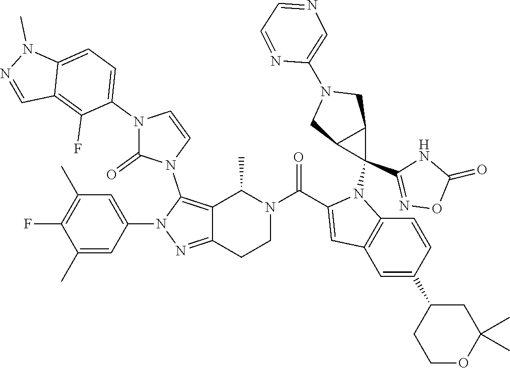 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(c5cnccn5)C[C@@H]42)CC3)cc(C)c1F] |
| C-26 | 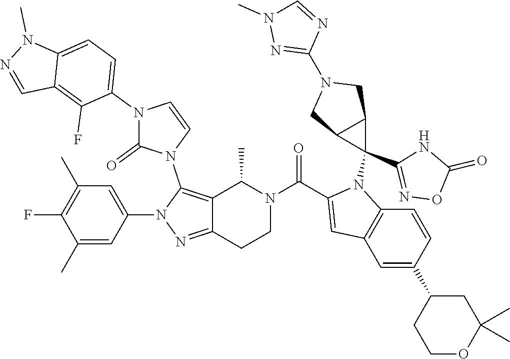 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(c5ncn(C)n5)C[C@@H]42)CC3)cc(C)c1F] |
| C-27 | 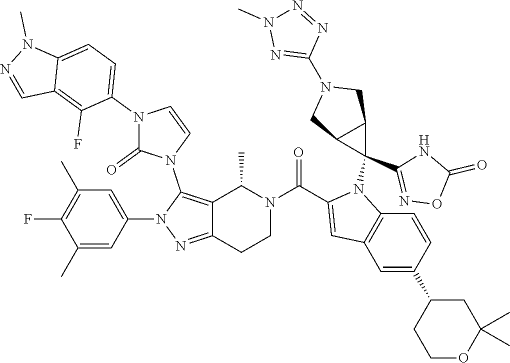 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(c5nnn(C)n5)C[C@@H]42)CC3)cc(C)c1F] |
| C-28 | 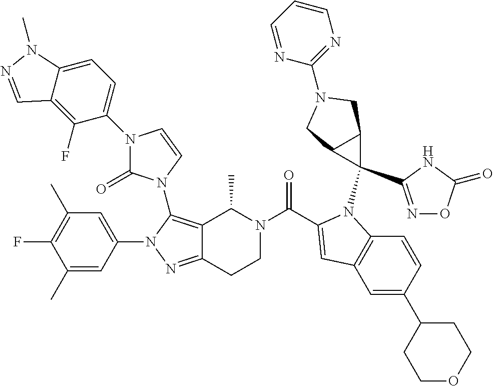 |
| C-29 | 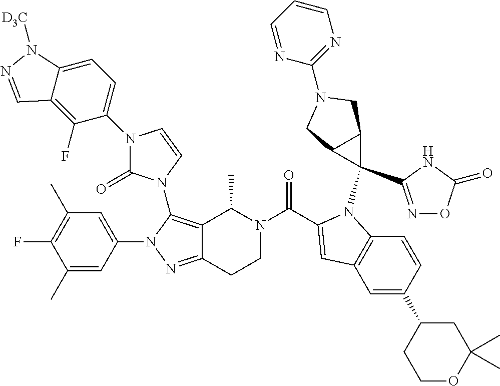 |
| C-30 | 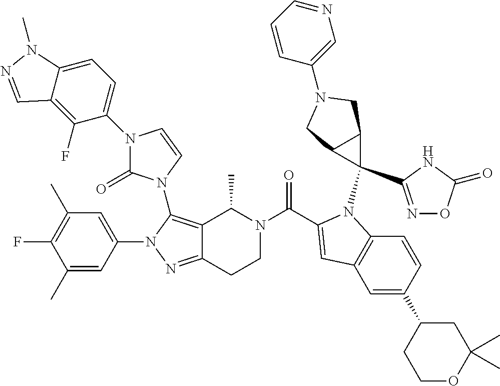 |
| C-31 | 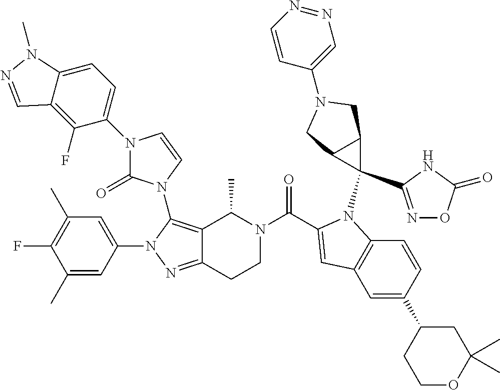 |
| C-32 | 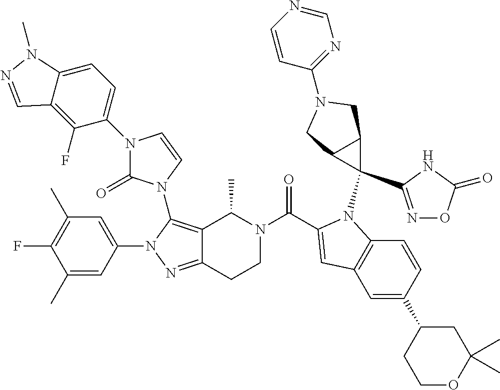 |
| C-33 | 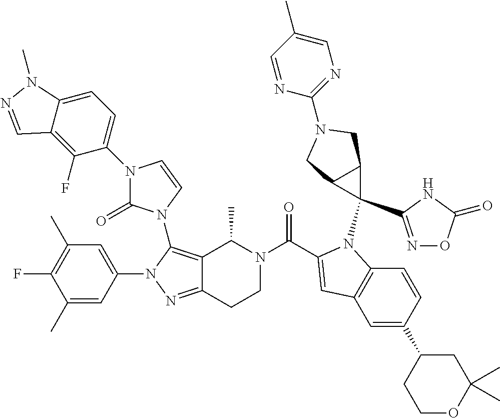 |
| C-34 | 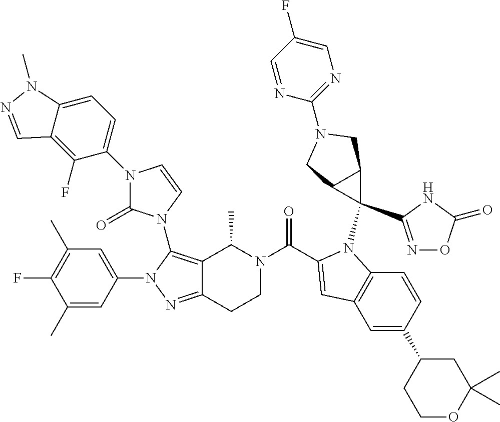 |
| C-35 | 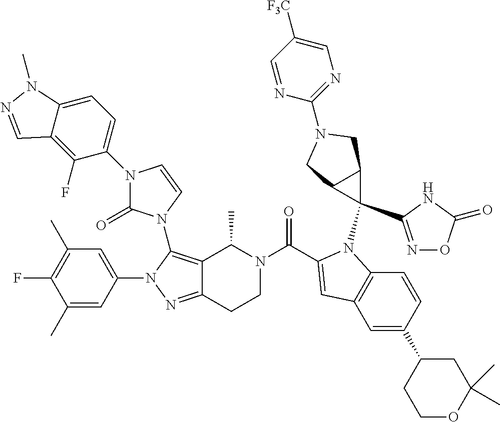 |
| C-36 | 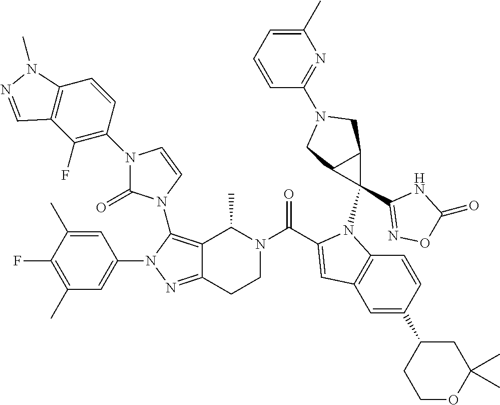 |
| C-37 | 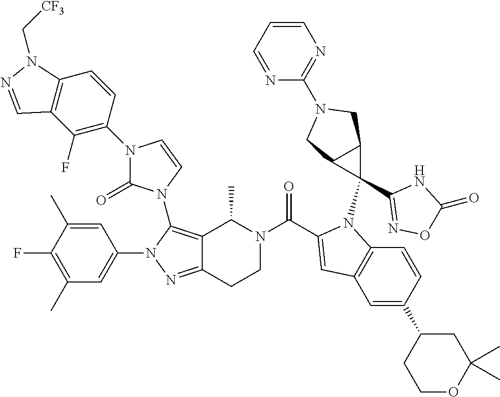 |
| C-38 | 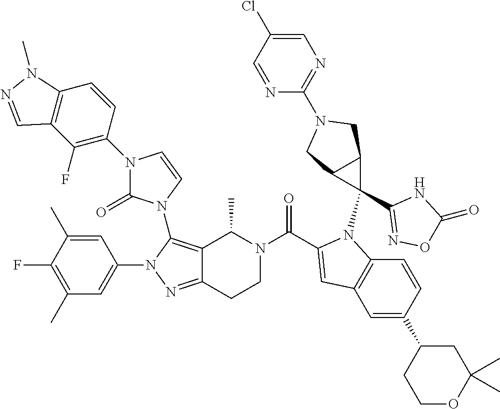 |
| C-39 | 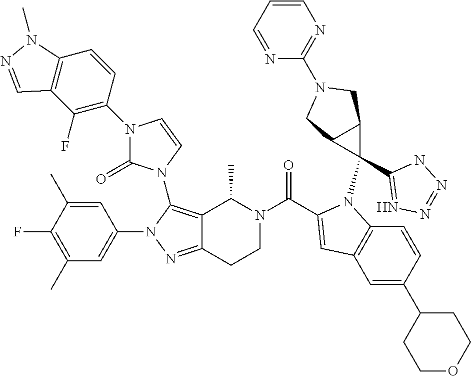 |
| C-40 | 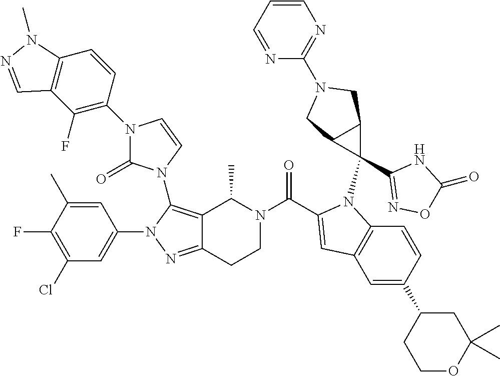 |
| C-41 | 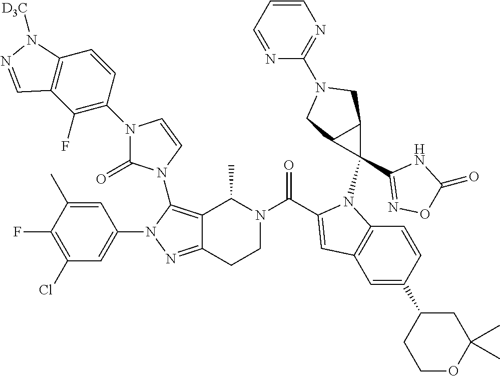 |
| C-42 | 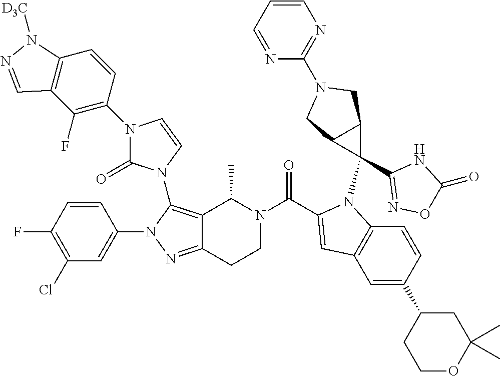 |
| C-43 | 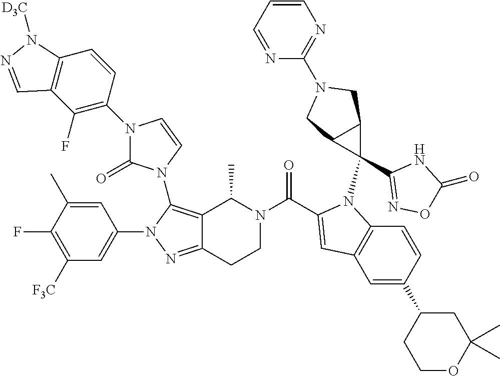 |
| C-44 | 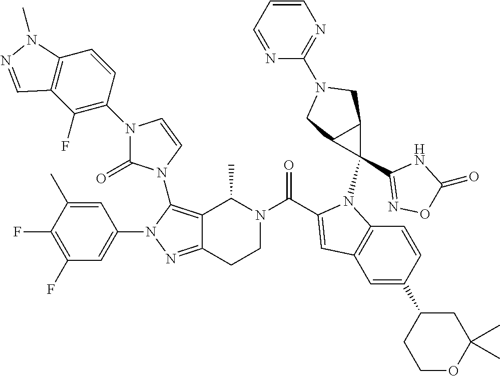 |
| C-45 | 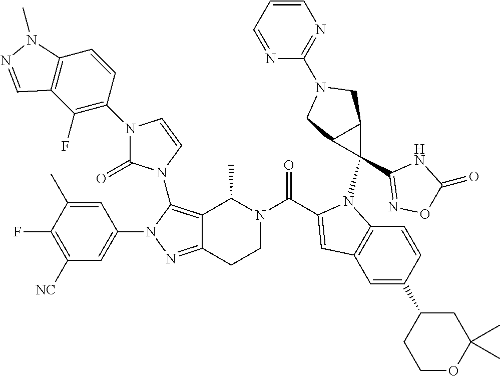 |
| C-46 | 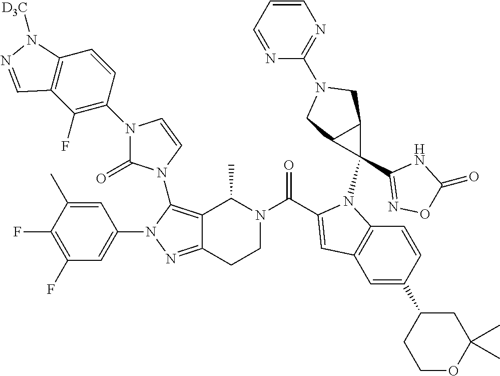 |
| C-47 | 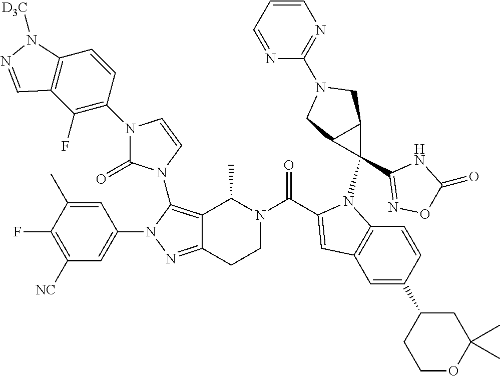 |
| C-48 | 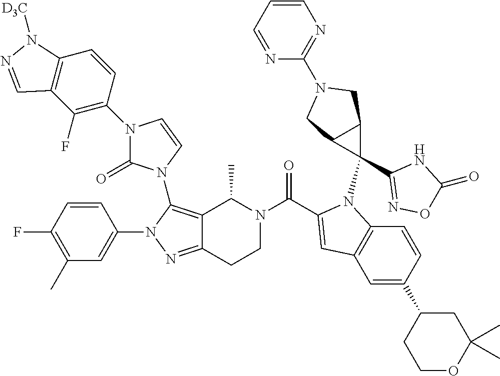 |
| C-49 | 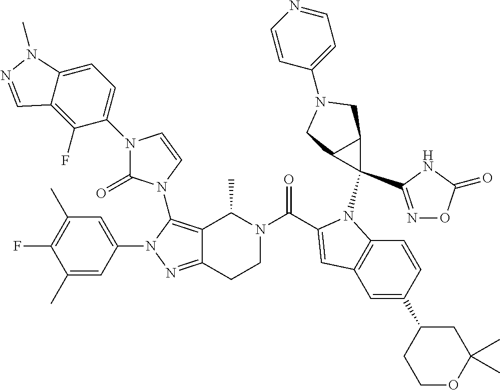 |
| C-50 | 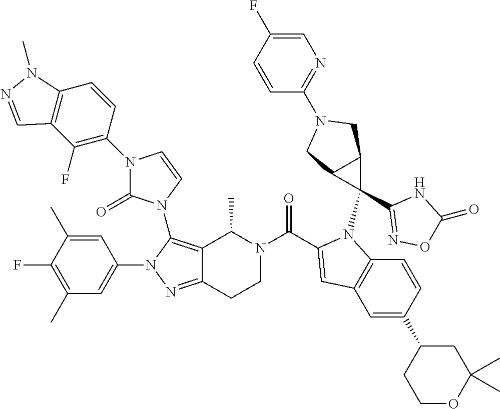 |
| C-51 | 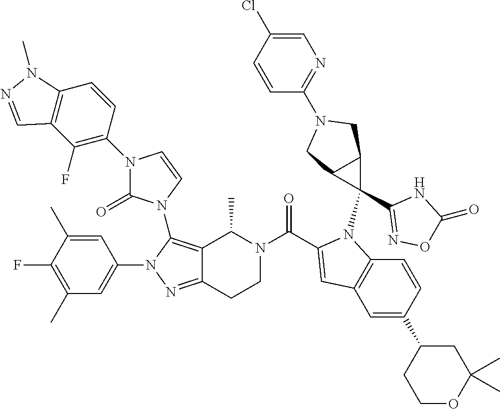 |
| C-52 | 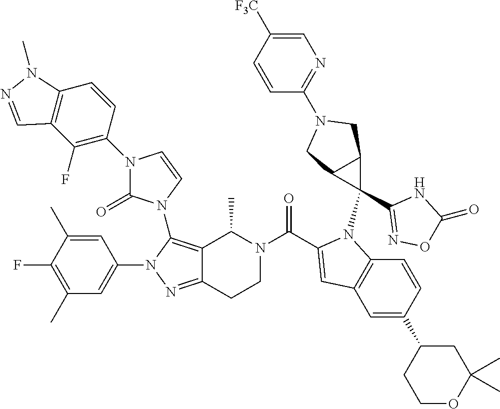 |
| C-53 | 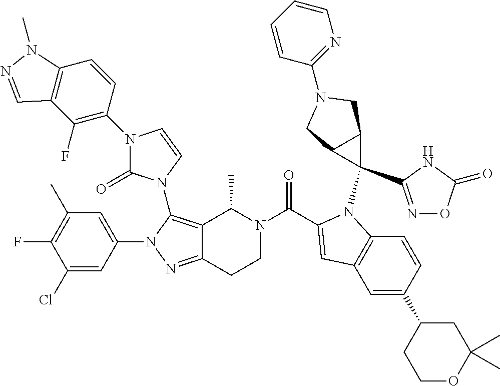 |
| C-54 | 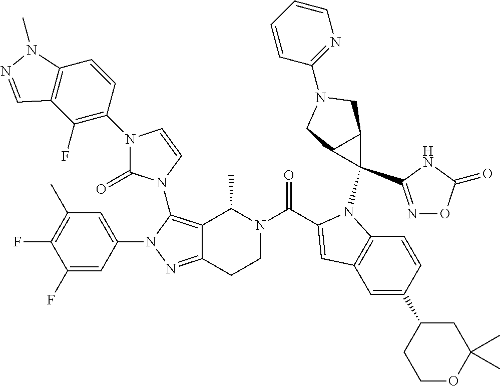 |
| C-55 | 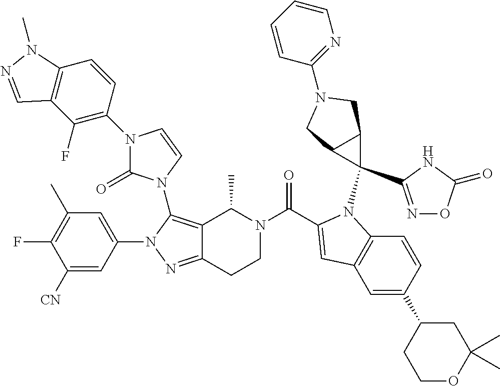 |
| C-56 | 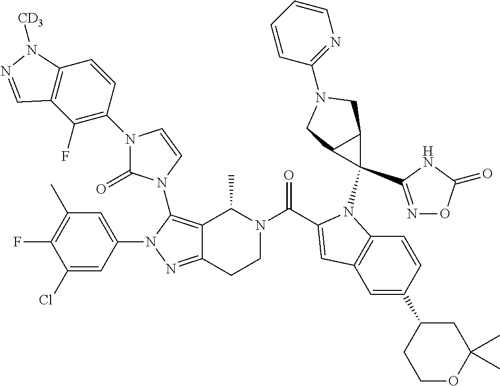 |
| C-57 | 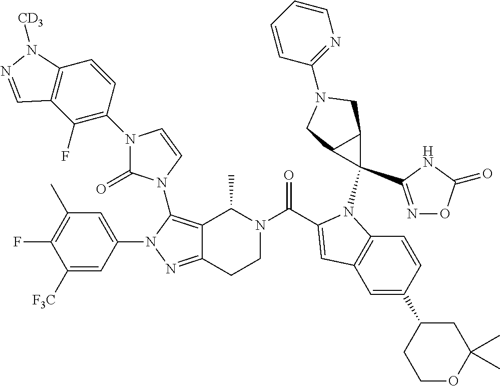 |
| C-58 | 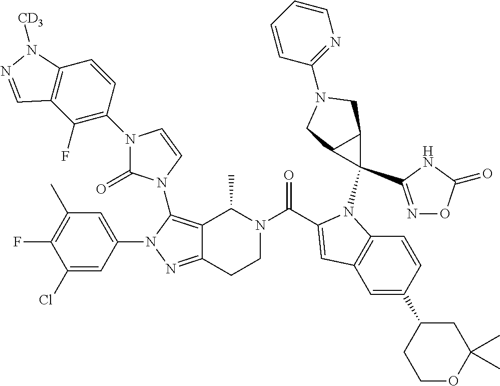 |
| C-59 | 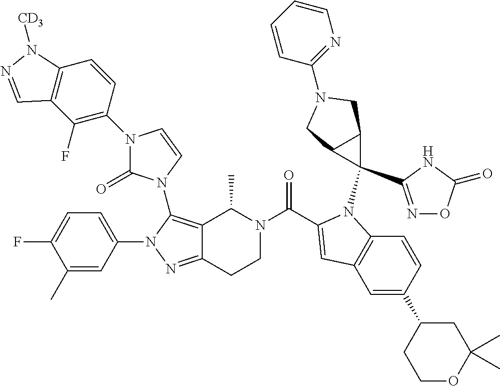 |
| C-60 | 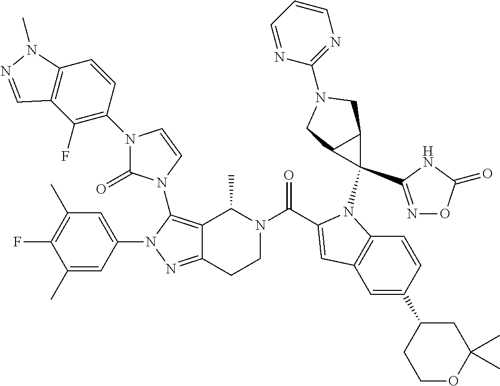 |

In some embodiments, a compound (e.g., any described herein, such as a compound of Formulas (I), (IIa)-(IIc), (IIIa)-(IIIc), (IV), (IVa), and (IVb), or in Table 1 or Table 3c) exhibits GLP-1 agonism activity (e.g., measured as an EC₅₀ value, in which such activity may constitute potent GLP-1 agonism activity). In some embodiments, the GLP-1 agonism activity is more than 0 nM. In some embodiments, the GLP-1 agonism activity is less than 20 nM. In some embodiments, the GLP-1 agonism activity is greater than 20 nM and less than 200 nM. In some embodiments, the GLP-1 agonism activity is greater than 200 nM. In some embodiments, GLP-1 agonism is provided in reference to human GLP-1 (e.g., UniProt Ref. No. P01275 for pro-glucagon, the pre-processed form of GLP-1) or human GLP-1 receptor (e.g., UniProt Ref No. P43220).

In some embodiments, a compound (e.g., any described herein, such as a compound of Formulas (I), (IIa)-(IIc), (IIIa)-(IIIc), (IV), (IVa), and (IVb), or in Table 1 or Table 3c) modulates GLP-1 activity. In some embodiments, a compound (e.g., any described herein, such as a compound of Formulas (I), (IIa)-(IIc), (IIIa)-(IIIc), (IV), (IVa), and (IVb), or in Table 1 or Table 3c) modulates (e.g., activates or stimulates) the GLP-1 receptor (e.g., human GLP-1 receptor).

### Pharmaceutical Compositions

The compounds herein (e.g., such as a compound of Formulas (I), (IIa)-(IIc), (IIIa)-(IIIc), (IV), (IVa), and (IVb), or in Table 1 or Table 3c) can be provided as a pharmaceutical composition having one or more pharmaceutically acceptable excipients. In some embodiments, the composition includes a structure of any one of Formulas (I), (IIa)-(IIc), (IIIa)-(IIIc), (IV), (IVa), and (IVb), or a structure provided in Table 1 or Table 3c, as well as pharmaceutically acceptable salts thereof, stereoisomers thereof, analogs thereof, solvates thereof, prodrugs thereof, or tautomers thereof with one or more pharmaceutically acceptable excipients.

The compositions can be administered by a variety of routes, depending upon whether local or systemic treatment is desired and upon the area to be treated. Administration can include oral, topical (e.g., transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), or parenteral (e.g., intravenous, intraarterial, subcutaneous, intraperitoneal, intramuscular, intracranial, intrathecal, intratumoral, or intraventricular delivery) modes.

The pharmaceutical composition can include any therapeutically effective amount of the one or more compounds for the disease, disorder, or condition to be treated.

In some embodiments, a compound herein (e.g., including a structure of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof) can be formulated within a composition to be administered at a dose (e.g., as a single dose, as separate doses, or as a combined dose).

### Methods of Treatment

The compounds and compositions herein can be used in any useful method. In any embodiment herein, the method can include administrating (e.g., to a subject in need thereof) a compound herein (e.g., including a structure of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof) or a composition herein (e.g., a pharmaceutical composition including a structure of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof with one or more pharmaceutically acceptable excipients).

Provided herein are methods of treating a GLP-1 associated disease, disorder, or condition. Without wishing to be limited by mechanism, GLP-1 is a peptide hormone that binds to the GLP-1 receptor and promotes secretion of insulin. In turn, GLP-1 agonists represent a class of compounds that bind to (e.g., and activate) the GLP-1 receptor. Such agonists can be employed to treat conditions that would benefit from activation of the GLP-1 receptor (e.g., enhanced release of insulin). For example and without limitation, type 2 diabetes can arise from insulin resistance or insufficient insulin production, and use of GLP-1 agonists can be used in treatment (either alone or in combination with other therapies).

Non-limiting examples of diseases, disorders, or conditions include one or more of the following: diabetes, obesity, overweight condition, hyperlipidemia, hypercholesteremia, hypertriglyceridemia, atherosclerosis, hypertension, stroke, coronary heart disease, congestive heart failure, cardiac arrhythmias, diabetic kidney disease, dementia, Parkinson's disease, Alzheimer's disease, and liver diseases such as nonalcoholic fatty liver disease (NAFLD), and nonalcoholic steatohepatitis (NASH).

Other non-limiting examples of diseases, disorders, or conditions include one or more of the following: type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity (including hypothalamic obesity and monogenic obesity), overweight, weight gain from use of other agents, idiopathic intracranial hypertension, Wolfram syndrome, gout, excessive sugar craving, hypertriglyceridemia, dyslipidemia, hyperlipidemia, malnutrition-related diabetes, gestational diabetes, kidney disease, adipocyte dysfunction, sleep apnea, visceral adipose deposition, eating disorders, cardiovascular disease, arteriosclerosis, coronary heart disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, brain infarction, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's disease, Alzheimer's disease, impaired cognition, dementia, schizophrenia, Polycystic Ovary Syndrome (PCOS), or any combination thereof.

In some embodiments, the present disclosure includes a method for treating or prophylactically treating a GLP-1 associated disease, disorder, or condition, the method comprising administering to a subject in need thereof an effective amount of a compound herein (e.g., including a structure of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof) or a composition herein (e.g., including a compound of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof with one or more pharmaceutically acceptable excipients). In some embodiments, the disease, disorder, or condition is any described herein (e.g., diabetes, obesity, overweight, etc.).

In some embodiments, the present disclosure includes a method of treating or prophylactically treating diabetes in a subject in need thereof, the method comprising administering to a subject in need thereof an effective amount of a compound herein (e.g., including a structure of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof) or a composition herein (e.g., including a compound of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof with one or more pharmaceutically acceptable excipients).

In some embodiments, the present disclosure includes a method of treating or prophylactically treating type 2 diabetes mellitus in a subject in need thereof, the method comprising administering to a subject in need thereof an effective amount of a compound herein (e.g., including a structure of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof) or a composition herein (e.g., including a compound of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof with one or more pharmaceutically acceptable excipients).

In some embodiments, the present disclosure includes a method of treating or prophylactically treating obesity or overweight in a subject in need thereof, the method comprising administering to a subject in need thereof an effective amount of a compound herein (e.g., including a structure of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof) or a composition herein (e.g., including a compound of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof with one or more pharmaceutically acceptable excipients).

In some embodiments, the present disclosure includes a method for modulating insulin levels in a subject in need of such modulating, the method comprising administering to a subject in need thereof an effective amount of a compound herein (e.g., including a structure of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof) or a composition herein (e.g., including a compound of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof with one or more pharmaceutically acceptable excipients).

In some embodiments, the present disclosure includes a method for modulating glucose levels in a subject in need of such modulating, the method comprising administering to a subject in need thereof an effective amount of a compound herein (e.g., including a structure of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof) or a composition herein (e.g., including a compound of any one of Formulas (I), (IIa)-(IIc), (IIIa)-(IIIc), (IV), (IVa), and (IVb) or a structure provided in Table 1 or Table 3c, as well as pharmaceutically acceptable salts thereof, stereoisomers thereof, analogs thereof, solvates thereof, prodrugs thereof, or tautomers thereof with one or more pharmaceutically acceptable excipients).

In any embodiment herein, the method can further include administering an additional therapy or therapeutic agent to the subject. In some embodiments, the additional therapy or therapeutic agent can be an anti-diabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or a combination thereof.

In some embodiments, the compounds described herein(for example, compound C-12 or C-22) exhibit improved pharmacokinetics in a subject, such as a longer half-life, higher exposure, lower clearance rate, and/or greater bioavailability. In some embodiments, the compounds described herein exhibit improved absorption profile in a subject. In some embodiments, the compounds described herein also exhibit improved solubility (compared to Orforglipron), weight loss, and blood sugar reduction.

### Biological Assays

The biological activities of the compounds of the present disclosure can be assessed with methods and assays known in the art. Exemplary methods are described in the Examples, such as GLP-1R cAMP assay.

### ENUMERATED EMBODIMENTS

Enumerated Embodiment 1. A compound of Formula (I):

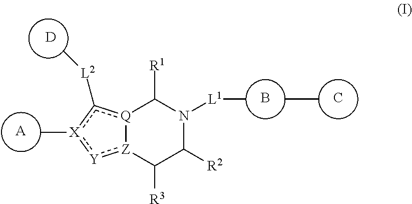

or a pharmaceutically acceptable salt thereof, stereoisomer thereof, analog thereof (e.g., deuterated analog thereof), or solvate thereof, wherein:
each of R¹, R², and R³ is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy);
each of L¹ and L² is, independently, a linker;
each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z);
A comprises an optionally substituted aryl (e.g., an optionally substituted C₆₋₁₀ aryl, such as optionally substituted phenyl) (e.g., wherein optionally substituents include halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these);
B comprises an optionally substituted heterocyclyl (e.g., an optionally substituted 5-10 membered heterocyclyl, such as optionally substituted bicyclic, 5-10 membered heterocyclyl) (e.g., wherein B may be optionally substituted with V¹, as described herein);
C comprises

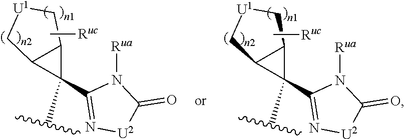

wherein U¹ is oxy (e.g., —O—), thio (e.g., —S—), sulfinyl (e.g., —S(O)—), sulfonyl (e.g., —S(O)₂—), optionally substituted amino (e.g., —NR^{ua}—), or optionally substituted alkylene (e.g., optionally substituted C₁₋₆ alkylene); U² is O or S; each of n1 and n2 is, independently, 0, 1, 2, or 3 (e.g., wherein at least one of n1 and n2 is not 0); each R^{ua} is, independently, hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl); and each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2} as described herein), or an isotope, wherein one or more R^{uc} can be present on the ring to which it is attached, and

D comprises optionally substituted heterocyclyl (e.g., an optionally substituted 5-membered heterocyclyl, such as optionally substituted bicyclic, 5-10 membered heterocyclyl) (e.g., wherein optionally substituents include halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

Enumerated Embodiment 2. The compound of Enumerated Embodiment 1, wherein each of R¹, R², and R³ is, independently, hydrogen (H) or optionally substituted alkyl.

Enumerated Embodiment 3. The compound of Enumerated Embodiment 1 or, wherein each of X and Y is N, each of Y and Z is N, or each of Y and Q is N.

Enumerated Embodiment 4. The compound of any one of Enumerated Embodiments 1-, wherein X is N, and wherein a first double bond is present between Q and the adjacent carbon atom and a second double bond is present between Y and Z.

Enumerated Embodiment 5. The compound of any one of Enumerated Embodiments 1-, wherein Z is N, and wherein a first double bond is present between Q and the adjacent carbon atom and a second double bond is present between X and Y.

Enumerated Embodiment 6. The compound of any one of Enumerated Embodiments 1-, wherein Q is N, and wherein a first double bond is present between X and the adjacent carbon atom and a second double bond is present between Y and Z.

Enumerated Embodiment 7. The compound of any one of Enumerated Embodiments 1-, wherein L¹ is carbonyl (e.g., —C(O)—), alkylene (e.g., optionally substituted C₁₋₆ alkylene), sulfinyl (e.g., —S(O)—), or sulfonyl (e.g., —S(O)₂—).

Enumerated Embodiment 8. The compound of any one of Enumerated Embodiments 1-, wherein L² is

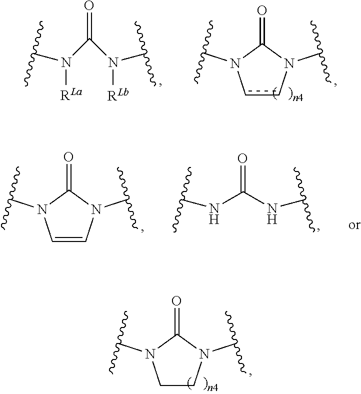

and wherein:
n4 is 1, 2, or 3, and the dashed line represents an optional double bond (e.g., wherein two double bonds are not attached to the same atom); and
each of R^{La} and R^{Lb}, if present, is, independently, hydrogen (H), halo, optionally substituted alkyl (e.g., optionally substituted C₁₋₆ alkyl), or optionally substituted alkoxy (e.g., optionally substituted C₁₋₆ alkoxy), and optionally wherein R^{La} and R^{Lb}, taken together with the nitrogen atoms to which each is attached, forms an optionally substituted heterocyclyl.

Enumerated Embodiment 9. The compound of any one of Enumerated Embodiments 1-, wherein A comprises an optionally substituted C₆₋₁₀ aryl, and wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, or C₃₋₈ halocycloalkyl.

Enumerated Embodiment 10. The compound of any one of Enumerated Embodiments 1-, wherein A comprises

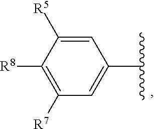

wherein each of R⁵, R⁶, and R⁷ is, independently, hydrogen (H), halo, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), or optionally substituted halocycloalkyl (e.g., optionally substituted C₃₋₈ halocycloalkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

Enumerated Embodiment 11. The compound of Enumerated Embodiment, wherein A comprises

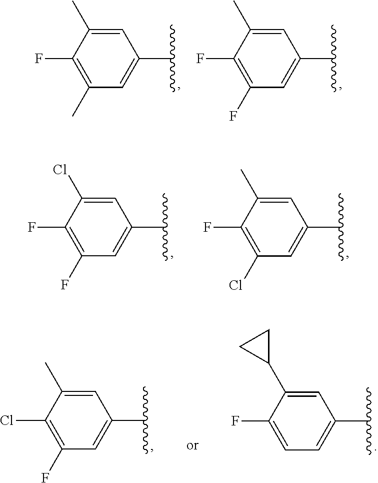

Enumerated Embodiment 12. The compound of any one of Enumerated Embodiments 1-, wherein B comprises

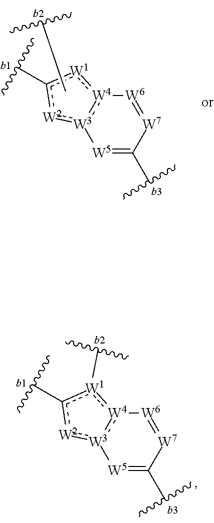

and
wherein:
each of W¹, W³, and W⁴ is, independently, C or N;
W² is CR^{wa}, NR^{wa}, N, O, or S, and the dashed line represents an optional double bond (e.g., wherein two double bonds are not attached to the same atom);
each of W⁵, W⁶, and W⁷ is, independently, CR^{wa} or N (e.g., wherein b1, b2, and b3 is connected to L¹, C, and V¹ in any order or position); and
each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

Enumerated Embodiment 13. The compound of Enumerated Embodiment, wherein B comprises

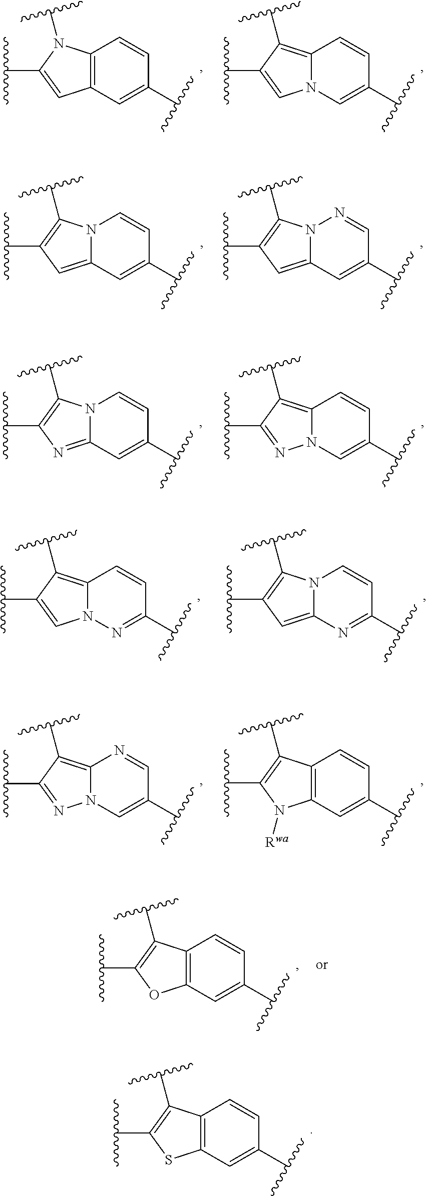

Enumerated Embodiment 14. The compound of any one of Enumerated Embodiments 1-, wherein B comprises

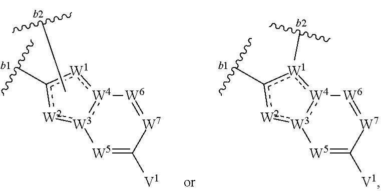

wherein:
each of W¹, W³, and W⁴ is, independently, C or N;
W² is CR^{wa}, NR^{wa}, N, O, or S, and the dashed line represents an optional double bond (e.g., wherein two double bonds are not attached to the same atom);
each of W⁵, W⁶, and W⁷ is, independently, CR^{wa} or N (e.g., wherein b1 and b2 is connected to L¹ and C in any order or position);
each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these); and
V¹ comprises an optionally substituted heterocyclyl (e.g., an optionally substituted heteroaryl).

Enumerated Embodiment 15. The compound of Enumerated Embodiment, wherein B comprises

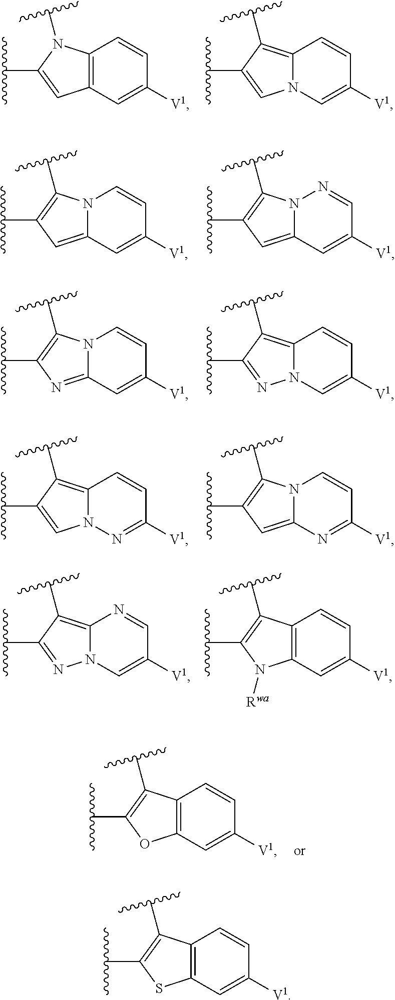

Enumerated Embodiment 16. The compound of Enumerated Embodiment or, wherein V¹ comprises

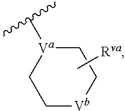

and
wherein:
V^{a} is N or CR^{vc};
V^{b} is NR^{Vc}, O, or S;
each of R^{va} and R^{vc}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these), and wherein one or more R^{va} may be present on V¹.

Enumerated Embodiment 17. The compound of Enumerated Embodiment or, wherein V¹ comprises

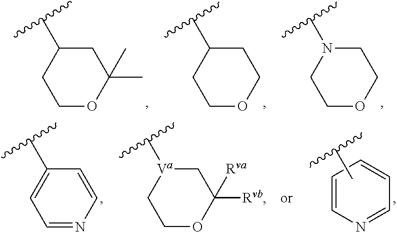

and wherein:
V^{a} is N or CR^{vc},
each of R^{va} R^{vb}, and R^{vc}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

Enumerated Embodiment 18. The compound of any one of Enumerated Embodiments 1-, wherein C comprises

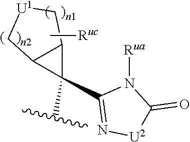

or a stereoisomer thereof.

Enumerated Embodiment 19. The compound of any one of Enumerated Embodiments 1-, wherein C comprises

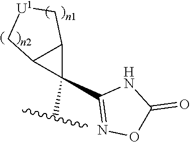

or a stereoisomer thereof.

Enumerated Embodiment 20. The compound of any one of Enumerated Embodiments 1-, wherein U¹ is an optionally substituted C₁₋₆ alkylene (e.g., —CR^{Ua}R^{Ub}— or —[CR^{ua}R^{ub}]ₙ₃—, wherein n3 is 1, 2, or 3, and wherein each of R^{ua} and R^{ub}, if present, is, independently, hydrogen (H), optionally substituted alkyl (e.g., optionally substituted C₁₋₆ alkyl), or optionally substituted alkoxy (e.g., optionally substituted C₁₋₆ alkoxy)).

Enumerated Embodiment 21. The compound of any one of Enumerated Embodiments 1-, wherein C comprises

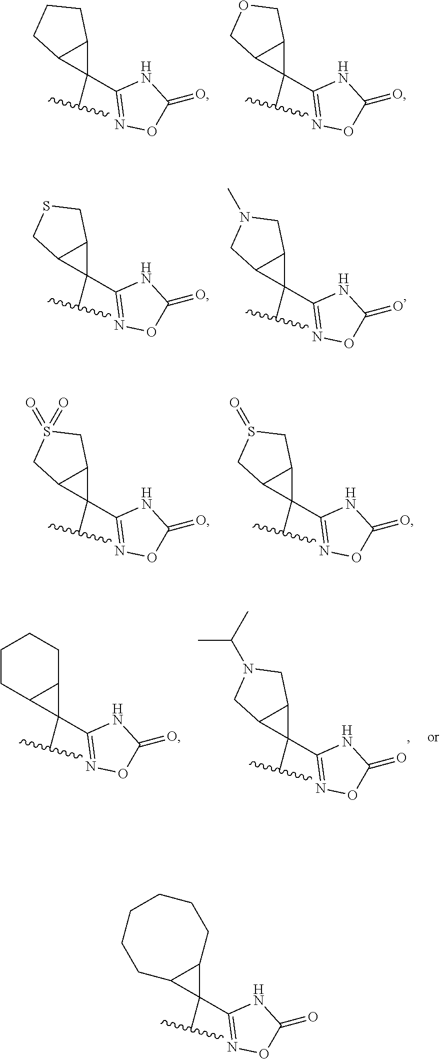

Enumerated Embodiment 22. The compound of any one of Enumerated Embodiments 1-, wherein D comprises

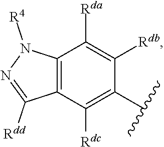

and wherein:
each of R^{da}, R^{bd}, R^{dc}, and R^{dd} is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy); and
R⁴ is hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl) (e.g., wherein optionally substituents include halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

Enumerated Embodiment 23. The compound of Enumerated Embodiment, wherein D comprises

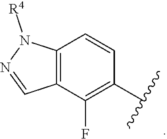

Enumerated Embodiment 24. The compound of Enumerated Embodiment, wherein D comprises

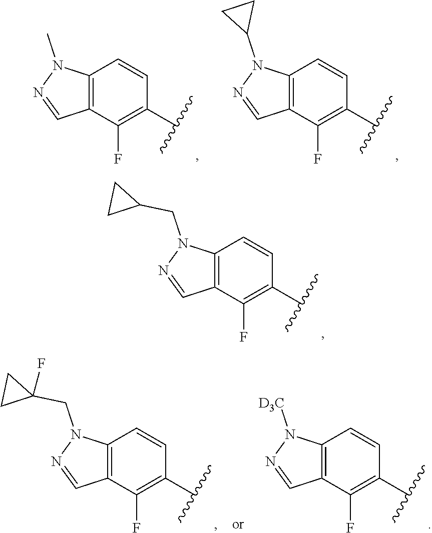

Enumerated Embodiment 25. The compound of Enumerated Embodiment 1, wherein the compound comprises a structure of Formula (IIa):

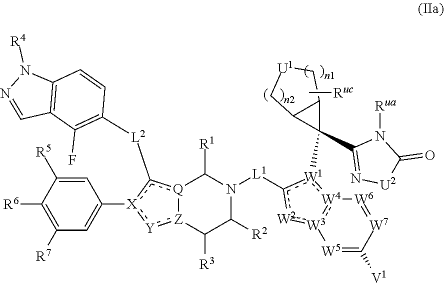

or a pharmaceutically acceptable salt thereof, stereoisomer thereof, analog thereof (e.g., deuterated analog thereof), or solvate thereof, wherein:
each of R¹, R², and R³ is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy);
R⁴ is hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl);
each of R⁵, R⁶, and R⁷ is, independently, hydrogen (H), halo, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), or optionally substituted halocycloalkyl (e.g., optionally substituted C₃₋₈ halocycloalkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these);
each of L¹ and L² is, independently, a linker;
each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z);
each of W¹, W³, and W⁴ is, independently, C or N;
W² is CR^{wa}, NR^{wa}, N, O, or S, and the dashed line represents an optional double bond (e.g., wherein two double bonds are not attached to the same atom);
each of W⁵, W⁶, and W⁷ is, independently, CR^{wa} or N;
each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these);
U¹ is oxy (e.g., —O—), thio (e.g., —S—), sulfinyl (e.g., —S(O)—), sulfonyl (e.g., —S(O)₂—), optionally substituted amino (e.g., —NR^{ua}—), or optionally substituted alkylene (e.g., optionally substituted C₁₋₆ alkylene);
U² is O or S;
each R^{ua} is, independently, hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl);
each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached;
each of n1 and n2 is, independently, 0, 1, 2, or 3 (e.g., wherein at least one of n1 and n2 is not 0); and
V¹ comprises an optionally substituted heterocyclyl (e.g., an optionally substituted heteroaryl).

Enumerated Embodiment 26. The compound of Enumerated Embodiment 1, wherein the compound comprises a structure of Formula (IIb):

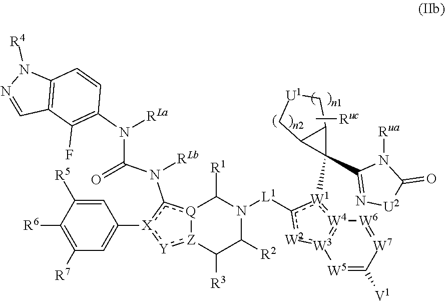

or a pharmaceutically acceptable salt thereof, stereoisomer thereof, analog thereof (e.g., deuterated analog thereof), or solvate thereof, wherein:
each of R¹, R², and R³ is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy);
R⁴ is hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl);
each of R⁵, R⁶, and R⁷ is, independently, hydrogen (H), halo, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), or optionally substituted halocycloalkyl (e.g., optionally substituted C₃₋₈ halocycloalkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these);
each of R^{La} and R^{Lb}, if present, is, independently, hydrogen (H), optionally substituted alkyl (e.g., optionally substituted C₁₋₆ alkyl), or optionally substituted alkoxy (e.g., optionally substituted C₁₋₆ alkoxy), and optionally wherein R^{La} and R^{Lb}, taken together with the nitrogen atoms to which each is attached, forms an optionally substituted heterocyclyl;
each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z);
each of W¹, W³, and W⁴ is, independently, C or N;
W² is CR^{wa}, NR^{wa}, N, O, or S, and the dashed line represents an optional double bond (e.g., wherein two double bonds are not attached to the same atom);
   a. each of W⁵, W⁶, and W⁷ is, independently, CR^{wa} or N;
each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these);
U¹ is oxy (e.g., —O—), thio (e.g., —S—), sulfinyl (e.g., —S(O)—), sulfonyl (e.g., —S(O)₂—), optionally substituted amino (e.g., —NR^{ua}—), or optionally substituted alkylene (e.g., optionally substituted C₁₋₆ alkylene);
U² is O or S;
each R^{ua} is, independently, hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl);
each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached;
each of n1 and n2 is, independently, 0, 1, 2, or 3 (e.g., wherein at least one of n1 and n2 is not 0); and
V¹ comprises an optionally substituted heterocyclyl (e.g., an optionally substituted heteroaryl).

Enumerated Embodiment 27. The compound of Enumerated Embodiment 1, wherein the compound comprises a structure of Formula (IIc):

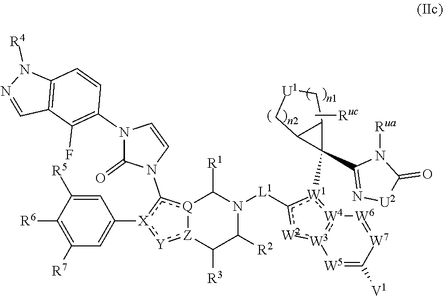

or a pharmaceutically acceptable salt thereof, stereoisomer thereof, analog thereof (e.g., deuterated analog thereof), or solvate thereof, wherein:
each of R¹, R², and R³ is, independently, hydrogen (H), halo, hydroxyl, optionally substituted alkyl (e.g., optionally substituted C₁₋₆ alkyl), or optionally substituted alkoxy (e.g., optionally substituted C₁₋₆ alkoxy);
R⁴ is hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl);
each of R⁵, R⁶, and R⁷ is, independently, hydrogen (H), halo, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), or optionally substituted halocycloalkyl (e.g., optionally substituted C₃₋₈ halocycloalkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these);
each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z);
each of W¹, W³, and W⁴ is, independently, C or N;
W² is CR^{wa}, NR^{wa}, N, O, or S, and the dashed line represents an optional double bond (e.g., wherein two double bonds are not attached to the same atom);
each of W⁵, W⁶, and W⁷ is, independently, CR^{wa} or N;
each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these);
U¹ is oxy (e.g., —O—), thio (e.g., —S—), sulfinyl (e.g., —S(O)—), sulfonyl (e.g., —S(O)₂—), optionally substituted amino (e.g., —NR^{ua}—), or optionally substituted alkylene (e.g., optionally substituted C₁₋₆ alkylene);
U² is O or S;
each R^{ua} is, independently, hydrogen (H), optionally substituted alkyl (e.g., optionally substituted C₁₋₆ alkyl), or optionally substituted alkoxy (e.g., optionally substituted C₁₋₆ alkoxy);
each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached;
each of n1 and n2 is, independently, 0, 1, 2, or 3 (e.g., wherein at least one of n1 and n2 is not 0); and
V¹ comprises an optionally substituted heterocyclyl (e.g., an optionally substituted heteroaryl).

Enumerated Embodiment 28. The compound of any one of Enumerated Embodiments 1-, wherein X is N; X and Y is N; Y and Z is N; or Y and W is N.

Enumerated Embodiment 29. The compound of any one of Enumerated Embodiments-, wherein at least one of W¹, W², W³, W⁴, W⁵, W⁶, or W⁷ is, independently, N or NR^{Wa}; and wherein R^{wa} is hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

Enumerated Embodiment 30. The compound of any one of Enumerated Embodiments 1-, wherein R¹ is optionally substituted alkyl (e.g., optionally substituted C₁₋₆ alkyl).

Enumerated Embodiment 31. The compound of any one of Enumerated Embodiments 1-, wherein at least one of R¹, R², and R³ is not H.

Enumerated Embodiment 32. The compound of any one of Enumerated Embodiments 1-, wherein R⁴ is C₁₋₆ alkyl, deuterated C₁₋₆ alkyl, C₃₋₈ cycloalkyl, C₃₋₈ halocycloalkyl, C₁₋₆ alkyl substituted with one or more C₃₋₈ cycloalkyl, or C₁₋₆ alkyl substituted with one or more C₃₋₈ halocycloalkyl.

Enumerated Embodiment 33. The compound of any one of Enumerated Embodiments 1-, wherein each of R⁵, R⁶, and R⁷ is, independently, halo, C₁₋₆ alkyl, C₃₋₈ cycloalkyl, C₃₋₈ halocycloalkyl, C₁₋₆ alkyl substituted with one or more C₃₋₈ cycloalkyl, or C₁₋₆ alkyl substituted with one or more C₃₋₈ halocycloalkyl.

Enumerated Embodiment 34. The compound of any one of Enumerated Embodiments 1-, wherein R⁶ is halo (e.g., fluoro or chloro).

Enumerated Embodiment 35. The compound of any one of Enumerated Embodiments 1-, wherein each of R⁵ and R⁷ is, independently, halo, optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl), (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

Enumerated Embodiment 36. The compound of any one of Enumerated Embodiments 1-, wherein at least one of R⁵, R⁶, and R⁷is not H.

Enumerated Embodiment 37. The compound of any one of Enumerated Embodiments 1-, wherein U¹ is O or methylene (CH₂).

Enumerated Embodiment 38. The compound of any one of Enumerated Embodiments 1-, wherein n1 is 1, and/or wherein n2 is 1.

Enumerated Embodiment 39. The compound of any one of Enumerated Embodiments 1-, wherein U² is 0, and/or wherein R^{ua} is H or C₁₋₆ alkyl.

Enumerated Embodiment 40. The compound of any one of Enumerated Embodiments 1-, wherein V¹ comprises

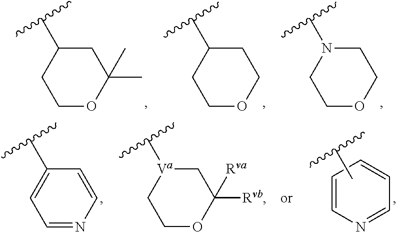

and wherein:
V^{a} is N or CR^{vc},
each of R^{va}, R^{vb}, and R^{vc}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

Enumerated Embodiment 41. The compound of Enumerated Embodiment 1, wherein the compound comprises a structure of Formula (IIIa):

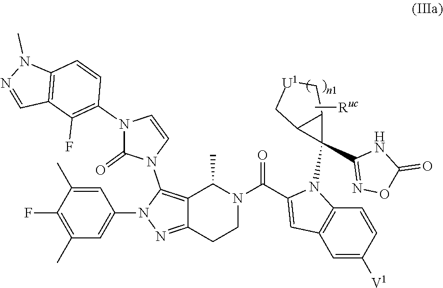

or a pharmaceutically acceptable salt thereof, stereoisomer thereof, analog thereof (e.g., deuterated analog thereof), or solvate thereof, wherein: U¹ is oxy (e.g., —O—), thio (e.g., —S—), sulfinyl (e.g., —S(O)—), sulfonyl (e.g., —S(O)₂—), optionally substituted amino (e.g., —NR^{ua}—), or optionally substituted alkylene (e.g., optionally substituted C₁₋₆ alkylene);
each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached;
n1 is 0, 1, 2, or 3; and
V¹ is an optionally substituted heterocyclyl.

Enumerated Embodiment 42. The compound of Enumerated Embodiment, wherein V¹ comprises

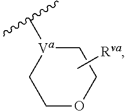

and
wherein:
V^{a} is N or CR^{vc},
each of R^{va} and R^{vc}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₃₋₈ cycloalkyl, or a combination of any of these), and wherein one or more R^{va} may be present on V¹.

Enumerated Embodiment 43. The compound of Enumerated Embodiment, wherein V¹ comprises

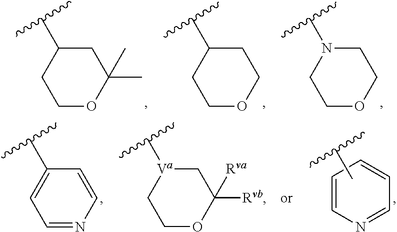

and wherein:
V^{a} is N or CR^{vc},
each of R^{va}, R^{vb}, and R^{vc}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these).

Enumerated Embodiment 44. The compound of Enumerated Embodiment 1, wherein the compound comprises a structure of Formula (IIb):

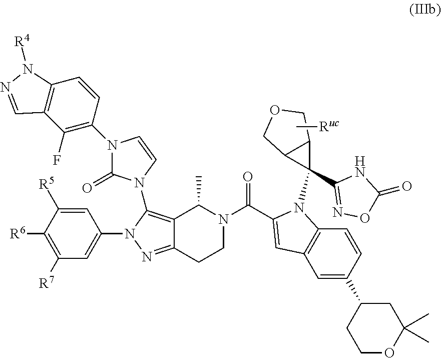

or a pharmaceutically acceptable salt thereof, stereoisomer thereof, analog thereof (e.g., deuterated analog thereof), or solvate thereof, wherein:
   R⁴ is hydrogen (H), optionally substituted alkyl (e.g., optionally substituted C₁₋₆ alkyl), optionally substituted alkoxy (e.g., optionally substituted C₁₋₆ alkoxy), or optionally substituted cycloalkyl (e.g., optionally substituted C₃₋₈ cycloalkyl);
   each of R⁵, R⁶, and R⁷ is, independently, hydrogen (H), halo, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), or optionally substituted halocycloalkyl (e.g., optionally substituted C₃₋₈ halocycloalkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these); and
   each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached.

Enumerated Embodiment 45. The compound of Enumerated Embodiment, wherein R⁴ is C₁₋₆ alkyl (e.g., methyl), deuterated C₁₋₆ alkyl (e.g., deuterated methyl, such as CD₃), C₃₋₈ cycloalkyl (e.g., cyclopropyl, such as

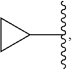 [SMILES: *C1CC1]

C₁₋₆ alkyl substituted with one or more C₃₋₈ cycloalkyl (e.g., cyclopropylalkyl, including cyclopropylmethyl, such as

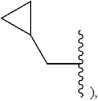 [SMILES: *CC1CC1]

C₃₋₈ halocycloalkyl (e.g., 1-halocyclopropyl, including 1-fluorocyclopropyl, such as

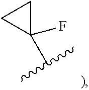 [SMILES: *C1(F)CC1]

or C₁₋₆ alkyl substituted with one or more C₃₋₈ halocycloalkyl (e.g., 1-halocyclopropylalkyl, including 1-fluorocyclopropylmethyl, such as

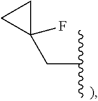 [SMILES: *CC1(F)CC1]

Enumerated Embodiment 46. The compound of Enumerated Embodiment or, wherein each of R⁵, R⁶, and R⁷ is, independently, halo (e.g., fluoro or chloro), C₁₋₆ alkyl (e.g., methyl), C₃₋₈ cycloalkyl (e.g., cyclopropyl, such as C₃₋₈ cycloalkyl (e.g., cyclopropyl), C₁₋₆ alkyl substituted with one or more C₃₋₈ cycloalkyl (e.g., cyclopropylalkyl, including cyclopropylmethyl), C₃₋₈ halocycloalkyl (e.g., 1-halocyclopropyl, including 1-fluorocyclopropyl), or C₁₋₆ alkyl substituted with one or more C₃₋₈ halocycloalkyl (e.g., 1-halocyclopropylalkyl, including 1-fluorocyclopropylmethyl).

Enumerated Embodiment 47. The compound of Enumerated Embodiment 1, wherein the compound comprises a structure of Formula (IIIc):

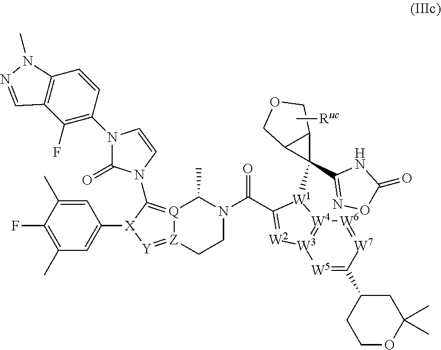

or a pharmaceutically acceptable salt thereof, stereoisomer thereof, analog thereof (e.g., deuterated analog thereof), or solvate thereof, wherein:
   each of X, Y, Z, and Q is, independently, C or N, and the dashed line represents an optional double bond (e.g., wherein two double bonds are not attached to the same atom, and optionally wherein a double bond is present between Q and the adjacent carbon atom and/or between Y and Z);
   each of W¹, W³, and W⁴ is, independently, C or N;
   W² is CR^{wa}, NR^{wa}, N, O, or S, and the dashed line represents an optional double bond (e.g., wherein two double bonds are not attached to the same atom);
   each of W⁵, W⁶, and W⁷ is, independently, CR^{wa} or N;
   each R^{wa}, if present, is, independently, hydrogen (H), halo, or optionally substituted alkyl (e.g., optionally substituted C₁₋₆ alkyl) (e.g., wherein optional substituents include one or more of the following: halo, C₁₋₆ alkyl, C₁₋₆ haloalkyl, C₁₋₆ alkoxy, C₁₋₆ haloalkoxy, C₃₋₈ cycloalkyl, C₃₋₈ halocycloalkyl, or a combination of any of these); and
   each R^{uc} is, independently, hydrogen (H), hydroxyl, optionally substituted alkyl (e.g., optionally substituted C₁₋₆ alkyl), optionally substituted haloalkyl (e.g., optionally substituted C₁₋₆ haloalkyl), optionally substituted alkoxy (e.g., optionally substituted C₁₋₆ alkoxy), optionally substituted haloalkoxy (e.g., optionally substituted C₁₋₆ haloalkoxy), optionally substituted cycloalkyl (e.g., optionally substituted C₃₋₈ cycloalkyl), optionally substituted halocycloalkyl (e.g., optionally substituted C₃₋₈ halocycloalkyl), amino (e.g., —NR^{N1}R^{N2}, as described herein), or an isotope, wherein one or more R^{uc} can be present on the ring to which it is attached.

Enumerated Embodiment 48. The compound of Enumerated Embodiment, wherein

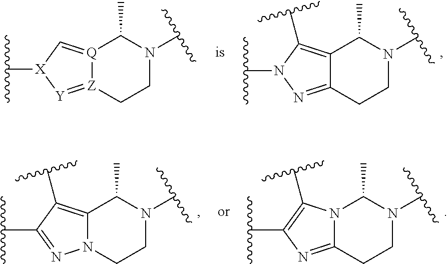

Enumerated Embodiment 49. The compound of Enumerated Embodiment or, wherein

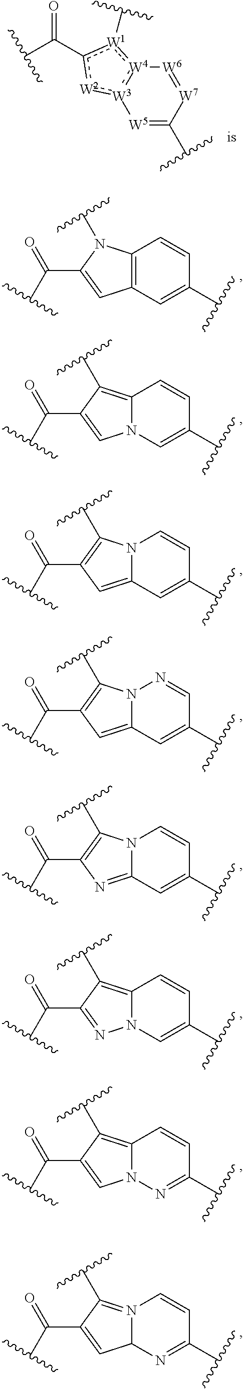

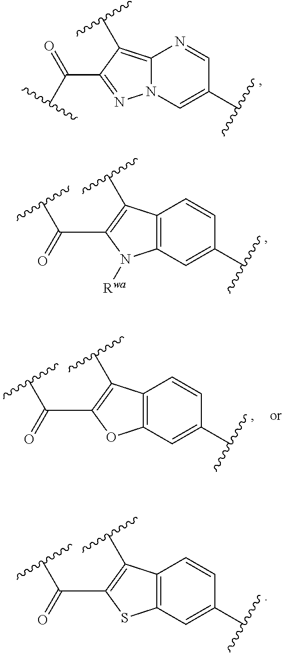

Enumerated Embodiment 50. A compound according to Table 1, or a pharmaceutically acceptable salt thereof, stereoisomer thereof, analog thereof (e.g., deuterated analog thereof), or solvate thereof.

Enumerated Embodiment 51. A pharmaceutical composition comprising a compound of any one of Enumerated Embodiments 1-, or a pharmaceutically acceptable salt thereof, stereoisomer thereof, analog thereof (e.g., deuterated analog thereof), or solvate thereof, and at least one pharmaceutically acceptable excipient.

Enumerated Embodiment 52. A method for treating or prophylactically treating a GLP-1 associated disease, disorder, or condition, the method comprising administering to a subject in need thereof an effective amount of the compound of any one of Enumerated Embodiments 1-, or a pharmaceutically acceptable salt thereof, stereoisomer thereof, analog thereof (e.g., deuterated analog thereof), or solvate thereof, or the pharmaceutical composition of Enumerated Embodiment.

Enumerated Embodiment 53. The method of Enumerated Embodiment, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity (including hypothalamic obesity and monogenic obesity), overweight, weight gain from use of other agents, idiopathic intracranial hypertension, Wolfram syndrome, gout, excessive sugar craving, hypertriglyceridemia, dyslipidemia, hyperlipidemia, malnutrition-related diabetes, gestational diabetes, kidney disease, adipocyte dysfunction, sleep apnea, visceral adipose deposition, eating disorders, cardiovascular disease, arteriosclerosis, coronary heart disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, brain infarction, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's disease, Alzheimer's disease, impaired cognition, dementia, schizophrenia, Polycystic Ovary Syndrome (PCOS), or any combination thereof.

Enumerated Embodiment 54. A method of treating or prophylactically treating diabetes in a subject in need thereof, the method comprising administering to a subject in need thereof an effective amount of the compound of any one of Enumerated Embodiments 1-, or a pharmaceutically acceptable salt thereof, stereoisomer thereof, analog thereof (e.g., deuterated analog thereof), or solvate thereof, or the pharmaceutical composition of Enumerated Embodiment.

Enumerated Embodiment 55. A method of treating or prophylactically treating type 2 diabetes mellitus in a subject in need thereof, the method comprising administering to a subject in need thereof an effective amount of the compound of any one of Enumerated Embodiments 1-, or a pharmaceutically acceptable salt thereof, stereoisomer thereof, analog thereof (e.g., deuterated analog thereof), or solvate thereof, or the pharmaceutical composition of Enumerated Embodiment.

Enumerated Embodiment 56. A method of treating or prophylactically treating obesity or overweight in a subject in need thereof, the method comprising administering to a subject in need thereof an effective amount of the compound of any one of Enumerated Embodiments 1-, or a pharmaceutically acceptable salt thereof, stereoisomer thereof, analog thereof (e.g., deuterated analog thereof), or solvate thereof, or the pharmaceutical composition of Enumerated Embodiment.

Enumerated Embodiment 57. A method for modulating insulin levels in a subject in need of such modulating, the method comprising administering to a subject in need thereof an effective amount of the compound of any one of Enumerated Embodiments 1-, or a pharmaceutically acceptable salt thereof, stereoisomer thereof, analog thereof (e.g., deuterated analog thereof), or solvate thereof, or the pharmaceutical composition of Enumerated Embodiment.

Enumerated Embodiment 58. A method for modulating glucose levels in a subject in need of such modulating, the method comprising administering to a subject in need thereof an effective amount of the compound of any one of Enumerated Embodiments 1-, or a pharmaceutically acceptable salt thereof, stereoisomer thereof, analog thereof (e.g., deuterated analog thereof), or solvate thereof, or the pharmaceutical composition of Enumerated Embodiment.

Enumerated Embodiment 59. The method of any one of Enumerated Embodiments-, further comprising administering an additional therapy or therapeutic agent to the subject.

Enumerated Embodiment 60. The method of Enumerated Embodiment, wherein the additional therapy or therapeutic agent is selected from the group consisting of an anti-diabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or a combination thereof.

Enumerated Embodiment 61. The compound of any one of Enumerated Embodiments 1-, or a pharmaceutically acceptable salt thereof, stereoisomer thereof, tautomer thereof, analog thereof (e.g., deuterated analog thereof), or solvate thereof, for use in treating or prophylactically treating a GLP-1 associated disease, disorder, or condition.

Enumerated Embodiment 62. Use of the compound of any one of Enumerated Embodiments 1-, or a pharmaceutically acceptable salt thereof, stereoisomer thereof, tautomer thereof, analog thereof (e.g., deuterated analog thereof), or solvate thereof in the manufacture of a medicament for treating or prophylactically treating a GLP-1 associated disease, disorder, or condition.

Enumerated Embodiment 63. The compound for use according to Enumerated Embodiment 61 or the use according to Enumerated Embodiment 62, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity (including hypothalamic obesity and monogenic obesity), overweight, weight gain from use of other agents, idiopathic intracranial hypertension, Wolfram syndrome, gout, excessive sugar craving, hypertriglyceridemia, dyslipidemia, hyperlipidemia, malnutrition-related diabetes, gestational diabetes, kidney disease, adipocyte dysfunction, sleep apnea, visceral adipose deposition, eating disorders, cardiovascular disease, arteriosclerosis, coronary heart disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, brain infarction, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's disease, Alzheimer's disease, impaired cognition, dementia, schizophrenia, Polycystic Ovary Syndrome (PCOS), or any combination thereof.

### EXAMPLES

Abbreviations (as used herein):

| | |
|---|---|
| h | hour |
| min | minute |
| EtOAc | ethyl acetate |
| DMSO | dimethyl sulfoxide |
| DMF | N,N-dimethylformamide |
| DMAC | N,N-dimethylacetamide |
| THF | tetrahydrofuran |
| NMP | N-methyl-2-pyrrolidone |
| n-BuLi | n-butyllithium |
| DBAD | di-tert-butyl azodicarboxylate |
| HCl | hydrochloric acid |
| TEA | triethylamine |
| EtOH | ethanol |
| Boc₂O | di-tert-butyl dicarbonate |
| t-BuOK | potassium tert-butoxide |
| MsOH | methanesulfonic acid |
| Py•HCl | pyridine hydrochloride |
| PhMe | toluene |
| K₃PO4 | potassium phosphate |
| CuI | cuprous iodide |
| K₂CO₃ | potassium carbonate |
| Zn | zinc |
| TMSCl | trimethylchlorosilane |
| BrCH₂CH₂Br | 1,2-dibromoethane |
| Pd(OAc)₂ | palladium acetate |
| Amphos | (4-N,N-dimethylamino)phenylbis-tert- |
| | butylphosphonium |
| NaOH | sodium hydroxide |
| aq. | aqueous |
| NaH | sodium hydride |
| SOCl₂ | thionyl chloride |
| DCM | dichloromethane |
| NaIO₄ | sodium periodate |
| RuCl₃ | ruthenium trichloride |
| ACN | acetonitrile |
| KHMDS | potassium bis(trimethylsilyl)amide |
| DIPEA | N,N-diisopropylethylamine |
| NH₂OH•HCl | hydroxylamine hydrochloride |
| NaHCO₃ | sodium bicarbonate |
| CDI | carbonyl diimidazole |
| DBU | 1,8-diazabicycloundec-7-ene |
| KOH | potassium hydroxide |
| EGME | 2-methoxyethanol |
| HATU | 2-(7-azabenzotriazol-l-yl)-N,N,N,N,- |
| | tetramethyluroniumhexafluorophosphate |
| SFC | supercritical fluid chromatogram |

In the following examples, when the structural formula of a compound does not match the chemical name, the structural formula of the compound shall prevail.

### Example 1: (S)-1-(4-fluoro-1-methyl-1H-indazol-5-yl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one (intermediate I-1)

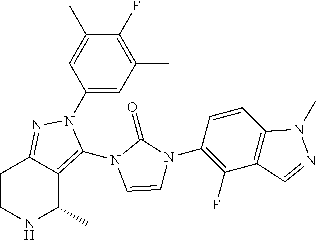 [SMILES: Cc1cc(-n2nc3c(c2-n2ccn(-c4ccc5c(cnn5C)c4F)c2=O)[C@H](C)NCC3)cc(C)c1F]

Scheme 1 shows non-limiting steps A to J to provide intermediate I-1.

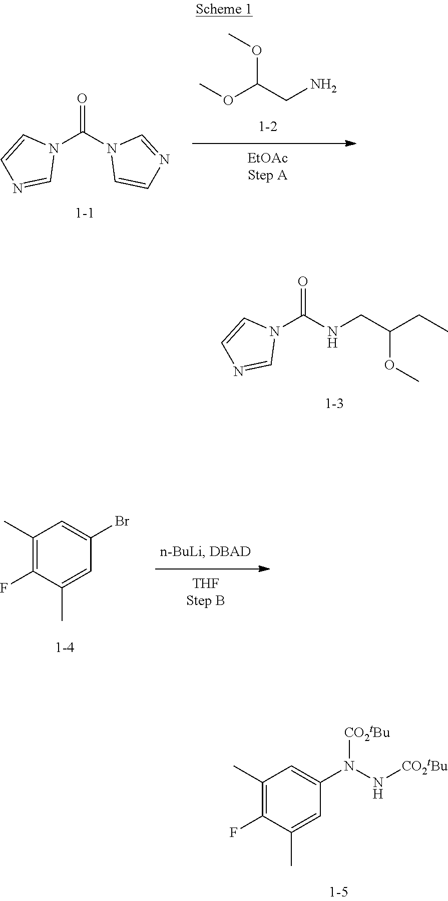

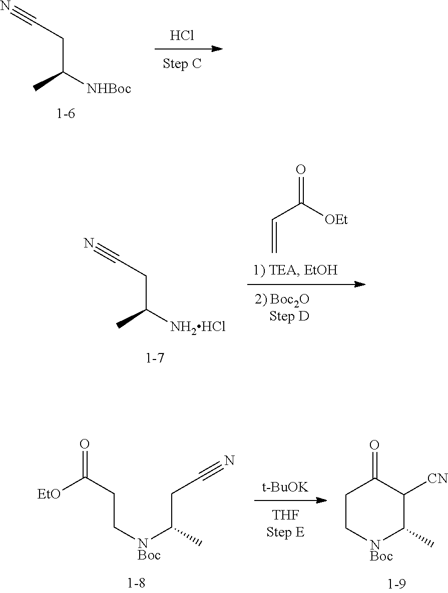

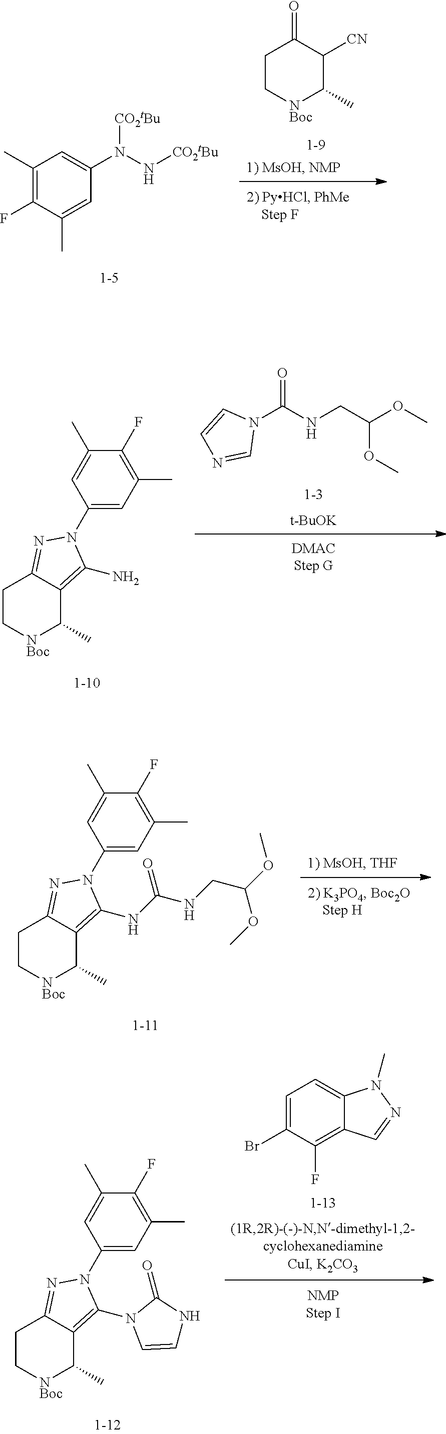

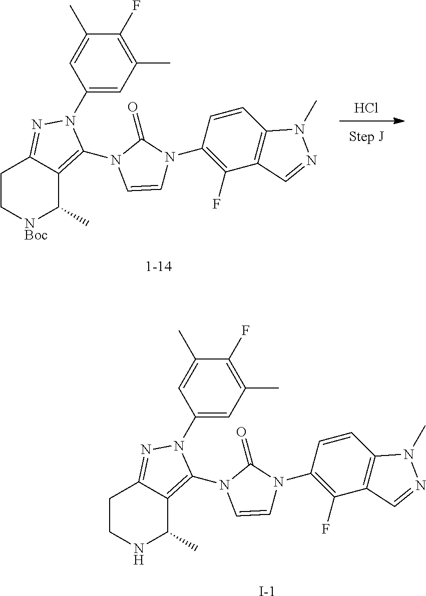 [SMILES: Cc1cc(-n2nc3c(c2-n2ccn(-c4ccc5c(cnn5C)c4F)c2=O)[C@H](C)N(C(=O)OC(C)(C)C)CC3)cc(C)c1F ; Cl ; Cc1cc(-n2nc3c(c2-n2ccn(-c4ccc5c(cnn5C)c4F)c2=O)[C@H](C)NCC3)cc(C)c1F ; [I-]]

### Step A: N-(2,2-dimethoxyethyl)-1H-imidazole-1-carboxamide (1-3)

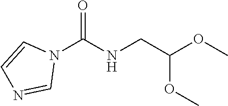

To a mixture of compound 1-1 (50.00 g, 308.36 mmol) in ethyl acetate (125 mL) was added compound 1-2 (22.69 g, 215.85 mmol) at −10° C. After the addition was completed, the reaction mixture was stirred at 25° C. for 2 h. The reaction mixture was cooled to 0° C. and filtered. The filtrate was concentrated, and the residue was purified by silica gel column chromatography (40-44% ethyl acetate/petroleum ether) to give compound 1-3 (42.00 g, 79%) as white solid.

LC-MS: ESI-MS (m/z): [M+H]⁺=200.1.

### Step B: di-tert-butyl 1-(4-fluoro-3,5-dimethylphenyl)hydrazine-1,2-dicarboxylate (1-5)

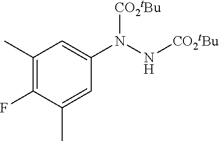 [SMILES: Cc1cc(N(NC(=O)OC(C)(C)C)C(=O)OC(C)(C)C)cc(C)c1F]

To a mixture of compound 1-4 (45.00 g, 221.62 mmol) in tetrahydrofuran (450 mL) was added dropwise n-butyllithium (85.1 mL, 2.5 M in hexane, 212.76 mmol) at −70° C. and stirred for 1 h. Then, di-tert-butyl azodicarboxylate (45.93 g, 199.46 mmol) was added dropwise at −40° C., and the mixture was stirred for 0.5 h. The reaction mixture was warmed to 25° C. over 1 h and quenched with saturated ammonium chloride solution (460 mL). The reaction mixture was extracted with heptane (230 mL). The organic layer was separated and concentrated. Heptane (70 mL) was added, and the mixture was heated to 70° C. to promote dissolution. The solution was cooled to 25° C., and white solid was precipitated for 1 h. The precipitate was collected by filtration, washed with heptane (23 mL), and dried to give compound 1-5 (42.00 g, 53%) as a white solid.

¹H NMR (400 MHz, DMSO-d₆): δ 9.56 (s, 1H), 7.07-6.96 (m, 2H), 2.19 (s, 6H), 1.50-1.37 (m, 18H).

### Step C: (S)-3-aminobutanenitrile hydrochloride (1-7)

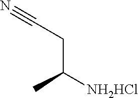

To a mixture of compound 1-6 (27 g, 146.55 mmol) in dichloromethane (150 mL) was added HCl (4 M in 1,4-dioxane, 50 mL) at 25° C. and stirred for 3 h. The reaction solution was concentrated to give crude compound 1-7 (28.0 g) as colorless oil.

### Step D: ethyl (S)-3-((tert-butoxycarbonyl)(1-cyanopropan-2-yl)amino)propanoate (1-8)

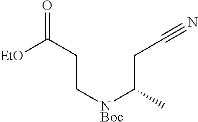 [SMILES: CCOC(=O)CCN(C(=O)OC(C)(C)C)[C@@H](C)CC#N]

To a mixture of compound 1-7 (17.67 g, 146.55 mmol) in ethanol (85 mL) was added triethylamine (29.66 g, 293.1 mmol) and ethyl acrylate (17.61 g, 175.86 mmol) at 25° C. The reaction mixture was stirred at 70° C. for 3 h and then cooled to 25° C. Di-tert-butyl dicarbonate (38.38 g, 175.86 mmol) was added, and the reaction mixture was stirred at 25° C. for 14 h. N-methylpiperazine (3 mL, 27.06 mmol) was added, and the mixture was stirred for 4 h. Diluted HCl solution (50 mL, 1M) was added, and the mixture was extracted by toluene (50 mL×3). The organic layer was separated and washed with aq. sodium chloride (50 mL, 15 wt %) and was concentrated to dryness under reduced pressure to give crude compound 1-8 (41.67 g) as brown oil.

ESI-MS (m/z): [M+H]⁺=284.9.

### Step E: tert-butyl (2S)-3-cyano-2-methyl-4-oxopiperidine-1-carboxylate (1-9)

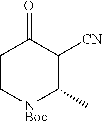 [SMILES: C[C@H]1C(C#N)C(=O)CCN1C(=O)OC(C)(C)C]

To a mixture of crude compound 1-8 (41.67 g) in tetrahydrofuran (410 mL) was added potassium tert-butoxide (16.44 g, 146.54 mmol) at 25° C., and the mixture was stirred for 1 h. Diluted HCl (1.2 L, 2M) was added, and the inner temperature was kept at 15° C. The reaction mixture was extracted with ethyl acetate (200 mL×3). The organic layer was separated and washed aq. sodium chloride (300 mL X 2, 15 wt %) to give crude compound 1-9 (36.00 g) as brown oil.

ESI-MS (m/z): [M+H]⁺=238.9.

¹H NMR (400 MHz, DMSO-d₆): δ 4.58-4.42 (m, 1H), 4.01-3.88 (m, 1H), 2.96 (s, 1H), 2.50 (s, 1H), 2.38-2.26 (m, 1H), 2.19 (dd, J=17.6, 3.4 Hz, 1H), 1.41 (s, 9H), 1.21 (d, J=6.6 Hz, 3H).

### Step F: tert-butyl (S)-3-amino-2-(4-fluoro-3,5-dimethylphenyl)-4-methyl-2,4,6,7-tetrahydro-5H-pyrazolo[4,3-c]pyridine-5-carboxylate (1-10)

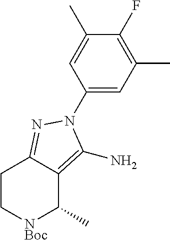 [SMILES: Cc1cc(-n2nc3c(c2N)[C@H](C)N(C(=O)OC(C)(C)C)CC3)cc(C)c1F]

To a mixture of compound 1-5 (42.00 g, 118.50 mmol) in N-methyl-2-pyrrolidinone (126 mL) was added methanesulfonic acid (22.78 g, 237 mmol), and the mixture was stirred at 80° C. for 7 h. The reaction mixture was cooled to 25° C.; and toluene (252 mL), potassium carbonate (18.02 g, 130.35 mmol) and water (252 mL) was added. The reaction mixture was stirred at 25° C. for 10 minutes (min). The organic layer was separated, to which was added a solution of compound 1-9 (27.82 g, 116.74 mmol) in toluene (126 mL), pyridine hydrochloride (1.35 g, 11.67 mmol) and toluene (84 mL). The reaction mixture was stirred at 90° C. for 1 h and cooled to 25° C. The reaction mixture was washed with aq. sodium hydroxide (252 mL, 1M). The organic layer was separated and concentrated to dryness under reduced pressure to give compound 1-10 (34.80 g, 80%) as brown oil.

ESI-MS (m/z): [M+H]⁺=374.9.

¹H NMR (400 MHz, DMSO-d₆): δ 7.22 (d, J=6.4 Hz, 2H), 5.18 (s, 2H), 5.07 (d, J=43.4 Hz, 1H), 4.24-4.02 (m, 1H), 3.14-2.90 (m, 1H), 2.48-2.38 (m, 2H), 2.25 (d, J=1.2 Hz, 6H), 1.43 (s, 9H), 1.24 (d, J=6.0 Hz, 3H).

### Step G: tert-butyl (S)-3-(3-(2,2-dimethoxyethyl)ureido)-2-(4-fluoro-3,5-dimethylphenyl)-4-methyl-2,4,6,7-tetrahydro-5H-pyrazolo[4,3-c]pyridine-5-carboxylate (1-11)

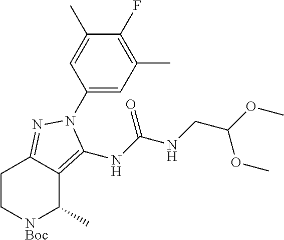 [SMILES: COC(CNC(=O)Nc1c2c(nn1-c1cc(C)c(F)c(C)c1)CCN(C(=O)OC(C)(C)C)[C@H]2C)OC]

To a mixture of compound 1-10 (11.00 g, 29.38 mmol) and compound 1-3 (17.56 g, 88.14 mmol) in N,N-dimethylacetamide (110 mL) was added potassium tert-butoxide (19.78 g, 176.28 mmol), and the mixture was stirred at 25° C. for 2 h. The reaction mixture was quenched with water (200 mL) at 0° C. and extracted by ethyl acetate (80 mL×3). The combined organic layers were washed with saturated sodium chloride (50 mL×3), dried over anhydrous sodium sulfate, and concentrated to driness under reduced pressure. The residue was purified by silica gel column chromatography (0-5% MeOH/dichloromethane) to give compound 1-11 (14.00 g, 94%) as yellow solid.

ESI-MS (m/z): [M+H]⁺=505.9.

### Step H: tert-butyl (S)-2-(4-fluoro-3,5-dimethylphenyl)-4-methyl-3-(2-oxo-2,3-dihydro-1H-imidazol-1-yl)-2,4,6,7-tetrahydro-5H-pyrazolo[4,3-c]pyridine-5-carboxylate (1-12)

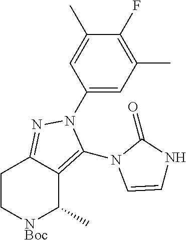 [SMILES: Cc1cc(-n2nc3c(c2-n2cc[nH]c2=O)[C@H](C)N(C(=O)OC(C)(C)C)CC3)cc(C)c1F]

To a mixture of compound 1-11 (14.00 g, 27.69 mmol) in tetrahydrofuran (140 mL) was added methanesulfonic acid (2.66 g, 27.69 mmol), and the mixture was stirred at 60° C. for 2 h. The reaction mixture was cooled to 25° C., and a solution of potassium phosphate (2.35 g, 11.08 mmol) in water (56 mL) and di-tert-butyl dicarbonate (4.83 g, 22.15 mmol) was added. The resulting mixture was stirred at 25° C. for 1 h, diluted with water (150 mL), and extracted with ethyl acetate (50 mL×3). The combined organic layers were washed with saturated sodium chloride (50 mL×3), dried over anhydrous sodium sulfate, and concentrated to driness under reduced pressure. The residue was then purified by silica gel column chromatography (20-30% EA/PE) to give compound 1-12 (8.00 g, 65%) as yellow solid.

ESI-MS (m/z): [M+H]⁺=442.2.

### Step I: tert-butyl (S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridine-5-carboxylate (1-14)

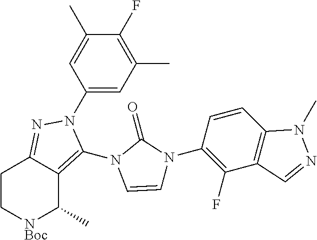 [SMILES: Cc1cc(-n2nc3c(c2-n2ccn(-c4ccc5c(cnn5C)c4F)c2=O)[C@H](C)N(C(=O)OC(C)(C)C)CC3)cc(C)c1F]

To a mixture of compound 1-12 (1.00 g, 2.27 mmol) and compound 1-13 (778 mg, 3.40 mmol) in N-methyl-2-pyrrolidone (10 mL) was added cuprous iodide (216 mg, 1.14 mmol), (1R,2R)-(−)-N,N-dimethyl-1,2-cyclohexanediamine (323 mg, 2.27 mmol), and potassium carbonate (941 mg, 6.81 mmol). The mixture was stirred under nitrogen atmosphere at 120° C. for 16 h. The reaction mixture was poured into water (50 mL), extracted with ethyl acetate (10 mL×3). The combined organic layers were washed with saturated sodium chloride, dried over anhydrous sodium sulfate, and concentrated to driness under reduced pressure. The residue was purified by silica gel column chromatography (50-62%, ethyl acetate/petroleum ether) to give compound 1-14 (1.10 g, 82%) as yellow solid.

ESI-MS (m/z): [M+H]⁺=590.0.

### Step J: (S)-1-(4-fluoro-1-methyl-1H-indazol-5-yl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one (I-1)

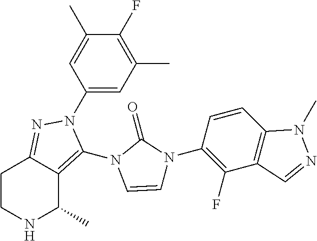 [SMILES: Cc1cc(-n2nc3c(c2-n2ccn(-c4ccc5c(cnn5C)c4F)c2=O)[C@H](C)NCC3)cc(C)c1F]

To a mixture of compound 1-14 (450 mg) in dichloromethane (4.5 mL) was added HCl (4 M in 1,4-dioxane, 1.9 mL), and the mixture was stirred at 25° C. for 3 h. The reaction mixture was concentrated to driness under reduced pressure. The residue was re-dissolved in ethyl acetate and washed with saturated NaHCO₃ solution. The organic layer was separated, dried over anhydrous sodium sulfate, concentrated to driness under reduced pressure to give compound I-1 (350 mg, 94%) as yellow solid.

ESI-MS (m/z): [M+H]⁺=489.9.

### Example 2: (S)-1-(cyanomethyl)-5-(2,2-dimethyltetrahydro-2H-pyran-4-yl)-N-methyl-N-phenyl-1H-indole-2-carboxamide (intermediate 1-2)

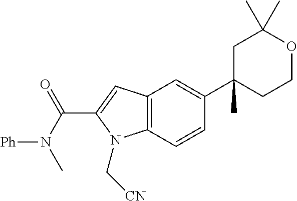 [SMILES: CN(C(=O)c1cc2cc([C@@]3(C)CCOC(C)(C)C3)ccc2n1CC#N)c1ccccc1]

Scheme 2 shows non-limiting steps A to E to provide intermediate I-2.

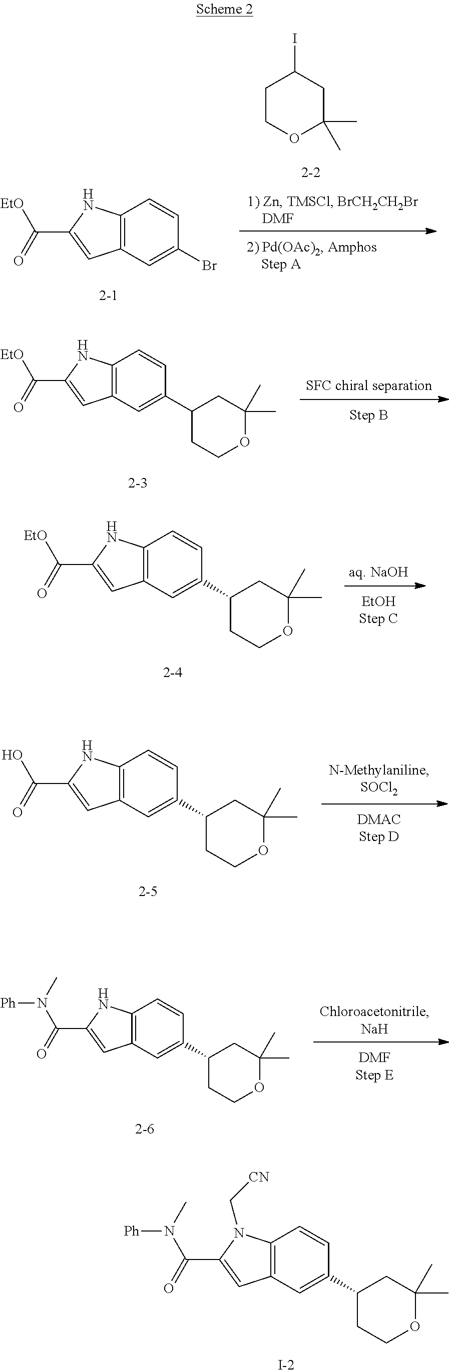

### Step A: ethyl 5-(2,2-dimethyltetrahydro-2H-pyran-4-yl)-1H-indole-2-carboxylate (2-3)

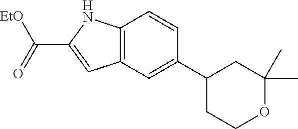 [SMILES: CCOC(=O)c1cc2cc(C3CCOC(C)(C)C3)ccc2[nH]1]

To a suspension of zinc powder (9.00 g, 138.5 mmol) in N, N-dimethylformamide (28 mL) was added trimethylchlorosilane (1.65 g, 15.2 mmol) and 1,2-dibromoethane (2.86 g, 15.2 mmol) under nitrogen atmosphere. The reaction mixture was stirred for 5 min at 25° C. A solution of compound 2-2 (25.00 g, 104.1 mmol) in N, N-dimethylformamide (42 mL) was added and the mixture was stirred for 20 min at 25° C. Palladium acetate (780 mg, 3.5 mmol), (4-N, N-dimethylamino) phenylbis-tert-butylphosphonium (1.84 g, 6.93 mmol), and compound 2-1 (9.31 g, 34.7 mmol) was added, and the mixture was heated to 55° C. for 2 h under nitrogen atmosphere. The reaction mixture was cooled to 25° C., quenched with diluted HCl (4 M), diluted with water (100 mL), and extracted with ethyl acetate (20 mL×3). The combined organic layers were washed with saturated sodium chloride, dried over anhydrous sodium sulfate, and concentrated to driness under reduced pressure. The residue was purified by silica gel column chromatography (0-15%, ethyl acetate/petroleum ether) to give compound 2-3 (9.12 g, 87%) as white solid.

ESI-MS (m/z): [M+H]⁺=302.1.

### Step B: ethyl (S)-5-(2,2-dimethyltetrahydro-2H-pyran-4-yl)-1H-indole-2-carboxylate (2-4)

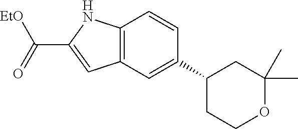 [SMILES: CCOC(=O)c1cc2cc([C@H]3CCOC(C)(C)C3)ccc2[nH]1]

Compound 2-3 (16.67 g) was separated by SFC to give compound 2-4 (8.24 g) and its enantiomer (7.28 g). Separation conditions were as follows:
Instrument: Kep300.
Column: Chiral ART Amylose KBN 5 m Column.
Temperature: 25° C.
Solvent: ethanol=100 (V). Flow rate: 100 mL/min.

Analysis conditions were as follows:
Instrument: Shimadzu LC 20CNZ with UV detector SPD M20A
Column: Chiral ART Amylose KBN (5 μm), 0.46 cm I.D.×25 cm L.
Column temperature: 25° C.
Solvent: ethanol=100 (V)
Flow rate: 1.0 mL/min
Retention time of compound 2-4: 15.980 min, retention time of enantiomer: 9.534 min.

### Step C: (S)-5-(2,2-dimethyltetrahydro-2H-pyran-4-yl)-1H-indole-2-carboxylic acid (2-5)

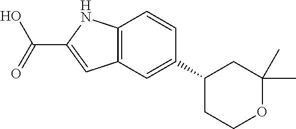 [SMILES: CC1(C)C[C@@H](c2ccc3[nH]c(C(=O)O)cc3c2)CCO1]

To a mixture of compound 2-4 (7.24 g, 24.0 mmol) in MeOH (109 mL) was added aq. sodium hydroxide (26.4 mL, 2M, 52.8 mmol), and the reaction mixture was heated to 65° C. and stirred for 2 h. The reaction mixture was cooled to 0° C., and diluted HCl (23.1 mL, 2.4 M) and water (54 mL) was slowly added. The reaction mixture was stirred for additional 1 h and filtered. The filter cake was dried to give compound 2-5 (5.62 g, 86%) as white solid.

ESI-MS (m/z): [M−H]⁻=272.0.

### Step D: (S)-5-(2,2-dimethyltetrahydro-2H-pyran-4-yl)-N-methyl-N-phenyl-1H-indole-2-carbo-xamide (2-6)

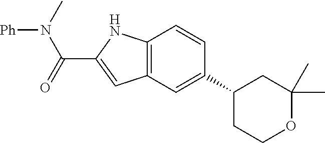 [SMILES: CN(C(=O)c1cc2cc([C@H]3CCOC(C)(C)C3)ccc2[nH]1)c1ccccc1]

To the mixture of compound 2-5 (4.23 g, 15.5 mmol) in N,N-dimethylacetamide (42 mL) under nitrogen atmosphere at 0° C. was added thionyl chloride (2.21 g, 18.6 mmol) dropwise, and the reaction mixture stirred for 1 h. N-methylaniline (1.99 g, 18.7 mmol) and triethylamine (3.12 g, 30.9 mmol) was added, and the reaction mixture was stirred at 250 C for 3 h. The reaction mixture was quenched with water (28 mL) and filtered, and the filter cake was dried to give compound 2-6 (5.39 g, 96%) as white solid.

ESI-MS (m/z): [M+H]⁺=363.2.

### Step E: (S)-1-(cyanomethyl)-5-(2,2-dimethyltetrahydro-2H-pyran-4-yl)-N-methyl-N-phenyl-1H-indole-2-carboxamide (I-2)

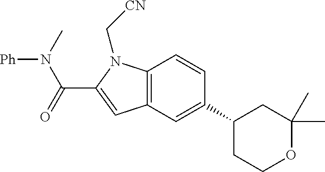 [SMILES: CN(C(=O)c1cc2cc([C@H]3CCOC(C)(C)C3)ccc2n1CC#N)c1ccccc1]

To a mixture of compound 2-6 (2.00 g, 5.5 mmol) in 1,3-dimethyl-2-imidazolidinone (30 mL) was added sodium hydride (0.33 g, 8.3 mmol) under nitrogen atmosphere at 0° C. and stirred for 40 min. Chloro-acetonitrile (0.83 g, 11.0 mmol) was added dropwise. The reaction mixture was heated to 75° C. and stirred for 12 h. The reaction was cooled to 25° C., quenched with water (50 mL), and extracted with ethyl acetate (20 mL×3). The combined organic layers were washed with saturated sodium chloride, concentrated to driness under reduced pressure. The residue was purified by silica gel column chromatography (0-20% ethyl acetate/petroleum ether) to give compound I-2 (2.12 g, 96%) as white solid.

ESI-MS (m/z): [M+H]⁺=402.2.

### Example 3: 3-(6-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-bicyclo[3.1.0]hexan-6-yl)-1,2,4-oxadiazol-5(4H)-one (compound 23)

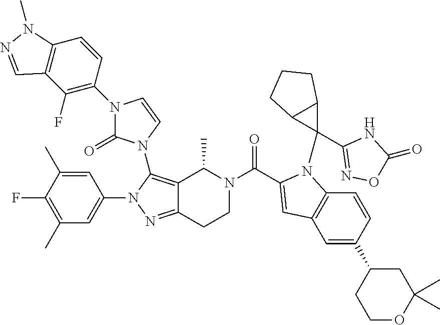 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2C2(c4noc(=O)[nH]4)C4CCCC42)CC3)cc(C)c1F]

Scheme 3 shows non-limiting steps A to F to provide compound 23.

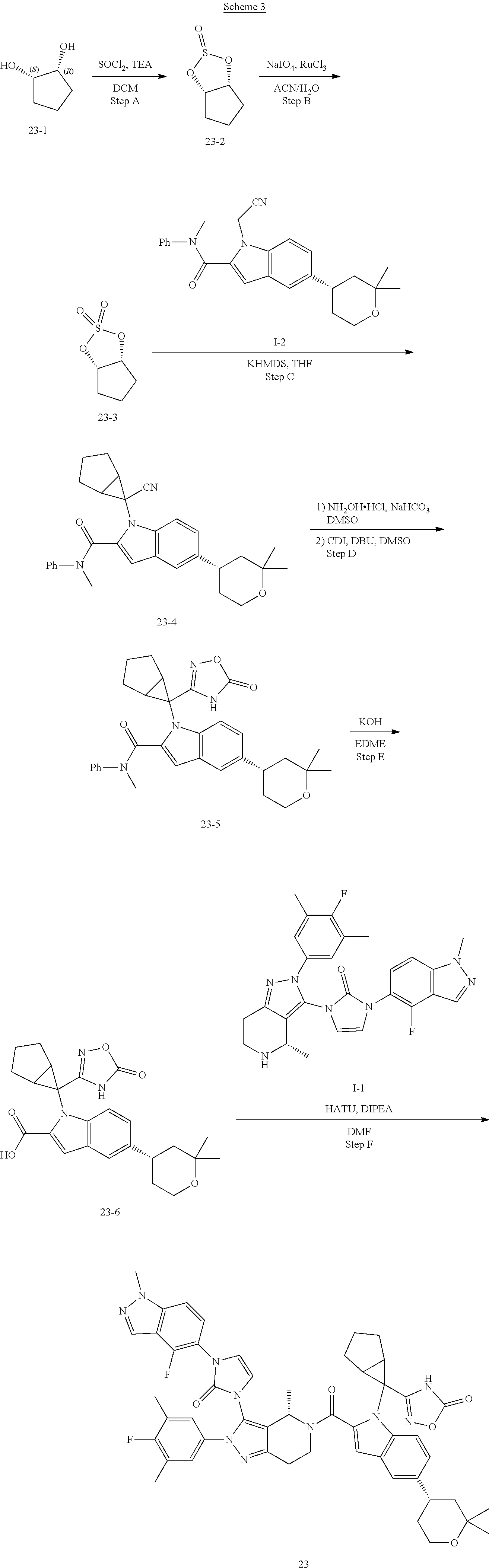

### Step A: (3aR,6aS)-tetrahydro-4H-cyclopenta[d][1,3,2]dioxathiole 2-oxide (23-2)

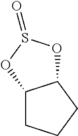

To a mixture of compound 23-1 (1.00 g, 9.8 mmol) and triethylamine (3.96 g, 39.2 mmol) in dichloromethane (20 mL) was added thionyl chloride (1.75 g, 14.7 mmol) dropwise at 0° C. and stirred for 2 h. The reaction mixture was quenched with water (20 mL) and extracted with dichloromethane (10 mL×3). The combined organic layers were concentrated to dryness under reduced pressure to give crude compound 23-2 (1.45 g) as brown oil.

### Step B: (3aR,6aS)-tetrahydro-4H-cyclopenta[d][1,3,2]dioxathiole 2,2-dioxide (23-3)

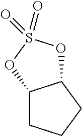

To a mixture of compound 23-2 (1.45 g, 9.8 mmol) in acetonitrile (10 mL) and water (10 mL) was added sodium periodate (2.51 g, 11.7 mmol) and ruthenium trichloride (40.6 mg, 0.2 mmol) at 0° C. The reaction mixture was stirred at 25° C. for 1 h and concentrated to driness under reduced pressure. The residue was dissolved in dichloromethane (20 mL), washed with water (20 mL), dried over anhydrous sodium sulfate, and concentrated to driness under reduced pressure. The residue was purified by silica gel column chromatography (5% methanol/dichloromethane) to give compound 23-3 (800 mg, 50%) as brown oil.

### Step C: 1-(6-cyanobicyclo[3.1.0]hexan-6-yl)-5-((S)-2,2-dimethyltetrahydro-2H-pyran-4-yl)-N-methyl-N-phenyl-1H-indole-2-carboxamide (23-4)

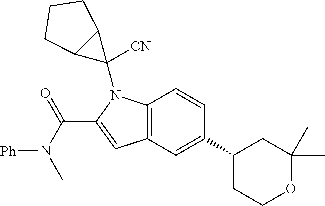 [SMILES: CN(C(=O)c1cc2cc([C@H]3CCOC(C)(C)C3)ccc2n1C1(C#N)C2CCCC21)c1ccccc1]

To a mixture of intermediate I-2 (200.0 mg, 0.5 mmol) and compound 23-3 (245.7 mg, 1.5 mmol) in tetrahydrofuran (4 mL) was added potassium bis(trimethylsilyl)amide (1.5 mL, 1 M in tetrahydrofuran, 1.5 mmol) dropwise under nitrogen atmosphere at 0° C. The reaction mixture was stirred at 25° C. for 2 h. The reaction mixture was quenched by water (20 mL), extracted with ethyl acetate (10 mL×3). The combined organic layers were concentrated to driness under reduced pressure, and the residue was purified by silica gel column chromatography (0-20% ethyl acetate/petroleum ether) to give compound 23-4 (168 mg, 72%) as yellow oil.

ESI-MS (m/z): [M+H]⁺=468.0.

### Step D: 5-((S)-2,2-dimethyltetrahydro-2H-pyran-4-yl)-N-methyl-1-(6-(5-oxo-4,5-dihydro-1,2,4-oxadiazol-3-yl)bicyclo[3.1.0]hexan-6-yl)-N-phenyl-1H-indole-2-carboxamide (23-5)

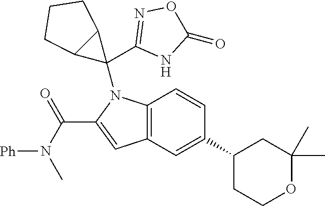 [SMILES: CN(C(=O)c1cc2cc([C@H]3CCOC(C)(C)C3)ccc2n1C1(c2noc(=O)[nH]2)C2CCCC21)c1ccccc1]

A mixture of compound 23-4 (168 mg, 0.36 mmol), hydroxylamine hydrochloride (249.7 mg, 3.6 mmol) and sodium bicarbonate (301.8 mg, 3.6 mmol) in dimethyl sulfoxide (3 mL) was heated to 60° C. and stirred for 13 h. The reaction mixture was cooled to 25° C., quenched with saturated sodium chloride (10 mL), and extracted with ethyl acetate (5 mL×3). The combined organic layers were dried over anhydrous sodium sulfate and concentrated to driness under reduced pressure. The residue was dissolved in dimethyl sulfoxide (2.5 mL), and then carbonyl diimidazole (93.9 mg, 0.58 mmol) and 1,8-diazabicycloundec-7-ene (110.0 mg, 0.72 mmol) was added. The reaction mixture was stirred at 25° C. for 1 h. The reaction mixture was quenched with saturated sodium chloride (10 mL) and extracted with ethyl acetate (5 mL×3). The combined organic layers were concentrated to driness under reduced pressure, and the residue was purified by silica gel column chromatography (0-30% ethyl acetate/petroleum ether) to give compound 23-5 (21 mg, 11%) as brown solid.

ESI-MS (m/z): [M−H]⁻=525.0.

### Step E: 5-((S)-2,2-dimethyltetrahydro-2H-pyran-4-yl)-1-(6-(5-oxo-4,5-dihydro-1,2,4-oxadiazol-3-yl)bicyclo[3.1.0]hexan-6-yl)-1H-indole-2-carboxylic acid (23-6)

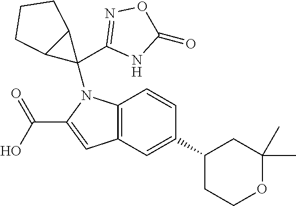 [SMILES: CC1(C)C[C@@H](c2ccc3c(c2)cc(C(=O)O)n3C2(c3noc(=O)[nH]3)C3CCCC32)CCO1]

To a mixture of compound 23-5 (21.0 mg, 0.04 mmol) in 2-methoxyethanol (1.5 mL) was added potassium hydroxide (30.0 mg, 0.53 mmol). The reaction mixture was heated to 100° C. and stirred for 7 h. The reaction mixture was cooled to 0° C., and diluted HCl (6 M) was slowly added (pH=2). The reaction mixture was extracted with ethyl acetate (5 mL×3). The combined organic layers were concentrated to driness under reduced pressure, and the residue was purified by silica gel column chromatography (5% methanol/dichloromethane) to give compound 23-6 (5 mg, 29%) as brown solid.

ESI-MS (m/z): [M−H]⁻=436.0.

### Step F: 3-(6-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-bicyclo[3.1.0]hexan-6-yl)-1,2,4-oxadiazol-5(4H)-one

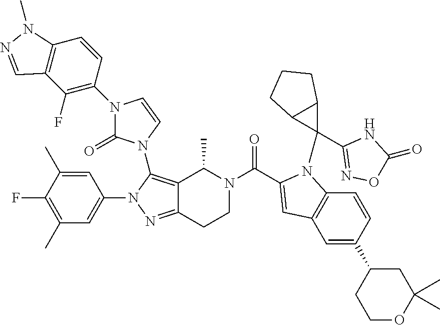 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2C2(c4noc(=O)[nH]4)C4CCCC42)CC3)cc(C)c1F]

To a mixture of compound 23-13 (5.0 mg, 0.011 mmol) in N,N-dimethylformamide (1.5 mL) was added 2-(7-azabenzotriazolyl)-N,N,N′,N′-tetramethyluronium hexafluorophosphate (3.9 mg, 0.017 mmol), intermediate I-1 (5.6 mg, 0.011 mmol) and N,N-diisopropylethylamine (7.4 mg, 0.057 mmol) and the reaction mixture was stirred at 25° C. for 1 h. The reaction mixture was quenched with water (5 mL) and extracted with ethyl acetate (5 mL×3). The combined organic layers were concentrated to driness under reduced pressure, and the residue was purified by silica gel column chromatography (5% methanol/dichloromethane) to give compound 23 (4.0 mg, 39%) as white solid.

ESI-MS(m/z): [M+H]+=909.7.

¹H NMR (400 MHz, CDCl₃): δ 11.41 (s, 1H), 8.13 (s, 1H), 7.67 (d, J=8.6 Hz, 1H), 7.50 (s, 1H), 7.30-7.26 (m, 2H), 7.16 (d, J=5.9 Hz, 2H), 6.66 (s, 1H), 6.61 (d, J=1.5 Hz, 1H), 6.31 (d, J=2.9 Hz, 1H), 5.79 (d, J=6.6 Hz, 1H), 4.44 (dd, J=13.4, 4.7 Hz, 1H), 4.12 (s, 3H), 3.92-3.78 (m, 2H), 3.62-3.51 (m, 1H), 3.17-2.94 (m, 3H), 2.29 (s, 6H), 2.06-1.96 (m, 1H), 1.84-1.52 (m, 12H), 1.34 (s, 3H), 1.28 (s, 3H), 1.26 (s, 3H).

### Example 4: 3-(6-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-3-oxabicyclo[3.1.0]hexan-6-yl)-1,2,4-oxadiazol-5(4H)-one (compound 29)

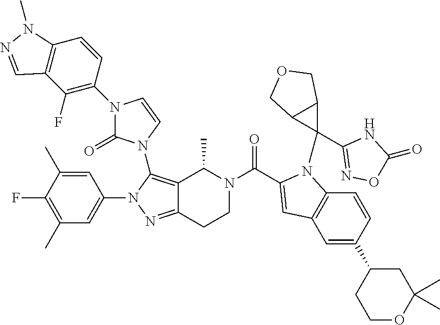 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2C2(c4noc(=O)[nH]4)C4COCC42)CC3)cc(C)c1F]

Scheme 4 shows non-limiting steps A to E to provide compound 29.

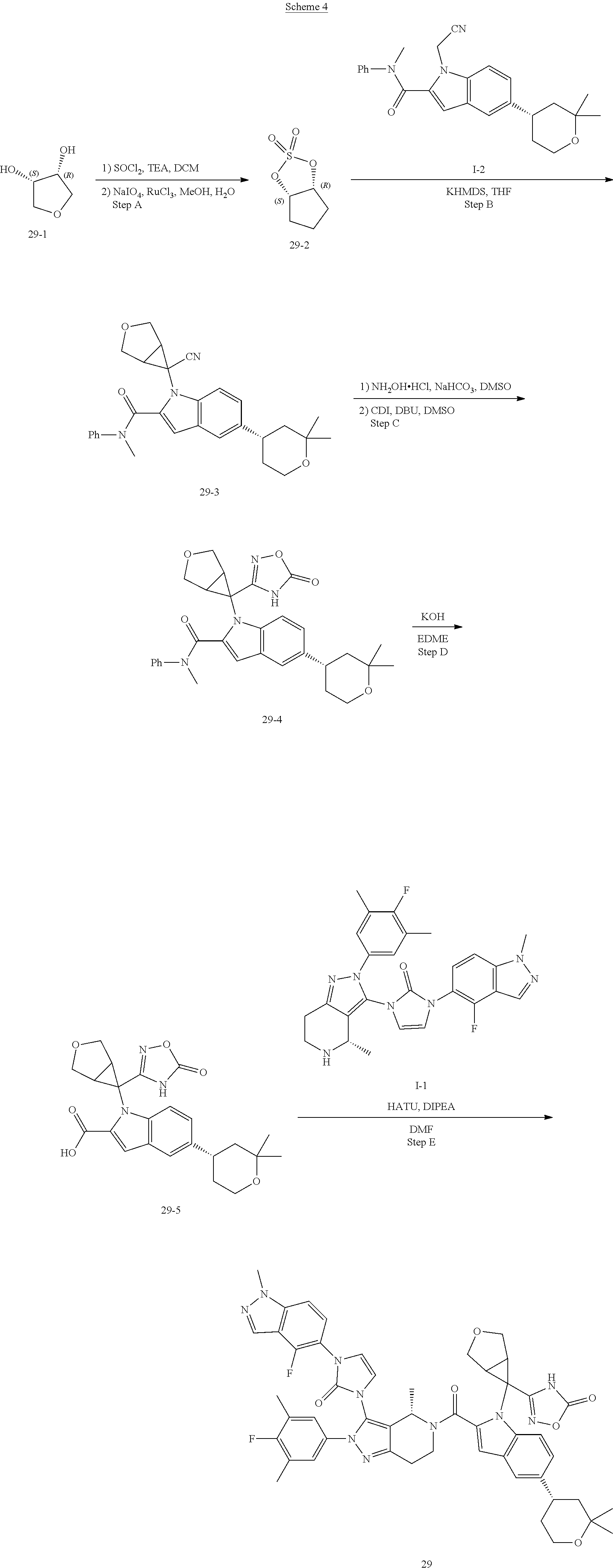

### Step A: (3aR,6aS)-terahydrofuro[3,4-d][1,3,2]dioxathiole 2,2-dioxide (29-2)

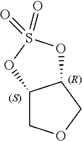

To a mixture of compound 29-1 (1.0 g, 9.61 mmol) and triethylamine (3.9 g, 38.42 mmol) in dichloromethane (20 mL) was added thionyl chloride (1.7 g, 14.41 mmol) dropwise at 0° C. and stirred for 1 h. The reaction mixture was quenched with water (20 mL), extracted with dichloromethane (10 mL×3). The combined organic layers were concentrated to dryness under reduced pressure. The residue was dissolved in methanol (8 mL) and water (2 mL) and sodium periodate (2.5 g, 11.59 mmol) and ruthenium trichloride (40.1 mg, 0.19 mmol) were added at 0° C. The reaction mixture was stirred at 25° C. for 2 h and concentrated to driness under reduced pressure. The residue was dissolved in dichloromethane (20 mL), washed with water (20 mL), dried over anhydrous sodium sulfate, and concentrated to driness under reduced pressure. The residue was purified by silica gel column chromatography (50% ethyl acetate/petroleum ether) to give compound 29-2 (1.1 g, 69%) as white solid.

### Step B: 1-(6-cyano-3-oxabicyclo[3.1.0]hexan-6-yl)-5-((S)-2,2-dimethyltetrahydro-2H-pyran-4-yl)-N-methyl-N-phenyl-1H-indole-2-carboxamide (29-3)

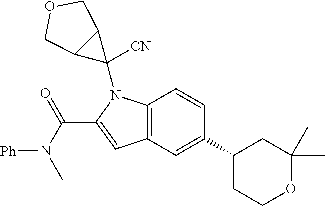 [SMILES: CN(C(=O)c1cc2cc([C@H]3CCOC(C)(C)C3)ccc2n1C1(C#N)C2COCC21)c1ccccc1]

To a mixture of intermediate I-2 (211.5 mg, 0.53 mmol) and compound 29-2 (350.0 mg, 2.11 mmol) in tetrahydrofuran (4 mL) was added potassium bis(trimethylsilyl)amide (1.5 mL, 1 M in tetrahydrofuran, 1.5 mmol) dropwise under nitrogen atmosphere at 0° C. The reaction mixture was stirred at 25° C. for 2 h. The reaction mixture was quenched by water (20 mL) and extracted with ethyl acetate (10 mL×3). The combined organic layers were concentrated to driness under reduced pressure, and the residue was purified by silica gel column chromatography (50% ethyl acetate/petroleum ether) to give compound 29-3 (58 mg, 24%) as yellow solid.

ESI-MS (m/z): [M+H]⁺=470.7.

### Step C: 5-((S)-2,2-dimethyltetrahydro-2H-pyran-4-yl)-N-methyl-1-(6-(5-oxo-4,5-dihydro-1,2,4-oxadiazol-3-yl)-3-oxabicyclo[3.1.0]hexan-6-yl)-N-phenyl-1H-indole-2-carboxamide (29-4)

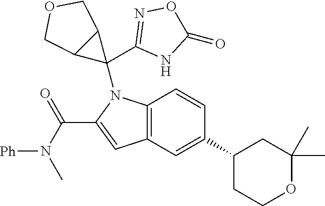 [SMILES: CN(C(=O)c1cc2cc([C@H]3CCOC(C)(C)C3)ccc2n1C1(c2noc(=O)[nH]2)C2COCC21)c1ccccc1]

A mixture of compound 29-3 (58.0 mg, 0.12 mmol), hydroxylamine hydrochloride (85.6 mg, 1.24 mmol) and N,N-diisopropylethanamine (159.6 mg, 1.24 mmol) in dimethyl sulfoxide (2 mL) was heated to 60° C. and stirred for 24 h. The reaction mixture was cooled to 25° C., quenched with saturated sodium chloride (10 mL), and extracted with ethyl acetate (5 mL×3). The combined organic layers were dried over anhydrous sodium sulfate and concentrated to driness under reduced pressure. The residue was dissolved in dimethyl sulfoxide (2 mL), and then carbonyl diimidazole (24.0 mg, 0.15 mmol) and 1,8-diazabicycloundec-7-ene (37.6 mg, 0.25 mmol) was added. The reaction mixture was stirred at 25° C. for 1 h. The reaction mixture was quenched with saturated sodium chloride (10 mL), extracted with ethyl acetate (5 mL×3). The combined organic layers were concentrated to driness under reduced pressure, and the residue was purified by silica gel column chromatography (0-30% ethyl acetate/petroleum ether) to give compound 29-4 (35 mg, 54%) as brown solid.

ESI-MS (m/z): [M+H]⁺=529.7.

### Step D: 5-((S)-2,2-dimethyltetrahydro-2H-pyran-4-yl)-1-(6-(5-oxo-4,5-dihydro-1,2,4-oxadiazol-3-yl)-3-oxabicyclo[3.1.0]hexan-6-yl)-1H-indole-2-carboxylic acid (29-5)

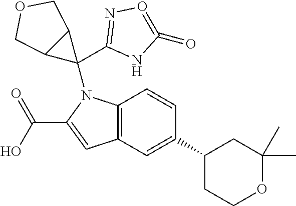

To a mixture of compound 29-4 (35.0 mg, 0.66 mmol) in 2-methoxyethanol (2 mL) was added potassium hydroxide (392.8 mg, 7.0 mmol). The reaction mixture was heated to 100° C. and stirred for 7 h. The reaction mixture was cooled to 0° C., and diluted HCl (6 M) was slowly added (pH=2). The reaction mixture was extracted with ethyl acetate (5 mL×3). The combined organic layers were concentrated to driness under reduced pressure, and the residue was purified by silica gel column chromatography (10% methanol/dichloromethane) to give compound 29-5(18.2 mg, 52%) as brown solid.

ESI-MS (m/z): [M+H]⁺=440.6.

### Step E: 3-(6-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-3-oxabicyclo[3.1.0]hexan-6-yl)-1,2,4-oxadiazol-5(4H)-one (29)

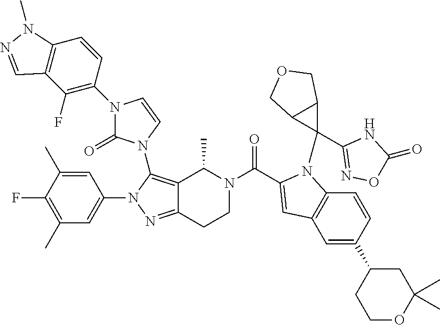 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2C2(c4noc(=O)[nH]4)C4COCC42)CC3)cc(C)c1F]

To a mixture of compound 29-5 (8.0 mg, 0.02 mmol) in N,N-dimethylformamide (2 mL) was added 2-(7-azabenzotriazolyl)-N,N,N′,N′-tetramethyluronium hexafluorophosphate (10.4 mg, 0.03 mmol), intermediate I-1 (8.9 mg, 0.02 mmol) and N,N-diisopropylethylamine (4.7 mg, 0.04 mmol), and the reaction mixture was stirred at 25° C. for 1 h. The reaction mixture was quenched with water (5 mL) and extracted with ethyl acetate (5 mL×3). The combined organic layers were concentrated to driness under reduced pressure, and the residue was purified by silica gel column chromatography (30% 0-30% ethyl acetate/petroleum ether) to give compound 29 (1.5 mg, 9%) as brown solid.

LC-MS: ESI-MS(m/z): [M+H]⁺=911.5.

¹H NMR (400 MHz, CDCl₃-d₆): δ 8.75 (s, 1H), 8.13 (s, 1H), 8.00 (s, 1H), 7.67 (d, J=8.4 Hz, 1H), 7.53 (s, 2H), 7.26 (s, 1H) 7.15 (d, J=6.0 Hz, 1H), 6.73 (s, 1H), 6.31 (s, 1H), 6.10 (s, 1H), 5.78 (s, 1H), 4.73 (d, J=9.2 Hz, 1H), 4.12 (s, 3H), 4.07-3.79 (m, 6H), 3.59 (s, 1H), 3.04 (s, 2H), 2.78-2.61 (m, 2H), 2.37-2.20 (m, 10H), 1.77-1.72 (m, 3H) 1.35 (s, 8H).

TABLE 3a
provides the structures of intermediate compounds A-1 to A-4.

| No. | Structure |
|---|---|
| A-1 | 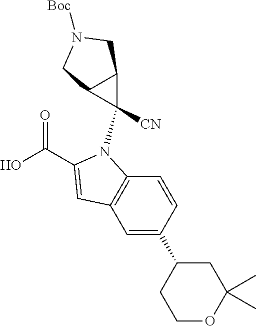 |
| A-2 | 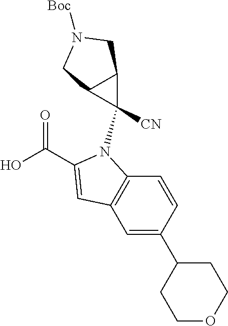 |
| A-3 | 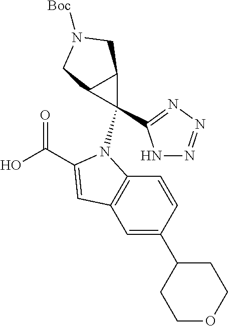 |
| A-4 | 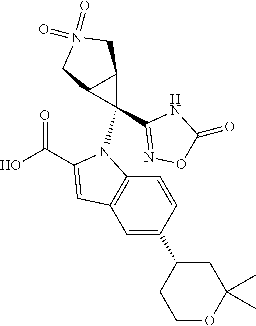 |

### Example 1a: 1-[(1R,5S,6S)-3-[(tert-butoxy)carbonyl]-6-cyano-3-azabicyclo[3.1.0]hexan-6-yl]-5-[(4S)-2,2-dimethyloxan-4-yl]-1H-indole-2-carboxylic acid (A-1)

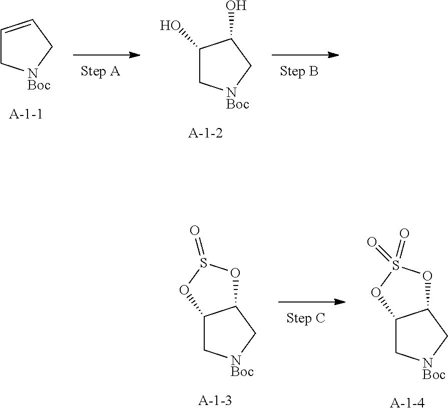

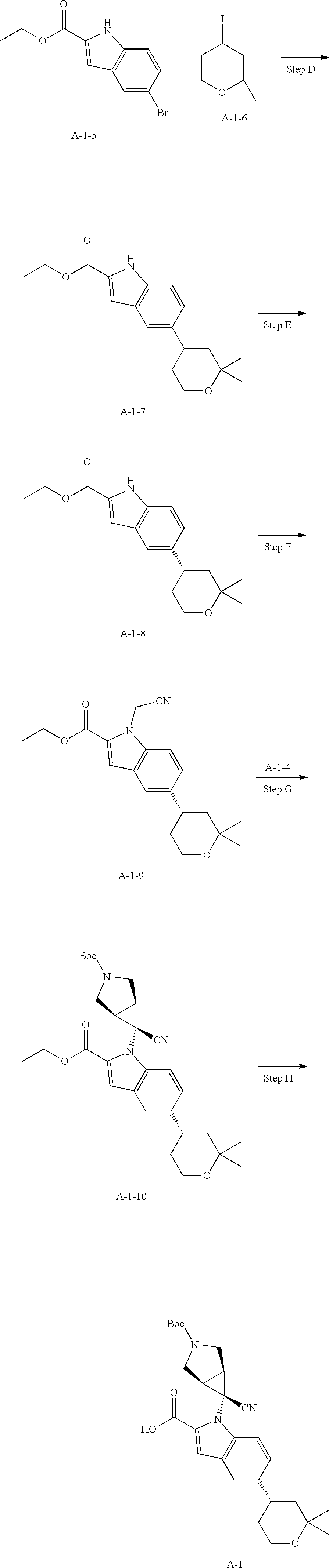

### Step A: tert-butyl (3S,4R)-3,4-dihydroxypyrrolidine-1-carboxylate (A-1-2)

A mixture of A-1-1 (50.00 g, 295.60 mmol) in acetone/water (500 mL/125 mL) was added aq. N-methylmorpholine oxide (208 mL, 104.00 g, 443.50 mmol, 50 wt %) and potassium osmate dihydrate (2.80 g, 8.90 mmol). The reaction mixture was stirred at 25° C. for 16 h. The reaction mixture was washed with saturated sodium bisulfite (200 mL), extracted with ethyl acetate (200 mL×3). The combined organic layers were dried over anhydrous sodium sulfate, concentrated to driness under reduced pressure. The residue was purified by silica gel column chromatography (dichloromethane/methanol=20/1) to obtain A-1-2 (51.60 g, 85%) as brown oil.

ESI-MS(m/z): [M−H]⁻=202.1.

### Step B: tert-butyl (3aR,6aS)-tetrahydro-5H-[1,3,2]dioxathiolo[4,5-c]pyrrole-5-carboxylate 2-oxide (A-1-3)

To a mixture of A-1-2 (51.60 g, 254.42 mmol) and triethylamine (141 mL, 1016.00 mmol) in dichloromethane (1550 mL) at 0° C., was added sulfuryl chloride (25 mL, 381.00 mmol) dropwise. The reaction mixture was stirred at 25° C. for 2 h, quenched with saturated brine (500 mL). The organic layer was separated, dried over anhydrous sodium sulfate, and concentrated to driness under reduced pressure to obtain A-1-3 (63.00 g) as brown oil.

ESI-MS(m/z): [M+H]⁺=250.2.

### Step C: tert-butyl (3aR,6aS)-tetrahydro-5H-[1,3,2]dioxathiolo[4,5-c]pyrrole-5-carboxylate 2,2-dioxide (A-1-4)

To a mixture of A-1-3 (63.00 g, 252.72 mmol) in acetonitrile/water (630 mL/630 mL) at 0° C., was added sodium periodate (65.00 g, 303.50 mmol) and ruthenium trichloride (1.10 g, 5.06 mmol). The reaction mixture was stirred at 25° C. for 16 h. The reaction mixture was quenched with water (200 mL), extracted with ethyl acetate (300 mL×3). The combined organic layers were dried over anhydrous sodium sulfate, concentrated to driness under reduced pressure. The residue was purified by silica gel column chromatography (petroleum ether/ethyl acetate=3/1) to obtain A-1-4 (40.0 g, 59%) as white solid.

ESI-MS(m/z): [M+H]⁺=266.2.

### Step D: ethyl 5-(2,2-dimethyltetrahydro-2H-pyran-4-yl)-1H-indole-2-carboxylate (A-1-7)

To a suspension of zinc powder (9.00 g, 138.5 mmol) in N, N-dimethylformamide (28 mL) was added trimethylchlorosilane (1.65 g, 15.2 mmol) and 1,2-dibromoethane (2.86 g, 15.2 mmol) under nitrogen atmosphere. The reaction mixture was stirred for 5 min at 25° C. A solution of compound A-1-6 (25.00 g, 104.1 mmol) in N,N-dimethylformamide (42 mL) was added and the mixture was stirred for 20 min at 25° C. Palladium acetate (780 mg, 3.5 mmol), (4-N,N-dimethylamino) phenylbis-tert-butylphosphonium (1.84 g, 6.93 mmol), and A-1-5 (9.31 g, 34.7 mmol) was added, and the mixture was heated to 55° C. for 2 h under nitrogen atmosphere. The reaction mixture was cooled to 25° C., quenched with diluted HCl (4 M), diluted with water (100 mL), and extracted with ethyl acetate (20 mL×3). The combined organic layers were washed with saturated sodium chloride, dried over anhydrous sodium sulfate, and concentrated to driness under reduced pressure. The residue was purified by silica gel column chromatography (0-15%, ethyl acetate/petroleum ether) to obtain A-1-7 (9.12 g, 87%) as white solid.

ESI-MS (m/z): [M+H]⁺=302.1.

### Step E: ethyl (S)-5-(2,2-dimethyltetrahydro-2H-pyran-4-yl)-1H-indole-2-carboxylate (A-1-8)

A-1-7 (16.70 g) was separated by preparative chial HPLC(SFC) to obtain A-1-8 (8.20 g) as white solid and its enantiomer (7.28 g). A-1-8 retention time: 15.980 min, enantiomer retention time: 9.534 min.

Separation conditions were as follows:
Equipment: Kep300
Column: Chiral ART Amylose KBN 5 m Column temperature: 25° C.
Solvent: Ethanol=100 (V)
Flow rate: 100 mL/minute

Analysis conditions were as follows:
Equipment: Shimadzu LC 20 CNZ with UV detector SPD M20A
Column: Chiral ART Amylose KBN (5 m), 0.46 cm I.D.×25 cm L
Column temperature: 25° C.
Solvent: Ethanol=100 (V)
Flow rate: 1.0 mL/minute

### Step F: ethyl (S)-1-(cyanomethyl)-5-(2,2-dimethyltetrahydro-2H-pyran-4-yl)-1H-indole-2-carboxylate (A-1-9)

To a mixture of A-1-8(2.00 g, 6.64 mmol) in N,N-dimethylformamide(40 mL) was added sodium hydride(0.24 g, 9.96 mmol) and chloroacetonitrile(1.00 g, 13.28 mmol) at 0° C. The reaction mixture was stirred at 25° C. for 16 h, quenched with water (40 mL), acidified with HCl to bring pH to 5, extracted with ethyl acetate (10 mL×3). The combined organic layers were washed with saturated brine (10 mL×2), concentrated to driness under reduced pressure. The residue was purified by by silica gel column chromatography (petroleum ether/ethyl acetate=85/15) to obtain A-1-9 (2.00 g, 89%) as white solid.

ESI-MS (m/z): [M+H]⁺=341.4

### Step G: ethyl 1-[(1R,5S,6S)-3-[(tert-butoxy)carbonyl]-6-cyano-3-azabicyclo[3.1.0]hexan-6-yl]-5-[(4S)-2,2-dimethyloxan-4-yl]-1H-indole-2-carboxylate

A mixture of A-1-9 (690.0 mg, 2.03 mmol), A-1-4 (1.10 g, 4.05 mmol), and 1,3-dimethyl-perhydro-2-pyrimidinone (1.10 g, 8.11 mmol) in tetrahydrofuran (10 mL), was slowly added lithium bis(trimethylsilyl)amide (1M in THF, 5.47 mL, 5.47 mmol) under nitrogen atmosphere at 0° C. The reaction mixture was stirred at 0° C. for 1 h, quenched with saturated ammonium chloride (10 mL), extracted with ethyl acetate (10 mL×2). The combined organic layers were washed with saturated brine (10 mL×2), concentrated to driness under reduced pressure. The residue was purified by by silica gel column chromatography (dichloromethane/ethyl acetate=95/5) to obtain A-1-10 (400.0 mg, 39%) as yellow solid.

ESI-MS(m/z): [M+H-Boc]⁺=408.3

### Step H: 1-[(1R,5S,6S)-3-[(tert-butoxy)carbonyl]-6-cyano-3-azabicyclo[3.1.0]hexan-6-yl]-5-[(4S)-2,2-dimethyloxan-4-yl]-1H-indole-2-carboxylic acid (A-1)

To a mixture of A-1-10 (400.0 mg, 0.79 mmol) in tetrahydrofuran/ethanol/water (5 mL/1 mL/1 mL), was added lithium hydroxide monohydrate (288.0 mg, 6.85 mmol). The reaction mixture was stirred at 50° C. for 3 h and cooled to 0° C., acidified with HCl(1M) to bring pH to 4, extracted with ethyl acetate (15 mL×2). The combined organic layers were concentrated to driness under reduced pressure to obtain A-1 (330.0 mg, 87%) as off-white solid.

ESI-MS (m/z): [M−H]⁻=478.2

### Example 2a: 1-[(1R,5S,6S)-3-[(tert-butoxy)carbonyl]-6-cyano-3-azabicyclo[3.1.0]hexan-6-yl]-5-(oxan-4-yl)-1H-indole-2-carboxylic acid (A-2)

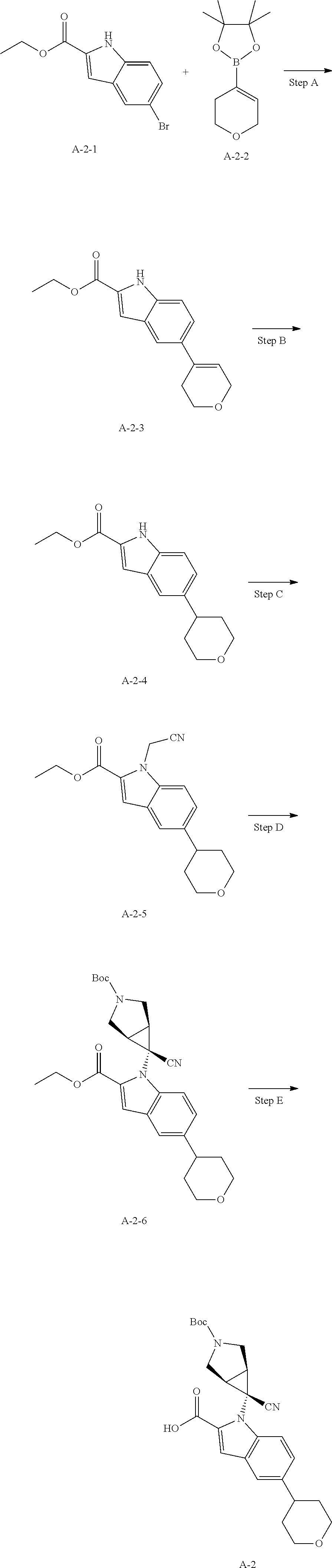

### Step A: ethyl 5-(3,6-dihydro-2H-pyran-4-yl)-1H-indole-2-carboxylate (A-2-3)

Under nitrogen atmosphere, a mixture of A-2-1 (8.00 g, 29.84 mmol), A-2-2 (7.50 g, 35.91 mmol), [1,1′-bis(diphenylphosphino)ferrocene]dichloropalladium (2.20 g, 2.98 mmol), and potassium carbonate (8.20 g, 59.68 mmol) in 1,4-dioxane/water (160 mL/40 mL)was stirred at 85° C. for 3 h. The reaction mixture was cooled to 25° C., quenched with water (200 mL), extracted with ethyl acetate (100 mL×3). The combined organic layers were concentrated to driness under reduced pressure. The residue was purified by silica gel column chromatography (petroleum ether/ethyl acetate=80/20) to obtain A-2-3 (9.6 g) as yellow solid.

ESI-MS (m/z): [M+H]⁺=271.9.

### Step B: ethyl 5-(tetrahydro-2H-pyran-4-yl)-1H-indole-2-carboxylate (A-2-4)

A mixture of A-2-3 (9.60 g, 35.38 mmol) and Pd/C (0.96 g, 10 wt %) in methanol/tetrahydrofuran (30 mL/90 mL) was stirred at 25° C. for 16 h under hydrogen atmosphere. The reaction mixture was filtered through Celite, and the filtrate was concentrated concentrated to driness under reduced pressure to obtain A-2-4 (7.30 g) as gray solid.

ESI-MS (m/z): [M+H]⁺=274.0.

### Step C: ethyl 1-(cyanomethyl)-5-(tetrahydro-2H-pyran-4-yl)-1H-indole-2-carboxylate (A-2-5)

To a mixture of A-2-4 (9.00 g, 33.04 mmol) in N,N-dimethylformamide (180 mL). was added sodium hydride (2.00 g, 49.56 mmol) at 0° C. The reaction mixture was stirred at 0° C. for 0.5 h. Chloroacetonitrile (5.00 g, 66.08 mmol) was added. The reaction mixture was stirred at 75° C. for 15 h and cooled to 25° C., quenched with water (200 mL), extracted with ethyl acetate (150 mL×3). The combined organic layers were washed with saturated brine (100 mL), dried over anhydrous sodium sulfate, and concentrated to driness under reduced pressure. The residue was slurried with a mixture of petroleum ether and ethyl acetate (30 mL/30 mL) for 1 h and filtered. The filter cake was dried to obtain A-2-5 (8.30 g, 80%) as off-white solid.

ESI-MS (m/z): [M+H]⁺=313.2.

### Step D: ethyl 1-[(1R,5S,6S)-3-[(tert-butoxy)carbonyl]-6-cyano-3-azabicyclo[3.1.0]hexan-6-yl]-5-(oxan-4-yl)-1H-indole-2-carboxylate (A-2-6)

Under nitrogen atmosphere, a mixture of A-2-5 (5.00 g, 16.01 mmol) and A-1-4 (6.40 g, 24.01 mmol) in tetrahydrofuran (50 mL) was slowly added bis(trimethylsilyl)amino potassium (1M in THF, 43.22 mL, 43.22 mmol) at 0° C. The reaction mixture was stirred at 0° C. for 0.5 h, quenched with water (200 mL), and extracted with ethyl acetate (50 mL×3). The combined organic layers were washed with saturated brine (30 mL×2), dried over anhydrous sodium sulfate, and concentrated to driness under reduced pressure. The residue was purified by silica gel column chromatography (petroleum ether/ethyl acetate=75/25) to obtain A-2-6 (2.60 g, 34%) as pale yellow solid.

ESI-MS (m/z): [M−H]⁻=480.6.

### Step E: 1-[(1R,5S,6S)-3-[(tert-butoxy)carbonyl]-6-cyano-3-azabicyclo[3.1.0]hexan-6-yl]-5-(oxan-4-yl)-1H-indole-2-carboxylic acid (A-2)

A mixture of A-2-6 (300 mg, 0.63 mmol) in tetrahydrofuran/methanol/water (5 mL/3 mL/1 mL) was added lithium hydroxide monohydrate (288.0 mg, 12.03 mmol). The reaction mixture was stirred at 25° C. for 1 h, quenched with water (10 mL), acidified with HCl (1 M) to bring pH to 4, and extracted with ethyl acetate (10 mL×3). The combined organic layers were washed with saturated brine (10 mL×2), dried over anhydrous sodium sulfate, and concentrated to driness under reduced pressure. The residue was purified by silica gel column chromatography (petroleum ether/ethyl acetate=60/40) to obtain A-2 (80.0 mg, 85%) as pale yellow solid.

ESI-MS (m/z): [M−H]⁻=452.5.

### Example 3a: 1-[(1R,5S,6S)-3-[(tert-butoxy)carbonyl]-6-(1H-1,2,3,4-tetrazol-5-yl)-3-azabicyclo[3.1.0]hexan-6-yl]-5-(oxan-4-yl)-1H-indole-2-carboxylic acid (A-3)

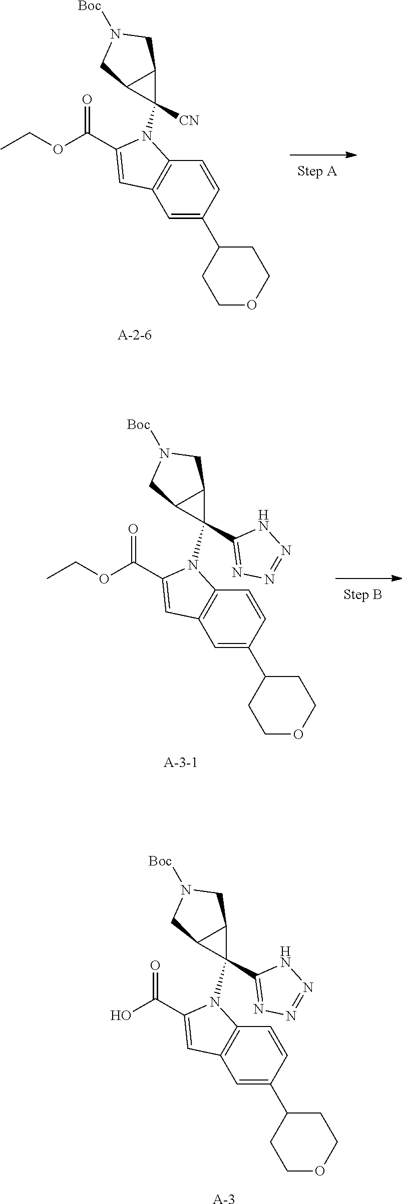 [SMILES: CCOC(=O)c1cc2cc(C3CCOCC3)ccc2n1[C@@]1(C#N)[C@@H]2CN(C(=O)OC(C)(C)C)C[C@@H]21 ; "A-2-6" ; CCOC(=O)c1cc2cc(C3CCOCC3)ccc2n1[C@@]1(c2nnn[nH]2)[C@@H]2CN(C(=O)OC(C)(C)C)C[C@@H]21 ; "A-3-1" ; CC(C)(C)OC(=O)N1C[C@@H]2[C@H](C1)[C@@]2(c1nnn[nH]1)n1c(C(=O)O)cc2cc(C3CCOCC3)ccc21 ; "A-3"]

### Step B: 1-[(1R,5S,6S)-3-[(tert-butoxy)carbonyl]-6-(1H-1,2,3,4-tetrazol-5-yl)-3-azabicyclo[3.1.0]hexan-6-yl]-5-(oxan-4-yl)-1H-indole-2-carboxylic acid (A-3)

A mixture of A-3-1 (0.10 g, 0.19 mmol) in tetrahydrofuran/ethanol/water (5 mL/1 mL/1 mL) was added lithium hydroxide monohydrate (288.0 mg, 6.85 mmol). The reaction mixture was stirred at 50° C. for 3 h and cooled to 25° C., acidified with HCl (1 M) to bring pH to 4, and extracted with ethyl acetate (5 mL×3). The combined organic layers were dried over anhydrous sodium sulfate, and concentrated to driness under reduced pressure to obtain A-3 (100.0 mg) as off-white solid.

ESI-MS (m/z): [M−H]⁻=493.2.

¹H NMR (400 MHz, DMSO-d6) δ 7.85 (t, J=7.8 Hz, 1H), 7.49 (s, 1H), 7.40-7.33 (m, 1H), 7.17 (d, J=7.5 Hz, 1H), 3.98-3.93 (m, 2H), 3.92-3.85 (m, 2H), 3.57-3.41 (m, 4H), 2.90-2.77 (m, 1H), 2.76-2.64 (m, 2H), 1.76-1.66 (m, 4H), 1.24 (d, J=8.2 Hz, 9H).

### Example 4a: 5-[(4S)-2,2-dimethyloxan-4-yl]-1-[(1R,5S,6R)-3,3-dioxo-6-(5-oxo-4,5-dihydro-1,2,4-oxadiazol-3-yl)-3λ⁶-thiabicyclo[3.1.0]hexan-6-yl]-1H-indole-2-carboxylic acid (A-4)

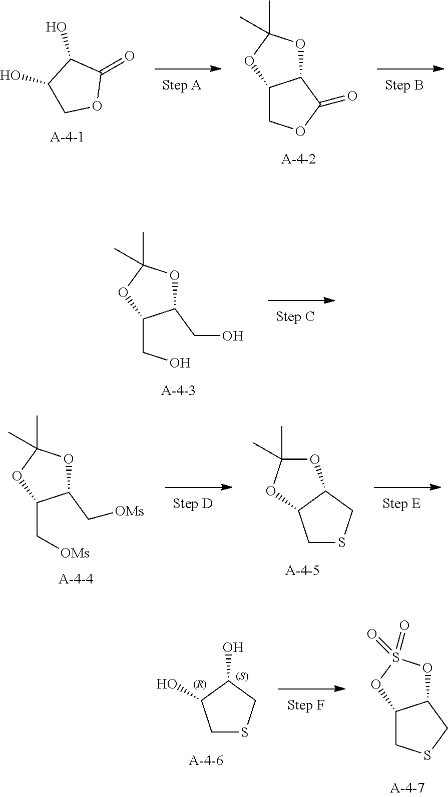

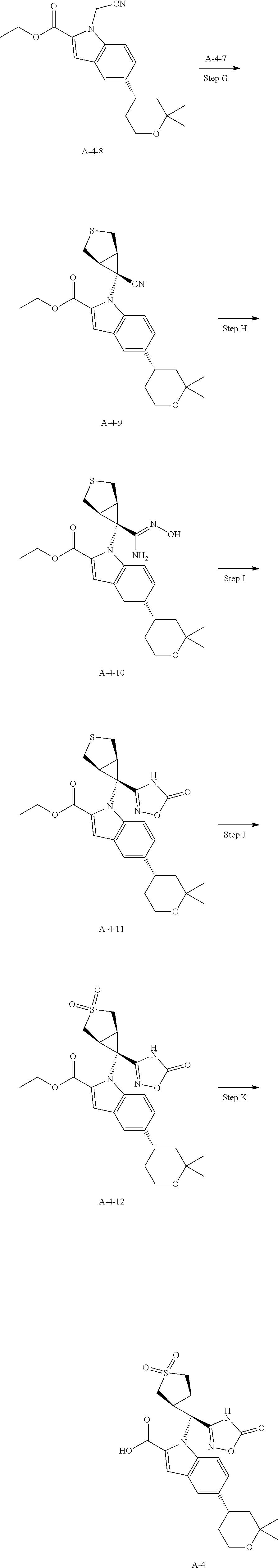

### Step A: (3aS,6aS)-2,2-Dimethyldihydrofuro[3,4-d][1,3]dioxol-4(3aH1)-one (A-4-2)

Under nitrogen atmosphere, to a mixture of A-4-1 (51.00 g, 431.87 mmol) in acetone (663 mL), was slowly added concentrated sulfuric acid (9.69 mL). The reaction mixture was stirred at 25° C. for 2 h and cooled to 0° C., quenched with saturated sodium bicarbonate (500 mL), and extracted with ethyl acetate (300 mL×3). The combined organic layers were washed with saturated brine (300 mL), dried over anhydrous sodium sulfate, and concentrated to driness under reduced pressure to obtain A-4-2 (65.00 g) as white solid.

ESI-MS (m/z): [M−H]⁻=157.3

### Step B: ((4S,5R)-2,2-Dimethyl-1,3-dioxolane-4,5-diyl)dimethanol (A-4-3)

To a mixture of A-4-2 (65.00 g, 411.00 mmol) in tetrahydrofuran (650 mL) was slowly added lithium aluminum hydride (31.20 g, 822.13 mmol) at 0° C. The reaction mixture was stirred at 25° C. for 1 h, cooled to 0° C. Sodium sulfate decahydrate (322 g, 1.00 mol) was added. The reaction mixture was filtered, and the filtrate was concentrated to driness under reduced pressure to obtain A-4-3 (48.00 g) as colorless oil.

ESI-MS (m/z): [M−H]⁻=161.1

### Step C: ((4S,5R)-2,2-Dimethyl-1,3-dioxolane-4,5-diyl)bis(methylene) dimethanesulfonate (A-4-4)

A mixture of A-4-3 (51.60 g, 318.17 mmol) in dichloromethane (516 mL), was added triethylamine (193.20 g, 1909.02 mmol) and methylsulfonyl chloride (109.30 g, 954.51 mmol) at 0° C. The reaction mixture was stirred at 0° C. for 1 h, quenched with saturated brine (500 mL). The organic layer was separated, dried over anhydrous sodium sulfate, and concentrated to driness under reduced pressure to obtain A-4-4 (101.00 g) as yellow oil.

ESI-MS (m/z): [M+H]⁺=339.2

### Step D: (3aR,6aS)-2,2-Dimethyltetrahydrothieno[3,4-d][1,3]dioxolane (A-4-5)

Under nitrogen atmosphere, a mixture of A-4-4 (101.00 g, 317.25 mmol) in N,N-dimethylformamide (1000 mL), was added sodium sulfide (29.70 g, 380.7 mmol). The reaction mixture was stirred at 100° C. for 8 h, cooled to 25° C., quenched with water (1500 mL), extracted with ethyl acetate (500 mL×3). The combined organic layers were washed with saturated brine (300 mL), dried over anhydrous sodium sulfate, and concentrated to driness under reduced pressure. The residue was purified by silica gel column chromatography (petroleum ether/ethyl acetate=85/15) to obtain A-4-5 (26.00 g, 51%) as white solid.

ESI-MS (m/z): [M+H]⁺=161.3

### Step E: (3R,4S)-Tetrahydrothiophene-3,4-diol (A-4-6)

To a mixture of A-4-5 (26.00 g, 162.27 mmol) in methanol (100 mL), was added glacial acetic acid (200 mL). The reaction mixture was stirred at reflux for 20 h, cooled to 25° C., and concentrated to driness under reduced pressure to obtain A-4-6 (18.00 g) as yellow solid.

ESI-MS (m/z): [M−H]⁻=119.0

### Step F: (3aR,6aS)-Tetrahydrothieno[3,4-d][1,3,2]dioxathiolo-2,2-dioxide (A-4-7)

To a mixture of A-4-6 (6.00 g, 49.93 mmol) and triethylamine (25.26 g, 249.65 mmol) in ethyl acetate (600 mL), was slowly added sulfuryl chloride (8.80 g, 64.91 mmol) at 0° C. The reaction mixture was stirred at 25° C. for 2 h, and filtered. The filtrate was concentrated to driness under reduced pressure. The residue was purified by silica gel column chromatography (petroleum ether/ethyl acetate=75/25) to obtain A-4-7 (3.00 g, 33%) as pale yellow solid.

ESI-MS (m/z): [M+H]⁺=183.1

### Step G: ethyl 1-[(1R,5S,6R)-6-cyano-3-thiabicyclo[3.1.0]hexan-6-yl]-5-[(4S)-2,2-dimethyloxan-4-yl]-1H-indole-2-carboxylate (A-4-9)

Under nitrogen atmosphere, to a mixture of A-4-8 (500.0 mg, 1.47 mmol) and A-4-7 (535.3 mg, 2.94 mmol) in tetrahydrofuran/1,3-dimethyl-perhydro-2-pyrimidinone (3 mL/3 mL), was slowly added lithium bis(trimethylsilyl)amide (1M in THF, 4.41 mL) at −40° C. The reaction mixture was stirred at −40° C. for 2 h, quenched with water (10 mL), extracted with ethyl acetate (5 mL×3). The combined organic layers were concentrated to driness under reduced pressure. The residue was purified by silica gel column chromatography (petroleum ether/ethyl acetate=80/20) to obtain A-4-9 (330.0 mg, 53%) as pale yellow solid.

ESI-MS (m/z): [M+H]⁺=425.0

### Step H: ethyl 5-[(4S)-2,2-dimethyloxan-4-yl]-1-[(1R,5S,6R)-6-(N′-hydroxycarbamimidoyl)-3-thiabicyclo[3.1.0]hexan-6-yl]-1H-indole-2-carboxylate (A-4-10)

To a mixture of A-4-9 (441.0 mg, 1.04 mmol) in ethanol (8 mL), was added hydroxylamine hydrochloride (721.8 mg, 10.39 mmol) and N,N-diisopropylethylamine (1.30 g, 10.39 mmol). The reaction mixture was stirred at 65° C. for 16 h, cooled to 25° C., and concentrated to driness under reduced pressure. The residue was treated with saturated brine (10 mL), extracted with ethyl acetate (5 mL×3). The combined organic layers were dried over anhydrous sodium sulfate, and concentrate under reduced pressure to obtain A-4-10 (472.0 mg) as light brown solid.

ESI-MS (m/z): [M+H]⁺=458.2

### Step I: ethyl 5-[(4S)-2,2-dimethyloxan-4-yl]-1-[(1R,5S,6R)-6-(5-oxo-4,5-dihydro-1,2,4-oxadiazol-3-yl)-3-thiabicyclo[3.1.0]hexan-6-yl]-1H-indole-2-carboxylate (A-4-11)

To a mixture of A-4-10 (472.0 mg, 1.03 mmol) in dimethyl sulfoxide (6.00 mL) was added N,N′-carbonyldiimidazole (334.9 mg, 2.06 mmol) and 1,8-diazabicyclo[5.4.0]undec-7-ene (393.1 mg, 2.58 mmol).The reaction mixture was stirred at 25° C. for 0.5 h, quenched with saturated sodium chloride (10 mL), extracted with ethyl acetate (5 mL×3). The combined organic layers were washed with saturated brine (10 mL), concentrated to driness under reduced pressure. The residue was purified by silica gel column chromatography (dichloromethane/methanol=98/2) to obtain A-4-11 (346.0 mg, 69%) as white solid.

ESI-MS (m/z): [M+H]⁺=484.2

### Step J: ethyl 5-[(4S)-2,2-dimethyloxan-4-yl]-1-[(1R,5S,6R)-3,3-dioxo-6-(5-oxo-4,5-dihydro-1,2,4-oxadiazol-3-yl)-3λ⁶-thiabicyclo[3.1.0]hexan-6-yl]-1H-indole-2-carboxylate (A-4-12)

To a mixture of A-4-11 (346.0 mg, 0.72 mmol) in methanol/dichloromethane (3 mL/6 mL), was added meta-chloroperoxybenzoic acid (435.8 mg, 2.15 mmol) at 0° C. The reaction mixture was stirred at 0° C. for 0.5 h and at 30° C. for 4 h. The reaction mixture was quenched with saturated sodium sulfite (10 mL), extracted with ethyl acetate (5 mL×3). The combined organic layers were concentrated to driness under reduced pressure. The residue was purified by silica gel column chromatography (dichloromethane/methanol=98/2) to obtain A-4-12 (327.0 mg, 89%) as yellow solid.

ESI-MS (m/z): [M+H]⁺=516.0

### Step K: 5-[(4S)-2,2-dimethyloxan-4-yl]-1-[(1R,5S,6R)-3,3-dioxo-6-(5-oxo-4,5-dihydro-1,2,4-oxadiazol-3-yl)-3λ⁶-thiabicyclo[3.1.0]hexan-6-yl]-1H-indole-2-carboxylic acid (A-4)

To a mixture of A-4-12 (327.0 mg, 0.63 mmol) in tetrahydrofuran/water (5 mL/2.5 mL), was added lithium hydroxide monohydrate (66.5 mg, 1.59 mmol). The reaction mixture was stirred at 40° C. for 16 h and cooled to 25° C., quenched with hydrochloric acid (1M) to bring pH to 3, extracted with ethyl acetate (5 mL×3). The combined organic layers were concentrated to driness under reduced pressure. The residue was purified by silica gel column chromatography (dichloromethane/methanol=95/5) to obtain A-4 (105.0 mg, 34%) as white solid.

ESI-MS (m/z): [M−H]⁻=486.0

¹H NMR (400 MHz, CDCl₃) δ 10.55 (s, 1H), 7.57 (d, J=8.8 Hz, 2H), 7.52 (s, 1H), 7.38 (s, 1H), 7.37 (d, J=8.8 Hz, 1H), 4.07 (d, J=12.8 Hz, 1H), 3.93-3.74 (m, 3H), 3.10 (d, J=15.6 Hz, 2H), 3.05-2.98 (m, 1H), 2.94-2.86 (m, 1H), 2.70-2.62 (m, 1H), 1.73 (d, J=15.6 Hz, 3H), 1.63 (t, J=12.8 Hz, 1H), 1.35 (s, 3H), 1.29 (s, 3H).

Table 3b provides the structures of intermediate compounds B-1 to B-13.

TABLE 3b

| No. | Structure |
|---|---|
| B-1 | 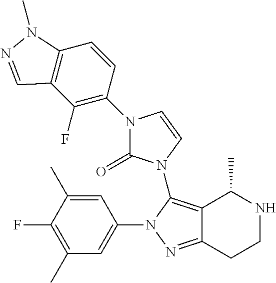 [SMILES: Cc1cc(-n2nc3c(c2-n2ccn(-c4ccc5c(cnn5C)c4F)c2=O)[C@H](C)NCC3)cc(C)c1F] |
| B-2 | 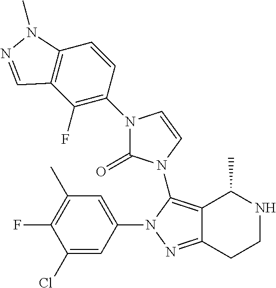 [SMILES: Cc1cc(-n2nc3c(c2-n2ccn(-c4ccc5c(cnn5C)c4F)c2=O)[C@H](C)NCC3)cc(Cl)c1F] |
| B-3 | 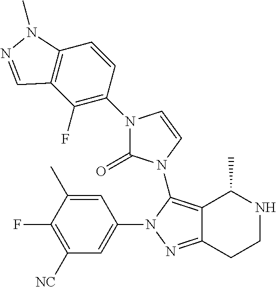 [SMILES: Cc1cc(-n2nc3c(c2-n2ccn(-c4ccc5c(cnn5C)c4F)c2=O)[C@H](C)NCC3)cc(C#N)c1F] |
| B-4 | 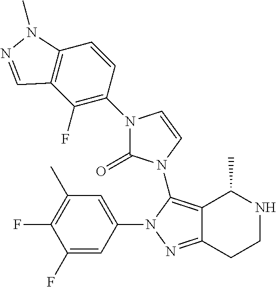 [SMILES: Cc1cc(-n2nc3c(c2-n2ccn(-c4ccc5c(cnn5C)c4F)c2=O)[C@H](C)NCC3)cc(F)c1F] |
| B-5 | 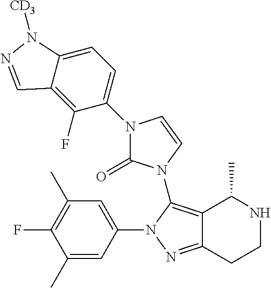 [SMILES: [2H]C([2H])([2H])n1ncc2c(F)c(-n3ccn(-c4c5c(nn4-c4cc(C)c(F)c(C)c4)CCN[C@H]5C)c3=O)ccc21] |
| B-6 | 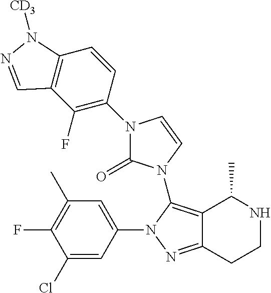 [SMILES: [2H]C([2H])([2H])n1ncc2c(F)c(-n3ccn(-c4c5c(nn4-c4cc(C)c(F)c(Cl)c4)CCN[C@H]5C)c3=O)ccc21] |
| B-7 | 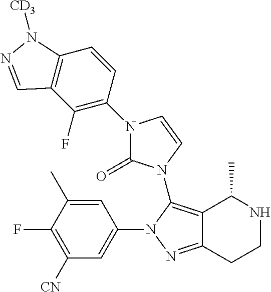 |
| B-8 | 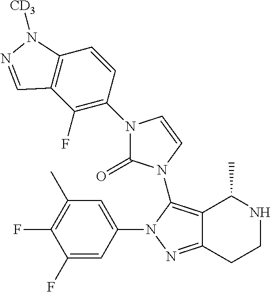 [SMILES: [2H]C([2H])([2H])n1ncc2c(F)c(-n3ccn(-c4c5c(nn4-c4cc(C)c(F)c(F)c4)CCN[C@H]5C)c3=O)ccc21] |
| B-9 | 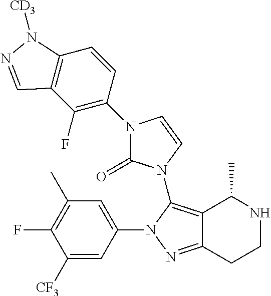 [SMILES: [2H]C([2H])([2H])n1ncc2c(F)c(-n3ccn(-c4c5c(nn4-c4cc(C)c(F)c(C(F)(F)F)c4)CCN[C@H]5C)c3=O)ccc21] |
| B-10 | 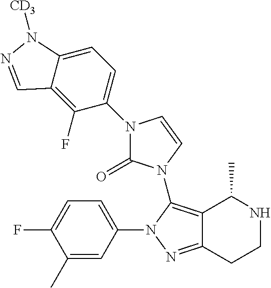 [SMILES: [2H]C([2H])([2H])n1ncc2c(F)c(-n3ccn(-c4c5c(nn4-c4ccc(F)c(C)c4)CCN[C@H]5C)c3=O)ccc21] |
| B-11 | 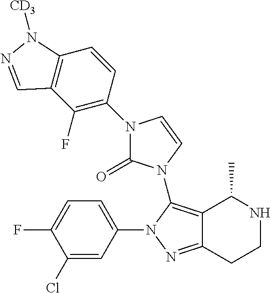 [SMILES: [2H]C([2H])([2H])n1ncc2c(F)c(-n3ccn(-c4c5c(nn4-c4ccc(F)c(Cl)c4)CCN[C@H]5C)c3=O)ccc21] |
| B-12 | 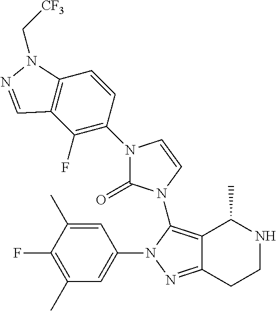 [SMILES: Cc1cc(-n2nc3c(c2-n2ccn(-c4ccc5c(cnn5CC(F)(F)F)c4F)c2=O)[C@H](C)NCC3)cc(C)c1F] |
| B-13 | 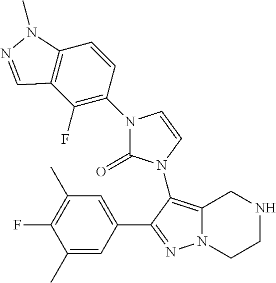 [SMILES: Cc1cc(-c2nn3c(c2-n2ccn(-c4ccc5c(cnn5C)c4F)c2=O)CNCC3)cc(C)c1F] |

### Example 5a: (S)-1-(4-fluoro-1-methyl-1H-indazole-5-yl)-3-(2-(4-fluoro-3,5-dimethylphenyl)-4-methyl-4,5,6,7-tetrahydro-2H-pyrazolo[4,3-c]pyridine-3-yl)-1,3-dihydro-2H-imidazol-2-one (B-1)

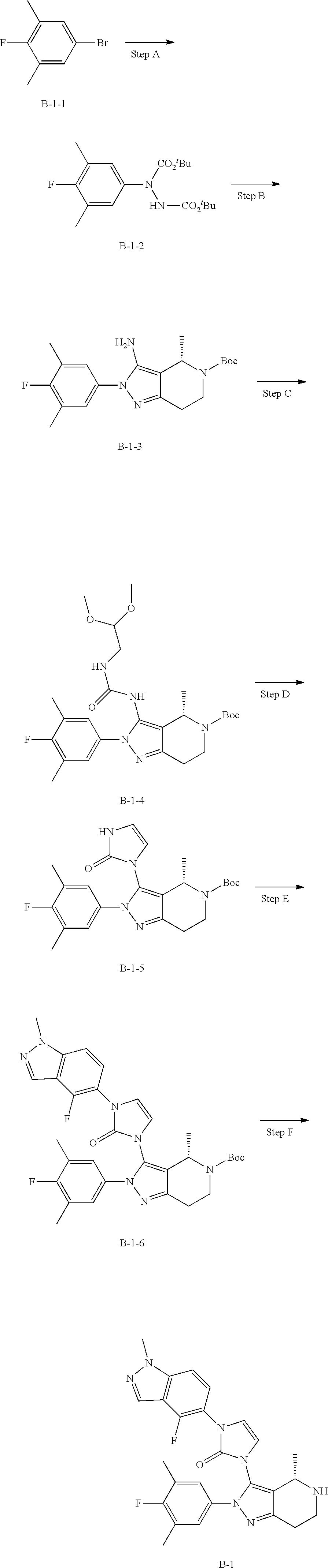

### Step A: di-tert-butyl 1-(4-fluoro-3,5-dimethylphenyl)hydrazine-1,2-dicarboxylate (B-1-2)

To a mixture of B-1-1 (45.00 g, 221.62 mmol) in tetrahydrofuran (450 mL) was added dropwise n-butyllithium (85.1 mL, 2.5 M in hexane, 212.76 mmol) at −70° C. and stirred for 1 h. Then, di-tert-butyl azodicarboxylate (45.93 g, 199.46 mmol) was added dropwise at −40° C., and the mixture was stirred for 0.5 h. The reaction mixture was warmed to 25° C. over 1 h and quenched with saturated ammonium chloride solution (460 mL). The reaction mixture was extracted with heptane (230 mL). The organic layer was separated and concentrated. Heptane (70 mL) was added, and the mixture was heated to 70° C. to promote dissolution. The solution was cooled to 25° C., and white solid was precipitated for 1 h. The precipitate was collected by filtration, washed with heptane (23 mL), and dried to give B-1-2(42.00 g, 53%) as a white solid.

¹H NMR (400 MHz, DMSO-d₆): δ 9.56 (s, 1H), 7.07-6.96 (m, 2H), 2.19 (s, 6H), 1.50-1.37 (m, 18H).

### Step B: tert-butyl (S)-3-amino-2-(4-fluoro-3,5-dimethylphenyl)-4-methyl-2,4,6,7-tetrahydro-5H-pyrazolo[4,3-c]pyridine-5-carboxylate (B-1-3)

To a mixture of B-1-2 (42.00 g, 118.50 mmol) in N-methyl-2-pyrrolidinone (126 mL) was added methanesulfonic acid (22.78 g, 237 mmol), and the mixture was stirred at 80° C. for 7 h. The reaction mixture was cooled to 25° C.; and toluene (252 mL), potassium carbonate (18.02 g, 130.35 mmol) and water (252 mL) was added. The reaction mixture was stirred at 25° C. for 10 min. The organic layer was separated, to which was added a solution of tert-butyl (2S)-3-cyano-2-methyl-4-oxopiperidine-1-carboxylate (27.82 g, 116.74 mmol) in toluene (126 mL), pyridine hydrochloride (1.35 g, 11.67 mmol) and toluene (84 mL). The reaction mixture was stirred at 90° C. for 1 h and cooled to 25° C. The reaction mixture was washed with aq. sodium hydroxide (252 mL, 1M). The organic layer was separated and concentrated under reduced pressure to give B-1-3 (34.80 g, 80%) as brown oil.

ESI-MS(m/z): [M+H]⁺=374.9.

¹H NMR (400 MHz, DMSO-d₆): δ 7.22 (d, J=6.4 Hz, 2H), 5.18 (s, 2H), 5.07 (d, J=43.4 Hz, 1H), 4.24-4.02 (m, 1H), 3.14-2.90 (m, 1H), 2.48-2.38 (m, 2H), 2.25 (d, J=1.2 Hz, 6H), 1.43 (s, 9H), 1.24 (d, J=6.0 Hz, 3H).

### Step C: tert-butyl (S)-3-(3-(2,2-dimethoxyethyl)ureido)-2-(4-fluoro-3,5-dimethylphenyl)-4-methyl-2,4,6,7-tetrahydro-5H-pyrazolo[4,3-c]pyridine-5-carboxylate (B-1-4)

To a mixture of B-1-3 (11.00 g, 29.38 mmol) and N-(2,2-dimethoxyethyl)-1H-imidazole-1-carboxamide (17.56 g, 88.14 mmol) in N,N-dimethylacetamide (110 mL) was added potassium tert-butoxide (19.78 g, 176.28 mmol), and the mixture was stirred at 25° C. for 2 h. The reaction mixture was quenched with water (200 mL) at 0° C. and extracted by ethyl acetate (80 mL×3). The combined organic layers were washed with saturated sodium chloride (50 mL×3), dried over anhydrous sodium sulfate, and concentrated under reduced pressure. The residue was purified by silica gel column chromatography (0-5% MeOH/dichloromethane) to give compound B-1-4 (14.00 g, 94%) as yellow solid.

ESI-MS(m/z): [M+H]⁺=505.9.

### Step D: tert-butyl (S)-2-(4-fluoro-3,5-dimethylphenyl)-4-methyl-3-(2-oxo-2,3-dihydro-1H-imidazol-1-yl)-2,4,6,7-tetrahydro-5H-pyrazolo[4,3-c]pyridine-5-carboxylate (B-1-5)

To a mixture of B-1-4 (14.00 g, 27.69 mmol) in tetrahydrofuran (140 mL) was added methanesulfonic acid (2.66 g, 27.69 mmol), and the mixture was stirred at 60° C. for 2 h. The reaction mixture was cooled to 25° C., and a solution of potassium phosphate (2.35 g, 11.08 mmol) in water (56 mL) and di-tert-butyl dicarbonate (4.83 g, 22.15 mmol) was added. The resulting mixture was stirred at 25° C. for 1 h, diluted with water (150 mL), and extracted with ethyl acetate (50 mL×3). The combined organic layers were washed with saturated sodium chloride (50 mL×3), dried over anhydrous sodium sulfate, and concentrated under reduced pressure. The residue was then purified by silica gel column chromatography (20-30% EA/PE) to give B-1-5 (8.00 g, 65%) as yellow solid.

ESI-MS(m/z): [M+H]⁺=442.2

### Step E: tert-butyl (S)-3-(3-(4-fluoro-1-methyl-1H-indazole-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridine-5-carboxylate (B-1-6)

To a mixture of B-1-5 (1.00 g, 2.27 mmol) and compound 1-13 (778 mg, 3.40 mmol) in N-methyl-2-pyrrolidone (10 mL) was added cuprous iodide (216 mg, 1.14 mmol), (1R,2R)-(−)-N,N′-dimethyl-1,2-cyclohexanediamine (323 mg, 2.27 mmol), and potassium carbonate (941 mg, 6.81 mmol). The mixture was stirred under nitrogen atmosphere at 120° C. for 16 h. The reaction mixture was poured into water (50 mL), extracted with ethyl acetate (10 mL×3). The combined organic layers were washed with saturated sodium chloride, dried over anhydrous sodium sulfate, and concentrated under reduced pressure. The residue was purified by silica gel column chromatography (50-62%, ethyl acetate/petroleum ether) to give B-1-6 (1.10 g, 82%) as yellow solid.

ESI-MS(m/z): [M+H]⁺=590.0

### Step F: (S)-1-(4-fluoro-1-methyl-1H-indazole-5-yl)-3-(2-(4-fluoro-3,5-dimethylphenyl)-4-methyl-4,5,6,7-tetrahydro-2H-pyrazolo[4,3-c]pyridine-3-yl)-1,3-dihydro-2H-imidazol-2-one hydrochloride (B-1)

To a mixture of B-1-6 (450 mg) in dichloromethane (4.5 mL) was added HCl (4 M in 1,4-dioxane, 1.9 mL), and the mixture was stirred at 25° C. for 3 h. The reaction mixture was concentrated under reduced pressure. The residue was re-dissolved in ethyl acetate and washed with saturated NaHCO₃ solution. The organic layer was separated, dried over anhydrous sodium sulfate, concentrated under reduced pressure to give B-1 (350 mg, 94%) as yellow solid.

ESI-MS(m/z): [M+H]⁺=489.9

¹H NMR (400 MHz, DMSO-d₆) δ 8.28 (s, 1H), 7.64-7.58 (m, 1H), 7.38 (t, J=7.8 Hz, 1H), 7.12 (d, J=6.2 Hz, 2H), 7.03-6.98 (m, 1H), 6.84 (s, 1H), 4.10 (s, 3H), 3.93-3.85 (m, 1H), 3.21-3.15 (m, 1H), 2.86-2.79 (m, 1H), 2.66-2.57 (m, 2H), 2.26-2.16 (m, 7H), 1.13 (d, J=6.6 Hz, 3H).

### Example 6: (S)-1-(2-(3-chloro-4-fluoro-5-methylphenyl)-4-methyl-4,5,6,7-tetrahydro-2H-pyrazolo[4,3-c]pyridine-3-yl)-3-(4-fluoro-1-methyl-1H-indazole-5-yl)-1,3-dihydro-2H-imidazol-2-one (B-2)

By replacing 4-bromo-2,6-dimethylfluorobenzene with 5-bromo-1-chloro-2-fluorotoluene, and following procedures similar to those of Example 5a, the title compound was obtained.

ESI-MS(m/z): [M+H]⁺=510.2.

¹H NMR (400 MHz, DMSO-d₆) δ 8.28 (s, 1H), 7.62 (d, J=9.0 Hz, 1H), 7.43-7.30 (m, 3H), 7.08 (d, J=3.3 Hz, 1H), 6.92 (d, J=3.0 Hz, 1H), 4.10 (s, 3H), 4.04 (q, J=6.6 Hz, 1H), 3.33-3.24 (m, 1H), 2.98-2.89 (m, 1H), 2.77-2.66 (m, 2H), 2.31 (d, J=2.1 Hz, 3H), 1.24 (d, J=6.9 Hz, 1H), 1.19 (d, J=6.6 Hz, 3H).

### Example 7: (S)-2-fluoro-5-(3-(3-(4-fluoro-1-methyl-1H-indazole-5-yl)-2-oxo-2,3-dihydro-1H-imidazol-1-yl)-4-methyl-4,5,6,7-tetrahydro-2H-pyrazolo[4,3-c]pyridine-2-yl)-3-methylbenzonitrile (B-3)

By replacing 4-bromo-2,6-dimethylfluorobenzene with 5-bromo-1-cyano-2-fluorotoluene, and following procedures similar to those of Example 5a, the title compound was obtained.

ESI-MS(m/z): [M+H]⁺=501.2.

¹H NMR (400 MHz, DMSO-d₆) δ 8.11 (s, 1H), 7.65-7.59 (m, 1H), 7.51-7.47 (m, 1H), 7.46-7.37 (m, 1H), 7.28-7.22 (m, 1H), 6.64-6.60 (m, 1H), 6.33-6.28 (m, 1H), 4.22-4.15 (m, 1H), 4.10 (s, 3H), 3.48-3.38 (m, 1H), 3.20-3.01 (m, 1H), 2.93-2.77 (m, 2H), 2.70-2.45 (m, 1H), 2.36-2.32 (m, 3H), 1.36 (d, J=6.6 Hz, 3H).

### Example 8: (S)-1-(2-(3,4-difluoro-5-methylphenyl)-4-methyl-4,5,6,7-tetrahydro-2H-pyrazolo[4,3-c]pyridine-3-yl)-3-(4-fluoro-1-methyl-1H-indazole-5-yl)-1,3-dihydro-2H-imidazol-2-one (B-4)

By replacing 4-bromo-2,6-dimethylfluorobenzene with 5-bromo-1,2-difluoro-3-methylbenzene, and following procedures similar to those of Example 5a, the title compound was obtained.

ESI-MS(m/z): [M+H]⁺=493.9.

¹H NMR (400 MHz, DMSO-d₆) δ 8.29 (s, 1H), 7.62 (d, J=8.9 Hz, 1H), 7.40 (t, J=8.0 Hz, 1H), 7.31-7.20 (m, 1H), 7.17 (s, 1H), 7.06 (s, 1H), 6.92 (s, 1H), 4.10 (s, 3H), 3.94-3.85 (m, 1H), 3.26-3.13 (m, 1H), 2.91-2.74 (m, 1H), 2.62 (s, 2H), 2.36 (s, 1H), 2.31 (d, J=1.8 Hz, 3H), 1.12 (d, J=6.6 Hz, 3H).

### Example 9: (S)-1-(4-fluoro-1-(methyl-d3)-1H-indazole-5-yl)-3-(2-(4-fluoro-3,5-dimethylphenyl)-4-methyl-4,5,6,7-tetrahydro-2H-pyrazolo[4,3-c]pyridine-3-yl)-1,3-dihydro-2H-imidazol-2-one (B-5)

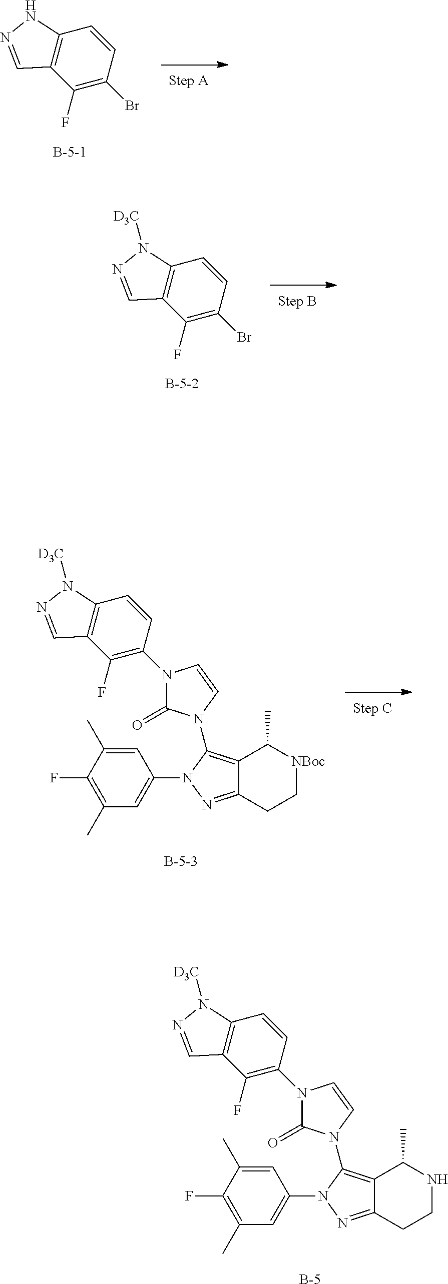

### Step A: 5-bromo-4-fluoro-1-(methyl-d3)-1H-indazole (B-5-2)

To a mixture of B-5-1 (0.5 g, 2.33 mmol) in N, N-dimethylformamide (3.5 mL), was added sodium hydride (112 mg, 2.80 mmol). The reaction mixture was stirred at 25° C. for 10 min. Then deuterated methyl iodide (0.41 g, 2.80 mmol) was added. The reaction mixture was stirred at 25° C. for 2 h, quenched with saturated ammonium chloride (2 mL), and extracted with ethyl acetate (10 mL×3). The combined organic layers were washed with saturated brine (5 mL), dried over anhydrous sodium sulfate, and concentrated to driness under reduced pressure. The residue was purified by silica gel column chromatography (petroleum ether/ethyl acetate=8/1) to obtain B-5-2 (350 mg, 65%) as white solid.

ESI-MS(m/z): [M+H]⁺=232.9.

### Step B: tert-butyl (S)-3-(3-(4-fluoro-1-(methyl-d3)-1H-indazole-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridine-5-carboxylate (B-5-3)

Under nitrogen atmosphere, a mixture of B-1-5 (450 mg, 1.02 mmol), B-5-2 (350 mg, 1.53 mmol), potassium carbonate (422 mg, 3.06 mmol), trans-(1S,2S)-N,N′-dimethylcyclohexane-1,2-diamine (145 mg, 1.02 mmol), and copper(I) iodide (97 mg, 0.51 mmol) in N-methylpyrrolidone (5 mL) was stirred at 120° C. for 8 h, and cooled to 25° C. The reaction mixture was quenched with water (10 mL), extracted with ethyl acetate (10 mL×2). The combined organic layers were washed with saturated brine (10 mL×2), dried over anhydrous sodium sulfate, concentrated to driness under reduced pressure. The residue was purified by silica gel column chromatography (petroleum ether/ethyl acetate/=5/3) to obtain B-5-3 (500 mg, 83%) as light gray solid.

ESI-MS(m/z): [M+H]⁺=592.9.

### Step C: (S)-1-(4-fluoro-1-(methyl-d3)-1H-indazole-5-yl)-3-(2-(4-fluoro-3,5-dimethylphenyl)-4-methyl-4,5,6,7-tetrahydro-2H-pyrazolo[4,3-c]pyridine-3-yl)-1,3-dihydro-2H-imidazol-2-one (B-5)

To a mixture of B-5-3 (200 mg, 0.34 mmol) in dichloromethane (2 mL) was added HCl (4M in 1,4-dioxane, 2 mL). The reaction mixture was stirred at 25° C. for 4 h and concentrated to driness under reduced pressure to obtain B-5 (200 mg, 98%) as light brown solid.

ESI-MS(m/z): [M+H]⁺=602.9.

¹H NMR (400 MHz, DMSO-d₆) δ 10.13 (s, 1H), 9.55-9.39 (m, 1H), 8.29 (s, 1H), 7.65-7.60 (m, 1H), 7.45-7.38 (m, 1H), 7.16 (d, J=6.2 Hz, 2H), 7.08-7.04 (m, 1H), 6.88-6.83 (m, 1H), 4.63-4.52 (m, 1H), 3.67-3.55 (m, 1H), 3.12-2.92 (m, 2H), 2.27-2.20 (m, 6H), 1.43 (d, J=6.8 Hz, 3H).

### Example 10: (S)-1-(2-(3-chloro-4-fluoro-5-methylphenyl)-4-methyl-4,5,6,7-tetrahydro-2H-pyrazolo[4,3-c]pyridine-3-yl)-3-(4-fluoro-1-(methyl-d3)-1H-indazole-5-yl)-1,3-dihydro-2H-imidazol-2-one (B-6)

By replacing 4-bromo-2,6-dimethylfluorobenzene with 5-bromo-1-chloro-2-fluorotoluene, and following procedures similar to those of Example 5a, the title compound was obtained.

ESI-MS(m/z): [M+H]⁺=513.2.

¹H NMR (400 MHz, DMSO-d₆) δ 8.29 (s, 1H), 7.62 (d, J=8.7 Hz, 1H), 7.42-7.28 (m, 3H), 7.11-7.06 (m, 1H), 6.93 (s, 1H), 4.01-3.89 (m, 1H), 3.35-3.05 (m, 1H), 2.92-2.75 (m, 1H), 2.72-2.56 (m, 2H), 2.38-2.21 (m, 3H), 1.16 (d, J=6.4 Hz, 3H).

### Example 11: (S)-2-fluoro-5-(3-(3-(4-fluoro-1-(methyl-d3)-1H-indazole-5-yl)-2-oxo-2,3-dihydro-1H-imidazol-1-yl)-4-methyl-4,5,6,7-tetrahydro-2H-pyrazolo[4,3-c]pyridine-2-yl)-3-methylbenzonitrile (B-7)

By replacing 4-bromo-2,6-dimethylfluorobenzene with 5-bromo-1-cyano-2-fluorotoluene, and following procedures similar to those of Example 5a, the title compound was obtained.

ESI-MS(m/z): [M+H]⁺=504.4.

¹H NMR (400 MHz, DMSO-d₆) δ 8.12 (s, 1H), 7.65-7.60 (m, 1H), 7.53-7.48 (m, 1H), 7.45-7.39 (m, 1H), 7.27-7.24 (m, 1H), 6.63 (d, J=1.6 Hz, 1H), 6.31 (d, J=2.4 Hz, 1H), 4.28-4.20 (m, 1H), 3.53-3.39 (m, 1H), 3.23-3.07 (m, 1H), 3.00-2.87 (m, 2H), 2.87-2.82 (m, 1H), 2.34 (d, J=1.6 Hz, 3H), 1.49-1.43 (m, 1H), 1.40 (d, J=6.4 Hz, 2H).

### Example 12: (S)-1-(2-(3,4-difluoro-5-methylphenyl)-4-methyl-4,5,6,7-tetrahydro-2H-pyrazolo[4,3-c]pyridine-3-yl)-3-(4-fluoro-1-(methyl-d3)-1H-indazole-5-yl)-1,3-dihydro-2H-imidazol-2-one (B-8)

By replacing 4-bromo-2,6-dimethylfluorobenzene with 5-bromo-1,2-difluoro-3-methylbenzene, and following procedures similar to those of Example 5a, the title compound was obtained.

ESI-MS(m/z): [M+H]⁺=497.0.

¹H NMR (400 MHz, DMSO-d₆) δ 8.29 (s, 1H), 7.62 (d, J=9.5 Hz, 1H), 7.48-7.36 (m, 1H), 7.30-7.20 (m, 1H), 7.20-7.14 (m, 1H), 7.07 (d, J=2.9 Hz, 1H), 6.92 (s, 1H), 4.02-3.89 (m, 1H), 3.26-3.15 (m, 1H), 2.93-2.78 (m, 1H), 2.70-2.55 (m, 2H), 2.31 (s, 3H), 1.14 (d, J=6.5 Hz, 3H).

### Example 13: (S)-1-(4-fluoro-1-(methyl-d3)-1H-indazole-5-yl)-3-(2-(4-fluoro-3-methyl-5-(trifluoromethyl)phenyl)-4-methyl-4,5,6,7-tetrahydro-2H-pyrazolo[4,3-c]pyridine-3-yl)-1,3-dihydro-2H-imidazol-2-one (B-9)

By replacing 4-bromo-2,6-dimethylfluorobenzene with 4-fluoro-3-bromo-5-(trifluoromethyl)bromobenzene, and following procedures similar to those of Example 5a, the title compound was obtained.

ESI-MS(m/z): [M+H]⁺=547.2.

¹H NMR (400 MHz, DMSO-d₆) δ 8.28 (s, 1H), 7.80-7.71 (m, 1H), 7.63 (d, J=8.7 Hz, 1H), 7.53-7.46 (m, 1H), 7.34 (t, J=7.2 Hz, 1H), 7.13-7.06 (m, 1H), 7.00-6.93 (m, 1H), 4.10-3.98 (m, 1H), 3.40-3.23 (m, 2H), 3.00-2.87 (m, 1H), 2.79-2.64 (m, 2H), 2.36 (d, J=1.5 Hz, 3H), 1.19 (d, J=6.3 Hz, 3H).

### Example 14: (S)-1-(4-fluoro-1-(methyl-d3)-1H-indazole-5-yl)-3-(2-(4-fluoro-3-methylphenyl)-4-methyl-4,5,6,7-tetrahydro-2H-pyrazolo[4,3-c]pyridine-3-yl)-1,3-dihydro-2H-imidazol-2-one (B-10)

By replacing 4-bromo-2,6-dimethylfluorobenzene with 5-bromo-2-fluorotoluene, and following procedures similar to those of Example 5a, the title compound was obtained.

ESI-MS(m/z): [M+H]⁺=479.2.

¹H NMR (400 MHz, DMSO-d₆) δ 8.11 (s, 1H), 7.50-7.39 (m, 1H), 7.34-7.30 (m, 1H), 7.26-7.23 (m, 1H), 7.23-7.19 (m, 1H), 7.04 (t, J=9.0 Hz, 1H), 6.55-6.50 (m, 1H), 6.21 (d, J=3.3 Hz, 1H), 4.35-4.25 (m, 1H), 3.54-3.42 (m, 1H), 3.22-3.09 (m, 1H), 3.05-2.78 (m, 3H), 2.29 (d, J=1.5 Hz, 3H), 1.42 (d, J=6.6 Hz, 3H).

### Example 15: (S)-1-(2-(3-chloro-4-fluorophenyl)-4-methyl-4,5,6,7-tetrahydro-2H-pyrazolo[4,3-c]pyridine-3-yl)-3-(4-fluoro-1-(methyl-d3)-1H-indazole-5-yl)-1,3-dihydro-2H-imidazol-2-one (B-11)

By replacing 4-bromo-2,6-dimethylfluorobenzene with 3-chloro-4-fluorobromobenzene, and following procedures similar to those of Example 5a, the title compound was obtained.

ESI-MS(m/z): [M+H]⁺=499.2.

¹H NMR (400 MHz, DMSO-d₆) δ 8.28 (s, 1H), 7.69-7.48 (m, 3H), 7.48-7.30 (m, 2H), 7.06 (s, 1H), 6.94 (s, 1H), 3.89 (d, J=6.7 Hz, 1H), 3.25-3.13 (m, 1H), 2.91-2.76 (m, 1H), 2.68-2.60 (m, 2H), 2.45-2.30 (m, 1H), 1.12 (d, J=6.4 Hz, 3H).

### Example 16: (S)-1-(4-fluoro-1-(2,2,2-trifluoroethyl)-1H-indazole-5-yl)-3-(2-(4-fluoro-3,5-dimethylphenyl)-4-methyl-4,5,6,7-tetrahydro-2H-pyrazolo[4,3-c]pyridine-3-yl)-1,3-dihydro-2H-imidazol-2-one (B-12)

By replacing deuterated methyl iodide with 2,2,2-trifluoroethyl trifluoromethanesulfonate, and following procedures similar to those of Example 9, the title compound was obtained.

ESI-MS(m/z): [M+H]⁺=558.5.

### Example 17: tert-butyl 3-(3-(4-fluoro-1-methyl-1H-indazol-5-yl)-2-oxo-2,3-dihydro-1H-imidazol-1-yl)-2-(4-fluoro-3,5-dimethylphenyl)-6,7-dihydropyrazolo[1,5-a]pyrazine-5(4H)-carboxylate (B-13)

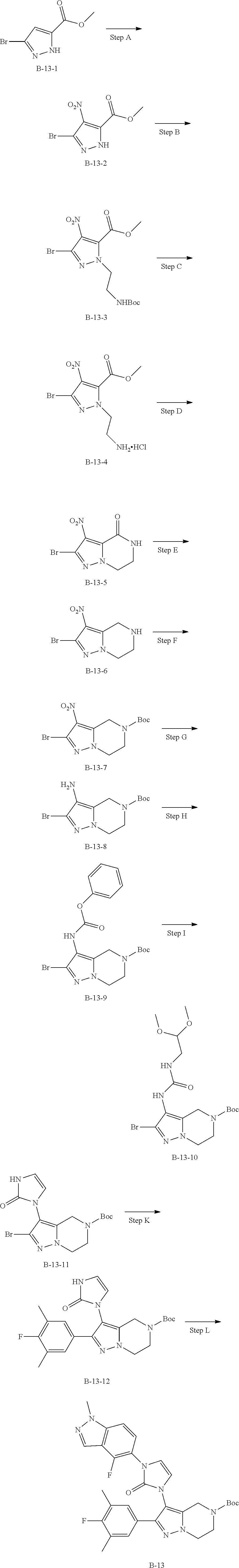

### Step A: methyl 3-bromo-4-nitro-1H-pyrazole-5-carboxylate (B-13-2)

To a mixture of B-13-1 (9.55 g, 46.60 mmol) in trifluoroacetic anhydride and chloroform (50 mL/50 mL), was added copper(II) nitrate trihydrate (13.51 g, 55.92 mmol) portionwise at 0° C. under nitrogen atmosphere. The reaction mixture was stirred at 25° C. for 3 h, cooled to 0° C. and slowly quenched with water (10 mL), and stirred for 2 h. The reaction mixture was extracted with dichloromethane (50 mL×3). The combined organic layers were concentrated to driness under reduced pressure. The residue was purified by silica gel column chromatography (dichloromethane/methanol=20/1) to obtain B-13-2 (13.33 g) as yellow solid.

ESI-MS(m/z): [M+H]⁺=250.0, 251.9.

### Step B: methyl 3-bromo-1-(2-((tert-butoxycarbonyl)amino)ethyl)-4-nitro-1H-pyrazole-5-carboxylate (B-13-3)

Under nitrogen atmosphere, a mixture of B-13-2 (12.76 g, 51.05 mmol) and N-(tert-butoxycarbonyl)ethanolamine (8.23 g, 51.05 mmol) in tetrahydrofuran (130 mL), was added triphenylphosphine (20.08 g, 76.57 mmol) and diethyl azodicarboxylate (20.64 g, 102.1 mmol) at 0° C. The reaction mixture was stirred at 0° C. for 2 h, quenched with water (50 mL), extracted with ethyl acetate (80 mL×3). The combined organic layers were concentrated to driness under reduced pressure. The residue was purified by silica gel column chromatography (petroleum ether/ethyl acetate=7/1) to obtain B-13-3 (31.93 g) as brown oil.

ESI-MS(m/z): [M+H]⁺=393.0, 395.0.

### Step C: methyl 1-(2-aminoethyl)-3-bromo-4-nitro-1H-pyrazole-5-carboxylate hydrochloride (B-13-4)

To a mixture of B-13-3 (8.73 g, 22.20 mmol) in dichloromethane (30 mL), was added HCl (4N in 1,4-dioxane, 60 mL). The reaction mixture was stirred at 25° C. for 1 h, and concentrated to obtain B-13-4 (12.07 g) as white solid.

ESI-MS(m/z): [M+H]⁺=293.0, 294.9.

### Step D: 2-Bromo-3-nitro-6,7-dihydro-pyrazolo[1,5-a]pyridazine-4(5H)-one (B-13-5)

To a mixture of B-13-4 (12.07 g, 36.63 mmol) in 1,4-dioxane (120 mL), was added triethylamine (14.83 g, 146.52 mmol). The reaction mixture was stirred at 80° C. for 1 h, and cooled to 25° C., concentrated to driness under reduced pressure. The residue was slurried with methanol (30 mL), filtered and dried to obtain B-13-5 (1.88 g) as white solid.

ESI-MS(m/z): [M+H]⁺=260.9, 263.0.

### Step E: 2-Bromo-3-nitro-4,5,6,7-tetrahydro-pyrazolo[1,5-a]pyridazine hydrochloride(B-13-6)

To a mixture of B-13-5 (1.78 g, 6.82 mmol) in tetrahydrofuran (18 mL), was added borane (1 M in THF, 17.1 mL, 17.10 mmol) under nitrogen atmosphere. The reaction mixture was stirred at 70° C. for 3 h, cooled to 25° C., quenched with HCl (4 M, 3 mL), and concentrated to driness under reduced pressure. The residue was slurried with methanol (8 mL), filtered and dried to obtain B-13-6 (2.16 g) as white solid.

ESI-MS(m/z): [M+H]⁺=247.0, 249.0.

### Step F: tert-butyl 2-bromo-3-nitro-6,7-dihydropyrazolo[1,5-a]pyrazine-5(4H)-carboxylate (B-13-7)

To a mixture of B-13-6 (2.16 g, 8.73 mmol) in dichloromethane (30 mL), was added triethylamine (2.66 g, 26.19 mmol) and di-tert-butyl dicarbonate (2.48 g, 11.35 mmol). The reaction mixture was stirred at 25° C. for 1 h, quenched with water (1 mL), extracted with ethyl acetate (30 mL×2). The combined organic layers were concentrated to driness under reduced pressure. The residue was purified by silica gel column chromatography (petroleum ether/ethyl acetate=8/1) to obtain B-13-7 (1.20 g) as white solid.

ESI-MS(m/z): [M+H]⁺=346.9, 348.8.

### Step G: tert-butyl 3-amino-2-bromo-6,7-dihydropyrazolo[1,5-a]pyrazine-5(4H)-carboxylate (B-13-8)

To a mixture of B-13-7 (0.9 g, 2.6 mmol) in ethanol and water (10 mL/10 mL), was added ammonium chloride (1.4 g, 26.0 mmol) and zinc powder (0.85 g, 13 mmol) under a nitrogen atmosphere. The reaction mixture was stirred at reflux for 3 h, cooled to 25° C., quenched with water (10 mL), and extracted with ethyl acetate (15 mL×3). The combined organic layers were washed with saturated brine, dried over anhydrous sodium sulfate, and concentrated to driness under reduced pressure. The residue was purified silica gel column chromatography (petroleum ether/ethyl acetate=10/3) to obtain B-13-8 (0.7 g, 82%) as white solid.

ESI-MS(m/z): [M+H]⁺=317.3.

### Step H: tert-butyl 2-bromo-3-((phenoxycarbonyl)amino)-6,7-dihydropyrazolo[1,5-a]pyrazine-5(4H)-carboxylate (B-13-9)

To a mixture of B-13-8 (620.0 mg, 1.95 mmol) in tetrahydrofuran (10 mL), was added diisopropylethylamine (504.0 mg, 3.9 mmol) and phenyl chloroformate (396.9 mg, 2.54 mmol) at 0° C. The reaction mixture was stirred at 25° C. for 2 h, and concentrated to driness under reduced pressure to obtain B-13-9 (600.0 mg, 80%) as yellow solid.

ESI-MS(m/z): [M+H]⁺=317.3.

### Step I: tert-butyl 2-bromo-3-(3-(2,2-dimethoxyethyl)ureido)-6,7-dihydropyrazolo[1,5-a]pyrazine-5(4H)-carboxylate (B-13-10)

To a mixture of B-13-9 (600.0 mg, 1.37 mmol) in pyridine (10 mL), was added 2,2-dimethoxyethyl urea (288.1 mg, 2.74 mmol). The reaction mixture was stirred at 25° C. for 2 h, quenched with water (10 mL), extracted with ethyl acetate (15 mL×3). The combined organic layers were washed with saturated brine, dried over anhydrous sodium sulfate, and concentrated to driness under reduced pressure. The residue was purified by silica gel column chromatography (petroleum ether/ethyl acetate=1/1) to obtain B-13-10 (600.0 mg) as white solid.

ESI-MS(m/z): [M+H]⁺=448.2.

### Step J: tert-butyl 2-bromo-3-(2-oxo-2,3-dihydro-1H-imidazol-1-yl)-6,7-dihydropyrazolo[1,5-a]pyrazine-5(4H)-carboxylate (B-13-11)

To a mixture of B-13-10 (580.0 mg, 1.29 mmol) in tetrahydrofuran (12 mL), was added methanesulfonic acid (99.2 mg, 1.03 mmol) at 0° C. The reaction mixture was stirred at 70° C. for 2 h, cooled to 25° C., quenched with water (10 mL), extracted with ethyl acetate (15 mL×3). The combined organic layers were washed with saturated brine, dried over anhydrous sodium sulfate, and concentrated under reduced pressure to obtain B-13-11 (480.0 mg) as white solid.

ESI-MS(m/z): [M+H]⁺=384.2.

### Step K: tert-Butyl 2-(4-fluoro-3,5-dimethylphenyl)-3-(2-oxo-2,3-dihydro-1H-imidazol-1-yl)-6,7-dihydro-1H-pyrazolo[1,5-a]pyrazine-5(4H)-carboxylate (B-13-12)

Under nitrogen atmosphere, to a mixture of B-13-11 (480.0 mg, 1.25 mmol) in 1,4-dioxane/water (8 mL/2 mL), was add 3,5-dimethyl-4-fluorophenylboronic acid (273.0 mg, 1.63 mmol), [1,1′-bis(diphenylphosphino)ferrocene]palladium dichloride (183.0 mg, 0.25 mmol), and potassium carbonate (518.3 mg, 3.75 mmol). The reaction mixture was stirred at 100° C. for 3 h, cooled to 25° C., quenched with water (5 mL), extracted with ethyl acetate (10 mL×3). The combined organic layers were washed with saturated brine, dried over anhydrous sodium sulfate, and concentrated to driness under reduced pressure. The residue was purified by silica gel column chromatography (petroleum ether/ethyl acetate=2/1) to obtain B-13-12 (440.0 mg, 82%) as white solid.

ESI-MS(m/z): [M+H]⁺=428.3.

### Step L: tert-butyl 3-(3-(4-fluoro-1-methyl-1H-indazol-5-yl)-2-oxo-2,3-dihydro-1H-imidazol-1-yl)-2-(4-fluoro-3,5-dimethylphenyl)-6,7-dihydropyrazolo[1,5-a]pyrazine-5(4H)-carboxylate (B-13)

Under nitrogen atmosphere, to a mixture of B-13-12 (100.0 mg, 0.23 mmol) in N-methyl-2-pyrrolidone (2 mL), was added 5-bromo-4-fluoro-1-methylindazole (100.0 mg, 0.23 mmol), copper iodide (4.4 mg, 0.02 mmol), trans-(1R,2R)-N,N′-dimethylcyclohexane-1,2-diamine (63.0 mg, 0.28 mmol), and potassium carbonate (48.0 mg, 0.35 mmol). The reaction mixture was stirred at 130° C. for 3 h, cooled to 25° C., quenched with water (3 mL), extracted with ethyl acetate (5 mL×3). The combined organic layers were washed with saturated brine, dried over anhydrous sodium sulfate, and concentrated to driness under reduced pressure. The residue was purified by silica gel column chromatography (dichloromethane/methanol=20/1) to obtain B-13 (79.6 mg, yield: 59%) as white solid.

ESI-MS(m/z): [M+H]⁺=576.2.

¹H NMR (400 MHz, DMSO-d6) δ 8.29 (s, 1H), 7.63 (d, J=8.8 Hz, 1H), 7.53 (t, J=7.2 Hz, 1H), 7.22 (d, J=7.2 Hz, 2H), 7.01 (d, J=2.8 Hz, 1H), 6.74 (d, J=2.8 Hz, 1H), 4.55 (s, 2H), 4.18 (t, J=5.6 Hz, 2H), 4.11 (s, 3H), 3.90 (t, J=5.6 Hz, 2H), 2.22 (s, 6H), 1.45 (s, 9H).

### Example 18: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-azabicyclo[3.1.0]hexan-6-yl]-4,5-dihydro-1,2,4-oxadiazol-5-one (C-1)

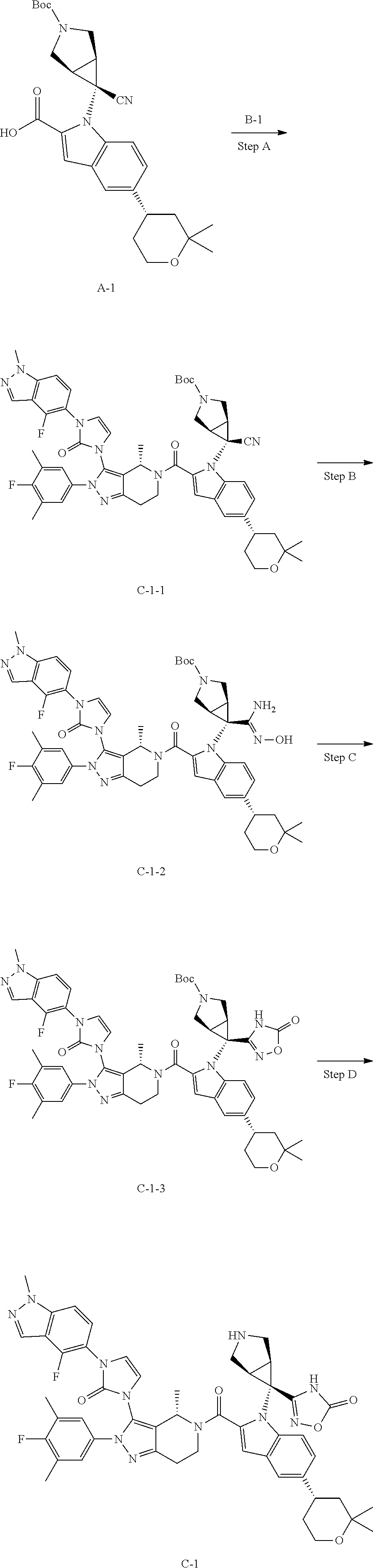

### Step A: tert-butyl (1R,5S,6S)-6-cyano-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-azabicyclo[3.1.0]hexane-3-carboxylate (C-1-1)

A mixture of A-1 (330.0 mg, 0.69 mmol), B-1 (361.9 mg, 0.69 mmol), 2-(7-aza-1H-benzotriazole)-N,N,N′,N′-tetramethyluronium hexafluorophosphate (392.5 mg, 1.03 mmol), and diisopropylethylamine (266.8 mg, 2.06 mmol) in N,N-dimethylformamide (10 mL) was stirred at 25° C. for 16 h. The reaction mixture was quenched with water (10 mL), extracted with ethyl acetate (20 mL×2). The combined organic layers were washed with saturated brine (10 mL), concentrated to driness under reduced pressure. The residue was purified by silica gel column chromatography (dichloromethane/methanol=99/1) to obtain C-1-1 (570.0 mg, 87%) as yellow solid.

ESI-MS(m/z): [M+H]⁺=951.5

### Step B: tert-butyl (1R,5S,6S)-6-{5-[(4S)-2,2-dimethyloxan-4-yl]-2-[(4S)-3-[3-(4-fluoro-i-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-6-(N′-hydroxycarbamimidoyl)-3-azabicyclo[3.1.0]hexane-3-carboxylate (C-1-2)

A mixture of C-1-1 (570.0 mg, 0.60 mmol), hydroxylamine hydrochloride (1.3 g, 17.98 mmol), and diisopropylethylamine (2.3 g, 17.98 mmol) in ethanol (10 mL) was stirred at 80° C. for 3 h, cooled to 25° C., quenched with saturated ammonium chloride (10 mL), and extracted with ethyl acetate (20 mL×2). The combined organic layers were concentrated to driness under reduced pressure to obtain C-1-2 (590.0 mg) as pale white solid.

ESI-MS(m/z): [M+H]⁺=984.5

### Step C: tert-butyl (1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-6-(5-oxo-4,5-dihydro-1,2,4-oxadiazol-3-yl)-3-azabicyclo[3.1.0]hexane-3-carboxylate (C-1-3)

A mixture of C-1-2 (590.0 mg, 0.60 mmol), N,N′-carbonyldiimidazole (194.3 mg, 1.20 mmol), and 1,8-diazabicyclo[5.4.0]undec-7-ene (228.1 mg, 1.5 mmol) in dimethyl sulfoxide (5 mL) was stirred at 25° C. for 3 h. The reaction mixture was quenched with saturated ammonium chloride (5 mL), extracted with ethyl acetate (10 mL×2). The combined organic layers were washed with saturated brine (10 mL), concentrated to driness under reduced pressure. The residue was purified by silica gel column chromatography (dichloromethane/methanol=99/1) to obtain C-1-3 (590.0 mg, 97%) as pale white solid.

ESI-MS(m/z): [M+H]⁺=1010.4

### Step D: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-azabicyclo[3.1.0]hexan-6-yl]-4,5-dihydro-1,2,4-oxadiazol-5-one (C-1)

To a mixture of C-1-3 (220.0 mg, 0.22 mmol) in dichloromethane (5 mL), was added HCl (4M in 1,4-dioxane, 2 mL). The reaction mixture was stirred at 25° C. for 1 h, quenched with saturated sodium bicarbonate (5 mL), extracted with ethyl acetate (10 mL×2). The combined organic layers were concentrated to driness under reduced pressure, and the residue was purified by silica gel column chromatography (dichloromethane/methanol=15/1) to obtain C-1 (190.0 mg) as pale white solid.

ESI-MS(m/z): [M+H]⁺=910.4

¹H NMR (400 MHz, DMSO-d₆) δ 8.32 (m, 1H), 8.21 (m, 1H), 7.65 (m, 1H), 7.47 (m, 4H), 7.26-7.07 (m, 4H), 7.04-6.77 (m, 1H), 6.66 (s, 1H), 5.63 (m, 1H), 4.13 (m, 2H), 3.79 (m, 2H), 3.49 (m, 2H), 3.12-2.64 (m, 6H), 2.26 (m, 4H), 1.77-1.48 (m, 4H), 1.29 (m, 15H).

### Example 19: 3-[(1R,5S,6S)-3-acetyl-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-azabicyclo[3.1.0]hexan-6-yl]-4,5-dihydro-1,2,4-oxadiazol-5-one (C-2)

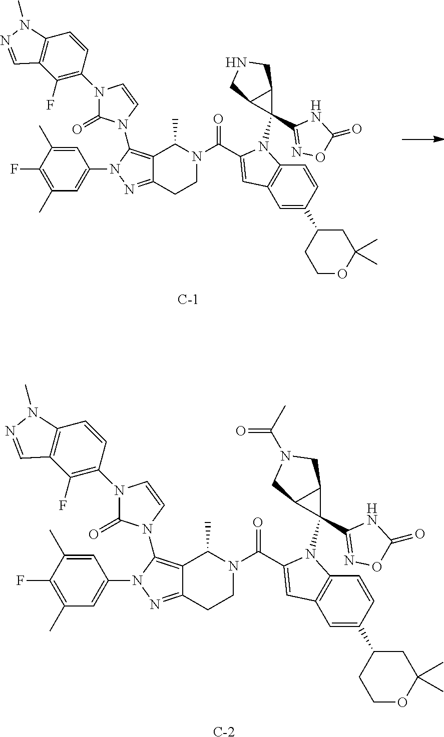

To a mixture of C-1 (20.0 mg, 0.022 mmol) and triethylamine (4.5 mg, 0.044 mmol) in dichloromethane (5 mL), was slowly added acetyl chloride (1.9 mg, 0.024 mmol) at 0° C. The reaction mixture was stirred at 0° C. for 1 h, quenched with saturated ammonium chloride (3 mL), and extracted with dichloromethane (5 mL×2). The combined organic layers were concentrated to driness under reduced pressure. The residue was purified by silica gel column chromatography (dichloromethane/methanol=95/5) to obtain C-2 (10.0 mg, 48%) as yellow solid.

ESI-MS(m/z): [M+H]⁺=952.4

¹H NMR (400 MHz, DMSO-d₆) δ 11.76 (s, 1H), 8.32 (s, 1H), 7.65 (m, 2H), 7.49 (m, 2H), 7.24 (m, 3H), 7.09 (s, 1H), 6.96 (m, 2H), 5.68 (m, 1H), 4.29 (m, 1H), 4.09 (m, 3H), 3.71 (m, 3H), 3.45-3.32 (m, 7H), 3.05 (m, 2H), 2.95-2.61 (m, 2H), 2.24 (m, 6H), 2.00 (m, 1H), 1.83 (m, 2H), 1.75-1.61 (m, 2H), 1.46 (m, 3H), 1.27 (s, 3H), 1.18 (s, 3H).

### Example 20: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-methanesulfonyl-3-azabicyclo[3.1.0]hexan-6-yl]-4,5-dihydro-1,2,4-oxadiazol-5-one (C-3)

ESI-MS(m/z): [M+H]⁺=988.5.

By replacing acetyl chloride with methanesulfonyl chloride, and following procedures similar to those of Example 19, the titled compound was obtained.

### Example 21: 3-[(1R,5S,6S)-3-(cyclopropanesulfonyl)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-azabicyclo[3.1.0]hexan-6-yl]-4,5-dihydro-1,2,4-oxadiazol-5-one (C-4)

By replacing acetyl chloride with cyclopropanesulfonyl chloride, and following procedures similar to those of Example 19, the titled compound was obtained.

ESI-MS(m/z): [M+H]⁺=1014.6

### Example 22: (1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-6-(5-oxo-4,5-dihydro-1,2,4-oxadiazol-3-yl)-3-azabicyclo[3.1.0]hexane-3-carboxamide (C-5)

By replacing acetyl chloride with phenylcarbamate, and following procedures similar to those of Example 19, the titled compound was obtained.

ESI-MS(m/z): [M+H]⁺=953.4.

¹H NMR (400 MHz, DMSO-d6) δ 8.32 (s, 1H), 7.65 (d, J=8.8 Hz, 1H), 7.55-7.42 (m, 2H), 7.35-7.12 (m, 4H), 7.11-7.06 (m, 1H), 6.97 (s, 1H), 5.83-5.60 (m, 2H), 4.15-4.03 (m, 4H), 3.86-3.66 (m, 3H), 3.53-3.39 (m, 2H), 3.03 (s, 2H), 2.88-2.63 (m, 3H), 2.27-2.24 (m, 6H), 1.72-1.64 (m, 2H), 1.62-1.48 (m, 3H), 1.47-1.40 (m, 3H), 1.31-1.22 (m, 5H), 1.19-1.15 (m, 3H).

### Example 23: 3-[(1R,5S,6S)-3-cyclopropanecarbonyl-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-azabicyclo[3.1.0]hexan-6-yl]-4,5-dihydro-1,2,4-oxadiazol-5-one (C-6)

By replacing acetyl chloride with cyclopropanecarbonyl chloride, and following procedures similar to those of Example 19, the titled compound was obtained.

ESI-MS(m/z): [M+H]⁺=978.4.

### Example 24: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(1-fluorocyclopropanecarbonyl)-3-azabicyclo[3.1.0]hexan-6-yl]-4,5-dihydro-1,2,4-oxadiazol-5-one (C-7)

A mixture of C-1 (20.0 mg, 0.022 mmol), 1-fluorocyclopropane carboxylic acid (2.8 mg, 0.026 mmol), 1-(3-dimethylaminopropyl)-3-ethylcarbodiimide hydrochloride (5.0 mg, 0.026 mmol), and 1-hydroxy-1H-benzotriazole in N,N-dimethylformamide (0.5 mL) was stirred at 25° C. for 2 h. The reaction mixture was purified by C-18 reverse-phase column chromatography (water/acetonitrile=60/40 to 30/70) to obtain C-7 (9.2 mg, 42%) as white solid.

ESI-MS(m/z): [M+H]⁺=996.4.

¹H NMR (400 MHz, DMSO d₆) δ 8.32 (s, 1H), 7.65 (d, J=8.9 Hz, 1H), 7.55-7.44 (m, 2H), 7.36-7.14 (m, 5H), 7.10-7.06 (m, 1H), 6.96 (s, 1H), 5.72-5.60 (m, 1H), 4.62-4.23 (m, 1H), 4.12 (s, 4H), 3.78-3.56 (m, 4H), 3.22-2.97 (m, 2H), 2.94-2.59 (m, 3H), 2.28-2.18 (m, 6H), 1.71-1.64 (m, 2H), 1.64-1.48 (m, 3H), 1.46-1.41 (m, 3H), 1.30-1.21 (m, 9H), 1.21-1.17 (m, 3H).

### Example 25: 1-[(4S)-5-{1-[(R,5S,6S)-3-acetyl-6-(1H-1,2,3,4-tetrazol-5-yl)-3-azabicyclo[3.1.0]hexan-6-yl]-5-(oxan-4-yl)-1H-indole-2-carbonyl}-2-(4-fluoro-3,5-dimethylphenyl)-4-methyl-2H,4H,5H,6H,7H-pyrazolo[4,3-c]pyridin-3-yl]-3-(4-fluoro-1-methyl-1H-indazol-5-yl)-2,3-dihydro-1H-imidazol-2-one (C-8)

By replacing A-1 with A-3, and following procedures similar to those of Examples 18 and 19, the titled compound was obtained.

ESI-MS(m/z): [M+H]⁺=908.4.

¹H NMR (400 MHz, DMSO-d₆) δ 8.28 (m, 1H), 7.91 (m, 1H), 7.66 (m, 1H), 7.58-7.46 (m, 1H), 7.39 (s, 1H), 7.22 (m, 4H), 7.09 (s, 1H), 6.97 (s, 1H), 6.65 (m, 1H), 4.61 (m, 1H), 4.09 (m, 3H), 3.96 (m, 3H), 3.76 (m, 2H), 3.44 (m, 3H), 2.94-2.59 (m, 5H), 2.23 (m, 6H), 2.07-1.90 (m, 2H), 1.73 (m, 4H), 1.53 (m, 6H).

### Example 26: 1-(4-fluoro-1-methyl-1H-indazol-5-yl)-3-[(4S)-2-(4-fluoro-3,5-dimethylphenyl)-4-methyl-5-[5-(oxan-4-yl)-1-[(1R,5S,6S)-6-(1H-1,2,3,4-tetrazol-5-yl)-3-azabicyclo[3.1.0]hexan-6-yl]-1H-indole-2-carbonyl]-2H,4H,5H,6H,7H-pyrazolo[4,3-c]pyridin-3-yl]-2,3-dihydro-1H-imidazol-2-one (C-9)

By replacing A-1 with A-3 and following the procedures similar to those of Example 18, the titled compound was obtained.

ESI-MS(m/z): [M+H]⁺=910.4.

¹H NMR (400 MHz, DMSO-d₆) δ 8.92 (s, 1H), 8.29 (m, 1H), 7.85 (s, 1H), 7.60 (m, 1H), 7.45 (m, 2H), 7.34 (s, 1H), 7.20-7.02 (m, 4H), 6.89 (m, 1H), 6.63 (m, 1H), 5.46 (m, 1H), 4.09 (m, 3H), 3.96 (m, 2H), 3.47 (m, 9H), 2.84 (m, 3H), 2.62 (m, 2H), 2.24 (s, 6H), 1.73 (s, 4H), 1.31 (m, 2H).

### Example 27: 1-(4-fluoro-1-methyl-1H-indazol-5-yl)-3-[(4S)-2-(4-fluoro-3,5-dimethylphenyl)-4-methyl-5-{1-[(1R,5S,6S)-3-methyl-6-(1H-1, 2, 3, 4-tetrazol-5-yl)-3-azabicyclo[3.1.0]hexan-6-yl]-5-(oxan-4-yl)-1H-indole-2-carbonyl}-2H,4H,5H,6H,7H-pyrazolo[4,3-c]pyridin-3-yl]-2,3-dihydro-1H-imidazol-2-one (C-10)

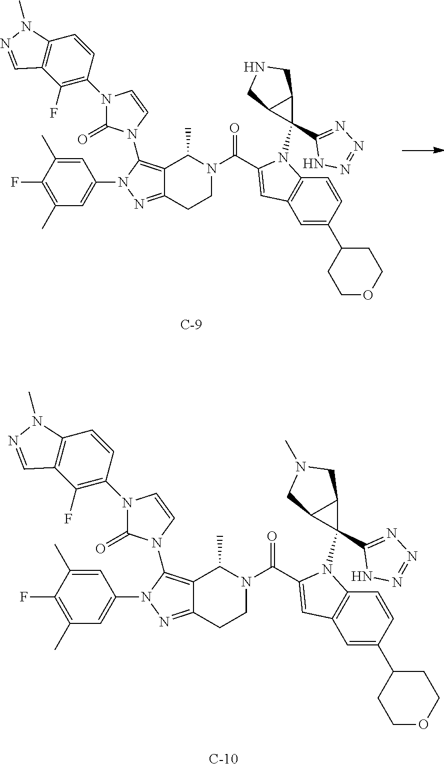

A mixture of C-9 (20.0 mg, 0.023 mmol), aqueous formaldehyde solution (2.3 mg, 0.028 mmol), and acetic acid (2.8 mg, 0.046 mmol) in methanol (5 mL) was stirred at 25° C. for 30 min. Sodium cyanoborohydride (2.9 mg, 0.046 mmol) was added. The reaction mixture was stirred at 25° C. for 30 min, quenched with saturated ammonium chloride (3 mL), extracted with dichloromethane (5 mL×2). The combined organic layers were concentrated to driness under reduced pressure. The residue was purified by silica gel column chromatography (dichloromethane/methanol=95/5) to obtain C-10 (10.0 mg, 48%) as white solid.

ESI-MS(m/z): [M+H]⁺=908.4.

¹H NMR (400 MHz, DMSO-d₆) δ 8.28 (m, 1H), 7.87-7.28 (m, 5H), 7.22-7.01 (m, 3H), 6.89 (m, 1H), 6.67 (m, 1H), 5.60 (m, 1H), 4.10 (m, 3H), 3.96 (m, 2H), 3.73-3.40 (m, 4H), 3.34 (m, 4H), 2.65 (m, 5H), 2.47 (m, 2H), 2.24 (s, 6H), 1.72 (s, 4H), 1.31 (m, 3H), 1.23 (s, 2H).

### Example 28: (1R,5S,6R)-6-{5-[(4S)-2,2-dimethyloxan-4-yl]-2-[(4S)-3-{3-[4-fluoro-1-(²H₃)methyl-1H-indazol-5-yl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-1H-indol-1-yl}-6-(5-oxo-4,5-dihydro-1,2,4-oxadiazol-3-yl)-3λ⁶-thiabicyclo[3.1.0]hexane-3, 3-dione (C-11)

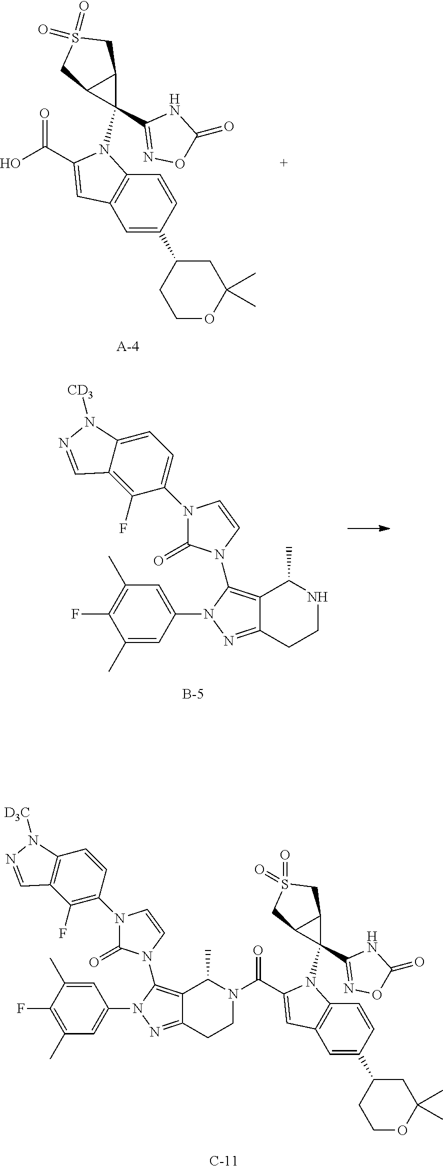

A mixture of A-4 (10.0 mg, 0.021 mmol), B-5 (10.1 mg, 0.021 mmol), 1-(3-dimethylaminopropyl)-3-ethylcarbodiimide hydrochloride (4.7 mg, 0.025 mmol), and 1-hydroxybenzotriazole (4.2 mg, 0.030 mmol) in N,N-dimethylformamide (0.2 mL) was stirred at 25° C. for 2 h. The reaction mixture was purified by C-18 reverse-phase column chromatography (water/acetonitrile=1/3) to obtain C-11 (12.3 mg, 62%) as white solid.

ESI-MS(m/z): [M+H]⁺=962.4

¹H NMR (400 MHz, DMSO-d₆) δ 8.31 (s, 1H), 7.64 (d, J=8.8 Hz, 1H), 7.59-7.40 (m, 2H), 7.30-7.12 (m, 4H), 7.08 (d, J=3.1 Hz, 1H), 6.99-6.89 (m, 2H), 5.70-5.45 (m, 1H), 4.63-4.39 (m, 1H), 3.85-3.55 (m, 5H), 3.23-3.12 (m, 1H), 3.10-2.99 (m, 1H), 2.94-2.77 (m, 2H), 2.76-2.61 (m, 1H), 2.30-2.19 (m, 6H), 1.73-1.64 (m, 3H), 1.64-1.48 (m, 2H), 1.42-1.32 (m, 3H), 1.30-1.22 (m, 5H), 1.22-1.14 (m, 3H).

### Example 29: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyrimidin-2-yl)-3-azabicyclo[3.1.0]hexan-6-yl]-4,5-dihydro-1,2,4-oxadiazol-5-one (C-12)

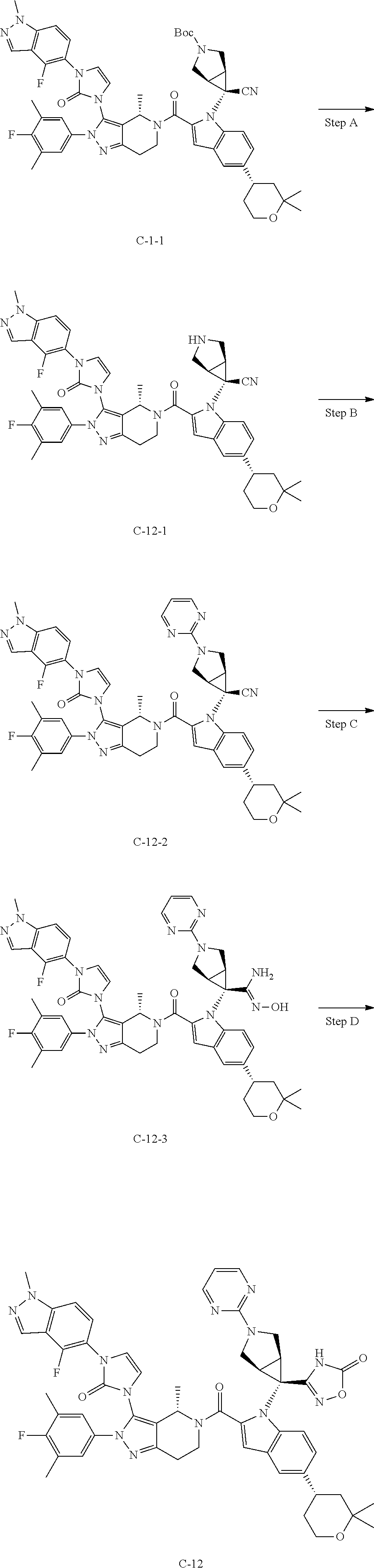

### Step A: (1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-azabicyclo[3.1.0]hexane-6-carbonitrile (C-12-1)

To a mixture of C-1-1 (500 mg, 0.53 mmol) in dichloromethane (8 mL), was added trifluoroacetic acid (2 mL). The reaction mixture was stirred at 25° C. for 2 h, and concentrated to driness under reduced pressure. The residue was treated with water, quenched with saturated sodium bicarbonate solution, extracted with dichloromethane (10 mL×3). The combined organic layers were washed with saturated brine (10 mL), dried over anhydrous sodium sulfate, and concentrated to driness under reduced pressure to obtain C-12-1 (400 mg) as brown solid.

ESI-MS(m/z): [M+H]⁺=850.9

### Step B: (1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyrimidin-2-yl)-3-azabicyclo[3.1.0]hexane-6-carbonitrile (C-12-2)

A mixture of C-12-1 (350 mg, 0.41 mmol), 2-bromopyrimidine (97.77 mg, 0.61 mmol), copper(I) iodide (7.81 mg, 0.041 mmol), 2-(2,6-dimethylphenylamino)-2-oxoacetic acid (15.84 mg, 0.082 mmol), and potassium phosphate (174.06 mg, 0.82 mmol) in N,N-dimethylformamide (7 mL) was stirred at 90° C. for 16 h under nitrogen atmosphere. The reaction mixture was cooled to 25° C., quenched with water (5 mL), extracted with dichloromethane (10 mL×3). The combined organic layers were washed with saturated brine (10 mL), dried over anhydrous sodium sulfate, and concentrated to driness under reduced pressure. The residue was purified by silica gel column chromatography (dichloromethane/methanol=100/3) to obtain C-12-2 (250 mg, 65%) as light yellow solid.

ESI-MS(m/z): [M+H]⁺=929.1

### Step C: (1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-N′-hydroxy-3-(pyrimidin-2-yl)-3-azabicyclo[3.1.0]hexane-6-carboximidamide (C-12-3)

To a mixture of C-12-2 (15 mg, 0.016 mmol) in ethanol (1 mL), was added sodium bicarbonate (14 mg, 0.16 mmol) and hydroxylamine hydrochloride (11 mg, 0.16 mmol). The reaction mixture was stirred at 70° C. for 6 h, cooled to 25° C., quenched with water (1 mL), extracted with ethyl acetate (5 mL×3). The combined organic layers were washed with saturated brine (3 mL), dried over anhydrous sodium sulfate, and concentrated to driness under reduced pressure to obtain C-12-3 (10 mg) as yellow solid.

ESI-MS(m/z): [M+H]⁺=962.3

### Step D: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyrimidin-2-yl)-3-azabicyclo[3.1.0]hexan-6-yl]-4,5-dihydro-1,2,4-oxadiazol-5-one (C-12)

To a mixture of C-12-3 (10 mg) in dimethyl sulfoxide (1 mL), was added N,N′-carbonyldiimidazole (5 mg, 0.032 mmol) and 1,8-diazabicyclo[5.4.0]undec-7-ene (6 mg, 0.040 mmol). The reaction mixture was stirred at 25° C. for 1 h, quenched with water (1 mL), extracted with ethyl acetate (5 mL×3). The combined organic layers were washed with saturated brine (3 mL), dried over anhydrous sodium sulfate, and concentrated to driness under reduced pressure. The residue was purified by silica gel column chromatography (dichloromethane/methanol=100/5) to obtain C-12 (5 mg, 31%) as white solid.

ESI-MS(m/z): [M+H]⁺=989.0

¹H NMR (400 MHz, DMSO-d₆) δ 8.36-8.21 (m, 3H), 7.88-7.59 (m, 2H), 7.57-7.45 (m, 2H), 7.33-7.26 (m, 1H), 7.23-7.09 (m, 3H), 6.99-6.77 (m, 2H), 6.69-6.62 (m, 1H), 5.75-5.61 (m, 1H), 4.62-4.30 (m, 1H), 4.21-4.06 (m, 4H), 3.82-3.51 (m, 6H), 3.16-2.58 (m, 5H), 2.39-2.12 (m, 7H), 1.72-1.65 (m, 2H), 1.63-1.53 (m, 2H), 1.52-1.43 (m, 3H), 1.30-1.23 (m, 4H), 1.19 (s, 3H).

### Example 30: 1-(4-fluoro-1-methyl-1H-indazol-5-yl)-3-[(4S)-2-(4-fluoro-3,5-dimethylphenyl)-4-methyl-5-[5-(oxan-4-yl)-1-[(1R,5S,6S)-3-(propan-2-yl)-6-(1H-1,2,3,4-tetrazol-5-yl)-3-azabicyclo[3.1.0]hexan-6-yl]-1H-indole-2-carbonyl]-2H,4H,5H,6H,7H-pyrazolo[4,3-c]pyridin-3-yl]-2,3-dihydro-1H-imidazol-2-one (C-13)

By replacing aqueous formaldehyde solution with acetone, and following procedures similar to those of Example 27, the titled compound was obtained.

ESI-MS(m/z): [M+H]⁺=908.4

¹H NMR (400 MHz, DMSO-d₆) δ 8.29 (m, 1H), 7.62 (m, 2H), 7.40 (m, 3H), 7.12 (m, 4H), 6.94 (s, 1H), 6.71 (s, 1H), 5.62 (s, 1H), 4.10 (m, 4H), 3.96 (m, 2H), 3.48 (m, 4H), 2.81 (m, 4H), 2.25 (s, 6H), 1.73 (s, 4H), 1.22 (m, 14H).

### Example 31: 1-(4-fluoro-1-methyl-1H-indazol-5-yl)-3-[(4S)-2-(4-fluoro-3,5-dimethylphenyl)-4-methyl-5-[5-(oxan-4-yl)-1-[(1R,5S,6S)-3-(oxetan-3-yl)-6-(1H-1,2,3,4-tetrazol-5-yl)-3-azabicyclo[3.1.0]hexan-6-yl]-1H-indole-2-carbonyl]-2H,4H,5H,6H,7H-pyrazolo[4,3-c]pyridin-3-yl]-2,3-dihydro-1H-imidazol-2-one (C-14)

By replacing the aqueous formaldehyde solution with 3-oxetanone, and following the procedures similar to those of Example 27, the titled compound was obtained

ESI-MS(m/z): [M+H]⁺=922.4

¹H NMR (400 MHz, DMSO-d₆) δ 8.33 (s, 1H), 8.00 (m, 1H), 7.66 (m, 1H), 7.57-7.34 (m, 3H), 7.29 (s, 1H), 7.22-7.07 (m, 3H), 6.98 (s, 1H), 6.75 (s, 1H), 5.74 (s, 1H), 4.85 (m, 1H), 4.56 (m, 1H), 4.27 (m, 3H), 4.12 (s, 3H), 3.96 (m, 3H), 3.44 (m, 4H), 3.11 (m, 2H), 2.84 (s, 2H), 2.26 (s, 6H), 1.72 (s, 4H), 1.47 (m, 4H), 1.23 (s, 3H).

### Example 32: 1-(4-fluoro-1-methyl-1H-indazol-5-yl)-3-[(4S)-2-(4-fluoro-3,5-dimethylphenyl)-4-methyl-5-[5-(oxan-4-yl)-1-[(1R,5S,6S)-3-(oxolan-3-yl)-6-(1H-1,2,3,4-tetrazol-5-yl)-3-azabicyclo[3.1.0]hexan-6-yl]-1H-indole-2-carbonyl]-2H,4H,5H,6H,7H-pyrazolo[4,3-c]pyridin-3-yl]-2,3-dihydro-1H-imidazol-2-one (C-15)

By replacing the aqueous formaldehyde solution with dihydro-3(2H)-furanone, and following the procedures similar to those of Example 27, the titled compound was obtained.

ESI-MS(m/z): [M+H]⁺=936.4

¹H NMR (400 MHz, DMSO-d₆) δ 15.32 (s, 1H), 8.29 (m, 1H), 7.94 (s, 1H), 7.66 (m, 1H), 7.54-7.44 (m, 1H), 7.42 (s, 1H), 7.27 (s, 1H), 7.19 (m, 2H), 7.10 (s, 1H), 6.98 (s, 1H), 6.73 (s, 1H), 5.73 (s, 1H), 4.10 (m, 5H), 3.96 (m, 2H), 3.56-3.40 (m, 6H), 3.26 (m, 2H), 3.22-2.97 (m, 2H), 2.82 (m, 3H), 2.67 (m, 2H), 2.26 (s, 6H), 1.72 (s, 5H), 1.44 (m, 3H), 1.23 (s, 2H).

### Example 33: 1-(4-fluoro-1-methyl-1H-indazol-5-yl)-3-[(4S)-2-(4-fluoro-3,5-dimethylphenyl)-4-methyl-5-[5-(oxan-4-yl)-1-[(1R,5S,6S)-3-(oxan-4-yl)-6-(1H-1,2,3,4-tetrazol-5-yl)-3-azabicyclo[3.1.0]hexan-6-yl]-1H-indole-2-carbonyl]-2H,4H,5H,6H,7H-pyrazolo[4,3-c]pyridin-3-yl]-2,3-dihydro-1H-imidazol-2-one (C-16)

By replacing the aqueous formaldehyde solution with tetrahydro-2H-pyran-4-one, and following procedures similar to those of Example 27, the titled compound was obtained.

ESI-MS(m/z): [M+H]⁺=949.9

¹H NMR (400 MHz, DMSO-d₆) δ 15.27 (m, 1H), 8.32 (s, 1H), 7.91-7.88 (m, 1H), 7.66-7.64 (m, 1H), 7.46-7.44 (m, 2H), 7.28-7.19 (m, 2H), 7.11-7.09 (m, 1H), 6.98-6.75 (m, 2H), 5.69 (s, 1H), 4.12 (m, 5H), 3.97-3.95 (m, 4H), 3.61-3.45 (m, 8H), 3.16 (m, 4H), 2.84-2.81 (m, 3H), 2.70-2.67 (m, 2H), 2.25 (m, 6H), 1.72 (m, 3H), 1.46 (m, 4H), 1.05 (m, 1H), 0.96-0.85 (m, 1H).

### Example 34: 1-(4-fluoro-1-methyl-1H-indazol-5-yl)-3-[(4S)-2-(4-fluoro-3,5-dimethylphenyl)-4-methyl-5-[5-(oxan-4-yl)-1-[(1R,5S,6S)-3-[(1r,4S)-4-hydroxycyclohexyl]-6-(1H-1, 2,3,4-tetrazol-5-yl)-3-azabicyclo[3.1.0]hexan-6-yl]-1H-indole-2-carbonyl]-2H,4H,5H,6H,7H-pyrazolo[4,3-c]pyridin-3-yl]-2,3-dihydro-1H-imidazol-2-one

### And 1-(4-fluoro-1-methyl-1H-indazol-5-yl)-3-[(4S)-2-(4-fluoro-3,5-dimethylphenyl)-4-methyl-5-[5-(oxan-4-yl)-1-[(1R,5S,6S)-3-[(1s,4R)-4-hydroxycyclohexyl]-6-(1H-1, 2,3,4-tetrazol-5-yl)-3-azabicyclo[3.1.0]hexan-6-yl]-1H-indole-2-carbonyl]-2H,4H,5H,6H,7H-pyrazolo[4,3-c]pyridin-3-yl]-2,3-dihydro-1H-imidazol-2-one (C-17 and C-18)

By replacing the aqueous formaldehyde solution with 4-hydroxycyclohexanone, and following the procedures similar to those of Example 27, two distereoisomeric compounds were obtained, arbitrarily designated as C-17 and C-18.

C-17:

ESI-MS(m/z): [M+H]⁺=963.9

¹H NMR (400 MHz, DMSO-d₆) δ 8.35-8.22 (m, 1H), 7.64-7.31 (m, 4H), 7.23-7.03 (m, 4H), 7.00-6.84 (m, 1H), 6.78-6.63 (m, 1H), 5.65-5.44 (m, 1H), 4.44-4.30 (m, 1H), 4.14-4.06 (m, 5H), 4.00-3.93 (m, 2H), 3.76-3.66 (m, 1H), 3.55-3.43 (m, 4H), 3.07-2.93 (m, 1H), 2.87-2.64 (m, 4H), 2.24 (s, 6H), 2.03-1.94 (m, 1H), 1.73 (s, 5H), 1.61-1.49 (m, 3H), 1.46-1.35 (m, 4H), 1.30-1.19 (s, 6H).

C-18:

ESI-MS(m/z): [M+H]⁺=963.9

¹H NMR (400 MHz, DMSO-d₆) δ 8.35-8.22 (m, 1H), 7.72-7.63 (m, 1H), 7.50-7.27 (m, 3H), 7.24-7.02 (m, 4H), 6.99-6.86 (m, 1H), 6.78-6.61 (m, 1H), 5.69-5.37 (m, 1H), 4.70-4.51 (m, 1H), 4.16-4.04 (m, 5H), 4.00-3.93 (m, 2H), 3.54-3.37 (m, 5H), 3.09-2.98 (m, 1H), 2.90-2.78 (m, 2H), 2.71-2.63 (m, 1H), 2.25 (s, 6H), 1.80-1.66 (m, 6H), 1.43-1.00 (m, 14H).

### Example 35: 1-[(4S)-5-{1-[(R,5S,6S)-3-(4,4-difluorocyclohexyl)-6-(1H-1, 2,3,4-tetrazol-5-yl)-3-azabicyclo[3.1.0]hexan-6-yl]-5-(oxan-4-yl)-1H-indole-2-carbonyl}-2-(4-fluoro-3,5-dimethylphenyl)-4-methyl-2H,4H,5H,6H,7H-pyrazolo[4,3-c]pyridin-3-yl]-3-(4-fluoro-1-methyl-1H-indazol-5-yl)-2,3-dihydro-1H-imidazol-2-one (C-19)

By replacing the aqueous formaldehyde solution with 4,4-difluorocyclohexanone, and following the procedures similar to those of Example 27, the titled compound was obtained.

ESI-MS(m/z): [M+H]⁺=984.8

¹H NMR (400 MHz, DMSO-d₆) δ 8.33 (s, 1H), 7.99 (d, J=8.4 Hz, 1H), 7.66 (d, J=8.8 Hz, 1H), 7.51-7.45 (m, 1H), 7.40 (s, 1H), 7.25 (s, 1H), 7.20 (d, J=6.0 Hz, 2H), 7.09 (s, 1H), 6.98 (s, 1H), 5.74 (d, J=6.4 Hz, 1H), 5.31-5.33 (m, 1H), 4.12 (s, 5H), 3.96 (d, J=10.8 Hz, 2H), 3.52-3.40 (m, 3H), 2.99-2.72 (m, 2H), 2.69-2.53 (m, 2H), 2.45-2.39 (m, 1H), 2.36-2.11 (m, 8H), 2.05-1.86 (m, 4H), 1.78-1.64 (m, 4H), 1.57-1.11 (m, 10H).

### Example 36: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(5-methyl-1,3,4-oxadiazol-2-yl)-3-azabicyclo[3.1.0]hexan-6-yl]-4,5-dihydro-1,2,4-oxadiazol-5-one(C-20)

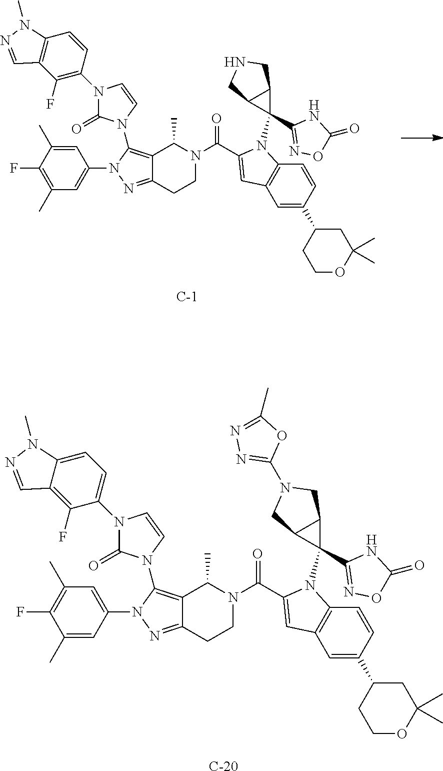

A mixture of C-1 (20.0 mg, 0.022 mmol), 2-bromo-5-methyl-1,3,4-oxadiazole (5.4 mg, 0.033 mmol), and potassium carbonate (6.1 mg, 0.044 mmol) in N,N-dimethylformamide (0.3 mL) was stirred at 90° C. for 12 h. The reaction mixture was cooled to 25° C., and purified by C-18 reverse-phase column chromatography (water/acetonitrile=1/4) to obtain C-7 (10.3 mg, 47%) as white solid.

ESI-MS(m/z): [M+H]⁺=992.7.

¹H NMR (400 MHz, DMSO d₆) δ 8.33 (s, 1H), 7.66 (d, J=8.9 Hz, 1H), 7.53-7.44 (m, 2H), 7.33-7.25 (m, 1H), 7.22-7.12 (m, 3H), 7.10 (d, J=3.1 Hz, 1H), 6.97 (s, 1H), 5.72-5.64 (m, 1H), 4.18-4.06 (m, 4H), 3.99-3.87 (m, 1H), 3.83-3.64 (m, 5H), 3.24-3.10 (m, 1H), 3.08-2.98 (m, 1H), 2.94-2.78 (m, 2H), 2.75-2.59 (m, 1H), 2.30 (s, 3H), 2.26 (s, 6H), 1.73-1.64 (m, 3H), 1.63-1.49 (m, 2H), 1.47-1.42 (m, 3H), 1.30-1.22 (m, 5H), 1.20-1.17 (m, 3H).

### Example 37: 1-[(4S)-5-{J-[(1R,5S,6S)-3-cyclopropyl-6-(1H-1, 2,3,4-tetrazol-5-yl)-3-azabicyclo[3.1.0]hexan-6-yl]-5-(oxan-4-yl)-1H-indole-2-carbonyl}-2-(4-fluoro-3,5-dimethylphenyl)-4-methyl-2H,4H,5H,6H,7H-pyrazolo[4,3-c]pyridin-3-yl]-3-(4-fluoro-1-methyl-1H-indazol-5-yl)-2,3-dihydro-1H-imidazol-2-one (C-21)

By replacing the aqueous formaldehyde solution with 1-ethoxy-1-(trimethylsilyloxy)cyclopropane, and following procedures similar to those of Example 27, the title compound was obtained.

ESI-MS(m/z): [M+H]⁺=905.9

¹H NMR (400 MHz, DMSO-d₆) δ 8.33 (s, 1H), 7.97 (d, J=8.4 Hz, 1H), 7.66 (d, J=8.8 Hz, 1H), 7.48 (s, 1H), 7.41 (s, 1H), 7.29 (s, 1H), 7.20 (d, J=6.0 Hz, 2H), 7.10 (s, 1H), 6.98 (s, 1H), 6.80-6.60 (m, 1H), 5.74 (s, 1H), 4.13 (s, 3H), 3.96 (d, J=10.8 Hz, 2H), 3.52-3.40 (m, 3H), 3.10 (d, J=8.4 Hz, 1H), 2.95-2.70 (m, 4H), 2.37-2.13 (m, 8H), 2.04-1.93 (m, 1H), 1.76-1.66 (m, 5H), 1.52-1.44 (m, 4H), 1.37-1.10 (m, 6H).

### Example 38: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyridin-2-yl)-3-azabicyclo[3.1.0]hexan-6-yl]-4,5-dihydro-1,2,4-oxadiazol-5-one (C-22)

By replacing 2-bromopyrimidine with 2-bromopyridine, and following procedures similar to those of Example 29, the title compound was obtained.

ESI-MS(m/z): [M+H]⁺=986.9

¹H NMR (400 MHz, DMSO-d₆) δ 8.33 (s, 1H), 8.00 (s, 1H), 7.82-7.68 (m, 1H), 7.66 (d, J=8.8 Hz, 1H), 7.54-7.36 (m, 2H), 7.32-7.15 (m, 2H), 7.14-7.06 (m, 2H), 6.98 (s, 1H), 6.70-6.26 (m, 4H), 5.69 (d, J=6.4 Hz, 1H), 4.12 (s, 3H), 3.72 (d, J=7.2 Hz, 2H), 3.64-3.48 (m, 4H), 3.10-2.95 (m, 2H), 2.92-2.62 (m, 2H), 2.35-2.15 (m, 7H), 2.00 (dd, J=14.4, 7.2 Hz, 1H), 1.76-1.39 (m, 6H), 1.28 (s, 3H), 1.23 (s, 3H), 1.19 (s, 3H).

### Example 39: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyrimidin-5-yl)-3-azabicyclo[3.1.0]hexan-6-yl]-4,5-dihydro-1,2,4-oxadiazol-5-one (C-23)

By replacing 2-bromopyrimidine with 5-bromopyrimidine, and following procedures similar to those of Example 29, the title compound was obtained.

ESI-MS(m/z): [M+H]⁺=988.8

¹H NMR (400 MHz, DMSO-d₆) δ 8.42 (s, 1H), 8.33 (s, 1H), 8.18-7.90 (m, 2H), 7.80-7.60 (m, 2H), 7.55-7.35 (m, 2H), 7.33-7.05 (m, 3H), 6.98 (s, 1H), 6.65 (s, 1H), 5.70 (d, J=6.0 Hz, 1H), 5.32 (t, J=4.8 Hz, 1H), 4.29-3.90 (m, 5H), 3.78-3.66 (m, 2H), 3.60-3.45 (m, 2H), 3.08-2.96 (m, 2H), 2.53 (d, J=5.6 Hz, 1H), 2.22-2.16 (m, 1H), 2.26 (s, 6H), 2.04-1.94 (m, 2H), 1.75-1.40 (m, 6H), 1.28 (s, 3H), 1.23 (s, 3H), 1.19 (s, 3H).

### Example 40: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyridazin-3-yl)-3-azabicyclo[3.1.0]hexan-6-yl]-4,5-dihydro-1,2,4-oxadiazol-5-one (C-24)

By replacing 2-bromopyrimidine with 3-chloropyridazine, and following procedures similar to those of Example 29, the title compound was obtained.

ESI-MS(m/z): [M+H]⁺=988.3

¹H NMR (400 MHz, DMSO-d₆) δ 8.52 (s, 1H), 8.33 (s, 1H), 7.67-7.65 (m, 2H), 7.52-7.48 (m, 2H), 7.33 (m, 3H), 7.21-7.19 (m, 2H), 7.10 (m, 1H), 6.99-6.92 (m, 2H), 5.70 (s, 1H), 4.35 (m, 1H), 4.12 (s, 3H), 3.74-3.71 (m, 6H), 3.05-2.89 (m, 4H), 2.33-2.21 (m, 7H), 1.69-1.50 (m, 7H), 1.28-1.19 (m, 8H).

### Example 41: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyrazin-2-yl)-3-azabicyclo[3.1.0]hexan-6-yl]-4,5-dihydro-1,2,4-oxadiazol-5-one (C-25)

By replacing 2-bromopyrimidine with 2-bromopyrazine, and following procedures similar to those of Example 29, the title compound was obtained.

ESI-MS(m/z): [M+H]⁺=988.5

¹H NMR (400 MHz, DMSO-d₆) δ 8.33 (s, 1H), 8.05-7.95 (m, 2H), 7.82 (s, 1H), 7.74-7.60 (m, 2H), 7.55-7.46 (m, 2H), 7.35-7.28 (m, 1H), 7.23-7.16 (m, 2H), 7.12-7.08 (m, 1H), 7.02-6.84 (m, 2H), 5.83-5.62 (m, 1H), 4.38-4.31 (m, 1H), 4.19-4.07 (s, 3H), 3.76-3.60 (m, 6H), 3.21-3.01 (m, 2H), 2.88-2.64 (m, 2H), 2.29-2.21 (m, 7H), 1.69-1.49 (m, 7H), 1.28 (s, 3H), 1.23 (s, 2H), 1.19 (s, 3H).

### Example 42: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(I-methyl-1H-1,2,4-triazol-3-yl)-3-azabicyclo[3.1.0]hexan-6-yl]-4,5-dihydro-1,2,4-oxadiazol-5-one (C-26)

By replacing 2-bromopyrimidine with 3-bromo-1-methyl-1,2,4-thiazole, and following procedures similar to those of Example 29, the title compound was obtained.

ESI-MS(m/z): [M+H]⁺=991.7

¹H NMR (400 MHz, DMSO-d₆) δ 8.32 (m, 1H), 7.99 (m, 1H), 7.67-7.64 (m, 2H), 7.49 (s, 2H), 7.29-7.10 (m, 5H), 6.97-6.88 (m, 1H), 5.69 (m, 1H), 4.12 (s, 3H), 3.73-3.71 (m, 2H), 3.64 (s, 3H), 3.51-3.46 (m, 2H), 3.12-2.98 (m, 2H), 2.93-2.66 (m, 2H), 2.26-2.21 (m, 7H), 2.02-1.98 (m, 1H), 1.69-1.46 (m, 9H), 1.28 (s, 3H), 1.25-1.23 (m, 2H), 1.18 (s, 3H).

### Example 43: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(2-methyl-2H-1,2,3,4-tetrazol-5-yl)-3-azabicyclo[3.1.0]hexan-6-yl]-4,5-dihydro-1,2,4-oxadiazol-5-one (C-27)

By replacing 2-bromopyrimidine with 5-bromo-2-methyl-2H-tetrazole, and following procedures similar to those of Example 29, the title compound was obtained.

ESI-MS(m/z): [M+H]⁺=992.6

¹H NMR (400 MHz, DMSO-d₆) δ 8.33 (s, 1H), 7.65 (d, J=8.8 Hz, 2H), 7.56-7.44 (m, 2H), 7.29 (s, 1H), 7.19 (d, J=6.0 Hz, 2H), 7.09 (s, 1H), 6.97 (s, 1H), 6.90 (s, 1H), 5.69 (d, J=5.6 Hz, 1H), 4.12 (s, 6H), 3.73-3.52 (m, 4H), 3.13-2.93 (m, 2H), 2.90-2.78 (m, 2H), 2.26 (s, 6H), 2.24-2.15 (m, 1H), 2.04-1.94 (m, 1H), 1.74-1.36 (m, 9H), 1.28 (s, 3H), 1.27-1.21 (m, 2H), 1.19 (s, 3H).

### Example 44: 3-[(1R,5S,6S)-6-{2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-5-(oxan-4-yl)-1H-indol-1-yl}-3-(pyrimidin-2-yl)-3-azabicyclo[3.1.0]hexan-6-yl]-4,5-dihydro-1,2,4-oxadiazol-5-one (C-28)

By replacing A-1 with A-2, and following procedures similar to those of Examples 18 and 29, the title compound was obtained.

ESI-MS(m/z): [M+H]⁺=961.3

¹H NMR (400 MHz, DMSO-d₆) δ 8.38 (d, J=20.0 Hz, 3H), 8.27 (s, 2H), 7.81 (d, J=8.8 Hz, 1H), 7.68 (d, J=8.8 Hz, 1H), 7.55 (s, 1H), 7.41 (s, 1H), 7.22 (d, J=6.8 Hz, 2H), 7.06 (d, J=40.4 Hz, 1H), 6.69 (s, 1H), 6.55 (d, J=5.6 Hz, 1H), 5.35 (s, 1H), 4.15 (s, 3H), 4.00 (d, J=10.4 Hz, 2H), 3.80-3.71 (m, 3H), 3.69-3.61 (m, 2H), 2.93-2.80 (m, 2H), 2.28 (s, 3H), 2.25-2.13 (m, 2H), 2.03 (d, J=8.0 Hz, 2H), 1.82-1.68 (m, 2H), 1.54-1.39 (m, 3H), 1.26 (s, 6H), 0.87 (d, J=6.8 Hz, 2H).

### Example 45: 3-[(1R,5S,6S)-6-{5-[(4S)-2,2-dimethyloxan-4-yl]-2-[(4S)-3-{3-[4-fluoro-1-(²H₃)methyl-1H-indazol-5-yl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyrimidin-2-yl)-3-azabicyclo[3.1.0]hexan-6-yl]-4,5-dihydro-1,2,4-oxadiazol-5-one (C-29)

By replacing B-1 with B-5, and following procedures similar to those of Examples 18 and 29, the title compound was obtained.

ESI-MS(m/z): [M+H]⁺=991.5

¹H NMR (400 MHz, DMSO-d₆) δ 8.36-8.21 (m, 3H), 7.88-7.59 (m, 2H), 7.57-7.45 (m, 2H), 7.33-7.26 (m, 1H), 7.23-7.09 (m, 3H), 6.99-6.77 (m, 2H), 6.69-6.62 (m, 1H), 5.75-5.61 (m, 1H), 4.21-4.06 (m, 1H), 3.82-3.51 (m, 6H), 3.16-2.58 (m, 5H), 2.39-2.12 (m, 7H), 1.72-1.65 (m, 2H), 1.63-1.53 (m, 2H), 1.52-1.43 (m, 3H), 1.30-1.23 (m, 4H), 1.19 (s, 3H).

### Example 46: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyridin-3-yl)-3-azabicyclo[3.1.0]hexan-6-yl]-4,5-dihydro-1,2,4-oxadiazol-5-one (C-30)

By replacing 2-bromopyrimidine with 3-bromopyridine, and following procedures similar to those of Example 29, the title compound was obtained.

ESI-MS(m/z): [M+H]⁺=987.4

¹H NMR (400 MHz, DMSO-d₆) δ 8.31 (s, 1H), 7.95-7.89 (m, 2H), 7.68-7.65 (m, 2H), 7.51-7.48 (m, 2H), 7.36-7.27 (m, 1H), 7.23-7.17 (m, 2H), 7.16-7.08 (m, 2H), 7.03-6.84 (m, 2H), 5.72 (s, 1H), 5.33-5.31 (m, 1H), 4.12-4.07 (m, 5H), 3.72 (m, 2H), 3.52-3.41 (m, 1H), 3.05 (s, 2H), 2.84 (s, 2H), 2.29-2.19 (m, 7H), 2.00 (m, 2H), 1.67 (s, 2H), 1.59-1.44 (m, 7H), 1.28 (s, 3H), 1.19 (s, 3H).

### Example 47: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyridazin-4-yl)-3-azabicyclo[3.1.0]hexan-6-yl]-4,5-dihydro-1,2,4-oxadiazol-5-one (C-31)

By replacing 2-bromopyrimidine with 4-bromopyridazine, and following procedures similar to those of Example 29, the title compound was obtained.

ESI-MS(m/z): [M+H]⁺=988.5

¹H NMR (400 MHz, DMSO-d₆) δ 11.57 (s, 1H), 8.31 (m, 1H), 7.91 (m, 1H), 7.66 (m, 2H), 7.49 (m, 2H), 7.30 (m, 1H), 7.20 (m, 2H), 7.12 (m, 3H), 7.05-6.87 (m, 3H), 5.32 (m, 1H), 4.17-4.04 (m, 5H), 3.72 (m, 2H), 3.05 (s, 2H), 2.84 (s, 2H), 2.29-2.19 (m, 7H), 2.00 (m, 2H), 1.67 (s, 2H), 1.59-1.44 (m, 6H), 1.28 (s, 4H), 1.19 (s, 3H).

### Example 48: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyrimidin-4-yl)-3-azabicyclo[3.1.0]hexan-6-yl]-4,5-dihydro-1,2,4-oxadiazol-5-one (C-32)

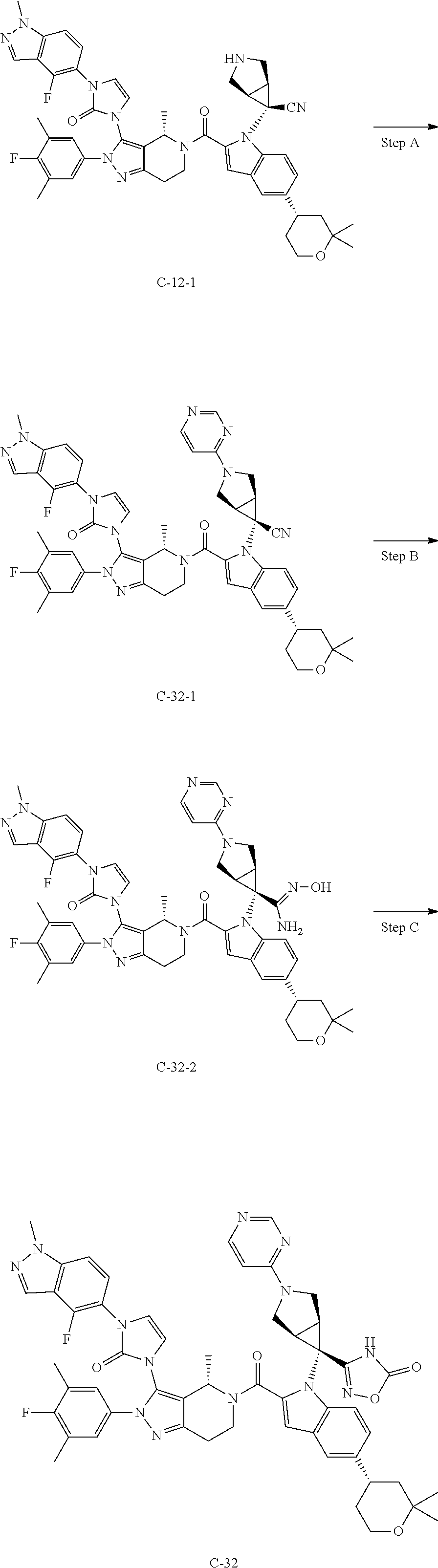

### Step A: (1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyrimidin-4-yl)-3-azabicyclo[3.1.0]hexane-6-carbonitrile (C-32-1)

Under nitrogen atmosphere, a mixture of C-12-1 (50.0 mg, 0.058 mmol), 4-chloropyrimidine hydrochloride (13.3 mg, 0.088 mmol), and diisopropylethylamine (22.8 mg, 0.018 mmol) in N,N-dimethylformamide (5 mL) was stirred at 120° C. for 16 h. The reaction mixture was cooled to 25° C., quenched with water (5 mL), extracted with ethyl acetate (10 mL×2). The combined organic layers were concentrated to driness under reduced pressure. The residue was purified by silica gel flash chromatography (dichloromethane/methanol=95/5) to obtain C-32-1 (30.0 mg, 55%) as white solid.

ESI-MS(m/z): [M+H]⁺=929.4

Steps B and C followed the procedures similar to those of Example 29, and the title compound was obtained.

ESI-MS(m/z): [M+H]⁺=988.4

¹H NMR (400 MHz, DMSO-d₆) δ 8.44 (s, 1H), 8.28 (m, 1H), 8.14 (s, 1H), 7.66 (m, 2H), 7.50 (m, 2H), 7.25 (m, 3H), 7.12 (m, 1H), 7.00 (m, 1H), 6.53 (s, 1H), 5.69 (m, 1H), 5.32 (m, 1H), 4.10 (s, 3H), 3.72 (m, 6H), 3.03 (m, 2H), 2.86 (s, 2H), 2.67 (s, 2H), 2.23 (m, 5H), 1.99 (m, 1H), 1.75-1.41 (m, 7H), 1.29 (s, 3H), 1.26-1.21 (m, 2H), 1.19 (s, 3H).

### Example 49: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(5-methylpyrimidin-2-yl)-3-azabicyclo[3.1.0]hexan-6-yl]-4,5-dihydro-1,2,4-oxadiazol-5-one (C-33)

By replacing 2-bromopyrimidine with 2-bromo-5-methylpyrimidine, and following procedures similar to those of Example 29, the title compound was obtained.

ESI-MS(m/z): [M/2+H]⁺=502.5

¹H NMR (400 MHz, DMSO-d₆) δ 8.31 (s, 2H), 7.88-7.59 (m, 2H), 7.57-7.45 (m, 2H), 7.33-7.26 (m, 1H), 7.23-7.09 (m, 3H), 6.99-6.77 (m, 2H), 6.69-6.62 (m, 1H), 5.75-5.61 (m, 1H), 4.62-4.30 (m, 1H), 4.21-4.06 (m, 4H), 3.82-3.51 (m, 6H), 3.16-2.58 (m, 5H), 2.39-2.12 (m, 7H), 1.72-1.65 (m, 2H), 1.63-1.53 (m, 2H), 1.52-1.43 (m, 3H), 1.30-1.23 (m, 6H), 1.19 (s, 3H).

### Example 50: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(5-fluoropyrimidin-2-yl)-3-azabicyclo[3.1.0]hexan-6-yl]-4,5-dihydro-1,2,4-oxadiazol-5-one (C-34)

By replacing 2-bromopyrimidine with 2-bromo-5-fluoropyrimidine, and following procedures similar to those of Example 29, the title compound was obtained.

ESI-MS(m/z): [M+H]⁺=1006.6.

¹H NMR (400 MHz, DMSO-d₆) δ 8.43 (s, 2H), 8.33 (s, 1H), 8.07 (s, 1H), 7.66 (d, J=8.8 Hz, 1H), 7.51-7.49 (m, 2H), 7.35-7.24 (m, 1H), 7.19 (d, J=5.6 Hz, 2H), 7.15-7.09 (m, 1H), 7.01-6.90 (m, 1H), 5.76-5.63 (m, 1H), 5.36-5.30 (m, 1H), 4.66 (s, 1H), 4.12 (s, 3H), 3.81-3.60 (m, 6H), 3.10-2.96 (m, 2H), 2.95-2.78 (m, 2H), 2.37-2.13 (m, 7H), 1.73-1.38 (m, 7H), 1.28 (s, 3H), 1.26-1.22 (m, 2H), 1.19 (s, 3H).

### Example 51: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-[5-(trifluoromethyl)pyrimidin-2-yl]-3-azabicyclo[3.1.0]hexan-6-yl]-4,5-dihydro-1,2,4-oxadiazol-5-one (C-35)

By replacing 4-chloropyrimidine hydrochloride with 2-chloro-5-trifluoromethylpyrimidine, and following procedures similar to those of Example 48, the title compound was obtained.

ESI-MS(m/z): [M+H]⁺=1056.9

¹H NMR (400 MHz, DMSO-d₆) δ 8.47 (s, 2H), 8.35 (s, 1H), 8.08 (s, 1H), 7.66 (d, J=8.8 Hz, 1H), 7.51-7.49 (m, 2H), 7.35-7.24 (m, 1H), 7.19 (d, J=5.6 Hz, 2H), 7.15-7.09 (m, 1H), 7.01-6.90 (m, 1H), 5.76-5.63 (m, 1H), 5.36-5.30 (m, 1H), 4.66 (s, 1H), 4.12 (s, 3H), 3.81-3.60 (m, 6H), 3.10-2.96 (m, 2H), 2.95-2.78 (m, 2H), 2.37-2.13 (m, 7H), 1.73-1.38 (m, 7H), 1.28 (s, 3H), 1.26-1.22 (m, 2H), 1.19 (s, 3H).

### Example 52: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(6-methylpyridin-2-yl)-3-azabicyclo[3.1.0]hexan-6-yl]-4, 5-dihydro-1,2,4-oxadiazol-5-one (C-36)

By replacing 2-bromopyrimidine with 2-methyl-6-chloropyridine, and following procedures similar to those of Example 29, the title compound was obtained.

ESI-MS(m/z): [M−H]⁻=998.9

¹H NMR (400 MHz, DMSO-d₆) δ 8.32 (s, 1H), 7.86-7.61 (m, 2H), 7.55-7.38 (m, 2H), 7.36-7.15 (m, 3H), 7.14-7.06 (m, 2H), 6.98 (s, 1H), 6.43 (s, 1H), 6.30-6.06 (m, 1H), 5.74-5.65 (m, 1H), 5.32 (t, J=4.8 Hz, 1H), 4.12 (s, 3H), 4.06 (d, J=5.6 Hz, 1H), 3.72 (d, J=7.2 Hz, 2H), 3.62-3.46 (m, 2H), 3.08-2.98 (m, 2H), 2.92-2.60 (m, 2H), 2.34-2.16 (m, 7H), 2.04-1.94 (m, 2H), 1.74-1.38 (m, 7H), 1.28 (s, 3H), 1.26-1.21 (m, 5H), 1.19 (s, 3H).

### Example 53: 3-[(1R,5S,6S)-6-{5-[(4S)-2,2-dimethyloxan-4-yl]-2-[(4S)-3-{3-[4-fluoro-1-(2,2,2-trifluoroethyl)-1H-indazol-5-yl]-2-oxo-2,3-dihydro-1H-imidazol-1-yl}-2-(4-fluoro-3,5-dimethylphenyl)-4-methyl-2H,4H,5H,6H,7H-pyrazolo[4,3-c]pyridine-5-carbonyl]-1H-indol-1-yl}-3-(pyrimidin-2-yl)-3-azabicyclo[3.1.0]hexan-6-yl]-4,5-dihydro-1,2,4-oxadiazol-5-one (C-37)

By replacing B-1 with B-12, and following procedures similar to those of Example 45, the title compound was obtained.

ESI-MS(m/z): [M+H]⁺=1056.5

¹H NMR (400 MHz, DMSO-d₆) δ 8.52 (s, 1H), 8.32 (d, J=4.4 Hz, 2H), 7.83 (d, J=8.8 Hz, 1H), 7.75-7.56 (m, 1H), 7.51 (s, 1H), 7.30 (s, 1H), 7.19 (d, J=6.0 Hz, 1H), 7.13 (d, J=2.4 Hz, 1H), 6.98 (s, 1H), 6.94-6.75 (m, 1H), 6.66 (s, 1H), 5.69 (s, 1H), 5.64-5.50 (m, 2H), 4.53-4.05 (m, 3H), 3.79-3.58 (m, 5H), 3.20-2.98 (m, 2H), 2.94-2.75 (m, 2H), 2.68-2.57 (m, 1H), 2.26 (s, 6H), 2.23-2.16 (m, 1H), 1.74-1.37 (m, 7H), 1.28 (s, 3H), 1.27-1.21 (m, 2H), 1.19 (s, 3H).

### Example 54: 3-[(1R,5S,6S)-3-(5-chloropyrimidin-2-yl)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-azabicyclo[3.1.0]hexan-6-yl]-4,5-dihydro-1,2,4-oxadiazol-5-one (C-38)

By replacing 2-bromopyrimidine with 2-bromo-5-chloropyrimidine, and following procedures similar to those of Example 29, the title compound was obtained.

ESI-MS(m/z): [M+H]⁺=1020.7

¹H NMR (400 MHz, DMSO-d₆) δ 8.41 (s, 2H), 8.32 (s, 1H), 7.66 (d, J=8.8 Hz, 1H), 7.50 (s, 2H), 7.29 (s, 1H), 7.19 (d, J=6.0 Hz, 2H), 7.10 (s, 1H), 6.97 (s, 1H), 6.94-6.85 (m, 1H), 5.69 (s, 1H), 5.33-5.31 (m, 1H), 4.51-4.23 (m, 1H), 4.12 (s, 3H), 3.72 (d, J=8.0 Hz, 4H), 3.05 (s, 2H), 2.85 (s, 2H), 2.67 (s, 1H), 2.35-2.17 (m, 8H), 1.71-1.37 (m, 7H), 1.46 (s, 3H), 1.28 (s, 2H), 1.19 (s, 3H).

### Example 55: 1-(4-fluoro-1-methyl-1H-indazol-5-yl)-3-[(4S)-2-(4-fluoro-3,5-dimethylphenyl)-4-methyl-5-[5-(oxan-4-yl)-1-[(1R,5S,6S)-3-(pyrimidin-2-yl)-6-(1H-1,2,3,4-tetrazol-5-yl)-3-azabicyclo[3.1.0]hexan-6-yl]-1H-indole-2-carbonyl]-2H,4H,5H,6H,7H-pyrazolo[4,3-c]pyridin-3-yl]-2,3-dihydro-1H-imidazol-2-one (C-39)

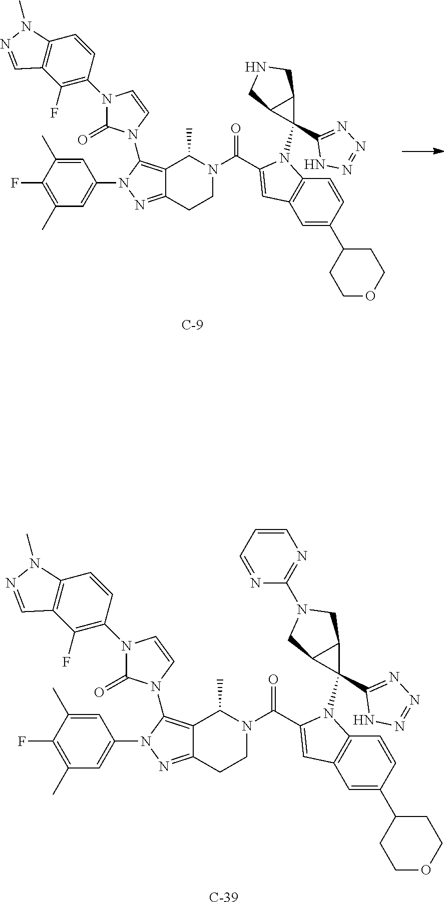

A mixture of C-9 (15.0 mg, 0.02 mmol), 2-bromopyrimidine (2.9 mg, 0.02 mmol), copper(I) iodide (0.3 mg, 0.002 mmol), (2,6-dimethoxyphenyl)amino](oxy)acetic acid (0.58 mg, 0.003 mmol), and potassium phosphate (6.4 mg, 0.03 mmol) in N,N-dimethylformamide (1 mL) was stirred at 90° C. for 4 h under nitrogen atmosphere. The reaction mixture was cooled to 25° C., quenched with water (1 mL), extracted with dichloromethane (5 mL×3). The combined organic layers were washed with saturated brine (3 mL), dried over anhydrous sodium sulfate, and concentrated to driness under reduced pressure. The residue was purified by silica gel column chromatography (dichloromethane/methanol=100/5) to obtain C-39 (10.0 mg, 62%) as white solid.

ESI-MS(m/z): [M+H]⁺=944.8

¹H NMR (400 MHz, DMSO-d₆) δ 8.34 (s, 1H), 8.19 (s, 2H), 7.94 (d, J=7.8 Hz, 1H), 7.66 (d, J=8.8 Hz, 1H), 7.50 (s, 2H), 7.23-7.08 (m, 4H), 6.99 (s, 1H), 6.54 (s, 1H), 5.71-5.46 (m, 2H), 4.17-4.03 (m, 4H), 3.97 (d, J=10.4 Hz, 4H), 3.66 (s, 2H), 3.46 (s, 2H), 3.17 (d, J=5.2 Hz, 1H), 2.85 (s, 2H), 2.28-2.19 (s, 8H), 1.91 (s, 4H), 1.52 (d, J=5.6 Hz, 2H), 1.24 (s, 3H).

### Example 56: 3-[(1R,5S,6S)-6-{2-[(4S)-2-(3-chloro-4-fluoro-5-methylphenyl)-3-[3-(4-fluoro-1-methyl-1H-indazol-5-yl)-2-oxo-2,3-dihydro-1H-imidazol-1-yl]-4-methyl-2H,4H,5H,6H,7H-pyrazolo[4,3-c]pyridine-5-carbonyl]-5-[(4S)-2,2-dimethyloxan-4-yl]-1H-indol-1-yl}-3-(pyrimidin-2-yl)-3-azabicyclo[3.1.0]hexan-6-yl]-4,5-dihydro-1,2,4-oxadiazol-5-one (C-40)

By replacing B-1 with B-2, and following procedures similar to those of Example 45, the title compound was obtained.

ESI-MS(m/z): [M+H]⁺=1008.6

¹H NMR (400 MHz, DMSO-d6) δ 8.35-8.27 (m, 3H), 7.66 (d, J=8.9 Hz, 1H), 7.56-7.49 (m, 2H), 7.46-7.39 (m, 1H), 7.37-7.27 (m, 2H), 7.23-7.19 (m, 1H), 7.17 (d, J=3.1 Hz, 1H), 7.12-7.03 (m, 1H), 6.69-6.58 (m, 1H), 5.76-5.66 (m, 1H), 4.52-4.28 (m, 1H), 4.17-4.02 (m, 4H), 3.76-3.59 (m, 5H), 3.25-3.13 (m, 1H), 3.08-2.98 (m, 1H), 2.94-2.77 (m, 2H), 2.74-2.60 (m, 1H), 2.44-2.35 (m, 3H), 1.73-1.65 (m, 3H), 1.63-1.50 (m, 2H), 1.48-1.42 (m, 3H), 1.30-1.21 (m, 5H), 1.20-1.16 (m, 3H).

### Example 57: 3-[(1R,5S,6S)-6-{2-[(4S)-2-(3-chloro-4-fluoro-5-methylphenyl)-3-{3-[4-fluoro-1-(²H₃)methyl-1H-indazol-5-yl]-2-oxo-2,3-dihydro-1H-imidazol-1-yl}-4-methyl-2H,4,5H,6H,7H-pyrazolo[4,3-c]pyridine-5-carbonyl]-5-[(4S)-2,2-dimethyloxan-4-yl]-1H-indol-1-yl}-3-(pyrimidin-2-yl)-3-azabicyclo[3.1.0]hexan-6-yl]-4,5-dihydro-1,2,4-oxadiazol-5-one (C-41)

By replacing B-1 with B-6, and following procedures similar to those of Example 45, the title compound was obtained.

ESI-MS(m/z): [M+H]⁺=1008.6

¹H NMR (400 MHz, DMSO-d6) δ 8.35-8.27 (m, 3H), 7.66 (d, J=8.9 Hz, 1H), 7.56-7.49 (m, 2H), 7.46-7.39 (m, 1H), 7.37-7.27 (m, 2H), 7.23-7.19 (m, 1H), 7.17 (d, J=3.1 Hz, 1H), 7.12-7.03 (m, 1H), 6.69-6.58 (m, 1H), 5.76-5.66 (m, 1H), 4.52-4.28 (m, 1H), 4.17-4.02 (m, 4H), 3.76-3.59 (m, 5H), 3.25-3.13 (m, 1H), 3.08-2.98 (m, 1H), 2.94-2.77 (m, 2H), 2.74-2.60 (m, 1H), 2.44-2.35 (m, 3H), 1.73-1.65 (m, 3H), 1.63-1.50 (m, 2H), 1.48-1.42 (m, 3H), 1.30-1.21 (m, 5H), 1.20-1.16 (m, 3H).

### Example 58: 3-[(1R,5S,6S)-6-{2-[(4S)-2-(3-chloro-4-fluorophenyl)-3-{3-[4-fluoro-1-(²H₃)methyl-1H-indazol-5-yl]-2-oxo-2,3-dihydro-1H-imidazol-1-yl}-4-methyl-21H,4H,5H,6H,7H-pyrazolo[4,3-c]pyridine-5-carbonyl]-5-[(4S)-2,2-dimethyloxan-4-yl]-1H-indol-1-yl}-3-(pyrimidin-2-yl)-3-azabicyclo[3.1.0]hexan-6-yl]-4,5-dihydro-1,2,4-oxadiazol-5-one (C-42)

By replacing B-1 with B-11, and following procedures similar to those of Example 45, the title compound was obtained.

ESI-MS(m/z): [M+H]⁺=997.5.

¹H NMR (400 MHz, DMSO d₆) δ 8.34-8.22 (m, 3H), 7.68-7.58 (m, 3H), 7.53-7.46 (m, 2H), 7.44-7.38 (m, 1H), 7.33-7.23 (m, 1H), 7.17-7.12 (m, 1H), 7.10-7.04 (m, 1H), 6.69-6.56 (m, 1H), 5.74-5.65 (m, 1H), 4.50-4.25 (m, 1H), 4.22-3.99 (m, 1H), 3.78-3.56 (m, 5H), 3.25-3.12 (m, 1H), 3.10-2.96 (m, 2H), 2.91-2.73 (m, 2H), 2.74-2.60 (m, 1H), 1.74-1.65 (m, 2H), 1.64-1.49 (m, 3H), 1.49-1.41 (m, 3H), 1.33-1.21 (m, 5H), 1.21-1.14 (m, 3H).

### Example 59: 3-[(1R,5S,6S)-6-{5-[(4S)-2,2-dimethyloxan-4-yl]-2-[(4S)-3-{3-[4-fluoro-1-(²H₃)methyl-1H-indazol-5-yl]-2-oxo-2,3-dihydro-1H-imidazol-1-yl}-2-[4-fluoro-3-methyl-5-(trifluoromethyl)phenyl]-4-methyl-2H,4H,5H,6H,7H-pyrazolo[4,3-c]pyridine-5-carbonyl]-1H-indol-1-yl}-3-(pyrimidin-2-yl)-3-azabicyclo[3.1.0]hexan-6-yl]-4,5-dihydro-1,2,4-oxadiazol-5-one (C-43)

By replacing B-1 with B-9, and following procedures similar to those of Example 45, the title compound was obtained.

ESI-MS(m/z): [M+H]⁺=1145.5.

¹H NMR (400 MHz, DMSO d₆) δ 8.35-8.27 (m, 3H), 7.81-7.76 (m, 1H), 7.66 (d, J=8.8 Hz, 1H), 7.59-7.39 (m, 4H), 7.35-7.25 (m, 1H), 7.18 (d, J=3.1 Hz, 1H), 7.10 (s, 1H), 6.69-6.61 (m, 1H), 5.72 (d, J=7.0 Hz, 1H), 4.52-4.28 (m, 1H), 4.23-4.09 (m, 1H), 3.76-3.58 (m, 5H), 3.26-3.11 (m, 1H), 3.09-2.99 (m, 1H), 2.95-2.75 (m, 2H), 2.73-2.58 (m, 1H), 2.42-2.31 (m, 4H), 1.72-1.65 (m, 2H), 1.64-1.50 (m, 2H), 1.50-1.43 (m, 3H), 1.29-1.22 (m, 5H), 1.19 (s, 3H).

### Example 60: 3-[(1R,5S,6S)-6-{2-[(4S)-2-(3,4-difluoro-5-methylphenyl)-3-[3-(4-fluoro-1-methyl-1H-indazol-5-yl)-2-oxo-2,3-dihydro-1H-imidazol-1-yl]-4-methyl-2H,4H,5H,6H,7H-pyrazolo[4,3-c]pyridine-5-carbonyl]-5-[(4S)-2,2-dimethyloxan-4-yl]-1H-indol-1-yl}-3-(pyrimidin-2-yl)-3-azabicyclo[3.1.0]hexan-6-yl]-4,5-dihydro-1,2,4-oxadiazol-5-one (C-44)

By replacing B-1 with B-4, and following procedures similar to those of Example 45, the title compound was obtained.

ESI-MS(m/z): [M+H]⁺=992.4.

¹H NMR (400 MHz, DMSO d₆) δ 8.36-8.26 (m, 3H), 7.66 (d, J=8.9 Hz, 1H), 7.55-7.47 (m, 2H), 7.37-7.18 (m, 4H), 7.15 (d, J=3.1 Hz, 1H), 7.06 (s, 1H), 6.65 (s, 1H), 5.73-5.64 (m, 1H), 4.54-4.28 (m, 1H), 4.21-4.05 (m, 4H), 3.75-3.58 (m, 5H), 3.26-3.12 (m, 1H), 3.10-2.99 (m, 1H), 2.96-2.75 (m, 2H), 2.73-2.58 (m, 1H), 2.36-2.26 (m, 4H), 1.74-1.64 (m, 2H), 1.63-1.51 (m, 2H), 1.51-1.42 (m, 3H), 1.31-1.21 (m, 5H), 1.21-1.16 (m, 3H).

### Example 61: 5-[(4S)-5-{5-[(4S)-2,2-dimethyloxan-4-yl]-1-[(1R,5S,6S)-6-(5-oxo-4,5-dihydro-1,2,4-oxadiazol-3-yl)-3-(pyrimidin-2-yl)-3-azabicyclo[3.1.0]hexan-6-yl]-1H-indole-2-carbonyl}-3-[3-(4-fluoro-1-methyl-1H-indazol-5-yl)-2-oxo-2,3-dihydro-1H-imidazol-1-yl]-4-methyl-2H,4H,5H,6H,7H-pyrazolo[4,3-c]pyridin-2-yl]-2-fluoro-3-methylbenzonitrile (C-45)

By replacing B-1 with B-3, and following procedures similar to those of Example 45, the title compound was obtained.

ESI-MS(m/z): [M+H]⁺=999.6

¹H NMR (400 MHz, DMSO d₆) δ 8.35-8.27 (m, 3H), 7.82-7.71 (m, 2H), 7.68-7.63 (m, 1H), 7.55-7.39 (m, 2H), 7.35-7.25 (m, 1H), 7.22-7.15 (m, 2H), 7.08 (s, 1H), 6.70-6.58 (m, 1H), 5.73 (s, 1H), 4.52-4.27 (m, 1H), 4.19-4.05 (m, 4H), 3.77-3.58 (m, 5H), 3.25-3.12 (m, 1H), 3.10-2.98 (m, 1H), 2.95-2.77 (m, 2H), 2.76-2.62 (m, 1H), 2.40-2.29 (m, 3H), 1.75-1.64 (m, 2H), 1.66-1.52 (m, 3H), 1.52-1.43 (m, 3H), 1.31-1.22 (m, 5H), 1.20-1.16 (m, 3H).

### Example 62: 3-[(1R,5S,6S)-6-{2-[(4S)-2-(3,4-difluoro-5-methylphenyl)-3-{3-[4-fluoro-1-(²H₃)methyl-1H-indazol-5-yl]-2-oxo-2,3-dihydro-1H-imidazol-1-yl}-4-methyl-21H,4H,5H,6H,7H-pyrazolo[4,3-c]pyridine-5-carbonyl]-5-[(4S)-2,2-dimethyloxan-4-yl]-1H-indol-1-yl}-3-(pyrimidin-2-yl)-3-azabicyclo[3.1.0]hexan-6-yl]-4,5-dihydro-1,2,4-oxadiazol-5-one (C-46)

By replacing B-1 with B-8, and following procedures similar to those of Example 45, the title compound was obtained.

ESI-MS(m/z): [M+H]⁺=999.5

¹H NMR (400 MHz, DMSO d₆) δ 8.39-8.26 (m, 3H), 7.66 (d, J=8.8 Hz, 1H), 7.56-7.45 (m, 2H), 7.39-7.18 (m, 3H), 7.15 (d, J=3.1 Hz, 1H), 7.06 (s, 1H), 6.72-6.54 (m, 2H), 5.76-5.66 (m, 1H), 4.52-4.27 (m, 1H), 4.21-4.02 (m, 1H), 3.79-3.58 (m, 5H), 3.26-3.12 (m, 1H), 3.10-2.98 (m, 1H), 2.96-2.74 (m, 2H), 2.72-2.59 (m, 1H), 2.37-2.26 (m, 3H), 1.73-1.64 (m, 2H), 1.64-1.49 (m, 3H), 1.50-1.41 (m, 3H), 1.32-1.21 (m, 5H), 1.21-1.15 (m, 3H).

### Example 63: 5-[(4S)-5-{5-[(4S)-2,2-dimethyloxan-4-yl]-1-[(1R,5S,6S)-6-(5-oxo-4,5-dihydro-1,2,4-oxadiazol-3-yl)-3-(pyrimidin-2-yl)-3-azabicyclo[3.1.0]hexan-6-yl]-1H-indole-2-carbonyl}-3-{3-[4-fluoro-1-(²H₃)methyl-1H-indazol-5-yl]-2-oxo-2,3-dihydro-JH-imidazol-1-yl}-4-methyl-2H,4H,5H,6H,7H-pyrazolo[4,3-c]pyridin-2-yl]-2-fluoro-3-methylbenzonitrile (C-47)

By replacing B-1 with B-7, and following procedures similar to those of Example 45, the title compound was obtained.

ESI-MS(m/z): [M+H]⁺=1002.6

¹H NMR (400 MHz, DMSO d₆) δ 8.38-8.25 (m, 3H), 7.72-7.61 (m, 2H), 7.58-7.38 (m, 2H), 7.36-7.15 (m, 3H), 7.13-7.05 (m, 1H), 6.68-6.52 (m, 2H), 5.75-5.68 (m, 1H), 4.54-4.26 (m, 1H), 4.23-3.98 (m, 1H), 3.79-3.57 (m, 5H), 3.24-3.14 (m, 1H), 3.07-2.99 (m, 1H), 2.92-2.75 (m, 2H), 2.74-2.62 (m, 1H), 2.37-2.27 (m, 3H), 1.74-1.64 (m, 2H), 1.64-1.50 (m, 3H), 1.49-1.43 (m, 3H), 1.32-1.22 (m, 5H), 1.21-1.15 (m, 3H).

### Example 64: 3-[(1R,5S,6S)-6-{5-[(4S)-2,2-dimethyloxan-4-yl]-2-[(4S)-3-{3-[4-fluoro-1-(²H₃)methyl-1H-indazol-5-yl]-2-oxo-2,3-dihydro-1H-imidazol-1-yl}-2-(4-fluoro-3-methylphenyl)-4-methyl-2H,4H,5H,6H,7H-pyrazolo[4,3-c]pyridine-5-carbonyl]-1H-indol-1-yl}-3-(pyrimidin-2-yl)-3-azabicyclo[3.1.0]hexan-6-yl]-4,5-dihydro-1,2,4-oxadiazol-5-one (C-48)

By replacing B-1 with B-10, and following procedures similar to those of Example 45, the title compound was obtained.

ESI-MS(m/z): [M+H]⁺=977.3

¹H NMR (400 MHz, DMSO d₆) δ 8.36-8.23 (m, 3H), 7.65 (d, J=8.8 Hz, 1H), 7.54-7.44 (m, 2H), 7.40 (d, J=6.8 Hz, 1H), 7.36-7.25 (m, 2H), 7.26-7.16 (m, 2H), 7.10 (d, J=3.1 Hz, 1H), 7.00 (s, 1H), 6.70-6.50 (m, 1H), 5.73-5.63 (m, 1H), 4.53-4.05 (m, 2H), 3.75-3.58 (m, 5H), 3.27-3.10 (m, 1H), 3.08-2.99 (m, 1H), 2.93-2.77 (m, 2H), 2.75-2.63 (m, 1H), 2.31-2.21 (m, 3H), 1.74-1.65 (m, 2H), 1.64-1.50 (m, 3H), 1.49-1.43 (m, 3H), 1.31-1.21 (m, 5H), 1.21-1.15 (m, 3H).

### Example 65: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-]-yl}-3-(pyridin-4-yl)-3-azabicyclo[3.1.0]hexan-6-yl]-4,5-dihydro-1,2,4-oxadiazol-5-one (C-49)

By replacing 2-bromopyrimidine with 4-bromopyridine, and following procedures similar to those of Example 29, the title compound was obtained.

ESI-MS(m/z): [M+H]⁺=987.3

¹H NMR (400 MHz, DMSO-d₆) δ 8.33 (s, 1H), 8.23 (s, 2H), 7.72-7.39 (m, 4H), 7.34-7.07 (m, 5H), 6.90 (m, 3H), 5.69 (m, 1H), 4.30 (m, 1H), 4.12 (s, 3H), 4.07 (s, 1H), 3.91-3.61 (m, 4H), 3.11-2.74 (m, 4H), 2.26 (s, 6H), 2.23-2.14 (m, 2H), 1.75-1.40 (m, 7H), 1.28 (s, 3H), 1.23 (s, 2H), 1.19 (s, 3H).

### Example 66: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-(5-fluoropyridin-2-yl)-3-azabicyclo[3.1.0]hexan-6-yl]-4,5-dihydro-1,2,4-oxadiazol-5-one (C-50)

By replacing 2-bromopyrimidine with 2-bromo-5-fluoropyridine, and following procedures similar to those of Example 29, the title compound was obtained.

ESI-MS(m/z): [M/2+H]⁺=503.5

¹H NMR (400 MHz, DMSO-d₆) δ 8.32 (s, 1H), 8.05 (m, 1H), 7.66 (m, 2H), 7.56-7.42 (m, 3H), 7.31 (s, 2H), 7.19 (m, 2H), 7.10 (s, 1H), 6.94 (m, 1H), 6.51 (s, 1H), 5.71 (s, 1H), 4.39-4.20 (m, 2H), 4.17-4.02 (m, 5H), 3.78-3.46 (m, 4H), 3.09-2.98 (m, 2H), 2.97-2.76 (m, 2H), 2.30-2.16 (m, 7H), 2.06-1.89 (m, 1H), 1.74-1.35 (m, 5H), 1.28 (s, 3H), 1.23 (s, 2H), 1.18 (s, 3H).

### Example 67: 3-[(1R,5S,6S)-3-(5-chloropyridin-2-yl)-6-{5-[(4S)-2,2-dimethyloxan-4-yl]-2-[(4S)-3-[3-(4-fluoro-1-methyl-1H-indazol-S-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-azabicyclo[3.1.0]hexan-6-yl]-4,5-dihydro-1,2,4-oxadiazol-5-one (C-51)

By replacing 2-bromopyrimidine with 2-bromo-5-chloropyridine, and following procedures similar to those of Example 29, the title compound was obtained.

ESI-MS(m/z): [M+H]⁺=1021.6

¹H NMR (400 MHz, DMSO-d₆) δ 8.33 (s, 1H), 8.04 (s, 1H), 7.87-7.41 (m, 5H), 7.30 (m, 2H), 7.20 (d, J=6.4 Hz, 2H), 7.11 (d, J=2.4 Hz, 1H), 7.03-6.36 (m, 2H), 5.70 (s, 1H), 4.45-4.22 (m, 2H), 4.17-4.02 (m, 5H), 3.78-3.51 (m, 4H), 3.24-2.97 (m, 2H), 2.95-2.62 (m, 2H), 2.26 (s, 6H), 2.23-2.10 (m, 1H), 2.06-1.89 (m, 1H), 1.76-1.37 (m, 5H), 1.28 (s, 3H), 1.23 (s, 2H), 1.19 (s, 3H).

### Example 68: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-2H,4H,5H,6H,7H-pyrazolo[4,3-c]pyridine-5-carbonyl]-1H-indol-1-yl}-3-[5-(trifluoromethyl)pyridin-2-yl]-3-azabicyclo[3.1.0]hexan-6-yl]-4,5-dihydro-1,2,4-oxadiazol-5-one (C-52)

By replacing 2-bromopyrimidine with 2-bromo-5-(trifluoromethyl)pyridine, and following procedures similar to those of Example 29, the title compound was obtained.

ESI-MS(m/z): [M+H]⁺=1055.6

¹H NMR (400 MHz, DMSO-d₆) δ 8.35 (m, J=14.4 Hz, 2H), 7.81-7.62 (m, 3H), 7.50 (m, J=7.2 Hz, 2H), 7.20 (m, 5H), 6.95 (m, 1H), 6.63 (m, 1H), 5.70 (m, 1H), 4.43-4.24 (m, 2H), 4.09-4.03 (m, 5H), 3.77-3.50 (m, 4H), 3.25-2.98 (m, 2H), 2.96-2.60 (m, 2H), 2.26 (s, 6H), 2.25-2.11 (m, 1H), 2.07-1.85 (m, 1H), 1.75-1.39 (m, 5H), 1.28 (s, 3H), 1.23 (s, 2H), 1.19 (s, 3H).

### Example 69: 3-[(1R,5S,6S)-6-{2-[(4S)-2-(3-chloro-4-fluoro-5-methylphenyl)-3-[3-(4-fluoro-1-methyl-1H-indazol-5-yl)-2-oxo-2,3-dihydro-1H-imidazol-1-yl]-4-methyl-2H,4H,5H,6H,7H-pyrazolo[4,3-c]pyridine-5-carbonyl]-5-[(4S)-2,2-dimethyloxan-4-yl]-1H-indol-1-yl}-3-(pyridin-2-yl)-3-azabicyclo[3.1.0]hexan-6-yl]-4,5-dihydro-1,2,4-oxadiazol-5-one (C-53)

By replacing B-1 with B-2, and following procedures similar to those of Examples 29 and 38, the title compound was obtained.

ESI-MS(m/z): [M+H]⁺=998.5

¹H NMR (400 MHz, DMSO-d₆) δ 8.34 (s, 1H), 8.03 (s, 1H), 7.67 (d, J=8.8 Hz, 2H), 7.49 (d, J=8.8 Hz, 3H), 7.36-7.27 (m, 3H), 7.18 (s, 1H), 7.10 (s, 1H), 6.91 (s, 1H), 6.60 (s, 1H), 6.48 (s, 1H), 5.73 (s, 1H), 4.39-4.24 (m, 1H), 4.12 (s, 3H), 4.10-4.04 (m, 2H), 3.72 (d, J=8.4 Hz, 2H), 3.60-3.48 (m, 2H), 3.12-2.98 (m, 2H), 2.95-2.80 (m, 2H), 2.40-2.25 (m, 4H), 2.03-1.96 (m, 2H), 1.74-1.39 (m, 5H), 1.28 (s, 3H), 1.23 (s, 2H), 1.19 (s, 3H).

### Example 70: 3-[(1R,5S,6S)-6-{2-[(4S)-2-(3,4-difluoro-5-methylphenyl)-3-[3-(4-fluoro-1-methyl-1H-indazol-5-yl)-2-oxo-2,3-dihydro-1H-imidazol-1-yl]-4-methyl-2H,4H,5H,6H,7H-pyrazolo[4,3-c]pyridine-5-carbonyl]-5-[(4S)-2,2-dimethyloxan-4-yl]-1H-indol-1-yl}-3-(pyridin-2-yl)-3-azabicyclo[3.1.0]hexan-6-yl]-4,5-dihydro-1,2,4-oxadiazol-5-one (C-54)

By replacing B-1 with B-4, and following procedures similar to those of Examples 29 and 38, the title compound was obtained.

ESI-MS(m/z): [M+H]⁺=992.3

¹H NMR (400 MHz, DMSO-d₆) δ 8.33 (s, 1H), 8.03 (s, 1H), 7.66 (d, J=8.8 Hz, 1H), 7.49 (d, J=8.0 Hz, 3H), 7.32 (s, 2H), 7.24 (s, 1H), 7.16 (s, 1H), 7.07 (s, 1H), 6.91 (s, 1H), 6.64 (d, 1H), 6.48 (s, 1H), 5.72 (s, 1H), 5.32 (t, J=4.8 Hz, 1H), 4.27 (s, 1H), 4.16-4.04 (m, 5H), 3.72 (d, J=7.6 Hz, 2H), 3.55 (s, 2H), 3.12-2.97 (m, 2H), 2.95-2.77 (m, 2H), 2.38-2.26 (m, 4H), 2.00 (d, J=7.6 Hz, 2H), 1.74-1.41 (m, 5H), 1.28 (s, 3H), 1.23 (s, 2H), 1.19 (s, 3H).

### Example 71: 5-[(4S)-5-{5-[(4S)-2,2-dimethyloxan-4-yl]-1-[(1R,5S,6S)-6-(5-oxo-4,5-dihydro-1,2,4-oxadiazol-3-yl)-3-(pyridin-2-yl)-3-azabicyclo[3.1.0]hexan-6-yl]-1H-indole-2-carbonyl}-3-[3-(4-fluoro-1-methyl-1H-indazol-5-yl)-2-oxo-2,3-dihydro-1H-imidazol-1-yl]-4-methyl-2H,4H,5H,6H,7H-pyrazolo[4,3-c]pyridin-2-yl]-2-fluoro-3-methylbenzonitrile (C-55)

By replacing B-1 with B-3, and following procedures similar to those of Examples 29 and 38, the title compound was obtained.

ESI-MS(m/z): [M+H]⁺=998.5

¹H NMR (400 MHz, DMSO-d₆) δ 8.32 (s, 1H), 8.03 (s, 1H), 7.76 (d, J=15.6 Hz, 2H), 7.67 (d, J=8.8 Hz, 1H), 7.49 (s, 3H), 7.31 (s, 1H), 7.18 (s, 1H), 7.09 (s, 1H), 6.92 (s, 1H), 6.64 (d, J=26.0 Hz, 1H), 6.48 (s, 1H), 5.74 (s, 1H), 5.33 (d, J=4.8 Hz, 1H), 4.40-4.21 (m, 1H), 4.18-4.03 (m, 5H), 3.78-3.65 (m, 2H), 3.62-3.49 (m, 2H), 3.13-2.96 (m, 2H), 2.94-2.75 (m, 1H), 2.41-2.26 (m, 4H), 1.99 (d, J=7.6 Hz, 2H), 1.74-1.41 (m, 5H), 1.28 (s, 3H), 1.23 (s, 2H), 1.19 (s, 3H).

### Example 72: 3-[(1R,5S,6S)-6-{2-[(4S)-2-(3-chloro-4-fluoro-5-methylphenyl)-3-{3-[4-fluoro-1-(²H₃)methyl-1H-indazol-5-yl]-2-oxo-2,3-dihydro-1H-imidazol-1-yl}-4-methyl-2H,4H,5H,6H,7H-pyrazolo[4,3-c]pyridine-5-carbonyl]-5-[(4S)-2,2-dimethyloxan-4-yl]-1H-indol-1-yl}-3-(pyridin-2-yl)-3-azabicyclo[3.1.0]hexan-6-yl]-4,5-dihydro-1,2,4-oxadiazol-5-one (C-56)

By replacing B-1 with B-6, and following procedures similar to those of Examples 29 and 38, the title compound was obtained.

ESI-MS(m/z): [M/2+H]⁺=507.7

¹H NMR (400 MHz, DMSO-d₆) δ 8.33 (s, 1H), 8.07 (s, 1H), 8.02 (s, 1H), 7.67 (d, J=8.8 Hz, 2H), 7.54-7.36 (m, 6H), 7.33 (s, 1H), 7.16 (s, 1H), 7.07 (s, 1H), 6.69-6.44 (m, 1H), 5.32 (t, J=4.8 Hz, 1H), 4.40-4.21 (m, 1H), 4.03-3.99 (m, 2H), 3.78-3.65 (m, 2H), 3.62-3.49 (m, 2H), 3.13-2.96 (m, 2H), 2.94-2.75 (m, 1H), 2.41-2.26 (m, 4H), 1.99 (d, J=7.6 Hz, 2H), 1.74-1.41 (m, 5H), 1.28 (s, 3H), 1.23 (s, 2H), 1.19 (s, 3H).

### Example 73: 3-[(1R,5S,6S)-6-{5-[(4S)-2,2-dimethyloxan-4-yl]-2-[(4S)-3-{3-[4-fluoro-1-(²H₃)methyl-1H-indazol-5-yl]-2-oxo-2,3-dihydro-1H-imidazol-1-yl}-2-[4-fluoro-3-methyl-5-(trifluoromethyl)phenyl]-4-methyl-2H,4H,5H,6H,7H-pyrazolo[4,3-c]pyridine-5-carbonyl]-1H-indol-1-yl}-3-(pyridin-2-yl)-3-azabicyclo[3.1.0]hexan-6-yl]-4,5-dihydro-1,2,4-oxadiazol-5-one (C-57)

By replacing B-1 with B-9, and following procedures similar to those of Examples 29 and 38, the title compound was obtained.

ESI-MS(m/z): [M+H]⁺=1044.6

¹H NMR (400 MHz, DMSO-d6) δ 8.32 (s, 1H), 8.03 (s, 1H), 7.84-7.62 (m, 3H), 7.60-7.37 (m, 4H), 7.36-7.15 (m, 3H), 7.14-6.85 (m, 1H), 6.69-6.44 (m, 2H), 5.74 (s, 1H), 4.50-3.84 (m, 3H), 3.72 (d, J=8.0 Hz, 2H), 3.63-3.47 (m, 2H), 3.11-2.97 (m, 2H), 2.96-2.76 (m, 2H), 2.45-2.29 (m, 4H), 2.07-1.94 (m, 2H), 1.76-1.40 (m, 5H), 1.28 (s, 3H), 1.23 (s, 2H), 1.19 (s, 3H).

### Example 74: 3-[(1R,5S,6S)-6-{2-[(4S)-2-(3-chloro-4-fluorophenyl)-3-{3-[4-fluoro-1-(²H₃)methyl-1H-indazol-5-yl]-2-oxo-2,3-dihydro-1H-imidazol-1-yl}-4-methyl-2H,4H,5H,6H,7H-pyrazolo[4,3-c]pyridine-5-carbonyl]-5-[(4S)-2,2-dimethyloxan-4-yl]-1H-indol-1-yl}-3-(pyridin-2-yl)-3-azabicyclo[3.1.0]hexan-6-yl]-4,5-dihydro-1,2,4-oxadiazol-5-one (C-58)

By replacing B-1 with B-11, and following procedures similar to those of Examples 29 and 38, the title compound was obtained.

ESI-MS(m/z): [M+H]⁺=996.4

¹H NMR (400 MHz, DMSO-d6) δ 8.32 (s, 1H), 7.98 (s, 2H), 7.84-7.35 (m, 7H), 7.34-7.04 (m, 2H), 6.76-6.44 (m, 2H), 6.31 (s, 1H), 5.71 (s, 1H), 5.35-5.29 (m, 1H), 4.27-3.81 (m, 3H), 3.78-3.66 (m, 2H), 3.64-3.47 (m, 2H), 3.11-2.92 (m, 2H), 2.89-2.56 (m, 2H), 2.06-1.92 (m, 3H), 1.75-1.38 (m, 5H), 1.28 (s, 3H), 1.23 (s, 2H), 1.19 (s, 3H).

### Example 75: 3-[(1R,5S,6S)-6-{5-[(4S)-2,2-dimethyloxan-4-yl]-2-[(4S)-3-{3-[4-fluoro-1-(²H₃)methyl-1H-indazol-5-yl]-2-oxo-2,3-dihydro-1H-imidazol-1-yl}-2-(4-fluoro-3-methylphenyl)-4-methyl-2H,4H,5H,6H,7H-pyrazolo[4,3-c]pyridine-5-carbonyl]-1H-indol-1-yl}-3-(pyridin-2-yl)-3-azabicyclo[3.1.0]hexan-6-yl]-4,5-dihydro-1,2,4-oxadiazol-5-one (C-59)

By replacing B-1 with B-10, and following procedures similar to those of Examples 29 and 38, the title compound was obtained.

ESI-MS(m/z): [M+H]⁺=976.5

¹H NMR (400 MHz, DMSO-d₆) δ 8.32 (s, 1H), 8.16 (s, 1H), 7.99 (d, J=3.2 Hz, 2H), 7.82-7.70 (m, 1H), 7.65 (d, J=8.8 Hz, 1H), 7.56-7.16 (m, 4H), 7.12-6.96 (m, 2H), 6.76-6.44 (m, 2H), 6.42-6.26 (m, 1H), 5.74-5.65 (m, 1H), 5.32 (t, J=4.8 Hz, 1H), 4.31-3.82 (m, 3H), 3.72 (d, J=8.0 Hz, 2H), 3.64-3.48 (m, 2H), 3.08-2.96 (m, 2H), 2.76-2.64 (m, 2H), 2.30-2.19 (m, 4H), 2.04-1.94 (m, 2H), 1.74-1.38 (m, 5H), 1.28 (s, 3H), 1.23 (s, 2H), 1.19 (s, 3H).

### Example 76: 3-[(1R,5S,6S)-6-{5-[(4S)-2,2-dimethyloxan-4-yl]-2-[(4S)-3-[3-(4-fluoro-1-methyl-1H-indazol-5-yl)-2-oxo-2,3-dihydro-1H-imidazol-1-yl]-2-(4-fluoro-3,5-dimethylphenyl)-4-methyl-4H,5H,6H,7H-pyrazolo[1,5-a]pyrazine-5-carbonyl]-1H-indol-1-yl}-3-(pyrimidin-2-yl)-3-azabicyclo[3.1.0]hexan-6-yl]-4,5-dihydro-1,2,4-oxadiazol-5-one (C-60)

By replacing B-1 with B-13, and following procedures similar to those of Examples 18 and 29, the title compound was obtained.

ESI-MS(m/z): [M+H]⁺=973.7

1H NMR (400 MHz, DMSO-d₆) δ 8.29 (s, 4H), 7.72 (d, J=7.6 Hz, 1H), 7.64 (s, 1H), 7.48 (s, 1H), 7.28 (d, J=22.4 Hz, 4H), 7.05 (s, 1H), 6.93 (s, 1H), 6.80 (s, 1H), 6.63 (s, 1H), 4.94 (s, 2H), 4.35 (s, 2H), 4.11 (s, 4H), 3.74-3.67 (m, 5H), 3.07-2.95 (m, 2H), 2.23 (s, 6H), 2.06-1.94 (m, 1H), 1.74-1.40 (m, 4H), 1.28 (s, 3H), 1.23 (s, 2H), 1.19 (s, 3H).

### Example 77: cAMP Accumulation Assays

CHO-K1 cell line stably expressing human GLP-1R was used to detect the cAMP levels using Hithunter cAMP Assay for small molecules platform according manufacturer's instructions (DiscoverX, Cat #90-0075SM2). Cells were plated in 96-well white plates (at destiny of 2.5×10⁴ per well) (Thermo, Cat #136101) and incubated in 1640 (Gibco, Cat #22400-071) supplemented with 10% FBS (Fetal Bovine Serum, Hyclone Cat #SH30406.05), 800 g/ml G418 (Gibco, Cat #10131-027) at 37° C., 5% CO₂. Briefly, cells were treated with compounds or GLP-1 peptide (10 point, 3-fold dilution) in PBS (Phosphate Buffer, Gibco, Cat #10010-023) contained with 1% BSA (Bovine Serum Albumin, Sigma Cat #V900933-100G). After a 30-minute incubation at 37° C., the cAMP antibody reagent, working solution (mixing 19-parts of lysis buffer, 5-parts of substrate reagent 1, 1-part of substrate reagent 2, and 25-parts of solution D (enzyme donor-labeled cAMP)), and solution A (enzyme receptor) were added and incubated at room temperature in the dark. The resulting competitive assays were measured using Spectramax i3x Multi-Mode Detection system (Molecular Devices, USA). A relative EC50 was derived by nonlinear regression analysis by fitting to a 4-parameter logistic model in GraphPad Prism 9 software.

Data for compounds and peptide are shown in Table 2 below.

TABLE 2
Functional cAMP potency (EC₅₀) for compounds
and comparators in the presence of BSA

| Compound | cAMP EC₅₀, nM |
|---|---|
| GLP-1 (7-36) | 0.004495 |
| Orforglipron | 0.4 |
| compound 23 | 6.332 |
| compound 29 | 1.264 |
| C-1 | 0.4 |
| C-2 | 0.6 |
| C-3 | 0.9 |
| C-4 | 0.7 |
| C-5 | 1.1 |
| C-6 | 0.8 |
| C-7 | 2.0 |
| C-8 | 1.0 |
| C-9 | 0.3 |
| C-10 | 0.4 |
| C-11 | 1.0 |
| C-12 | 0.5 |
| C-13 | 0.2 |
| C-14 | 1.2 |
| C-15 | 0.4 |
| C-16 | 0.2 |
| C-17 | 0.2 |
| C-18 | 0.6 |
| C-19 | 2.4 |
| C-20 | 1.3 |
| C-21 | 0.9 |
| C-22 | 0.7 |
| C-23 | 1.5 |
| C-24 | 1.0 |
| C-25 | 0.7 |
| C-26 | 0.7 |
| C-27 | 0.6 |
| C-28 | 5.4 |
| C-29 | 3.3 |
| C-30 | 1.5 |
| C-31 | 2.8 |
| C-32 | 1.4 |
| C-33 | 1.5 |
| C-34 | 2.9 |
| C-35 | 3.0 |
| C-36 | 1.3 |
| C-37 | 1.2 |
| C-38 | 3.5 |
| C-39 | 1.1 |
| C-40 | 3.7 |
| C-41 | 1.2 |
| C-42 | >1000 |
| C-43 | 2.3 |
| C-44 | 3.9 |
| C-45 | 1.0 |
| C-46 | 2.3 |
| C-47 | 1.0 |
| C-48 | 4.0 |
| C-49 | 1.2 |
| C-50 | 2.4 |
| C-51 | 6.1 |
| C-52 | 4.0 |
| C-53 | 4.7 |
| C-54 | 3.5 |
| C-55 | 1.8 |
| C-56 | 2.4 |
| C-57 | 8.4 |
| C-58 | >1000 |
| C-59 | 18.8 |
| C-60 | 11.3 |

### Example 78: Pharmacokinetic Testing in Crab-Eating Macaques (Cynomolgus Monkey)

Crab-eating macaques were administered the compound of the present invention via intravenous injection and oral gavage, respectively. Plasma samples were collected at various time points. The drug concentration of the test compound in the plasma tissue of the crab-eating macaques was detected using an LC-MS/MS analytical method, and a pharmacokinetic analysis was conducted.

### 1. Preparation of the Dosing Formulation

Solvent for intravenous injection dosing formulation: 500 dimethyl sulfoxide (DMSO)+5% polyethylene glycol (PEG)-15 hydroxystearate+90% o physiological saline.

Preparation steps for the intravenous injection dosing solution: Accurately weigh the test compound and add it to the aforementioned solvent for intravenous injection dosing to prepare a solution with a final concentration of 0.25 mg/mL.

Solvent for oral dosing formulation: 5% dimethyl sulfoxide (DMSO)+5% polyethylene glycol (PEG)-15 hydroxystearate+90% physiological saline.

Preparation steps for the oral dosing solution: Accurately weigh the test compound and add it to the aforementioned solvent for oral dosing to prepare a solution or suspension with a final concentration of 0.4 mg/mL.

### 2. Administration

Intravenous injection(IV): Administered via tail vein injection. After inserting the needle, slowly draw back the syringe to check for blood return, indicating that the needle is in the vein, then inject the drug solution.

Oral (PO): Select an appropriately sized gavage needle and syringe, insert the gavage needle into the esophagus from the base of the tongue, until it is close to the cardia of the stomach, then inject the drug formulation and remove the gavage needle. When using disposable plastic gavage needles, use one per group.

### 3. Blood Sample Collection and Processing

Blood is collected via a cephalic vein or other suitable vein, with approximately 1 mL per sample, anticoagulated with K2-EDTA, and placed on ice after collection. After collection, blood samples are kept on ice and centrifuged to separate the plasma within 1 hour (centrifugation conditions: 2200 g, 10 minutes, 2-8° C.). Plasma samples are stored at −80° C. before analysis and the remaining plasma samples after analysis are continued to be stored at −80° C.
4. Sample Detection Plasma concentration detection: The developed LC-MS/MS method is used to detect the compound in the plasma in this experiment.
5. Data Analysis A non-compartmental model is used to analyze the blood concentration-time data using Phoenix® WinNonlin® (Version 7.0, Pharsight, Mountain View, CA).

TABLE 4

| | Administration | Dose | T_{1/2} | C₀ | Cₘₐₓ | AUC_{(0-24 h)} | Cl | F |
|---|---|---|---|---|---|---|---|---|
| | Route | (mg/kg) | (h) | (ng/mL) | (ng/mL) | (h*ng/mL) | (mL/min/kg) | % |
| Orforglipron | IV | 0.5 | 3.4 | 1169.1 | \ | 1785.0 | 4.6 | \ |
| | PO | 2 | 6.2 | \ | 37.5 | 452.9 | \ | 6.3 |
| C-12 | IV | 0.5 | 6.3 | 6728.7 | \ | 6289.0 | 1.3 | \ |
| | PO | 2 | 5.9 | \ | 513.8 | 6164.8 | \ | 24.5 |
| C-22 | IV | 0.5 | 10.3 | 3807.4 | \ | 7555.9 | 0.9 | \ |
| | PO | 2 | 13.6 | \ | 236.5 | 3594.6 | \ | 11.9 |

Based on the test data, compounds C-12 and C-22 demonstrate a longer half-life, higher exposure, lower clearance rate, and greater bioavailability compared to Orforglipron in crab-eating macaques. This indicates that the compounds of the present patent exhibit better absorption profiles in crab-eating macaques. Additional benefits of these compound may include improved solubility, weight loss, and/or blood sugar reduction.

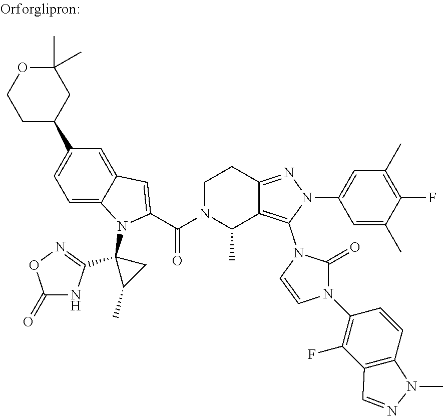 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references (including those listed above), scientific articles, patent publications, and any other documents cited herein are hereby incorporated by reference for the substance of their disclosure.

1-91. (canceled)

92. A compound of Formula (IVa) 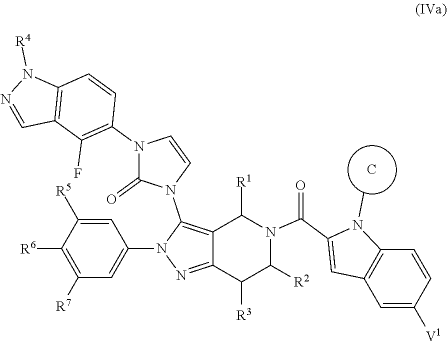
or a pharmaceutically acceptable salt thereof, stereoisomer thereof, tautomer thereof, deuterated analog thereof, or solvate thereof, wherein:
each of R¹, R², and R³ is independently hydrogen or C₁₋₆ alkyl;
R⁴ is C₁₋₆ alkyl;
each of R⁵, R⁶, and R⁷ is independently hydrogen, halo, or C₁₋₆ alkyl;
C is 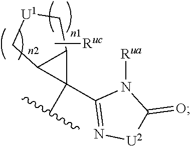
U¹ is —N(R^{ua})—;
U² is O or S;
each R^{ua} is a 5-10 membered heterocyclyl optionally substituted with halo, oxo, hydroxyl, amino, C₁₋₆ alkyl, C₁₋₆ haloalkyl, or C₃₋₈ cycloalkyl;
each R^{uc} is hydrogen;
each of n1 and n2 is 1; and
V¹ is

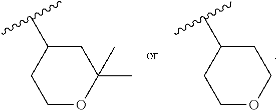

93. The compound of claim 92, wherein the compound of Formula (IVa) is a compound of Formula (IVb) 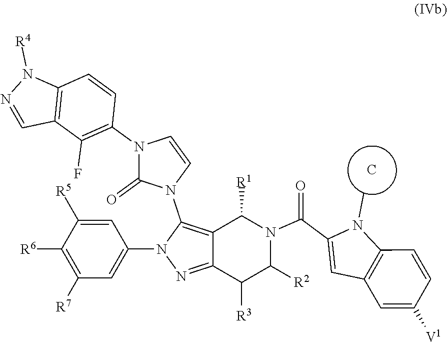
or a pharmaceutically acceptable salt thereof, stereoisomer thereof, tautomer thereof, deuterated analog thereof, or solvate thereof.

94. The compound of claim 92, or a pharmaceutically acceptable salt thereof, stereoisomer thereof, tautomer thereof, deuterated analog thereof, or solvate thereof, wherein C is

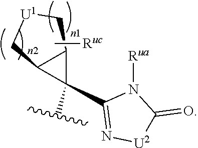

95. The compound of claim 92, or a pharmaceutically acceptable salt thereof, stereoisomer thereof, tautomer thereof, deuterated analog thereof, or solvate thereof, wherein R⁴ is —CH₃.

96. The compound of claim 92, or a pharmaceutically acceptable salt thereof, stereoisomer thereof, tautomer thereof, deuterated analog thereof, or solvate thereof, wherein R⁵ and R⁷ are each —CH₃.

97. The compound of claim 92, or a pharmaceutically acceptable salt thereof, stereoisomer thereof, tautomer thereof, deuterated analog thereof, or solvate thereof, wherein R⁶ is halo.

98. The compound of claim 97, or a pharmaceutically acceptable salt thereof, stereoisomer thereof, tautomer thereof, deuterated analog thereof, or solvate thereof, wherein R⁶ is F.

99. The compound of claim 92, or a pharmaceutically acceptable salt thereof, stereoisomer thereof, tautomer thereof, deuterated analog thereof, or solvate thereof, wherein R^{ua} is 6 membered heterocyclyl.

100. The compound of claim 92, or a pharmaceutically acceptable salt thereof, stereoisomer thereof, tautomer thereof, deuterated analog thereof, or solvate thereof, wherein U² is O.

101. The compound of claim 92, or a pharmaceutically acceptable salt thereof, stereoisomer thereof, tautomer thereof, deuterated analog thereof, or solvate thereof, wherein C is

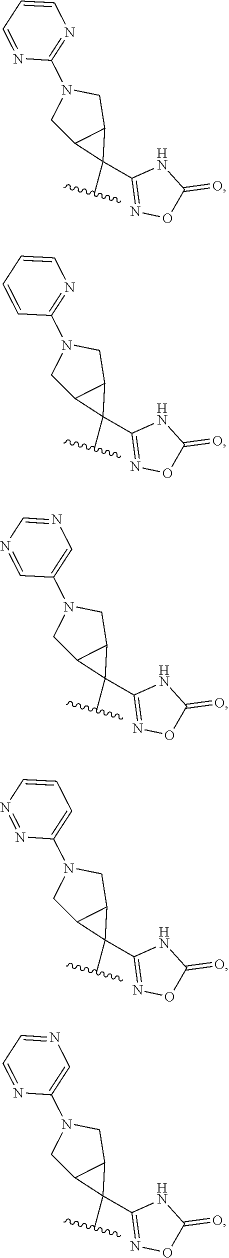 [SMILES: *C1(c2noc(=O)[nH]2)C2CN(c3ncccn3)CC21 ; *C1(c2noc(=O)[nH]2)C2CN(c3ccccn3)CC21 ; *C1(c2noc(=O)[nH]2)C2CN(c3cncnc3)CC21 ; *C1(c2noc(=O)[nH]2)C2CN(c3cccnn3)CC21 ; *C1(c2noc(=O)[nH]2)C2CN(c3cnccn3)CC21]

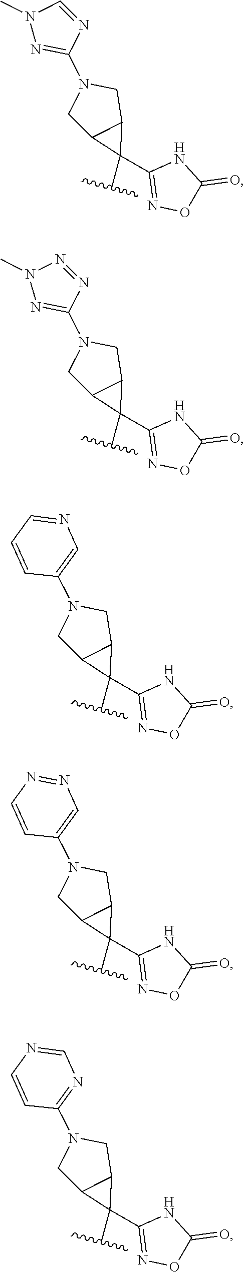 [SMILES: *C1(c2noc(=O)[nH]2)C2CN(c3ncn(C)n3)CC21 ; *C1(c2noc(=O)[nH]2)C2CN(c3nnn(C)n3)CC21 ; *C1(c2noc(=O)[nH]2)C2CN(c3cccnc3)CC21 ; *C1(c2noc(=O)[nH]2)C2CN(c3ccnnc3)CC21 ; *C1(c2noc(=O)[nH]2)C2CN(c3ccncn3)CC21]

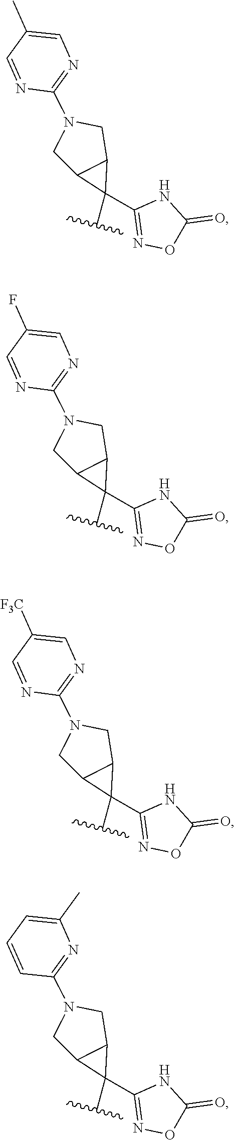 [SMILES: *C1(c2noc(=O)[nH]2)C2CN(c3ncc(C)cn3)CC21 ; *C1(c2noc(=O)[nH]2)C2CN(c3ncc(F)cn3)CC21 ; *C1(c2noc(=O)[nH]2)C2CN(c3ncc(C(F)(F)F)cn3)CC21 ; *C1(c2noc(=O)[nH]2)C2CN(c3cccc(C)n3)CC21]

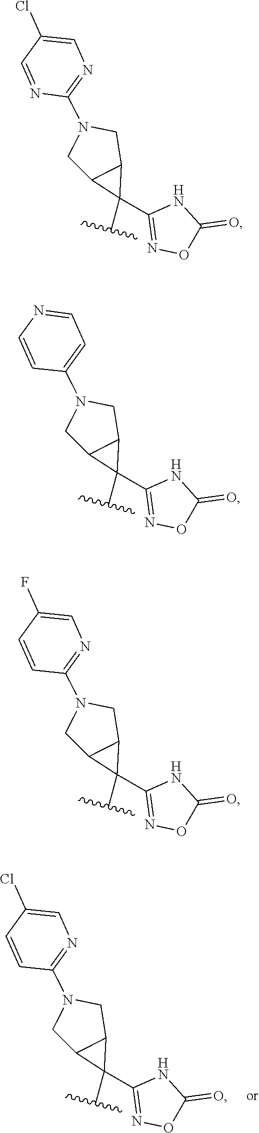 [SMILES: *C1(c2noc(=O)[nH]2)C2CN(c3ncc(Cl)cn3)CC21 ; *C1(c2noc(=O)[nH]2)C2CN(c3ccncc3)CC21 ; *C1(c2noc(=O)[nH]2)C2CN(c3ccc(F)cn3)CC21 ; *C1(c2noc(=O)[nH]2)C2CN(c3ccc(Cl)cn3)CC21]

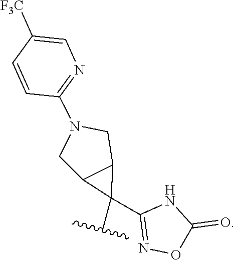 [SMILES: *C1(c2noc(=O)[nH]2)C2CN(c3ccc(C(F)(F)F)cn3)CC21]

102. The compound of claim 92, wherein the compound of Formula (IVa) is a compound of Formula (IIIa): 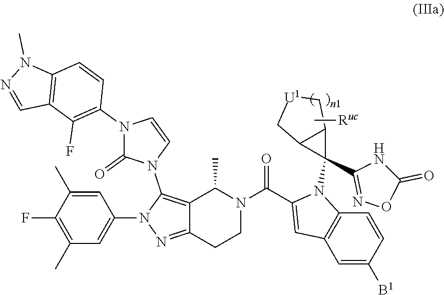
or a pharmaceutically acceptable salt thereof, stereoisomer thereof, tautomer thereof, deuterated analog thereof, or solvate thereof, wherein:
U¹ is —N(R^{ua});
each R^{uc} is hydrogen;
R^{ua} is oxadiazolyl, pyrazinyl, pyridazinyl, pyridinyl, pyrimidinyl, tetrazolyl, or triazolyl, and R^{ua} is independently optionally substituted with halo, oxo, hydroxyl, amino, C₁₋₆ alkyl, C₁₋₆ haloalkyl, or C₃₋₈ cycloalkyl;
n1 is 1; and
V¹ is

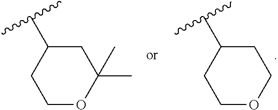

103. The compound of claim 92, or a pharmaceutically acceptable salt thereof, stereoisomer thereof, tautomer thereof, deuterated analog thereof, or solvate thereof, which is selected from:

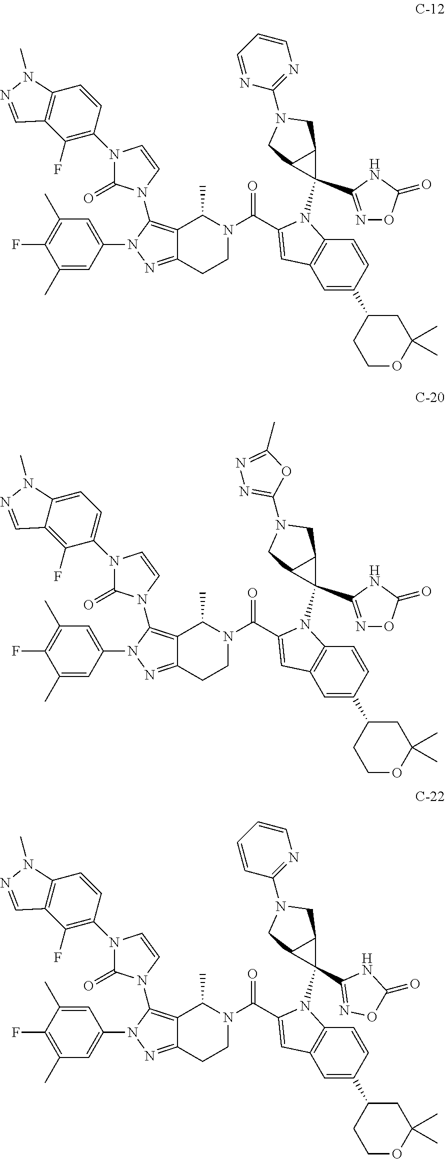

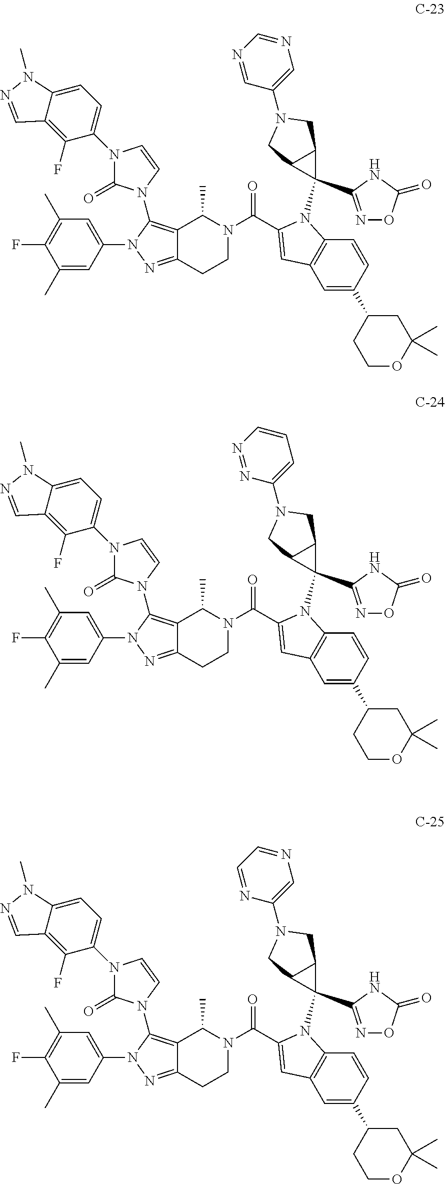

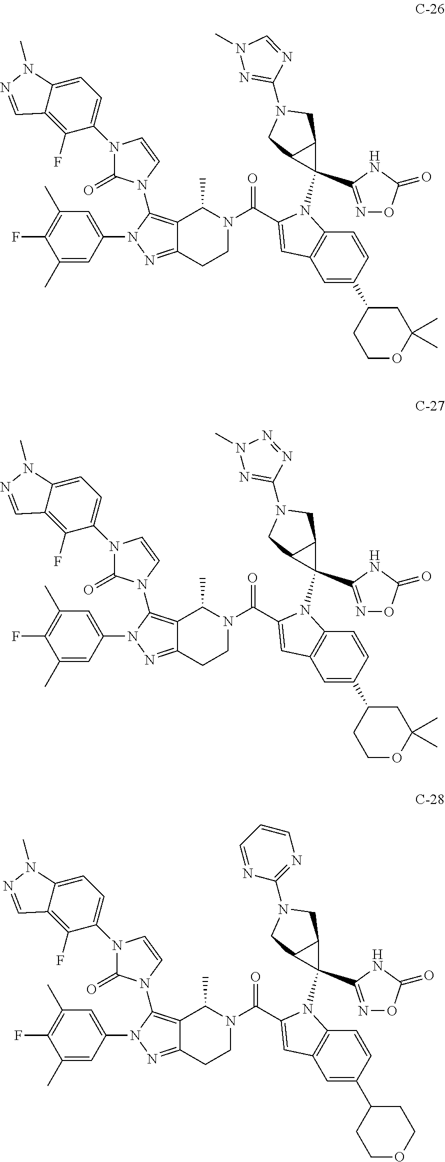

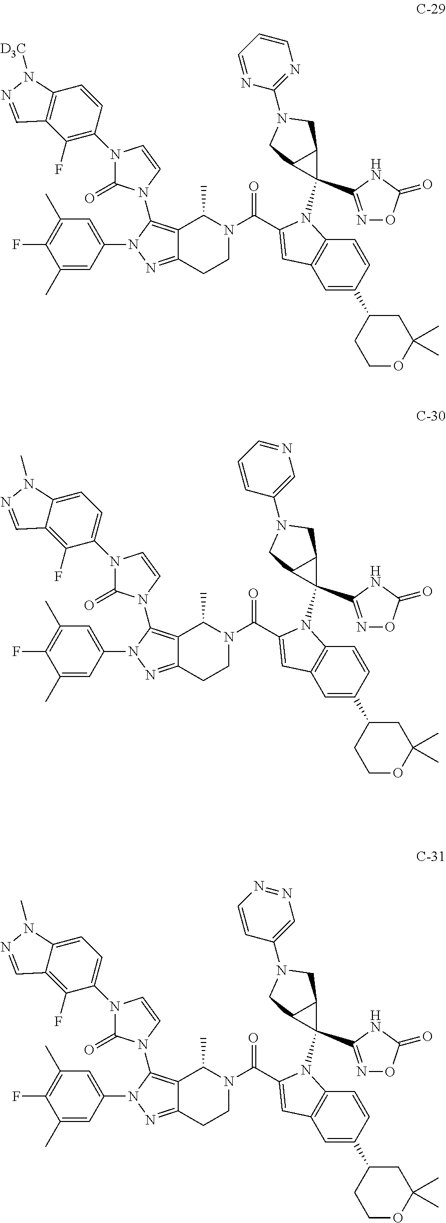

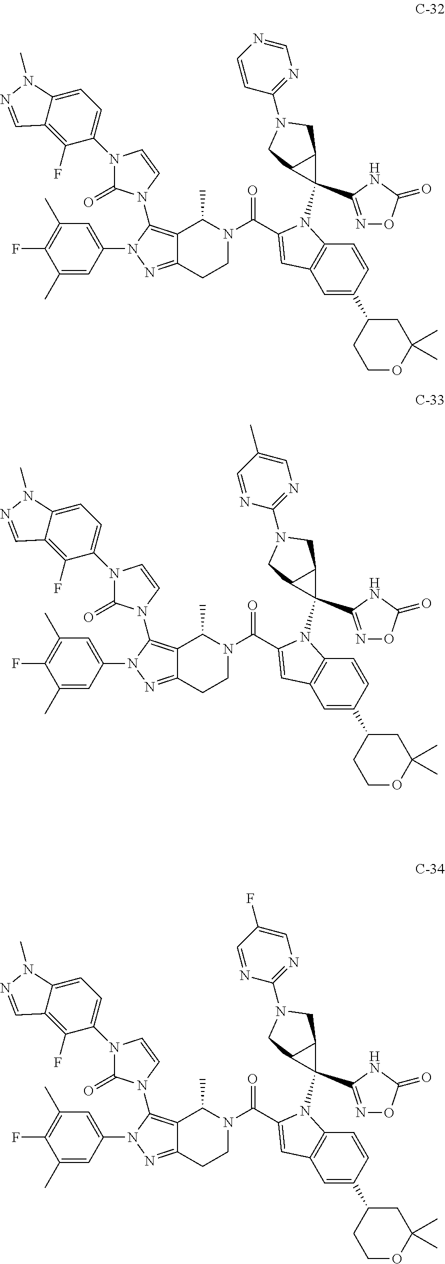

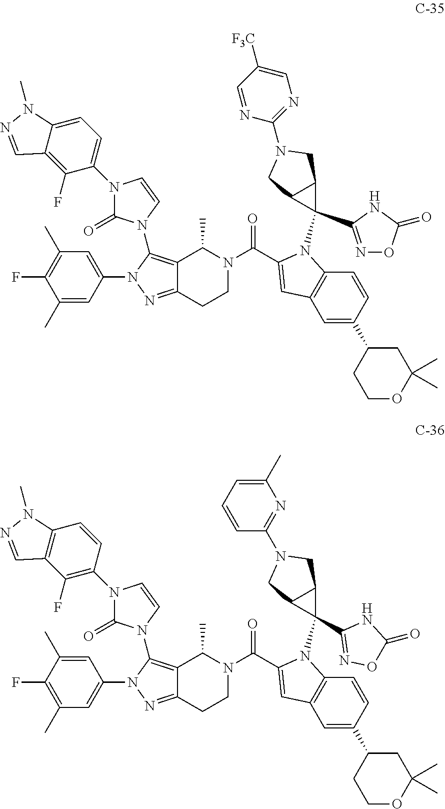

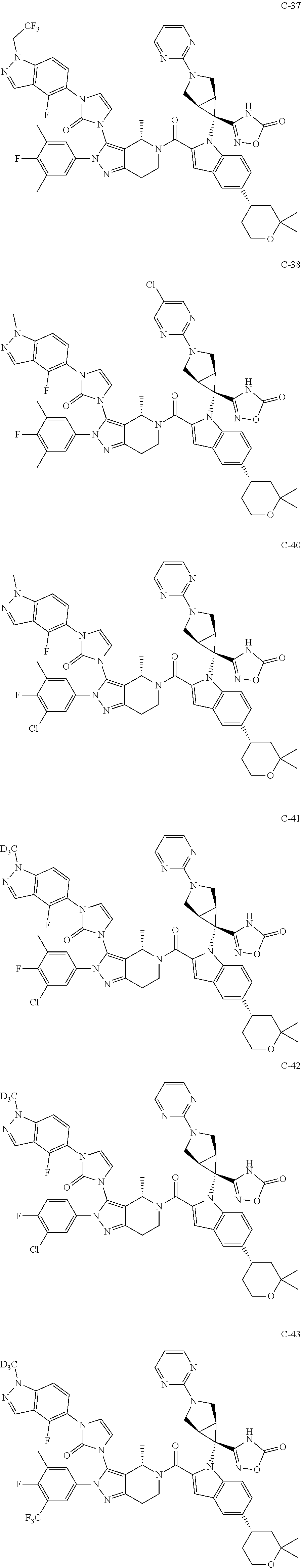

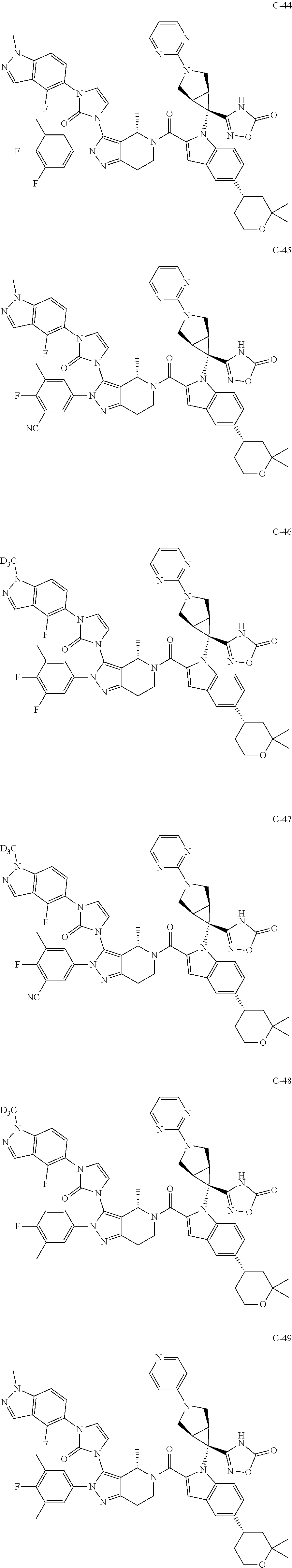

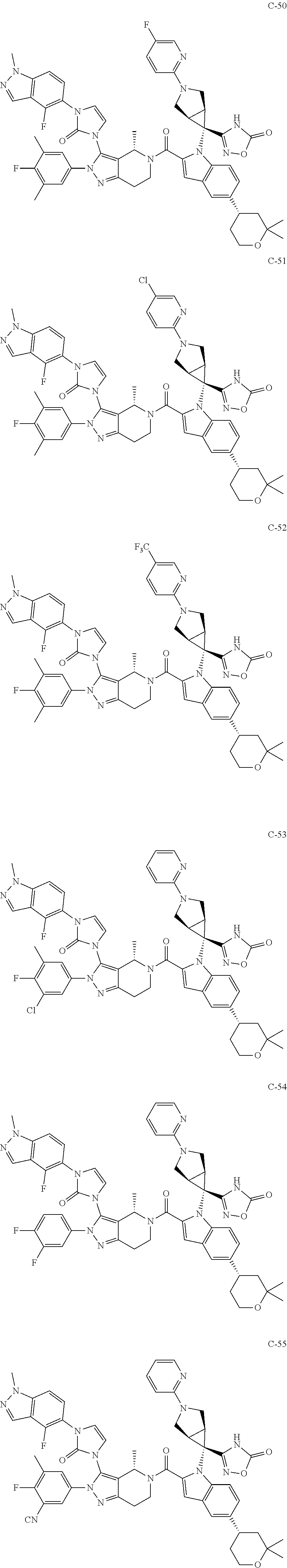

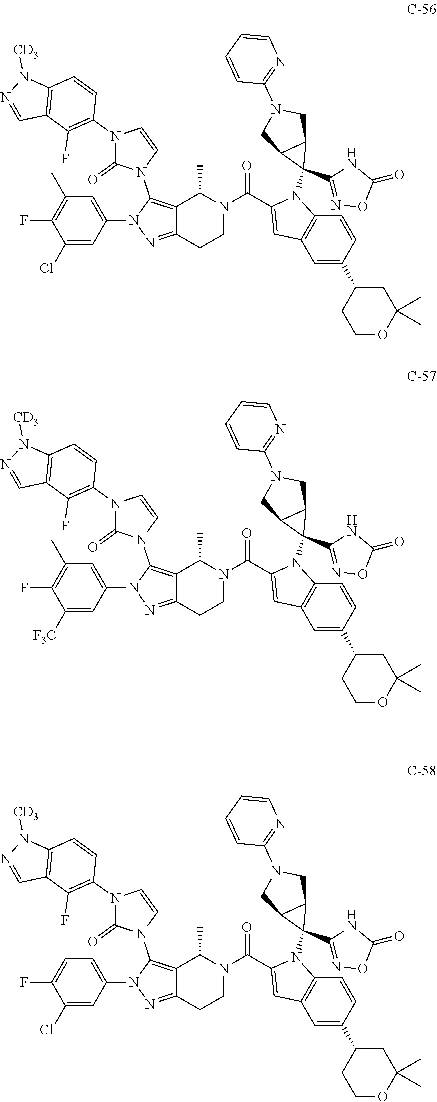

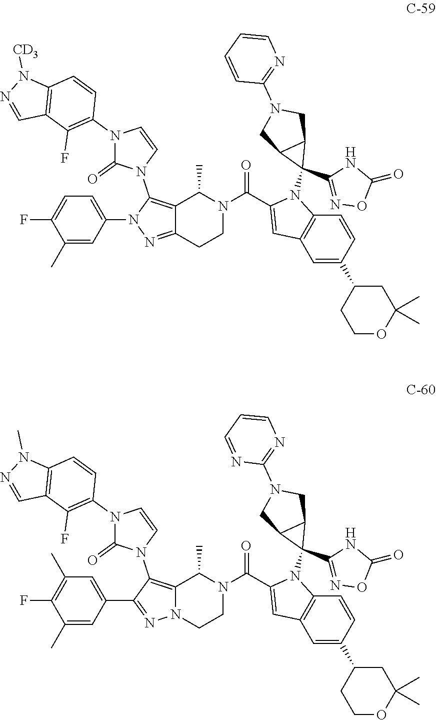 [SMILES: [2H]C([2H])([2H])n1ncc2c(F)c(-n3ccn(-c4c5c(nn4-c4ccc(F)c(C)c4)CCN(C(=O)c4cc6cc([C@H]7CCOC(C)(C)C7)ccc6n4[C@@]4(c6noc(=O)[nH]6)[C@@H]6CN(c7ccccn7)C[C@@H]64)[C@H]5C)c3=O)ccc21 ; Cc1cc(-c2nn3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)[C@@H]4CN(c5ncccn5)C[C@@H]42)CC3)cc(C)c1F]

104. A pharmaceutical composition comprising a compound of claim 92, or a pharmaceutically acceptable salt thereof, stereoisomer thereof, tautomer thereof, deuterated analog thereof, or solvate thereof, and at least one pharmaceutically acceptable excipient.

105. A method for treating or prophylactically treating a GLP-1 associated disease, disorder, or condition in a subject in need of, the method comprising administering to a subject in need thereof an effective amount of the compound of claim 92, or a pharmaceutically acceptable salt thereof, stereoisomer thereof, tautomer thereof, deuterated analog thereof, or solvate thereof, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, and overweight.

106. The method of claim 105, wherein the disease, disorder, or condition is diabetes.

107. The method of claim 105, wherein the disease, disorder, or condition is type 2 diabetes mellitus.

108. The method of claim 105, wherein the disease, disorder, or condition is obesity or overweight.
